(19) United States
(12) Patent Application Publication
(10) Pub. No.: US 2024/0245658 A1
(43) Pub. Date: Jul. 25, 2024

(54) TAAR1 AND SEROTONIN MODULATORS, AND PHARMACEUTICAL COMPOSITIONS, AND METHODS OF USE THEREOF

(71) Applicant: Sunovion Pharmaceuticals Inc., Marlborough, MA (US)

(72) Inventors: Kevin Julian Hodgetts, Framingham, MA (US); Linghong Xie, Southborough, MA (US)

(21) Appl. No.: 18/285,975

(22) PCT Filed: Apr. 8, 2022
(86) PCT No.: PCT/US2022/071613

§ 371 Date: Oct. 6, 2023

Related U.S. Application Data
(60) Provisional application No. 63/173,368, filed on Apr. 10, 2021.

(51) Int. Cl. A61K 31/4353; A61K 31/335; A61K 45/06; C07D 313/12; C07D 491/044

(52) U.S. Cl. CPC A61K 31/4353; A61K 31/335; A61K 45/06; C07D 313/12; C07D 491/044

(57) ABSTRACT

Provided herein is a compound of Formula (I): or a pharmaceutically acceptable salt thereof, wherein values for the variables (e.g., R¹, R², X¹, X², Y¹, Y², Y³, Y⁴, Y⁵, Y⁶, Y⁷, and Y⁸) are as disclosed herein. Also provided herein are pharmaceutical compositions comprising a compound of Formula (I), or a pharmaceutically acceptable salt thereof, and methods of using the compounds, pharmaceutically acceptable salts thereof and pharmaceutical compositions of the foregoing, e.g., to treat a neurological or psychiatric disease or disorder.

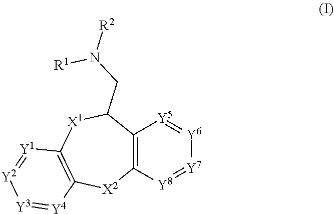

### RELATED APPLICATION

The application claims the benefit of U.S. Provisional Application No. 63/173,368, filed on Apr. 10, 2021. The entire teachings of this application are incorporated herein by reference.

### FIELD

The present disclosure relates to compounds, pharmaceutical compositions, and methods of use thereof, including methods of treating a neurological or psychiatric disease or disorder.

### BACKGROUND

Treatments for neurological or psychiatric diseases and disorders typically target certain neurotransmitter sites. For example, the D₂ dopamine receptor has been a primary target for both typical and atypical antipsychotic agents used to treat a variety of neurological or psychiatric diseases or disorders. Wang et al. NATURE 555, 269-273 (2018). However, many of the drugs that target the D₂ dopamine receptor can cause serious or potentially life-threatening side effects. Wang et al. NATURE 555, 269-273 (2018). Despite decades of research on non-D₂ dopamine receptor mechanisms of action, developing non-D₂ dopamine receptor therapies that are both safe and effective has been challenging. Girgis et al., J. PSYCHIATRIC RES. (2018), https://doi.org/10.1016/j.jpsychires.2018.07.006. After performing a comprehensive review of literature relating to experimental treatments for schizophrenia (as one of many neurological or psychiatric diseases and disorders), including 250 studies conducted between 1970 to 2017 with glutamatergic, serotonergic, cholinergic, neuropeptidergic, hormone-based, dopaminergic, metabolic, vitamin/naturopathic, histaminergic, infection/inflammation-based, and otherwise miscellaneous mechanisms for treating schizophrenia, Girgis states, “Despite there being several promising [non-D₂ dopamine receptor] targets, such as allosteric modulation of the NMDA and a7 nicotinic receptors, we cannot confidently state that any of the mechanistically novel experimental treatments covered in this review are definitely effective for the treatment of schizophrenia and ready for clinical use.”

Accordingly, there is a need for therapeutic agents for treating neurological and psychiatric diseases and disorders.

### SUMMARY

In one aspect, the present disclosure provides a compound of Formula I.

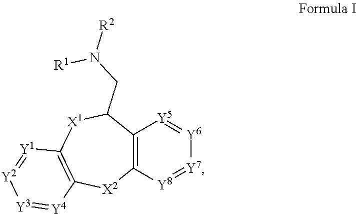

or a pharmaceutically acceptable salt thereof, wherein values for the variables (e.g., X¹, X², Y¹, Y², Y³, Y⁴, Y⁵, Y⁶, Y⁷, Y⁸, R¹, R²) are as disclosed herein.

In another aspect, the present disclosure provides a pharmaceutical composition comprising a compound of the present disclosure and one or more pharmaceutically acceptable excipients.

In yet another aspect, the present disclosure provides a pharmaceutical combination comprising a compound of the present disclosure and one or more additional therapeutic agents.

In another aspect, the present disclosure provides a method of treating a neurological or psychiatric disease or disorder, such as a neurological or psychiatric disease or disorder disclosed herein, in a subject in need thereof, comprising administering to the subject a therapeutically effective amount of a compound of the present disclosure, or a pharmaceutical composition or combination disclosed herein.

In another aspect, the present disclosure provides a method of agonizing TAAR1 in a subject in need thereof, comprising administering to the subject a compound of the present disclosure, or a pharmaceutical composition or combination disclosed herein, in an amount sufficient to agonize TAAR1 in the subject.

In another aspect, the present disclosure provides a method of antagonizing 5-HT2A, 5-HT7, or 5-HT2A and 5-HT7 in a subject in need thereof, comprising administering to the subject a compound of the present disclosure, or a pharmaceutical composition or combination disclosed herein, in an amount sufficient to antagonize 5-HT2A, 5-HT7, or 5-HT2A and 5-HT7, respectively, in the subject.

In another aspect, the present disclosure provides a compound of the present disclosure, or a pharmaceutical composition or combination disclosed herein, for use in treating a disease or disorder disclosed herein (e.g., a neurological or psychiatric disease or disorder) in a subject. Another aspect is use of a compound of the present disclosure, or a pharmaceutical composition or combination disclosed herein, for the manufacture of a medicament for treating a disease or disorder disclosed herein (e.g., a neurological or psychiatric disease or disorder).

### DETAILED DESCRIPTION

A description of embodiments follows.

Provided herein are definitions to assist with interpreting this disclosure. Whenever appropriate, terms used in the singular will also include the plural. Unless the context clearly indicates otherwise, terms used herein have the following meanings.

All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the present disclosure otherwise claimed.

### Definitions

The terms “a,” “an,” “the” and similar terms used in the context of the present disclosure (especially in the context of the claims) are to be construed to cover both the singular and plural unless otherwise indicated herein or clearly contradicted by the context.

As used herein, the term “alkyl” refers to a branched or straight-chain, monovalent, hydrocarbon radical having the specified number of carbon atoms, and the general formula CₙH₂ₙ₊₁. Thus, the term “(C₁-C₆)alkyl” refers to a branched or straight-chain, monovalent, hydrocarbon radical of the general formula CₙH₂ₙ₊₁ wherein n is 1, 2, 3, 4, 5 or 6. Examples of alkyl include, but are not limited to, methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, and the like.

As used herein, the term “alkenyl” refers to a branched or straight-chain, monovalent, hydrocarbon radical containing at least one carbon-carbon double bond and having from 2 to 4 carbon atoms (i.e., C₂-C₄ alkenyl). Examples of alkenyl groups include ethenyl, propenyl, and butadienyl (including 1,2-butadienyl, and 1,3-butadienyl).

As used herein, the term “alkynyl” refers to a branched or straight-chain, monovalent, hydrocarbon radical containing at least one carbon-carbon triple bond and having from 2 to 4 carbon atoms (i.e., C₂-C₄ alkynyl). The term “alkynyl” also includes those groups having one triple bond and one double bond.

The term “alkoxy,” as used herein, refers to an alkyl radical attached through an oxygen linking atom, wherein alkyl is as described herein. “(C₁-C₆)alkoxy” refers to an alkoxy group in which a (C₁-C₆)alkyl is attached through an oxygen linking atom. Examples of alkoxy groups include, but are not limited to, methoxy, ethoxy, propoxy (e.g., n-propoxy and iso-propoxy), and butoxy (e.g., t-butoxy).

“Halogen” and “halo,” as used herein, refer to fluorine, chlorine, bromine or iodine. In some embodiments, halogen is fluoro, chloro or bromo. In some embodiments, halogen is fluoro or chloro. In some embodiments, halogen is fluoro.

“Haloalkyl,” as used herein, refers to an alkyl radical wherein one or more hydrogen atoms is each independently replaced by a halogen, wherein alkyl and halogen are as described herein. “Haloalkyl” includes mono-, poly- and perhaloalkyl groups. “(C₁-C₆)haloalkyl” refers to a (C₁-C₆)alkyl wherein one or more hydrogen atoms is each independently replaced by a halogen. Examples of haloalkyl include, but are not limited to, fluoromethyl, difluoromethyl, trifluoromethyl, trichloromethyl, pentafluoroethyl, pentachloroethyl, 2,2,2-trifluoroethyl, heptafluoropropyl, and heptachloropropyl.

“Haloalkoxy,” as used herein, refers to a haloalkyl radical attached through an oxygen linking atom, wherein haloalkyl is as described herein. “(C₁-C₆)haloalkoxy” refers to a haloalkoxy group in which a (C₁-C₆)haloalkyl is attached through an oxygen linking atom. Examples of haloalkoxy include, but are not limited to, trifluoromethoxy, difluoromethoxy, 2,2,2 trifluoroethoxy, and pentafluoroethoxy.

“Cyano” or “—CN” as used herein, means —C≡N.

The term “substituted,” as used herein, means that at least one (e.g., one, two, three, four, five, six, etc., from one to five, from one to three, one or two) hydrogen atom is replaced with a non-hydrogen substituent, provided that normal valencies are maintained and that the substitution results in a stable compound. Unless otherwise indicated, an “optionally substituted” group can have a substituent at each substitutable position of the group and, when more than one position in any given structure may be substituted with more than one substituent selected from a specified group, the substituent can be the same or different at every position. Alternatively, an “optionally substituted group” can be unsubstituted.

When there is a nitrogen atom(s) in a compound of the present disclosure, the nitrogen atom(s) may be independently converted to N-oxide(s) by treatment with an oxidizing agent (e.g., mCPBA and/or hydrogen peroxide) to afford other compounds of the present disclosure. Thus, shown and claimed nitrogen atoms are considered to cover both the shown nitrogen and its N-oxide (N→O) derivative.

When any variable occurs more than one time in any constituent or formula for a compound, its definition at each occurrence is independent of its definition at every other occurrence. Thus, for example, the value of each R³ in a compound of Formula I is independent of its value at every other occurrence, such one occurrence of C(R³)₂ may be C(H)₂ or C(CH₃)(H).

Combinations of substituents and/or variables are permissible only if such combinations result in stable compounds.

As a person of ordinary skill in the art would understand, for example, a ketone (—C(H)C(O)) group in a molecule may tautomerize to its enol form (—C═C(OH)). This disclosure is intended to cover all possible tautomers even when a structure depicts only one of them.

The phrase “pharmaceutically acceptable” means that the substance or composition the phrase modifies must be, within the scope of sound medical judgment, suitable for use in contact with the tissues of humans and lower animals without undue toxicity, irritation, allergic response and the like, and are commensurate with a reasonable benefit/risk ratio. If a substance is part of a composition or formulation, the substance must also be compatible chemically and/or toxicologically with the other ingredients in the composition or formulation.

Unless specified otherwise, the term “compounds of the present disclosure” refers to a compound of any structural formula depicted herein (e.g., a compound of Formula I, a subformula of a compound of Formula I, such as a compound of Formula I(A), I(B), II, III(A), III(B), III(C), III(D), III(E), III(F), III(G), III(H), IV, V(A), V(B), V(C), V(D), V(E), V(F), V(G), and/or V(H)), as well as isomers, such as stereoisomers (including diastereoisomers, enantiomers and racemates), geometrical isomers, conformational isomers (including rotamers and atropisomers), tautomers, isotopically labeled compounds (including deuterium substitutions), and inherently formed moieties (e.g., polymorphs and/or solvates, such as hydrates) thereof. When a moiety is present that is capable of forming a salt, then salts are included as well, in particular, pharmaceutically acceptable salts.

Compounds of the present disclosure may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemic mixtures, individual isomers (e.g., diastereomers, enantiomers, geometrical isomers, conformational isomers (including rotamers and atropisomers), tautomers) and intermediate mixtures, with all possible isomers and mixtures thereof being included in the present disclosure.

As used herein, the term “isomers” refers to different compounds that have the same molecular formula but differ in arrangement and configuration of the atoms.

“Enantiomers” are a pair of stereoisomers that are non-superimposable mirror images of each other. A 1:1 mixture of a pair of enantiomers is a “racemic” mixture. “Racemate” or “racemic” is used to designate a racemic mixture where appropriate. When designating stereochemistry for a compound of the present disclosure, a single stereoisomer with known relative and absolute configuration of two chiral centers is designated using the conventional RS system (e.g., (1S,2S)); a single stereoisomer with known relative configuration but unknown absolute configuration is designated with asterisks (e.g., (R*), (S*), (1R*,2R*)); and a racemate with two letters (e.g., (1RS,2RS) as a racemic mixture of (1R,2R) and (1S,2S); (1RS,2SR) as a racemic mixture of (1R,2S) and (1S,2R)). “Diastereoisomers” are stereoisomers that have at least two asymmetric atoms, and which are not mirror-images of each other. The absolute stereochemistry can be specified according to the Cahn-Ingold-Prelog R-S system.

When a compound is a pure enantiomer, the stereochemistry at each chiral carbon may be specified by either R or S. Resolved compounds can be designated (+) or (−) depending on the direction (dextro- or levorotatory) which they rotate plane polarized light at the wavelength of the sodium D line. Alternatively or in addition, resolved compounds can be defined by the respective retention times for the corresponding enantiomers/diastereomers via chiral HPLC.

Graphic representations of racemic, ambiscalemic and scalemic or enantiomerically pure compounds used herein are a modified version of the denotations taken from Maehr J. Chem. Ed. 62, 114-120 (1985): simple lines provide no information about stereochemistry and convey only connectivity; solid and broken wedges are used to denote the absolute configuration of a chiral element; solid and broken bold lines indicated relative stereochemistry of indeterminate absolute configuration. For example, the graphic representation:

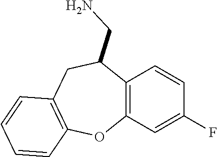 [SMILES: NC[C@@H]1Cc2ccccc2Oc2cc(F)ccc21]

indicates an enantiomer, that is, either of the two representations below:

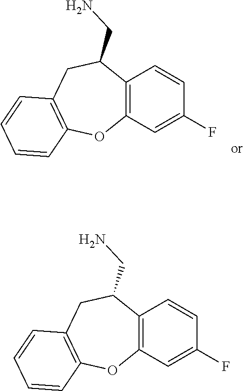 [SMILES: NC[C@@H]1Cc2ccccc2Oc2cc(F)ccc21 ; NC[C@H]1Cc2ccccc2Oc2cc(F)ccc21]

in any ratio, and likewise,

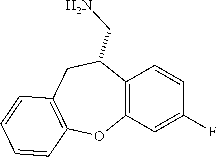 [SMILES: NC[C@H]1Cc2ccccc2Oc2cc(F)ccc21]

is the other enantiomer of

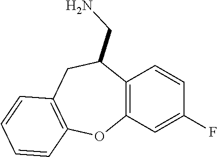 [SMILES: NC[C@@H]1Cc2ccccc2Oc2cc(F)ccc21]

and is either of the two representations below:

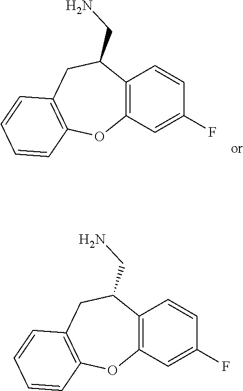 [SMILES: NC[C@@H]1Cc2ccccc2Oc2cc(F)ccc21 ; NC[C@H]1Cc2ccccc2Oc2cc(F)ccc21]

in any ratio, while the representation:

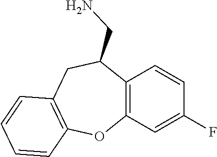 [SMILES: NC[C@@H]1Cc2ccccc2Oc2cc(F)ccc21]

indicates a single enantiomer with the absolute configuration depicted, e.g., (R)-(7-fluoro-10,11-dihydrodibenzo[b,f]oxepin-10-yl)methanamine in the illustration above.

The “enantiomeric excess” or “% enantiomeric excess” or “% ee” of a composition can be calculated using the equation shown below. In the example shown below, a composition contains 90% of one enantiomer, e.g., the S enantiomer, and 10% of the other enantiomer, e.g., the R enantiomer. In this example, % ee=(90−10)/100=80%. Thus, a composition containing 90% of one enantiomer and 10% of the other enantiomer is said to have an enantiomeric excess of 80%. Some compositions described herein contain an enantiomeric excess of a compound of the present disclosure of at least about 50%, 75%, 90%, 95%, or 99%. Some compositions described herein, particularly those compositions containing a compound of the present disclosure possessing a single chiral center, such as 7-fluoro-10,11-dihydrodibenzo[b,f]oxepin-10-yl)methanamine, depicted above, contain an enantiomeric excess of at least about 50%, 75%, 90%, 95%, or 99% of the S enantiomer. In other words, the compositions contain an enantiomeric excess of the S enantiomer over the R enantiomer. In other embodiments, compositions described herein, particularly those compositions containing a compound of the present disclosure possessing a single chiral center, such as 7-fluoro-10,11-dihydrodibenzo[b,f]oxepin-10-yl)methanamine, depicted above, contain an enantiomeric excess of at least about 50%, 75%, 90%, 95%, or 99% of the R enantiomer. In other words, the compositions contain an enantiomeric excess of the R enantiomer over the S enantiomer.

For instance, an isomer (e.g., diastereomer)/enantiomer can, in some embodiments, be provided substantially free of the corresponding isomer(s) (e.g., diastereomer(s))/enantiomer, and can also be referred to as “optically enriched,” “enantiomerically enriched,” “enantiomerically pure” and “non-racemic,” all of which are used interchangeably herein. These terms refer to compositions in which the percent by weight of one isomer (e.g., diastereomer)/enantiomer is greater than the amount of that one isomer (e.g., diastereomer)/enantiomer in a control mixture of the racemic composition (e.g., greater than 1:1 by weight). For example, an enantiomerically enriched preparation of an S enantiomer, means a preparation of the compound having greater than about 50% by weight of the S enantiomer relative to the R enantiomer, such as at least about 75% by weight, further such as at least about 80% by weight. In some embodiments, the enrichment can be much greater than about 80% by weight, providing a “substantially enantiomerically enriched,” “substantially enantiomerically pure” or “substantially non-racemic” preparation, which refers to preparations of a compound of the disclosure which have at least about 85% by weight of one enantiomer relative to the other isomer(s) (e.g., diastereomer(s))/enantiomer, such as at least about 90% by weight, and further such as at least 95% by weight. In certain embodiments, the compound of the present disclosure is made up of at least about 90% by weight of one enantiomer. In other embodiments, the compound of the present disclosure is made up of at least about 95%, 98%, or 99% by weight of one enantiomer.

In some embodiments, the compound of the present disclosure is present in a diastereomeric or enantiomeric excess (e.g., enantiomeric excess) of greater than about 55%, about 60%, about 65%, about 70%, about 75%, about 80%, about 85%, about 90%, about 95%, about 96%, about 97%, about 98%, about 99%, about 99.5%, or more. In some embodiments, the compound of the present disclosure is present in a diastereomeric or enantiomeric excess (e.g., enantiomeric excess) of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, in some embodiments, particularly those wherein the compound possesses a single chiral center, such as 7-fluoro-10,11-dihydrodibenzo[b,f]oxepin-10-yl)methanamine, depicted above, the compound mixture has an (S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, particularly those wherein the compound possesses a single chiral center, such as 7-fluoro-10,11-dihydrodibenzo[b,f]oxepin-10-yl)methanamine, depicted above, the compound mixture has an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

In other embodiments, particularly those wherein the compound possesses a single chiral center, such as 7-fluoro-10,11-dihydrodibenzo[b,f]oxepin-10-yl)methanamine, depicted above, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, particularly those wherein the compound possesses a single chiral center, such as 7-fluoro-10,11-dihydrodibenzo[b,f]oxepin-10-yl)methanamine, depicted above, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

In other embodiments, the compound mixture contains identical chemical entities except for their stereochemical orientations, namely (S)- or (R)-isomers. For example, if a compound disclosed herein has —CH(R)— unit, and R is not hydrogen, then the —CH(R)— is in an (S)- or (R)-stereochemical orientation for each of the identical chemical entities. In some embodiments, the mixture of identical chemical entities is a racemic mixture of (S)- and (R)-isomers. In another embodiment, the mixture of the identical chemical entities (except for their stereochemical orientations), contain predominately (S)-isomers or predominately (R)-isomers, e.g., at the carbon atom to which —CH₂NR¹R² is attached in a compound of Formula I. For example, the (S)-isomers in the mixture of identical chemical entities, particularly those isomers in a mixture of a compound of Formula I having an (S) configuration at the carbon atom to which —CH₂NR¹R² is attached in the compound of Formula I, are present at about 55%, about 60%, about 65%, about 70%, about 75%, about 80%, about 85%, about 90%, about 95%, about 96%, about 97%, about 98%, about 99%, about 99.5%, or more, relative to the (R)-isomers. In some embodiments, the (S)-isomers in the mixture of identical chemical entities, particularly those isomers in a mixture of a compound of Formula I having an (S) configuration at the carbon atom to which —CH₂NR¹R² is attached in the compound of Formula I, are present at an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

In another embodiment, the (R)-isomers in the mixture of identical chemical entities (except for their stereochemical orientations), particularly those isomers in a mixture of a compound of Formula I having an (R) configuration at the carbon atom to which —CH₂NR¹R² is attached in the compound of Formula I, are present at about 55%, about 60%, about 65%, about 70%, about 75%, about 80%, about 85%, about 90%, about 95%, about 96%, about 97%, about 98%, about 99%, about 99.5%, or more, relative to the (S)-isomers. In some embodiments, the (R)-isomers in the mixture of identical chemical entities (except for their stereochemical orientations), particularly those isomers in a mixture of a compound of Formula I having an (R) configuration at the carbon atom to which —CH₂NR¹R² is attached in the compound of Formula I, are present at a (R)-enantiomeric excess greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

Geometric isomers may occur when a compound contains a double bond or some other feature that gives the molecule a certain amount of structural rigidity. If the compound contains a double bond, the double bond may be E- or Z-configuration. If the compound contains a disubstituted cycloalkyl, the cycloalkyl substituent may have a cis- or trans-configuration.

Conformational isomers (or conformers) are isomers that can differ by rotations about one or more bonds. Rotamers are conformers that differ by rotation about only a single bond.

The term “atropisomer,” as used herein, refers to a structural isomer based on axial or planar chirality resulting from restricted rotation in the molecule.

Optically active (R)- and (S)- isomers may be prepared using chiral synthons or chiral reagents, or resolved using conventional techniques (e.g., separated on chiral SFC or HPLC chromatography columns, such as CHIRALPAK® and CHIRALCEL® columns available from DAICEL Corp. or other equivalent columns, using the appropriate solvent or mixture of solvents to achieve suitable separation).

The compounds of the present disclosure can be isolated in optically active or racemic forms. Optically active forms may be prepared by resolution of racemic forms or by synthesis from optically active starting materials. All processes used to prepare compounds of the present disclosure and intermediates made therein are considered to be part of the present disclosure. When enantiomeric or diastereomeric products are prepared, they may be separated by conventional methods, for example, by chromatography or fractional crystallization.

Depending on the process conditions, the end products of the present disclosure are obtained either in free (neutral) or salt form. Both the free form and the salts of these end products are within the scope of the present disclosure. If so desired, one form of a compound may be converted into another form. A free base or acid may be converted into a salt; a salt may be converted into the free compound or another salt; a mixture of isomeric compounds of the present disclosure may be separated into the individual isomers.

Pharmaceutically acceptable salts are preferred. However, other salts may be useful, e.g., in isolation or purification steps which may be employed during preparation, and thus, are contemplated to be within the scope of the present disclosure.

The phrase “pharmaceutically acceptable” means that the substance or composition the phrase modifies must be, within the scope of sound medical judgment, suitable for use in contact with the tissues of humans and lower animals without undue toxicity, irritation, allergic response and the like, and are commensurate with a reasonable benefit/risk ratio. If a substance is part of a composition or formulation, the substance must also be compatible chemically and/or toxicologically with the other ingredients in the composition or formulation.

As used herein, “pharmaceutically acceptable salts” refers to salts derived from suitable inorganic and organic acids and bases that are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable acid addition salts include, but are not limited to, acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethanedisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate/hydroxymalonate, mandelate, mesylate, methylsulphate, mucate, naphthoate, napsylate, nicotinate, nitrate, octadecanoate, oleate, oxalate, palmitate, pamoate, phenylacetate, phosphate/hydrogen phosphate/dihydrogen phosphate, polygalacturonate, propionate, salicylates, stearate, succinate, sulfamate, sulfosalicylate, tartrate, tosylate, trifluoroacetate and xinafoate salts.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, or copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Examples of organic amines include, but are not limited to, isopropylamine, benzathine, cholinate, diethanolamine, diethylamine, lysine, meglumine, piperazine and tromethamine.

A salt, such as a pharmaceutically acceptable salt of a compound of the present disclosure, can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Allen, L. V., Jr., ed., Remington: The Science and Practice of Pharmacy, 22nd Edition, Pharmaceutical Press, London, UK (2012), the relevant disclosure of which is hereby incorporated by reference in its entirety.

Compounds of the present disclosure that contain groups capable of acting as donors and/or acceptors for hydrogen bonds may be capable of forming co-crystals with suitable co-crystal formers. These co-crystals may be prepared from compounds of the present disclosure by known co-crystal forming procedures. Such procedures include grinding, heating, co-subliming, co-melting, or contacting in solution compounds of the present disclosure with the co-crystal former under crystallization conditions and isolating co-crystals thereby formed. Suitable co-crystal formers include those described in WO 2004/078163. Hence, the present disclosure further provides co-crystals comprising a compound of the present disclosure and a co-crystal former.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorous, fluorine, chlorine and iodine, such as ²H, ³H, ¹¹C, ¹³C, ¹⁴C, ¹⁵N, ¹⁸F, ³¹P, ³²P, ³⁵S, ³⁶Cl, ¹²³I, ¹²⁴I and ¹²⁵I, respectively. The present disclosure includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies.

Further, substitution with heavier isotopes, particularly deuterium (i.e., ²H or D) may afford certain therapeutic advantages resulting from greater metabolic stability, for example, increased in vivo half-life or reduced dosage requirements or an improvement in therapeutic index. It is understood that deuterium in this context is regarded as a substituent of a compound of the present disclosure. The concentration of such a heavier isotope, specifically deuterium, may be defined by the isotopic enrichment factor. The term “isotopic enrichment factor,” as used herein, means the ratio between the isotopic abundance and the natural abundance of a specified isotope. If a substituent in a compound of this present disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (670.5% deuterium incorporation), at least 5000 (75% deuterium incorporation), at least 5500 (820.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically labeled compounds of the present disclosure can generally be prepared by conventional techniques known to those skilled in the art or by processes disclosed in the schemes or in the examples and preparations described below (or analogous processes to those described herein below), by substituting an appropriate or readily available isotopically labeled reagent for a non-isotopically labeled reagent otherwise employed. Such compounds have a variety of potential uses, e.g., as standards and reagents in determining the ability of a potential pharmaceutical compound to bind to target proteins or receptors, or for imaging compounds of this disclosure bound to biological receptors in vivo or in vitro.

A “pharmaceutically acceptable carrier” refers to media generally accepted in the art for the delivery of biologically active agents to animals, in particular, mammals, including, generally recognized as safe (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, buffering agents (e.g., maleic acid, tartaric acid, lactic acid, citric acid, acetic acid, sodium bicarbonate, sodium phosphate, and the like), disintegration agents, lubricants, sweetening agents, flavoring agents, dyes, and the like, and combinations thereof, as would be known to those skilled in the art (see, for example, Allen, L. V., Jr. et al., Remington: The Science and Practice of Pharmacy (2 Volumes), 22nd Edition, Pharmaceutical Press (2012).

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog)), or bird (e.g., commercially relevant bird, such as chicken, duck, goose, or turkey). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease or disorder. A subject (e.g., a human) is “in need of” a treatment if such subject would benefit biologically, medically or in quality of life from such treatment, e.g., if the subject has a disease or disorder, such as a disease or disorder disclosed herein.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be effected by administering medication or medical care to a subject having a disease or disorder, such as a disease or disorder disclosed herein. In some embodiments, treatment may be effected by administering medication or medical care to a subject after one or more symptoms have developed. In other embodiments, treatment may be effected by administering medication or medical care to a subject in the absence of symptoms. For example, medication or medical care may be administered to a susceptible individual prior to the onset of symptoms (e.g., in light of a history of symptoms and/or in light of genetic or other susceptibility factors). Administration of medication or medical care may also be continued after symptoms have resolved, for example to prevent or delay their recurrence.

The term “therapeutically effective amount,” as used herein, refers to an amount of a therapeutic agent, such as a compound of the present disclosure, that, when administered to a subject, such as a human, is sufficient to effect treatment. The amount of a therapeutic agent that constitutes a “therapeutically effective amount” will vary depending, e.g., on the therapeutic agent, the condition being treated and its severity, the manner of administration, the duration of treatment, or the subject to be treated (e.g., age, weight, fitness of the subject), but can be determined routinely by one of ordinary skill in the art based on his own knowledge and this disclosure. In embodiments, a “therapeutically effective amount” effects treatment as measured by a statistically significant change in one or more indications, symptoms, signs, diagnostic tests, vital signs, and the like. In other embodiments, a “therapeutically effective amount” manages or prevents a condition, as measured by a lack of a statistically significant change in one or more indications, symptoms, signs, diagnostic tests, vital signs, and the like.

The regimen of administration can affect what constitutes a therapeutically effective amount. For example, several divided doses, as well as staggered doses, can be administered daily or sequentially, or the dose can be continuously infused, or can be a bolus injection. Further, doses can be proportionally increased or decreased as indicated by the exigencies of the therapeutic or prophylactic situation.

### Compounds

In one aspect, the present disclosure provides compounds of Formula I

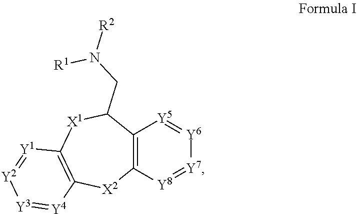

or a pharmaceutically acceptable salt thereof, wherein:
one of X¹ and X² is O, and the other is independently C(R³)₂ or O;
   each R³ is independently H, (C₁-C₄)alkyl, (C₂-C₄)alkenyl, or (C₂-C₄)alkynyl;
Y¹, Y², Y³, Y⁴, Y⁵, Y⁶, Y⁷, and Y⁸ are each independently C(R⁴) or N, and no more than one of Y¹, Y², Y³, Y⁴, Y⁵, Y⁶, Y⁷, and Y⁸ are N;
   each R⁴ is independently H, halogen, —CN, (C₁-C₄)alkyl, (C₂-C₄)alkenyl, (C₂-C₄)alkynyl, (C₁-C₄)haloalkyl, (C₁-C₄)alkoxy, or (C₁-C₄)haloalkoxy; and
R¹ and R² are each independently H, (C₁-C₄)alkyl, (C₂-C₄)alkenyl, or (C₂-C₄)alkynyl.

In some embodiments, X¹ is C(R³)₂ or O, and X² is O.

In some embodiments, X¹ is O. In some embodiments, X¹ is C(R³)₂.

In some embodiments, X² is O. In some embodiments, X² is C(R³)₂.

In some embodiments, X¹ and X² are each O. In some embodiments, X¹ is O and X² is C(R³)₂. In some embodiments, X² is O and X¹ is C(R³)₂.

In some embodiments, each R³ is independently H or (C₁-C₄)alkyl. In some embodiments, each R³ is independently H or methyl. In some embodiments, each R³ is H.

In some embodiments, one R³ is H and one R³ is (C₁-C₄)alkyl. In some embodiments, one R³ is H and one R³ is methyl.

In some embodiments, each R³ is independently (C₁-C₄)alkyl. In some embodiments, each R³ is methyl.

In some embodiments, Y¹, Y², Y³, Y⁴, Y⁵, Y⁶, Y⁷, and Y⁸ are each C(R⁴).

In some embodiments, one of Y¹, Y², Y³, Y⁴, Y⁵, Y⁶, Y⁷, and Y⁸ is N, and the rest are each C(R⁴). In some embodiments, one of Y¹, Y³ and Y⁵ is N. In some embodiments, one of Y⁵, Y⁶, Y⁷ and Y⁸ is N.

In some embodiments, Y¹ is N. In some embodiments, Y¹ is C(H). In some embodiments, Y¹ is C(R⁴). In some embodiments, Y¹ is C(R⁴) wherein R⁴ is H or halogen. In some embodiments, Y¹ is C(R⁴) wherein R⁴ is H or F. In some embodiments, Y¹ is C(R⁴) wherein R⁴ is H.

In some embodiments, Y² is N. In some embodiments, Y² is C(H). In some embodiments, Y² is C(R⁴). In some embodiments, Y² is C(R⁴) wherein R⁴ is H or halogen. In some embodiments, Y² is C(R⁴) wherein R⁴ is H or F. In some embodiments, Y² is C(R⁴) wherein R⁴ is H.

In some embodiments, Y³ is N. In some embodiments, Y³ is C(H) or C(F). In some embodiments, Y³ is C(H). In some embodiments, Y³ is C(F). In some embodiments, Y³ is C(R⁴). In some embodiments, Y³ is C(R⁴) wherein R⁴ is H or halogen. In some embodiments, Y³ is C(R⁴) wherein R⁴ is H or F. In some embodiments, Y³ is C(R⁴) wherein R⁴ is H. In some embodiments, Y³ is C(R⁴) wherein R⁴ is F.

In some embodiments, Y⁴ is N. In some embodiments, Y⁴ is C(H). In some embodiments, Y⁴ is C(R⁴). In some embodiments, Y⁴ is C(R⁴) wherein R⁴ is H or halogen. In some embodiments, Y⁴ is C(R⁴) wherein R⁴ is H or F. In some embodiments, Y⁴ is C(R⁴) wherein R⁴ is H.

In some embodiments, Y⁵ is N. In some embodiments, Y⁵ is C(H). In some embodiments, Y⁵ is C(R⁴). In some embodiments, Y⁵ is C(R⁴) wherein R⁴ is H or halogen. In some embodiments, Y⁵ is C(R⁴) wherein R⁴ is H or F. In some embodiments, Y⁵ is C(R⁴) wherein R⁴.

In some embodiments, Y⁶ is N. In some embodiments, Y⁶ is C(H). In some embodiments, Y⁶ is C(R⁴). In some embodiments, Y⁶ is C(R⁴) wherein R⁴ is H or halogen. In some embodiments, Y⁶ is C(R⁴) wherein R⁴ is H or F. In some embodiments, Y⁶ is C(R⁴) wherein R⁴ is H.

In some embodiments, Y⁷ is N. In some embodiments, Y⁷ is C(H) or C(F). In some embodiments, Y⁷ is C(H). In some embodiments, Y⁷ is C(F). In some embodiments, Y⁷ is C(R⁴). In some embodiments, Y⁷ is C(R⁴) wherein R⁴ is H or halogen. In some embodiments, Y⁷ is C(R⁴) wherein R⁴ is H or F. In some embodiments, Y⁷ is C(R⁴) wherein R⁴ is H. In some embodiments, Y⁷ is C(R⁴) wherein R⁴ is F.

In some embodiments, Y⁸ is N. In some embodiments, Y⁸ is C(H). In some embodiments, Y⁸ is C(R⁴). In some embodiments, Y⁸ is C(R⁴) wherein R⁴ is H or halogen. In some embodiments, Y⁸ is C(R⁴) wherein R⁴ is H or F. In some embodiments, Y⁸ is C(R⁴) wherein R⁴ is H.

In some embodiments, R¹ and R² are each independently H, (C₁-C₄)alkyl or (C₁-C₄)alkenyl. In some embodiments, R¹ and R² are each independently H or (C₁-C₄)alkyl. In some embodiments, R¹ and R² are each independently H or methyl. In some embodiments, R¹ and R² are each H.

In some embodiments, R¹ is H and R² is (C₁-C₄)alkyl. In some embodiments, R¹ is H and R² is methyl.

In some embodiments, each R⁴ is independently H, halogen, —CN, (C₁-C₄)alkyl or (C₁-C₄)alkoxy. In some embodiments, each R⁴ is independently H or halogen. In some embodiments, each R⁴ is independently H or F. In some embodiments, each R⁴ is H.

In some embodiments, one R⁴ is H, halogen, —CN, (C₁-C₄)alkyl or (C₁-C₄)alkoxy, and the rest are H. In some embodiments, one R⁴ is halogen, —CN or (C₁-C₄)alkyl, and the rest are H. In some embodiments, one R⁴ is fluoro, chloro, bromo, methyl, ethyl or cyano, and the rest are H. In some embodiments, one R⁴ is halogen, and the rest are H. In some embodiments, one R⁴ is fluoro, and the rest are H.

In some embodiments, provided herein is a compound of Formula I, or a pharmaceutically acceptable salt thereof, wherein:
one of X¹ and X² is O, and the other is independently C(R³)₂ or O;
each R³ is H;
Y¹, Y², Y³, Y⁴, Y⁵, Y⁶, Y⁷, and Y⁸ are each independently C(R⁴) or N, and no more than one of Y¹, Y², Y³, Y⁴, Y⁵, Y⁶, Y⁷, and Y⁸ are N;
each R⁴ is independently H or halogen; and
R¹ and R² are each independently H or (C₁-C₄)alkyl.

In some embodiments, provided herein is a compound of Formula I, or a pharmaceutically acceptable salt thereof, wherein:
X¹ is C(R³)₂ or O, and X² is O;
each R³ is independently H or methyl;
Y¹, Y², Y³, Y⁴, Y⁵, Y⁶, Y⁷, and Y⁸ are each independently C(R⁴) or N, and no more than one of Y¹, Y², Y³, Y⁴, Y⁵, Y⁶, Y⁷, and Y⁸ are N;
one R⁴ is H, halogen, —CN, (C₁-C₄)alkyl or (C₁-C₄)alkoxy, and the rest are H;
R¹ is H; and
R² is (C₁-C₄)alkyl.

In some embodiments, X¹ is C(R³)₂ and X² is O. In some embodiments, X¹ is C(H)₂ and X² is O. In some embodiments, X¹ is O and X² is O. In some embodiments, Y¹, Y², Y³, Y⁴, Y⁵, Y⁶, Y⁷, and Y⁸ are each C(R⁴). In some embodiments, one of Y¹, Y², Y³, Y⁴, Y⁵, Y⁶, Y⁷, and Y⁸ is N, and the rest are each C(R⁴). In some embodiments, one of Y¹, Y³ and Y⁵ is N. In some embodiments, Y¹ is N. In some embodiments, one of Y⁵, Y⁶, Y⁷ and Y⁸ is N. In some embodiments, Y⁵ is N. In some embodiments, each R⁴ is H. In some embodiments, one R⁴ is halogen, —CN or (C₁-C₄)alkyl, and the rest are H. In some embodiments, one R⁴ is fluoro, chloro, bromo, methyl, ethyl or cyano, and the rest are H. In some embodiments, one R⁴ is halogen, and the rest are H. In some embodiments, one R⁴ is fluoro, and the rest are H. In some embodiments, R² is methyl.

In another aspect, the present disclosure provides compounds of Formula I(A):

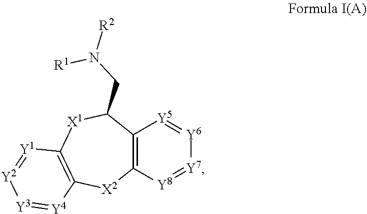

or a pharmaceutically acceptable salt thereof, wherein R¹, R², X¹, X², Y¹, Y², Y³, Y⁴, Y⁵, Y⁶, Y⁷ and Y⁸ are as defined herein.

In another aspect, the present disclosure provides compounds of Formula I(B):

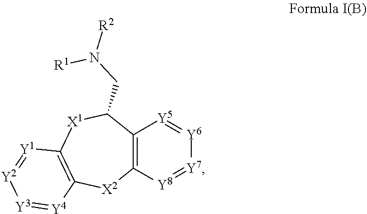

or a pharmaceutically acceptable salt thereof, wherein R¹, R², X¹, X², Y¹, Y², Y³, Y⁴, Y⁵, Y⁶, Y⁷ and Y⁸ are as defined herein.

In another aspect, the present disclosure provides compounds of Formula II:

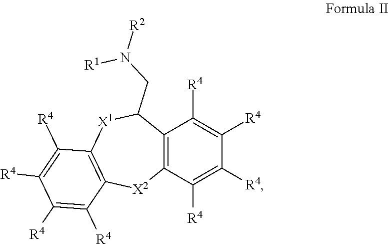

or a pharmaceutically acceptable salt thereof, wherein R¹, R², X¹, X², and R⁴ are as defined herein.

In another aspect, the present disclosure provides a compound of Formula III(A), Formula III(B), Formula III(C), Formula III(D), Formula III(E), Formula III(F), Formula III(G), or Formula III(H):

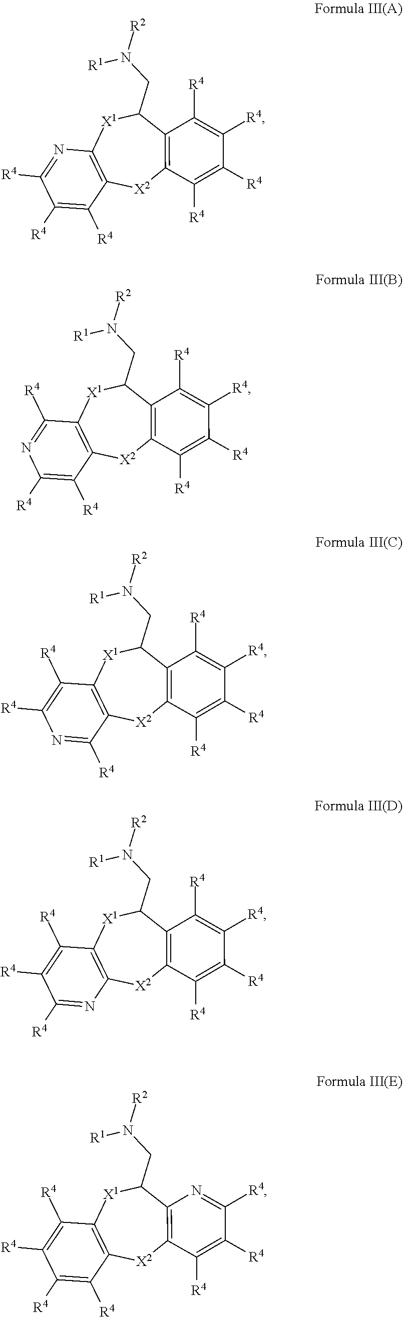

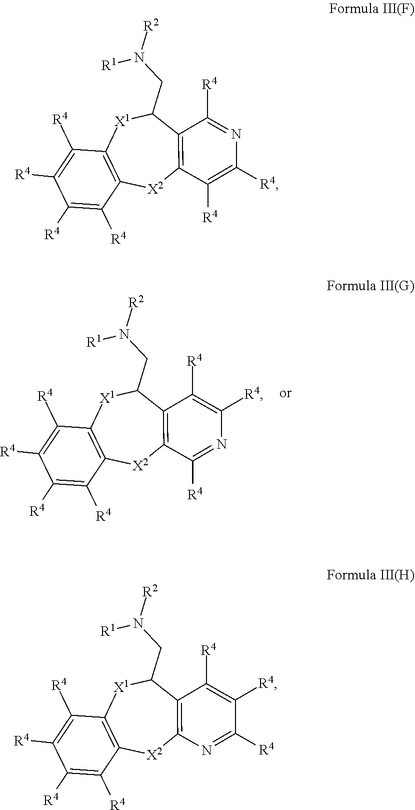

or a pharmaceutically acceptable salt thereof, wherein R¹, R², X¹, X², and R⁴ are as defined herein.

In another aspect, the present disclosure provides compounds of Formula IV:

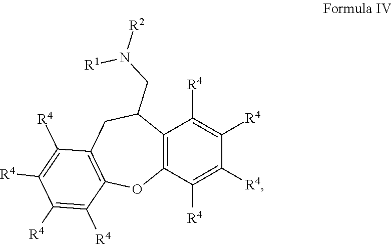

or a pharmaceutically acceptable salt thereof, wherein R¹, R², and R⁴ are as defined herein.

In another aspect, the present disclosure provides a compound of Formula V(A), Formula V(B), Formula V(C), Formula V(D), Formula V(E), Formula V(F), Formula V(G), or Formula V(H):

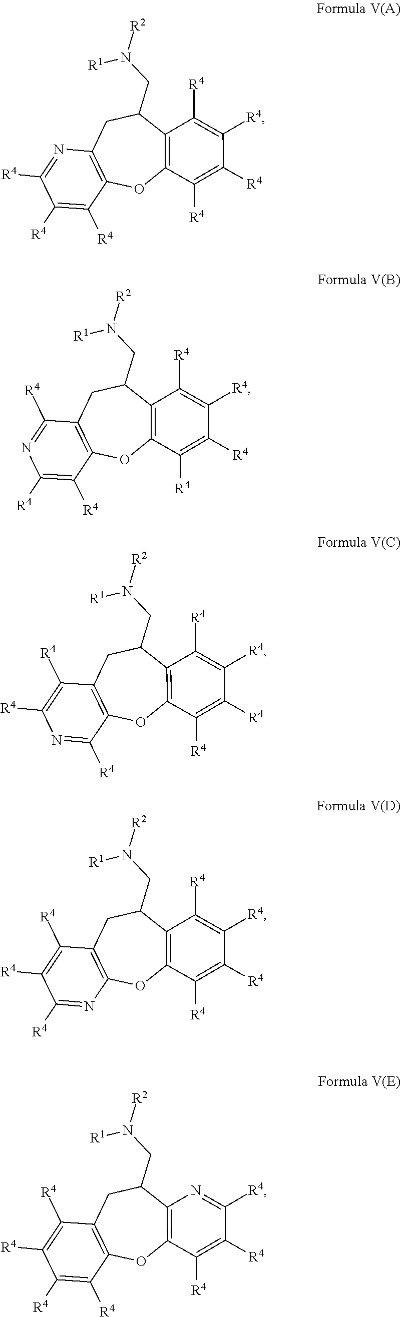

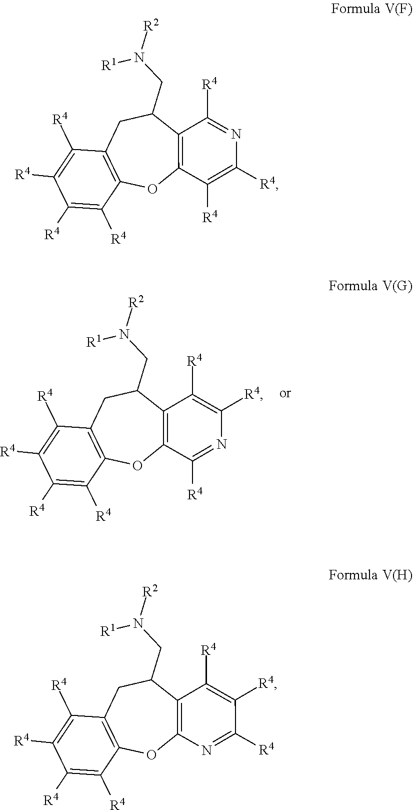

or a pharmaceutically acceptable salt thereof, wherein R¹, R², and R⁴ are as defined herein.

In one embodiment, provided is a compound selected from Table 1, or a pharmaceutically acceptable salt thereof.

In one embodiment, the compound is not N-((10,11-dihydrodibenzo[b,f]oxepin-10-yl)methyl)-N-methylprop-2-yn-1-amine, or a salt thereof.

### Pharmaceutical Compositions, Combinations and Kits

Compounds of the present disclosure are typically used, e.g., in accordance with the methods described herein, in a pharmaceutical composition (e.g., a pharmaceutical composition comprising a compound of the present disclosure and one or more pharmaceutically acceptable carriers).

In certain embodiments, provided herein is a composition (e.g., a pharmaceutical composition) comprising a compound of the present disclosure (e.g., a therapeutically effective amount of a compound of the present disclosure) and one or more pharmaceutically acceptable carriers. Examples of carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (e.g., a compound of the present disclosure or pharmaceutical composition thereof) or aid in the manufacturing of the pharmaceutical product (e.g., medicament).

Preferably, pharmaceutically acceptable carriers are sterile. The pharmaceutical composition can be formulated for particular routes of administration such as oral administration, parenteral administration (e.g., intravenous administration) and rectal administration, etc. In addition, the pharmaceutical compositions of the present disclosure can be made up in a solid form (including, without limitation, capsules, tablets, pills, granules, powders or suppositories), or in a liquid form (including, without limitation, solutions, suspensions or emulsions). The pharmaceutical compositions can be subjected to conventional pharmaceutical operations, such as sterilization, and/or can contain conventional inert diluents, lubricating agents, or buffering agents, as well as adjuvants, such as preservatives, stabilizers, wetting agents, emulsifiers and buffers, etc. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol;
c) binders, e.g., magnesium aluminum silicate, starch paste, gelatin, tragacanth, methylcellulose, sodium carboxymethylcellulose and/or polyvinylpyrrolidone;
d) disintegrants, e.g., starches, agar, alginic acid or its sodium salt, or effervescent mixtures; and
e) absorbents, colorants, flavors and sweeteners.
   Tablets may be either film-coated or enteric-coated according to methods known in the art.

Compositions of the present disclosure may be administered orally, parenterally, by inhalation, topically, rectally, nasally, buccally, sublingually, vaginally or via an implanted reservoir. The term “parenteral,” as used herein, includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. Pharmaceutically acceptable compositions of this disclosure may be orally administered in any orally acceptable dosage form including capsules, tablets, aqueous suspensions or solutions.

Suitable compositions for oral administration include a compound of the present disclosure (e.g., a compound of Formula I, or a subformula thereof, or a pharmaceutically acceptable salt of the foregoing) in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions comprise a compound of the present disclosure (e.g., a compound of Formula I, or a subformula thereof, or a pharmaceutically acceptable salt of the foregoing) in the form of an aqueous isotonic solution or suspension, and certain suppositories comprising a compound of the present disclosure (e.g., a compound of Formula I, or a subformula thereof, or a pharmaceutically acceptable salt of the foregoing) are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include a compound of the present disclosure (e.g., a compound of Formula I, or a subformula thereof, or a pharmaceutically acceptable salt of the foregoing) with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Suitable compositions comprising a compound of the present disclosure (e.g., a compound of Formula I, or a subformula thereof, or a pharmaceutically acceptable salt of the foregoing) for topical application, e.g., to the skin and eyes, include aqueous solutions, suspensions, ointments, creams, gels or sprayable formulations, e.g., for delivery by aerosol or the like. Such topical delivery systems will, in particular, be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein, a topical application may also pertain to an inhalation or to an intranasal application. A composition suitable for inhalation or intranasal administration may be conveniently delivered in the form of a dry powder (either alone, as a mixture, for example a dry blend with lactose, or a mixed component particle, for example, with phospholipids) from a dry powder inhaler, or an aerosol spray presentation from a pressurised container, pump, spray, atomizer or nebuliser, with or without the use of a suitable propellant.

The present disclosure further provides anhydrous pharmaceutical compositions and dosage forms comprising a compound provided herein (e.g., a compound of Formula I, or a subformula thereof), or a pharmaceutically acceptable salt thereof, since water may facilitate the degradation of certain compounds. Anhydrous pharmaceutical compositions and dosage forms of the disclosure can be prepared using anhydrous or low moisture-containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The present disclosure further provides pharmaceutical compositions and dosage forms that comprise one or more agents that reduce the rate by which the compound of the present disclosure (e.g., a compound of Formula I, or a subformula thereof, or a pharmaceutically acceptable salt of the foregoing) as an active ingredient will decompose. Such agents, which are referred to herein as “stabilizers,” include, but are not limited to, antioxidants such as ascorbic acid, pH buffers, or salt buffers, etc.

A compound of the present disclosure (e.g., a compound of Formula I, or a subformula thereof, or a pharmaceutically acceptable salt of the foregoing) is typically formulated into pharmaceutical dosage forms to provide an easily controllable dosage of the drug and to give the patient an elegant and easily handleable product. The dosage regimen for the compounds of the present disclosure will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration; the renal and hepatic function of the patient; and the effect desired. Compounds of the present disclosure may be administered in a single daily dose, or the total daily dosage may be administered in divided doses, e.g., two, three, or four times daily.

In certain instances, it may be advantageous to administer a compound of the present disclosure (e.g., a compound of Formula I, or a subformula thereof, or a pharmaceutically acceptable salt of the foregoing) in combination with one or more additional therapeutic agents.

The term “combination therapy” refers to the administration of two or more therapeutic agents to treat a disease or disorder described herein. Such administration encompasses co-administration of the therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Such administration also encompasses use of each type of therapeutic agent in a sequential manner, either at approximately the same time or at different times. A compound of the present disclosure (e.g., a compound of Formula I, or a subformula thereof, or a pharmaceutically acceptable salt of the foregoing) and an additional therapeutic agent(s) can be administered via the same administration route or via different administration routes. Powders and/or liquids may be reconstituted or diluted to a desired dose prior to administration. Typically, the treatment regimen will provide beneficial effects of the drug combination in treating the diseases or disorders described herein.

Compositions for use in combination therapies will either be formulated together as a pharmaceutical combination, or provided for separate administration (e.g., associated in a kit). Accordingly, a further embodiment is a pharmaceutical combination comprising a compound of the present disclosure (e.g., a compound of Formula I, or a subformula thereof, or a pharmaceutically acceptable salt of the foregoing) (e.g., a therapeutically effective amount of a compound of the present disclosure), and one or more additional therapeutic agents (e.g., a therapeutically effective amount of one or more other therapeutic agents). A pharmaceutical combination can further comprise one or more pharmaceutically acceptable carriers, such as one or more of the pharmaceutically acceptable carriers described herein.

A further embodiment is a kit comprising a compound of the present disclosure (e.g., a pharmaceutical composition comprising a compound of the present disclosure) and one or more additional therapeutic agents (e.g., one or more pharmaceutical composition(s) comprising one or more additional therapeutic agents). A kit of the present disclosure typically comprises directions for administration of the therapeutic agents contained therein, e.g., to treat a disease or disorder described herein.

In the combination therapies of the present disclosure, the compound of the present disclosure and the other therapeutic agent may be manufactured and/or formulated by the same or different manufacturers. Moreover, the compound of the present disclosure and the other therapeutic agent may be brought together into a combination therapy: (i) prior to release of the combination product to physicians (e.g., in the case of a kit comprising the compound of the present disclosure and the other therapeutic agent); (ii) by the physician (or under the guidance of a physician) shortly before administration; (iii) in the patient themselves, e.g., during sequential administration of the compound of the present disclosure and the other therapeutic agent.

Suitable pharmaceutical agents that may be used in combination with the compounds of the present disclosure include anti-Parkinson's drugs, anti-Alzheimer's drugs, anti-depressants, anti-psychotics, anti-ischemics, CNS depressants, anti-cholinergics, nootropics, epilepsy medication, attention (e.g., ADD/ADHD) medications, sleep-promoting medications, wakefulness-promoting medications, and pain medications.

Suitable anti-Parkinson's drugs include dopamine replacement therapy (e.g., L-DOPA, carbidopa, COMT inhibitors such as entacapone or tolcapone), dopamine agonists (e.g., D1 agonists, D2 agonists, mixed D1/D2 agonists, bromocriptine, pergolide, cabergoline, ropinirole, pramipexole, piribedil, or apomorphine in combination with domperidone), histamine H2 antagonists, monoamine oxidase inhibitors (such as selegiline, rasagiline, safinamide, and tranylcypromine), certain atypical antipsychotics such as pimavanserin (a non-dopaminergic atypical antipsychotic and inverse agonist of the serotonin 5-HT_{2A} receptor), and amantadine.

Compounds of the present disclosure can be used in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexyl)hydrochloride, COMT inhibitors such as entacapone or tolcapone, MAO A/B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexole are commonly used in a non-salt form.

Suitable anti-Alzheimer's drugs include beta-secretase inhibitors, gamma-secretase inhibitors, cholinesterase inhibitors such as donepezil, galantamine or rivastigmine, HMG-CoA reductase inhibitors, NSAID's including ibuprofen, vitamin E, and anti-amyloid antibodies. In some embodiments, an anti-Alzheimer's drug is memantine.

Suitable anti-depressants and anti-anxiety agents include norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT1A agonists or antagonists, especially 5-HT1A partial agonists, and corticotropin releasing factor (CRF) antagonists.

Specific suitable anti-depressant and anti-anxiety agents include amitriptyline, clomipramine, doxepin, imipramine and trimipramine; amoxapine, desipramine, citalopram, escitalopram, maprotiline, nortriptyline and protriptyline; fluoxetine, fluvoxamine, paroxetine and sertraline; isocarboxazid, phenelzine, tranylcypromine and selegiline; moclobemide: venlafaxine; desvenlafaxine, duloxetine; aprepitant; bupropion, vilazodone, mirtazapine, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, reboxetine, vortioxetine, clorazepate, and ketamine and pharmaceutically acceptable salts thereof. In some embodiments, suitable anti-depressant and anti-anxiety agents are tianeptine, or pharmaceutically acceptable salts thereof.

Suitable anti-psychotic and mood stabilizer agents include D2 antagonists, 5HT2A antagonists, atypical antipsychotics, lithium, and anticonvulsants.

Specific suitable anti-psychotic and mood stabilizer agents include chlorpromazine, fluphenazine, haloperidol, amisulpride, perphenazine, thioridazine, trifluoperazine, aripiprazole, asenapine, clozapine, olanzapine, paliperidone, brexpiprazole, paliperidone, cariprazine, pimavanserin, illoperidone, lumateperone, MIN-101, quetiapine, risperidone, ziprasidone, lurasidone, flupentixol, levomepromazine, pericyazine, perphenazine, pimozide, prochlorperazine, zuclopenthixol, olanzapine and fluoxetine, lithium, carbamazepine, lamotrigine, valproic acid, iloperidone, thiothixene, gabapentin, tiagabine and pharmaceutically acceptable salts thereof.

Suitable epilepsy medications include levetiracetam, oxcarbazepine, clobazam, retigabine, zonisamide, felbamate, esclicarbazepine acetate, lacosamide, carbamazepine, tiagabine, methsuximide, progabide, valproic acid, lamotrigine, brivaracetam, rufinamide, topiramate and perampanel.

Suitable attention medications include methyl phenidate, atomoxetine, guanfacine, D-amphetamine, lisdexamphetamine, methylamphetamine, and clonidine.

Suitable sleep-promoting medications include ramelteon, triazolam, zopiclone, eszopiclone, zolpidem, temazepam, and trazodone.

Suitable wakefulness-promoting medications include modafinil, D-amphetamine, caffeine, and armodafinil.

Suitable pain medications include dextromethorphan, tapentadol, buprenorphine, codeine, fentanyl, hydrocodone, hydromorphone, morphine, naloxegol, oxycodone, tramadol, gabapentil, difluprednate, pregabalin, acetyl salicyclic acid, bromfenac, diclofenac, diflunisal, indomethacin, ketorolac, meoxican, and naproxen.

In some embodiments, compounds of the present disclosure and compositions disclosed herein may be used in combination with other therapies. Suitable therapies include psychotherapy, cognitive behavioral therapy, electroconvulsive therapy, transcranial magnetic stimulation, vagus nerve stimulation, and deep-brain stimulation.

The compounds and compositions of the disclosure are preferably formulated in dosage unit form for ease of administration and uniformity of dosage. The expression “dosage unit form,” as used herein, refers to a physically discrete unit of agent appropriate for the subject to be treated. It will be understood, however, that the total daily usage of the compounds and compositions of the present disclosure will be decided by the attending physician within the scope of sound medical judgment.

The amount of a compound of the present disclosure that may be combined with a carrier material(s) to produce a composition in a single dosage form will vary depending upon a variety of factors, including, for example, the host treated and the particular mode of administration. For example, a dosage unit form may contain from about 1 to about 1000 mg of active ingredient(s) for a subject of from about 50 to about 70 kg, or from about 1 to about 500 mg, from about 1 to about 250 mg, from about 1 to about 150 mg, from about 0.5 to about 100 mg, or from about 1 to about 50 mg of active ingredient(s) for a subject of from about 50 to about 70 kg. It should also be understood that a specific dosage and treatment regimen for any particular subject will depend upon a variety of factors, including, for example, the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

In some embodiments, the concentration of one or more therapeutic agents provided in a pharmaceutical composition is less than 100%, 90%, 80%, 70%, 60%, 50%, 40%, 30%, 20%, 19%, 18%, 17%, 16%, 15%, 4, 13%, %12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more therapeutic agents provided in a pharmaceutical composition is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more therapeutic agents provided in a pharmaceutical composition is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% w/w, w/v or v/v.

In some embodiments, the concentration of one or more therapeutic agents provided in a pharmaceutical composition is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v.

### Methods of Use

It has now been found that the compounds of the present disclosure modulate (e.g., agonize) TAAR1. Accordingly, provided herein are methods of modulating (e.g., agonizing) TAAR1 in a cell (e.g., a cell expressing TAAR1), comprising contacting the cell with a compound of the present disclosure (e.g., a compound of Formula I, or a subformula thereof, or a pharmaceutically acceptable salt thereof, such as a therapeutically effective amount of a compound of Formula I, or a subformula thereof, or a pharmaceutically acceptable salt thereof).

When a method described herein comprises contacting a cell with a compound of the present disclosure, it will be understood that the method can be conducted in vitro, ex vivo or in vivo. Thus, some embodiments comprise contacting the cell in vitro. Some embodiments comprise contacting the cell ex vivo. Some embodiments comprise contacting the cell in vivo as, for example, when the cell is in a subject, such as a human.

Thus, also provided herein is a method of modulating (e.g., agonizing) TAAR1 in a subject in need thereof (e.g., a subject having a disease or disorder described herein, such as a neurological or psychiatric disease or disorder described herein), comprising administering to the subject a compound of the present disclosure (e.g., a compound of Formula I, or a subformula thereof, or a pharmaceutically acceptable salt thereof). Some embodiments comprise administering to the subject a therapeutically effective amount of the compound of the present disclosure. Some embodiments comprise administering the compound of the present disclosure in an amount sufficient to modulate (e.g., agonize) TAAR1 in the subject.

A compound of the present disclosure may modulate (e.g., agonize) TAAR1 selectively or it may exhibit other activities instead of or in addition to the TAAR1-modulating activity. For example, it has been found that certain compounds of the present disclosure selectively modulate (e.g., agonize) TAAR1, and certain compounds of the present disclosure modulate (e.g., agonize) TAAR1 and modulate (e.g., antagonize) 5-HT2A, 5-HT7 or 5-HT2A and 5-HT7.

Thus, in certain embodiments, the compound of the present disclosure is selective for TAAR1, e.g., selectively agonizes TAAR1 in a cell or subject. When a compound is described herein as being “selective” for a particular target, such as TAAR1, the compound binds to the indicated target to a greater extent than another target, such as 5-HT2A and/or 5-HT7, or other potential targets, e.g., encountered in a cell. Selectivity may be measured by the quotient of the EC₅₀ or IC₅₀ value of the compound in modulating (e.g., agonizing, inhibiting) the activity of a particular target over the EC₅₀ or IC₅₀ value of the compound in modulating (e.g., agonizing, inhibiting) the activity of another target. Selectivity may also be measured by the quotient of the K_{d} value of an adduct of the compound and a particular target over the K_{d} value of an adduct of the compound and another target. Selectivity may be at least 2-fold, at least 3-fold, at least 5-fold, at least 10-fold, at least 30-fold, at least 50-fold, at least 100-fold or greater than 100-fold, under comparable testing conditions.

In other embodiments, the compound of the present disclosure modulates (e.g., antagonizes) 5-HT2A. In some embodiments, the compound of the present disclosure modulates (e.g., antagonizes) 5-HT7. In some embodiments, the compound of the present disclosure modulates (e.g., antagonizes) 5-HT2A and 5-HT7.

Also provided herein are methods of modulating (e.g., antagonizing) 5-HT2A, 5-HT7, or 5-HT2A and 5-HT7 in a cell (e.g., a cell expressing 5-HT2A, 5-HT7, or 5-HT2A and 5-HT7), comprising contacting the cell with a compound of the present disclosure (e.g., a compound of Formula I, or a subformula thereof, or a pharmaceutically acceptable salt thereof, such as a therapeutically effective amount of a compound of Formula I, or a subformula thereof, or a pharmaceutically acceptable salt thereof).

Also provided herein is a method of modulating (e.g., antagonizing) 5-HT2A, 5-HT7, or 5-HT2A and 5-HT7 in a subject in need thereof (e.g., a subject having a disease or disorder described herein, such as a neurological or psychiatric disease or disorder described herein), comprising administering to the subject a compound of the present disclosure (e.g., a compound of Formula I, or a subformula thereof, or a pharmaceutically acceptable salt thereof). Some embodiments comprise administering to the subject a therapeutically effective amount of the compound of the present disclosure. Some embodiments comprise administering the compound of the present disclosure in an amount sufficient to modulate (e.g., antagonize) 5-HT2A, 5-HT7, or 5-HT2A and 5-HT7, respectively, in the subject. In some embodiments, the method is a method of modulating (e.g., antagonizing) 5-HT2A. In some embodiments, the method is a method of modulating (e.g., antagonizing) 5-HT7. In some embodiments, the method is a method of modulating (e.g., antagonizing) 5-HT2A and 5-HT7. In some embodiments, the compound of the present disclosure is selective for 5-HT2A over 5-HT7.

The Diagnostic and Statistical Manual of Mental Disorders, Fifth Ed., (the “DSM-5”), published by the American Psychiatric Association in 2013, and as amended or supplemented, provides a standard diagnostic system upon which persons of skill rely for diagnosis of various diseases and disorders, and is hereby incorporated by reference in its entirety. The DSM-5 attempts to capture the large proportion of patients with subsyndromal mixed symptoms with the inclusion of the mixed specifier. Additionally, the International Statistical Classification of Diseases (ICD 10) coding system is a recognized system to communicate about specific diagnoses (e.g., in the United States for billing purposes), and is hereby incorporated by reference in its entirety. For example, Chapter 6 of the ICD 10 is directed to codes for diseases of the nervous system.

The methods of the disclosure relate to the use of compounds of the present disclosure and compositions disclosed herein to treat neurological or psychiatric diseases or disorders. Accordingly, provided herein is a method of treating a neurological or psychiatric disease or disorder in a subject in need thereof, comprising administering to the subject a compound of the present disclosure (e.g., a therapeutically effective amount of a compound of the present disclosure). In some embodiments, the neurological or psychiatric disease or disorder is described in the DSM-5, as amended or supplemented, or the International Statistical Classification of Diseases (ICD 10) coding system.

Non-limiting examples of classes of neurological or psychiatric diseases or disorders include Movement Disorders, Cognitive Disorders, Pain, Neurodevelopmental Disorders; Schizophrenia Spectrum and Other Psychotic Disorders; Bipolar and Related Disorders; Depressive Disorders; Anxiety Disorders; Obsessive-Compulsive and Related Disorders; Trauma- and Stressor-Related Disorders; Dissociative Disorders; Somatic Symptom and Related Disorders; Feeding and Eating Disorders; Elimination Disorders; Sleep-Wake Disorders; Sexual Dysfunctions; Gender Dysphoria; Disruptive, Impulse-Control, and Conduct Disorders; Substance-Related and Addictive Disorders; Neurocognitive Disorders; Personality Disorders; Paraphilic Disorders; Other Mental Disorders; and Medication-Induced Movement Disorders and Other Adverse Effects of Medication.

Non-limiting examples of classes of neurological or psychiatric diseases or disorders include:

### Movement Disorders

Tremor; Dyskinesia; Dystonia; Tics; Dysphonia; Ataxia (e.g., spinocerebellar ataxia); Myoclonus; Essential Tremor; Epilepsy; Tardive Dyskinesia; Restless Leg Syndrome; Tourette Syndrome; Multiple System Atrophy (MSA); Multiple Sclerosis; Huntington's Disease; Parkinson's Disease; Parkinsonism; Atypical Parkinsonisms (including, for example, Parkinson's Disease Tremor); Wilson's Disease; Stroke. Examples of akinesias and akinetic-rigid syndromes include Parkinson's disease, drug-induced Parkinsonism, postencephalitic Parkinsonism, secondary Parkinsonism, Parkinson plus syndromes, atypical Parkinsonism, idiopathic Parkinsonism, progressive supranuclear palsy, multiple system atrophy, corticobasal degeneration, Parkinsonism-ALS dementia complex and basal ganglia calcification, medication-induced Parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, muscular spasms and disorders associated with muscular spasticity or weakness including tremors. Examples of dyskinesias include drug (e.g. L-DOPA) induced dyskinesia tremor (such as rest tremor, postural tremor, intention tremor), chorea (such as Sydenham's chorea, Huntington's disease, benign hereditary chorea, neuroacanthocytosis, symptomatic chorea, drug-induced chorea and hemiballism), myoclonus (including generalized myoclonus and focal myoclonus), tics (including simple tics, complex tics and symptomatic tics). Examples of dystonias include generalized dystonia, idiopathic dystonia, drug-induced dystonia, symptomatic dystonia, paroxysmal dystonia, focal dystonia, blepharospasm, oromandibular dystonia, spasmodic dysphonia, spasmodic torticollis, axial dystonia, dystonic writer's cramp and hemiplegic dystonia. Other examples of movement diseases or disorders include stereotypic movement disorder, persistent (chronic) motor disorder, medication-Induced movement disorder, psychogenic movement disorders, substance/medication-Induced movement disorder, extrapyramidal movement disorders, hyperkinetic movement disorders, hypokinetic movement disorders, alternating hemiplegia, Angelman syndrome, Hallervorden-Spatz Disease, ataxia, dentate cerebellar ataxia, ataxia telangiectasia (Louis-Bar syndrome), Friedreich's Ataxia, hereditary spinal ataxia, hereditary spinal sclerosis, Machado-Joseph Disease, spinocerebellar ataxia, progressive myoclonic ataxia, athetosis, ballismus, blepharospasm (eye twitching), cerebral palsy, tardive dystonia, tardive dyskinesia, idiopathic torsion dystonia, torsion dystonia, focal dystonia, idiopathic familial dystonia, Idiopathic nonfamilial dystonia, cervical dystonia (spasmodic torticollis), primary dystonia, orofacial dystonia, developmental coordination disorder, bulbospinal muscular atrophy (Kennedy's Disease), Shy-Drager Syndrome, and Stiff-Person (Stiff-Man) Syndrome. In some embodiments, the present disclosure provides a method of treating one or more symptoms of epilepsy and/or seizures, including abdominal epilepsy, absence seizure, acquired epilepsy, acquired epileptiform aphasia, Aicardi syndrome, Alpers' disease, Alpers-Huttenlocher syndrome, Angelman syndrome, benign focal epilepsy, benign focal epilepsy of childhood, benign intracranial hypertension, benign rolandic epilepsy (BRE), CDKL5 disorder, childhood absence epilepsy, dentate cerebellar ataxia, Doose syndrome, Dravet syndrome, dyscognitive focal seizure, epilepsy with grand mal seizures, epilepsy with myoclonic-absences, epileptic hemiplegia, febrile seizures, focal seizure, frontal lobe epilepsy, generalized tonic-clonic seizures, genetic epilepsy, Glut1 deficiency syndrome, hypothalamic hamartoma, idiopathic epilepsy, idiopathic generalized epilepsy, idiopathic localization-related epilepsies, idiopathic partial epilepsy, idiopathic seizure, juvenile absence epilepsy, juvenile myoclonic epilepsy, Lafora disease, Lafora progressive myoclonus epilepsy, Landau-Kleffner syndrome, Lassueur-Graham-Little syndrome, Lennox syndrome, Lennox-Gastaut syndrome, medically refractory epilepsy, mesial-temporal lobe sclerosis, myoclonic seizure, neonatal epilepsy, occipital lobe epilepsy, Ohtahara syndrome, Panayiotopoulos syndrome, parietal lobe epilepsy, PCDH19 epilepsy, photosensitive epilepsy, progressive myoclonic epilepsies, Rasmussen's encephalitis, Rasmussen's syndrome, refractory epilepsy, seizure disorder, status epilepticus, Sturge-Weber syndrome, symptomatic generalized epilepsy, symptomatic partial epilepsy, TBCK-related ID syndrome, temporal lobe epilepsy, temporal lobe seizures, tonic-clonic seizure, West syndrome, tremor, cerebellar tremor, cerebellar outflow tremor, intention tremor, essential tremor, benign essential tremor, Parkinsonian tremor, and medication-induced postural tremor.

### Cognitive Disorders

Alzheimer's disease; Cognitive Impairments; Dementia (including, e.g., Semantic Dementia; Frontotemporal Dementia; Dementia with Depressive Features; Persisting, Subcortical Dementia; Dementia with Lewy Bodies; Parkinsonism-ALS Dementia Complex; Dementia Associated with another disease or disorder, including Alzheimer's Disease; Ischemia; Multi-Infarct Dementia; Trauma; Vascular Problems; Stroke; HIV Disease; Parkinson's Disease; Huntington's Disease; Down Syndrome; Pick's Disease; Creutzfeldt-Jacob Disease; Perinatal Hypoxia, or Substance abuse), Delirium; Amnestic Disorders; or Age Related Cognitive Decline. Cognitive Disorders includes a decline in cognitive functions or cognitive domains, e.g., working memory, attention and vigilance, verbal learning and memory, visual learning and memory, reasoning and problem solving (e.g., executive function, speed of processing and/or social cognition). In particular, cognitive impairment may indicate deficits in attention, disorganized thinking, slow thinking, difficulty in understanding, poor concentration, impairment of problem solving, poor memory, difficulties in expressing thoughts, and/or difficulties in integrating thoughts, feelings and behavior, or difficulties in extinction of irrelevant thoughts. Cognitive Disorders can manifest as a deficit in cognition (cognitive domains as defined by the DSM-5 are: complex attention, executive function, learning and memory, language, perceptual-motor, social cognition); and is sometimes associated with a deficit in dopamine signaling; and is sometimes associated with basal ganglia dysfunction; and is sometimes associated with dysregulated locomotor activity; and is sometimes associated with impairment of prefrontal cortex functioning.

### Pain

Fibromyalgia; Neuropathic Pain (including, e.g., post herpetic (or post-shingles) neuralgia, reflex sympathetic dystrophy/causalgia or nerve trauma, phantom limb pain, carpal tunnel syndrome, and peripheral neuropathy (such as diabetic neuropathy or neuropathy arising from chronic alcohol use)), Sensitization Accompanying Neuropathic Pain, Inflammatory Pain; Acute Pain; Nociceptive Pain; Arthritis Pain; Rheumatoid Arthritis; Osteoarthritis; Joint Pain; Musculoskeletal Pain; Back Pain; Dorsalgia; Bulging Disc; Hip Pain; Visceral Pain; Headache; Tension Headache; Acute Tension Headache; Chronic Tension Headache; Chronic Cluster Headache; Common Migraine; Classic Migraine; Cluster Headache; Mixed Headache; Post-Traumatic Headache; Eye Strain Headache; Short-Lasting Unilateral Neuralgiform (SUNCT) Headache; SUNCT Syndrome, Herpes Zoster; Acute Herpes Zoster; Shingles; Postherpetic Neuralgia (Shingles); Causalgia; Central Pain; Central Pain Syndrome; Chronic Back Pain; Neuralgia; Neuropathic Pain Syndrome; Neuropathy; Diabetic Neuropathy; Diabetes-Related Neuropathy; Diabetes-Related Nerve Pain; Fibrositis; Peripheral Neuropathy Caused by Chemotherapy; Peripheral Nerve Disease; Peripheral Neuropathy; Nerve Pain; Nerve Trauma; Sensitization Accompanying Neuropathic Pain; Complex Regional Pain Syndrome; Compression Neuropathy; Craniofacial Pain; Chronic Joint Pain; Chronic Knee Pain; Chronic Pain Syndrome; Cancer Pain; Trigeminal Neuralgia; Tic Doloreaux; Reflex Sympathetic Causalgia; Painful Peripheral Neuropathy; Spinal Nerve Injury; Arachnoiditis; Spinal Pain; Bernhardt-Roth Syndrome (Meralgia Parasthetica); Carpal Tunnel Syndrome; Cerebrospinal Fluid Syndrome; Charcot-Marie-Tooth Disease; Hereditary Motor and Sensory Neuropathy; Peroneal Muscular Atrophy; Cluster-Tic Syndrome; Coccygeal Pain Syndromes; Compartment Syndrome; Degenerative Disc Disease; Failed Back Surgery Syndrome; Genito-Pelvic Pain/Penetration Disorder; Gout; Inflammatory Pain; Lumbar Radiculopathy; Neuroma (Painful Scar); Pain Associated with Multiple Sclerosis; Pelvic Floor Disorders; Phantom Limb Pain; Piriformis Syndrome; Psychogenic Pain; Radicular Pain Syndrome; Raeder's Syndrome; Referred Pain; Reflex Sympathetic Dystrophy Syndrome; Sciatica; Sciatica Pain: Scoliosis; Slipped Disc; Somatic Pain; Spinal Stenosis; Stiff-Person Syndrome/Stiff-Man Syndrome; Stump Pain; Sympathetically Maintained Pain; Tolosa-Hunt Syndrome; Whiplash; Pain Associated with Lyme Disease.

### Neurodevelopmental Disorders

Intellectual Disability (Intellectual Developmental Disorder); Global Developmental Delay; Unspecified Intellectual Disability (Intellectual Developmental Disorder); Language Disorder; Speech Sound Disorder; Childhood-Onset Fluency Disorder (Stuttering); Social (Pragmatic) Communication Disorder; Unspecified Communication Disorder; Autism Spectrum Disorder (including, e.g., Asperger's syndrome; Pervasive Developmental Disorder; Rett Syndrome; and Fragile X Syndrome); Attention-Deficit/Hyperactivity Disorder; Other Specified Attention-Deficit/Hyperactivity Disorder; Unspecified Attention-Deficit/Hyperactivity Disorder; Specific Learning Disorder; Childhood Learning Disorder; Developmental Coordination Disorder; Stereotypic Movement Disorder; Tic Disorders; Other Specified Tic Disorder; Unspecified Tic Disorder; Other Specified Neurodevelopmental Disorder; Unspecified Neurodevelopmental Disorder

### Schizophrenia Spectrum and Other Psychotic Disorders

Schizotypal (Personality) Disorder; Delusional Disorder; Brief Psychotic Disorder; Shared Psychotic Disorder Schizophreniform Disorder; Schizophrenia (paranoid, disorganized, catatonic, or undifferentiated); Schizoaffective Disorder; Substance/Medication-Induced Psychotic Disorder; Psychotic Disorder Due to Another Medical Condition; Catatonia Associated With Another Mental Disorder (Catatonia Specifier); Catatonic Disorder Due to Another Medical Condition; Unspecified Catatonia; Other Specified Schizophrenia Spectrum and Other Psychotic Disorder; Unspecified Schizophrenia Spectrum and Other Psychotic Disorder. Schizophrenia is a disorder of unknown origin, which usually appears for the first time in early adulthood and is marked by characteristics such as psychotic symptoms, phasic progression and development, and/or deterioration in social behavior and professional capability. Characteristic psychotic symptoms are disorders of thought content (e.g., multiple, fragmentary, incoherent, implausible or simply delusional contents, or ideas of persecution) and of mentality (e.g., loss of association, flight of imagination, incoherence up to incomprehensibility), as well as disorders of perceptibility (e.g., hallucinations), emotions (e.g., superficial or inadequate emotions), self-perceptions, intentions, impulses, and/or inter-human relationships, and psychomotoric disorders (e.g., catatonia). Other symptoms are also associated with this disorder. Schizophrenia is classified into subgroups: the paranoid type, characterized by delusions and hallucinations and absence of thought disorder, disorganized behavior, and affective flattening; the disorganized type, also named “hebephrenic schizophrenia,” in which thought disorder and flat affect are present together; the catatonic type, in which prominent psychomotor disturbances are evident, and symptoms may include catatonic stupor and waxy flexibility; and the undifferentiated type, in which psychotic symptoms are present but the criteria for paranoid, disorganized, or catatonic types have not been met. The symptoms of schizophrenia normally manifest themselves in three broad categories: positive, negative and cognitive symptoms. Positive symptoms are those which represent an “excess” of normal experiences, such as hallucinations and delusions. Negative symptoms are those where the subject suffers from a lack of normal experiences, such as anhedonia and lack of social interaction. The cognitive symptoms relate to cognitive impairment in schizophrenics, such as lack of sustained attention and deficits in decision making.

### Bipolar and Related Disorders

Bipolar I Disorder; Bipolar II Disorder; Cyclothymic Disorder; Substance/Medication-Induced Bipolar and Related Disorder; Bipolar and Related Disorder Due to Another Medical Condition; Other Specified Bipolar and Related Disorder; Unspecified Bipolar and Related Disorder; Specifiers for Bipolar and Related Disorders. Bipolar disorders (including both bipolar I and bipolar II) are serious psychiatric disorders that have a prevalence of approximately 2% of the population, and affects both genders alike. It is a relapsing-remitting condition characterized by cycling between elevated (i.e., manic) and depressed moods, which distinguishes it from other disorders such as major depressive disorder and schizophrenia. Bipolar I is defined by the occurrence of a full manic episode, although most individuals experience significant depression. Symptoms of mania include elevated or irritable mood, hyperactivity, grandiosity, decreased need for sleep, racing thoughts and in some cases, psychosis. The depressive episodes are characterized by anhedonia, sad mood, hopelessness, poor self-esteem, diminished concentration and lethargy. Bipolar II is defined as the occurrence of a major depressive episode and hypomanic (less severe mania) episode although subjects spend considerably more time in the depressive state. Other related conditions include cyclothymic disorder.

### Depressive Disorders

Depression, Disruptive Mood Dysregulation Disorder; Major Depressive Disorder (MDD) (Unipolar Depression); Persistent Depressive Disorder (Dysthymia); Premenstrual Dysphoric Disorder; Substance/Medication-Induced Depressive Disorder; Treatment-Resistant Depression; Depressive Disorder Due to Another Medical Condition; Other Specified Depressive Disorder; Unspecified Depressive Disorder; Adjunctive Major Depressive Disorder.

### Anxiety Disorders

Anxiety; Separation Anxiety Disorder; Selective Mutism; Specific Phobia; Social Anxiety Disorder (Social Phobia); Panic Disorder; Panic Attack Specifier; Agoraphobia; Generalized Anxiety Disorder; Substance/Medication-Induced Anxiety Disorder; Anxiety Disorder Due to Another Medical Condition; Other Specified Anxiety Disorder; Unspecified Anxiety Disorder. Anxiety disorders are characterized by fear, worry, and uneasiness, usually generalized and unfocused as an overreaction to a situation. Anxiety disorders differ in the situations or types of objects that induce fear, anxiety, or avoidance behavior, and the associated cognitive ideation. Anxiety differs from fear in that anxiety is an emotional response to a perceived future threat while fear is associated with a perceived or real immediate threat. They also differ in the content of the associated thoughts or beliefs. Examples of anxiety disorders include separation anxiety disorder, selective mutism, specific phobia, social anxiety disorder (social phobia), panic disorder, panic attack specifier, agoraphobia, generalized anxiety disorder, substance/medication-induced anxiety disorder, anxiety disorder due to another medical condition, illness anxiety disorder, social (pragmatic) communication disorder, other specified anxiety disorder, and unspecified anxiety disorder; stressor-related disorders, including reactive attachment disorder, disinhibited social engagement disorder, posttraumatic stress disorder (PTSD), acute stress disorder, and adjustment disorders.

### Obsessive-Compulsive and Related Disorders

Obsessive-Compulsive Disorder; Body Dysmorphic Disorder; Hoarding Disorder; Trichotillomania (Hair-Pulling Disorder); Excoriation (Skin-Picking) Disorder; Substance/Medication-Induced Obsessive-Compulsive and Related Disorder; Obsessive-Compulsive and Related Disorder Due to Another Medical Condition; Other Specified Obsessive-Compulsive and Related Disorder; Unspecified Obsessive-Compulsive and Related Disorder

### Trauma- and Stressor-Related Disorders

Reactive Attachment Disorder; Disinhibited Social Engagement Disorder; Posttraumatic Stress Disorder; Acute Stress Disorder; Adjustment Disorders; Other Specified Trauma- and Stressor-Related Disorder; Unspecified Trauma- and Stressor-Related Disorder.

### Dissociative Disorders

Dissociative Identity Disorder; Dissociative Amnesia; Depersonalization/Derealization Disorder; Other Specified Dissociative Disorder; Unspecified Dissociative Disorder.

### Somatic Symptom and Related Disorders

Somatic Symptom Disorder; Illness Anxiety Disorder; Conversion Disorder (Functional Neurological Symptom Disorder); Psychological Factors Affecting Other Medical Conditions; Factitious Disorder; Other Specified Somatic Symptom and Related Disorder; Unspecified Somatic Symptom and Related Disorder.

### Feeding and Eating Disorders

Pica; Rumination Disorder; Avoidant/Restrictive Food Intake Disorder; Anorexia Nervosa; Bulimia Nervosa; Binge-Eating Disorder; Other Specified Feeding or Eating Disorder; Unspecified Feeding or Eating Disorder.

### Elimination Disorders

Enuresis; Encopresis; Other Specified Elimination Disorder; Unspecified Elimination Disorder.

### Sleep-Wake Disorders

Insomnia Disorder; Hypersomnolence Disorder; Narcolepsy; Obstructive Sleep Apnea Hypopnea; Central Sleep Apnea; Sleep-Related Hypoventilation; Circadian Rhythm Sleep-Wake Disorders; Non-Rapid Eye Movement Sleep Arousal Disorders; Nightmare Disorder; Rapid Eye Movement (REM) Sleep Behavior Disorder; Restless Legs Syndrome; Substance/Medication-Induced Sleep Disorder; Other Specified Insomnia Disorder; Unspecified Insomnia Disorder; Other Specified Hypersomnolence Disorder; Unspecified Hypersomnolence Disorder; Other Specified Sleep-Wake Disorder; Unspecified Sleep-Wake Disorder.

### Sexual Dysfunctions

Delayed Ejaculation; Erectile Disorder; Female Orgasmic Disorder; Female Sexual Interest/Arousal Disorder; Genito-Pelvic Pain/Penetration Disorder; Male Hypoactive Sexual Desire Disorder; Premature (Early) Ejaculation; Substance/Medication-Induced Sexual Dysfunction; Other Specified Sexual Dysfunction; Unspecified Sexual Dysfunction.

### Gender Dysphoria

Gender Dysphoria; Other Specified Gender Dysphoria; Unspecified Gender Dysphoria.

### Disruptive, Impulse-Control, and Conduct Disorders

Social Disorder; Oppositional Defiant Disorder; Intermittent Explosive Disorder; Conduct Disorder; Antisocial Personality Disorder; Pyromania; Kleptomania; Other Specified Disruptive, Impulse-Control, and Conduct Disorder; Unspecified Disruptive; Impulse-Control, and Conduct Disorder.

### Substance-Related and Addictive Disorders

Addiction; Alcohol Use Disorder; Alcohol Intoxication; Alcohol Withdrawal; Unspecified Alcohol-Related Disorder; Fetal Alcohol Syndrome; Caffeine Intoxication; Caffeine Withdrawal; Unspecified Caffeine-Related Disorder; Cannabis Use Disorder; Cannabis Intoxication; Cannabis Withdrawal; Unspecified Cannabis-Related Disorder; Phencyclidine Use Disorder; Other Hallucinogen Use Disorder; Phencyclidine Intoxication; Other Hallucinogen Intoxication; Hallucinogen Persisting Perception Disorder; Unspecified Phencyclidine-Related Disorder; Unspecified Hallucinogen-Related Disorder; Inhalant Use Disorder; Inhalant Intoxication; Unspecified Inhalant-Related Disorder; Opioid Use Disorder; Opioid Intoxication; Opioid Withdrawal; Unspecified Opioid-Related Disorder; Sedative, Hypnotic, or Anxiolytic Use Disorder; Sedative, Hypnotic, or Anxiolytic Intoxication; Sedative, Hypnotic, or Anxiolytic Withdrawal; Unspecified Sedative-, Hypnotic-, or Anxiolytic-Related Disorder; Stimulant Use Disorder; Stimulant Intoxication; Stimulant Withdrawal; Unspecified Stimulant-Related Disorder; Tobacco Use Disorder; Tobacco Withdrawal; Unspecified Tobacco-Related Disorder; Other (or Unknown) Substance Use Disorder; Other (or Unknown) Substance Intoxication; Other (or Unknown) Substance Withdrawal; Unspecified Other (or Unknown) Substance-Related Disorder; Gambling Disorder.

### Neurocognitive Disorders

Delirium; Other Specified Delirium; Unspecified Delirium; Major and Mild Neurocognitive Disorders; Major or Mild Neurocognitive Disorder Due to Alzheimer's Disease; Major or Mild Frontotemporal Neurocognitive Disorder; Major or Mild Neurocognitive Disorder With Lewy Bodies; Major or Mild Vascular Neurocognitive Disorder; Major or Mild Neurocognitive Disorder Due to Traumatic Brain Injury; Substance/Medication-Induced Major or Mild Neurocognitive Disorder; Major or Mild Neurocognitive Disorder Due to HIV Infection; Major or Mild Neurocognitive Disorder Due to Prion Disease; Major or Mild Neurocognitive Disorder Due to Parkinson's Disease; Major or Mild Neurocognitive Disorder Due to Huntington's Disease; Major or Mild Neurocognitive Disorder Due to Another Medical Condition; Major or Mild Neurocognitive Disorder Due to Multiple Etiologies; Unspecified Neurocognitive Disorder.

### Personality Disorders

Dimensional Models for Personality Disorders; General Personality Disorder; Paranoid Personality Disorder; Schizoid Personality Disorder; Schizotypal Personality Disorder; Antisocial Personality Disorder; Borderline Personality Disorder; Histrionic Personality Disorder; Narcissistic Personality Disorder; Avoidant Personality Disorder; Dependent Personality Disorder; Obsessive-Compulsive Personality Disorder; Personality Change Due to Another Medical Condition; Other Specified Personality Disorder; Unspecified Personality Disorder.

### Paraphilic Disorders

Voyeuristic Disorder; Exhibitionistic Disorder; Frotteuristic Disorder; Sexual Masochism Disorder; Sexual Sadism Disorder; Pedophilic Disorder; Fetishistic Disorder; Transvestic Disorder; Other Specified Paraphilic Disorder; Unspecified Paraphilic Disorder.

### Other Mental Disorders

Other Specified Mental Disorder Due to Another Medical Condition; Unspecified Mental Disorder Due to Another Medical Condition; Other Specified Mental Disorder; Unspecified Mental Disorder.

### Medication-Induced Movement Disorders and Other Adverse Effects of Medication

Neuroleptic-Induced Parkinsonism Other Medication-Induced Parkinsonism; Neuroleptic Malignant Syndrome; Medication-Induced Acute Dystonia; Medication-Induced Acute Akathisia; Tardive Dyskinesia; Tardive Dystonia Tardive Akathisia; Medication-Induced Postural Tremor; Other Medication-Induced Movement Disorder; Antidepressant Discontinuation Syndrome; Other Adverse Effect of Medication.

### Symptoms of Neurological or Psychiatric Diseases and Disorders

Neurological or psychiatric diseases or disorders can manifest as a variety of symptoms. Non-limiting examples of symptoms of neurological or psychiatric diseases or disorders include symptoms such as apathy, depression, anxiety, cognitive impairment, psychosis, aggression, agitation, impulse control disorders, sleep disorders, elevated or irritable mood, hyperactivity, grandiosity, decreased need for sleep, racing thoughts and in some cases, psychosis, anhedonia, sad mood, hopelessness, poor self-esteem, diminished concentration and lethargy, amyotrophic lateral sclerosis, primary lateral sclerosis, progressive muscular atrophy, progressive bulbar (atrophy) palsy, pseudobulbar palsy spinal muscular atrophy diseases (e.g., SMA type I, also called Werdnig-Hoffmann disease, SMA type II, SMA type III, also called Kugelberg-Welander disease, and Kennedy Disease, also called progressive spinobulbar muscular atrophy), Hallervorden-Spatz disease, Seitelberger disease (Infantile Neuroaxonal Dystrophy), adrenoleukodystrophy, Alexander Disease, autosomal dominant cerebellar ataxia (ADCA), pure autonomic failure (Bradbury-Eggleston Syndrome), CADASIL Syndrome, and neuronal ceroids lipofuscinose disorders such as Batten Disease (Spielmeyer-Vogt-Sjögren)), senile dementia, Early Onset Alzheimer's Disease, Alzheimer's type dementia, cognition, memory loss, amnesia/amnestic syndrome, disturbances of consciousness, coma, lowering of attention, speech disorder, agnosia, aphasia, apraxia, Mild Cognitive Impairment (MCI), benign forgetfulness, mild neurocognitive disorder, major neurocognitive disorder, neurocognitive disorder due to disease (e.g., Huntington's Disease, Parkinson's disease, Prion Disease, Traumatic Brain Injury, HIV or AIDS), Binswanger's Disease (subcortical leukoencephalopathy), and Capgras Syndrome; or any other symptoms associated with a neurological or psychiatric disease or disorder disclosed herein.

TAAR1 agonists are also useful for metabolic control. Molecular Metabolism 5 (2016) 47-56.

In some embodiments, the neurological or psychiatric disease or disorder is schizophrenia.

In some embodiments, the neurological or psychiatric disease or disorder is a bipolar disorder.

In some embodiments, the neurological or psychiatric disease or disorder is Parkinson's disease.

In some embodiments, the neurological or psychiatric disease or disorder is Alzheimer's disease.

In some embodiments, the neurological or psychiatric disease or disorder is autism spectrum disorder.

In some embodiments, the neurological or psychiatric disease or disorder is a substance-related or addictive disorder.

In some embodiments, the neurological or psychiatric disease or disorder is a metabolic disease. Examples of metabolic disease include, but are not limited to, impaired glucose tolerance; elevated blood glucose; elevated fasting glucose; insulin resistance; insulin insensitivity; hyperglycemia; overweightness or increased weight; increased body mass index; metabolic syndrome; diabetes, including type 1 diabetes and type 2 diabetes.

A therapeutically effective amount of a therapeutic agent (e.g., a compound of the present disclosure) to be administered to a subject in accordance with the methods described herein can be determined by a clinician of ordinary skill using the guidance provided herein and other methods known in the art. For example, suitable dosages may range, depending on the route of administration, among other things, from about 0.1 mg/kg to about 500 mg/kg, or from about 1 mg/kg to about 100 mg/kg.

A therapeutic agent described herein, including a compound of the present disclosure, can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound and the particular disease to be treated. Administration can be local or systemic as indicated. The preferred mode of administration can vary depending on the particular compound chosen. In some embodiments, the compound of the present disclosure is administered orally. In some embodiments, the compound of the present disclosure is administered intravenously.

In some embodiments, the methods further comprise administering to the subject one or more other therapies (e.g., psychotherapy, cognitive behavioral therapy, electroconvulsive therapy, transcranial magnetic stimulation, vagus nerve stimulation, and deep-brain stimulation). In some embodiments, the methods further comprise administering to the subject one or more additional therapeutic agents (e.g., a therapeutically effective amount of one or more additional therapeutic agents). Examples of suitable additional therapeutic agents include anti-Parkinson's drugs, anti-Alzheimer's drugs, anti-depressants, anti-psychotics, anti-ischemics, CNS depressants, anti-cholinergics, nootropics, epilepsy medication, attention (e.g., ADD/ADHD) medications, sleep-promoting medications, wakefulness-promoting medications, and pain medications. Other suitable additional therapies and therapeutic agents for use in the methods disclosed herein include those discussed herein in connection with combination therapy and pharmaceutical combinations.

When administered in combination with another therapy, the compound of the present disclosure can be administered before, after or concurrently with the other therapy (e.g., additional therapeutic agent(s)). When two or more therapeutic agents are co-administered simultaneously (e.g., concurrently), the compound of the present disclosure and other therapeutic agent(s) can be in separate formulations or the same formulation. Alternatively, the compound of the present disclosure and other therapy can be administered sequentially (e.g., as separate compositions) within an appropriate time frame as determined by a skilled clinician (e.g., a time sufficient to allow an overlap of the pharmaceutical effects of the compound of the present disclosure and the other therapy).

### EXEMPLIFICATION

The compounds of the present disclosure can be prepared in a number of ways known to one skilled in the art of organic synthesis in view of the methods, reaction schemes and examples provided herein. The compounds of the present disclosure can be synthesized using the methods described below, together with synthetic methods known in the art of synthetic organic chemistry, or by variations thereon, as appreciated by those skilled in the art. Preferred methods include, but are not limited to, those described below. The reactions are performed in a solvent or solvent mixture appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the disclosure. Examples are depicted with relative stereochemistry except where specifically stated otherwise.

The starting materials are generally available from commercial sources such as Sigma Aldrich or other commercial vendors, or are prepared as described in this disclosure, or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), Larock, R. C., Comprehensive Organic Transformations, 2^{nd} ed., Wiley-VCH Weinheim, Germany (1999), or Beilsteins Handbuch der organischen Chemie, 4, Aufl. ed. Springer-Verlag, Berlin, including supplements (also available via the Beilstein online database).

For illustrative purposes, the reaction schemes depicted below provide potential routes for synthesizing the compounds of the present disclosure as well as key intermediates. Those skilled in the art will appreciate that other synthetic routes may be used to synthesize the compounds of the present disclosure. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the present disclosure, protection of remote functionality of intermediates may be necessary. The need for such protection will vary depending on the nature of the remote functionality and the conditions of the preparation methods. The need for such protection is readily determined by one skilled in the art. For a general description of protecting groups and their use, see Greene, T. W. et al., Protecting Groups in Organic Synthesis, 4th Ed., Wiley (2007). Protecting groups incorporated in making of the compounds of the present disclosure, such as the trityl protecting group, may be shown as one regioisomer but may also exist as a mixture of regioisomers.

The following abbreviations used hereinbelow have the corresponding meanings:

| | | | |
|---|---|---|---|
| Ac | acetyl; | | |
| ACN | acetonitrile; | Aq | aqueous; |
| BSA | bovine serum albumin; | Boc | tert-butyloxycarbonyl; |
| C. | Celsius; | CH₂Cl₂ | dichloromethane; |
| Cs₂CO₃ | cesium carbonate; | d | doublet; |
| dd | doublet of doublets; | dba | dibenzylideneacetone |
| DCM | dichloromethane; | DEA | diethylamine; |
| DEAD | diethyl azodicarboxylate; | DIPEA/DIEA | N,N-diisopropylethylamine; |
| DIAD | diisopropyl azodicarboxylate; | | |
| DMA | dimethylacetamide | DMF | N,N-dimethylformamide; |
| DMSO | dimethylsulfoxide; | | |
| EDC | 1-ethyl-3-(3-dimethylaminopropyl)carbodiimide; | | |
| EDTA | EthyleneDiamineTetraacetic Acid; | | |
| EtOAc | ethyl acetate; | EtOH | ethanol; |
| g | gram; | h | hour(s); |
| HEPES | (4-(2-hydroxyethyl)-1-piperazineethanesulfonic acid); | | |
| HPLC | high pressure liquid chromatography; | | |
| HTRF | Homogeneous Time Resolved Fluorescence; | | |
| kg | kilogram; | KRH | Krebs-Ringers Henseleit buffer; |
| L | liter; | LC | liquid chromatography; |
| LCMS | liquid chromatography and mass spectrometry; | | |
| LiOH | Lithium hydroxide; | MeOH | methanol; |
| MS | mass spectrometry; | M | molar; |
| m | multiplet; | m-CPBA | 3-Chloroperoxybenzoic acid; |
| Me | methyl; | min/min. | minutes; |
| mL | milliliter(s); | μM | micromolar; |
| m/z | mass to charge ratio; | nm | nanometer; |
| nM | nanomolar; | N | normal; |
| NMP | N-methylpyrrolidone; | NMR | nuclear magnetic resonance; |
| PBS-EDTA | Phosphate Buffered Saline-EthyleneDiamineTetraacetic Acid; | | |
| Pd(OAc)₂ | palladium(II) acetate; | PG | protecting group; |
| Ph | phenyl; | PS | polymer-supported; |
| rac | racemic; | rt | room temperature |
| s | singlet; | sat. | saturated; |
| SFC | supercritical fluid chromatography; | | |
| t | triplet; | t-Bu | tert-butyl; |
| TEA | triethylamine; | tol | toluene; |
| TFA | trifluoroacetic acid; | TFE | trifluoroethanol; |
| THF | tetrahydrofuran; | TLC | thin layer chromatography |
| X-Phos | 2-dicyclohexylphosphino-2′,4′,6′-triisopropylbiphenyl. | | |

General Synthetic Schemes. The following Examples were prepared, isolated and characterized using the methods disclosed herein. The following Examples demonstrate a partial scope of the disclosure and are not meant to limit the scope of the disclosure.

Unless specified otherwise, starting materials are generally available from a non-limiting commercial source such as TCI Fine Chemicals (Japan), Shanghai Chemhere Co., Ltd. (Shanghai, China), Aurora Fine Chemicals LLC (San Diego, CA), FCH Group (Ukraine), Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), AstraZeneca Pharmaceuticals (London, England), Chembridge Corporation (USA), Matrix Scientific (USA), Conier Chem & Pharm Co., Ltd (China), Enamine Ltd (Ukraine), Combi-Blocks, Inc. (San Diego, USA), Oakwood Products, Inc. (USA), Apollo Scientific Ltd. (UK), Allichem LLC. (USA) and Ukrorgsyntez Ltd (Latvia).

Schemes 1-4 (shown below) describe potential routes for preparing the compounds of the present disclosure which include compounds of Formula I and subformulas thereof. The starting materials for the below reaction scheme are commercially available or can be prepared according to methods known to one skilled in the art or by methods disclosed herein. Compounds of Formula I can be made substantially optically pure by either using substantially optically pure starting material or by separation chromatography, recrystallization or other separation techniques well-known in the art.

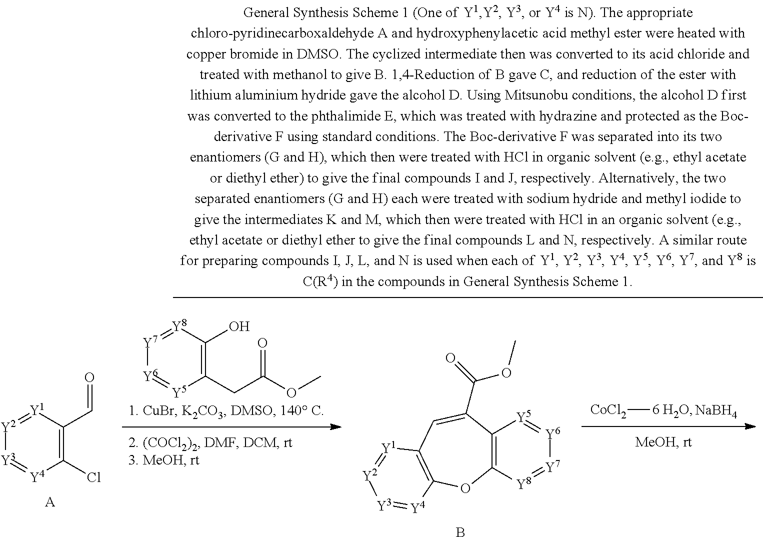

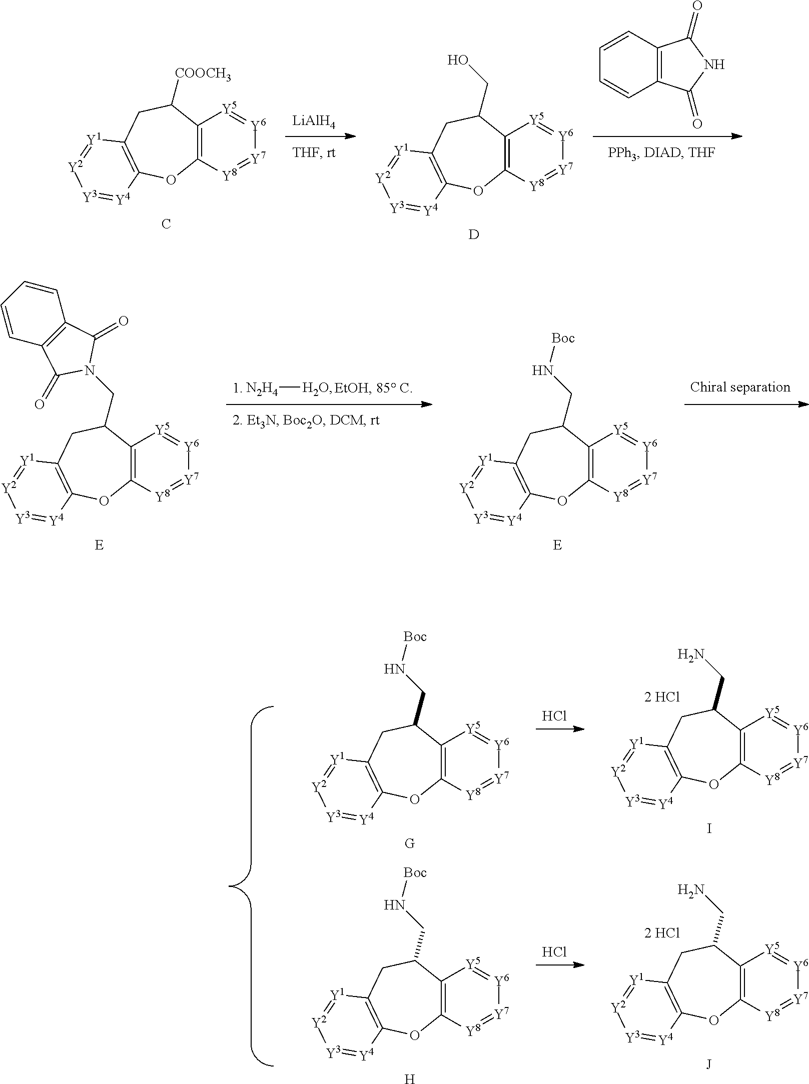

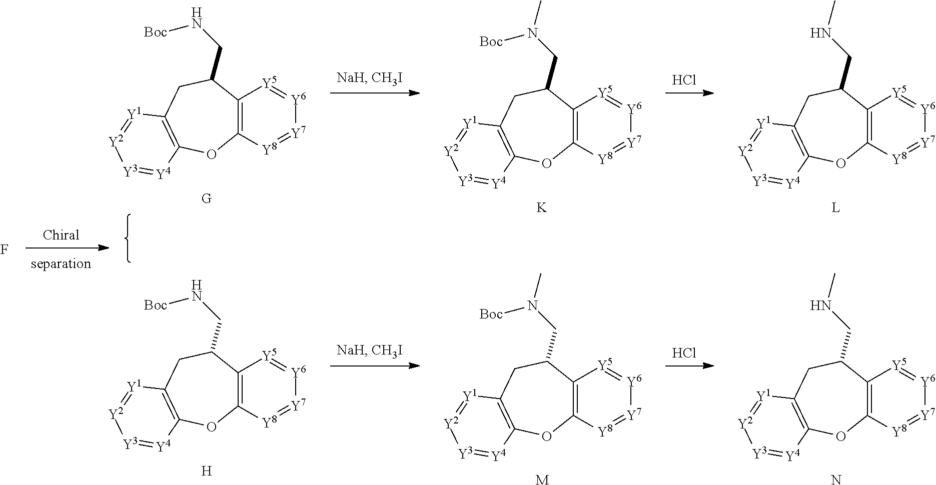

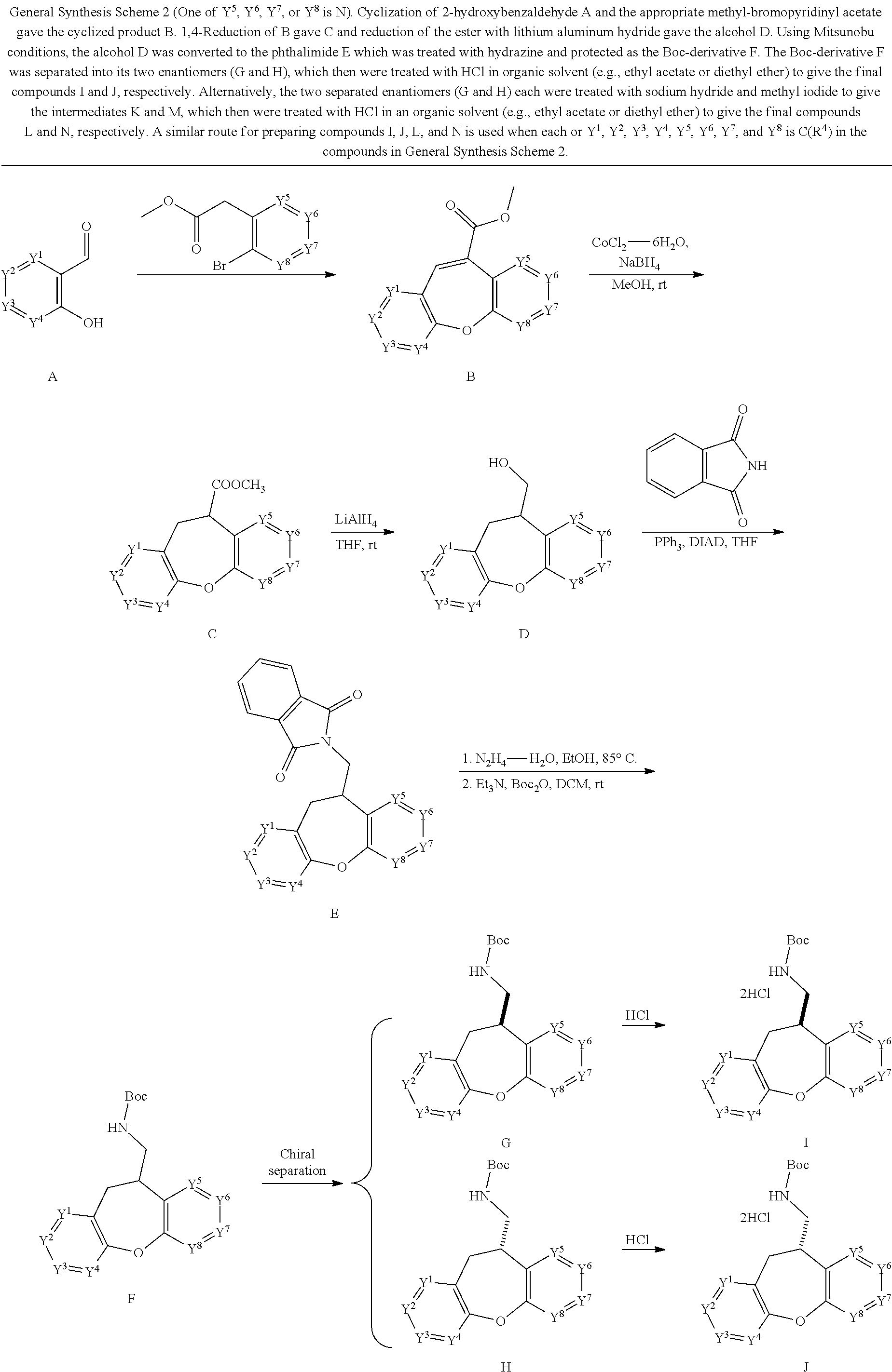

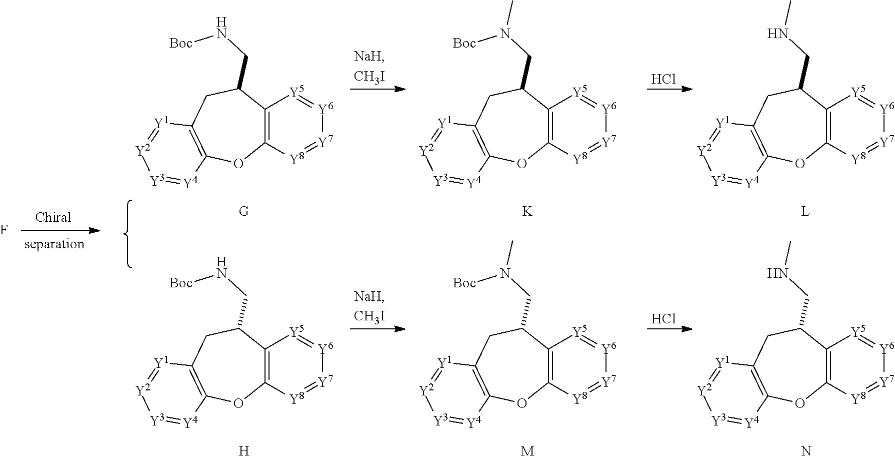

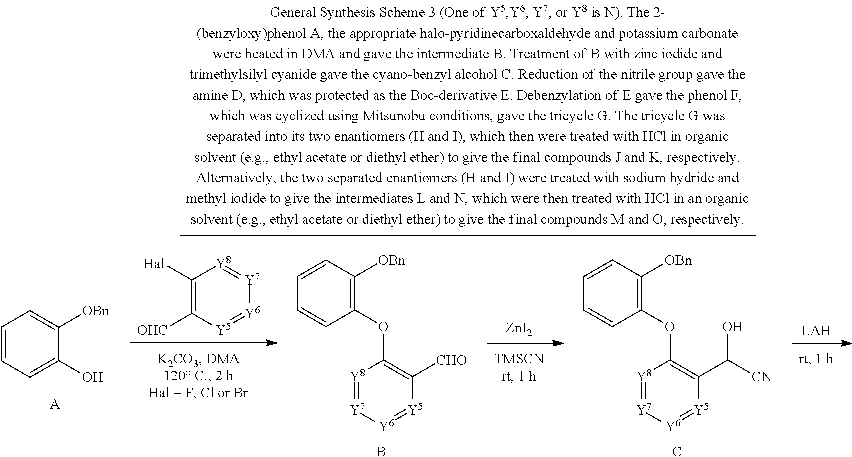

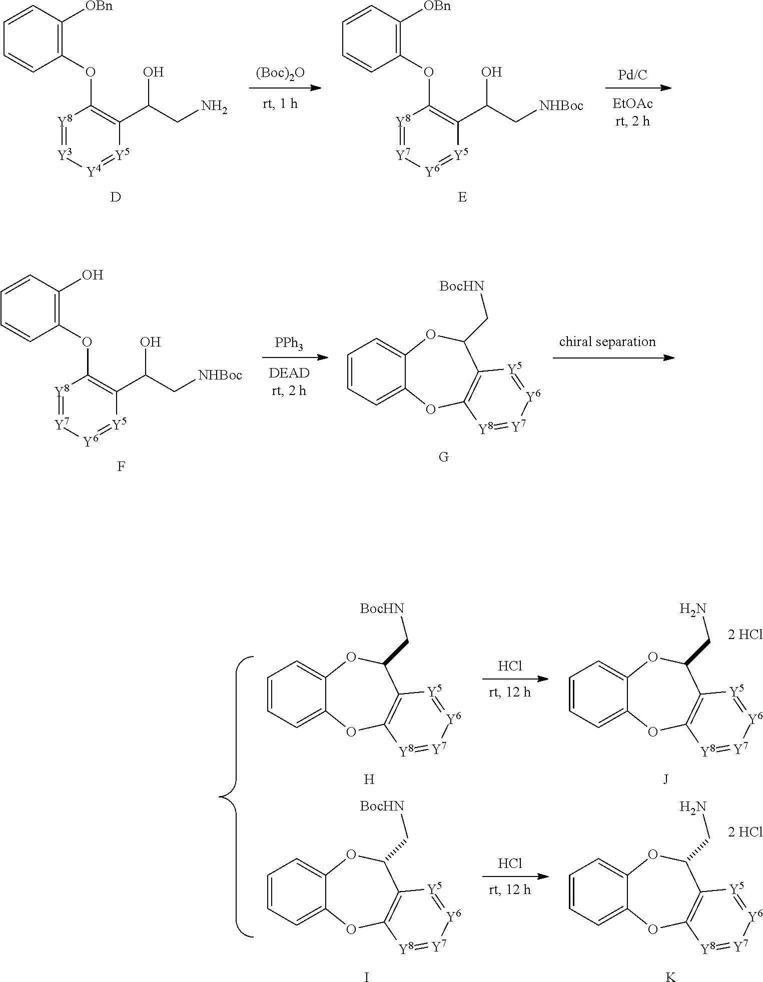

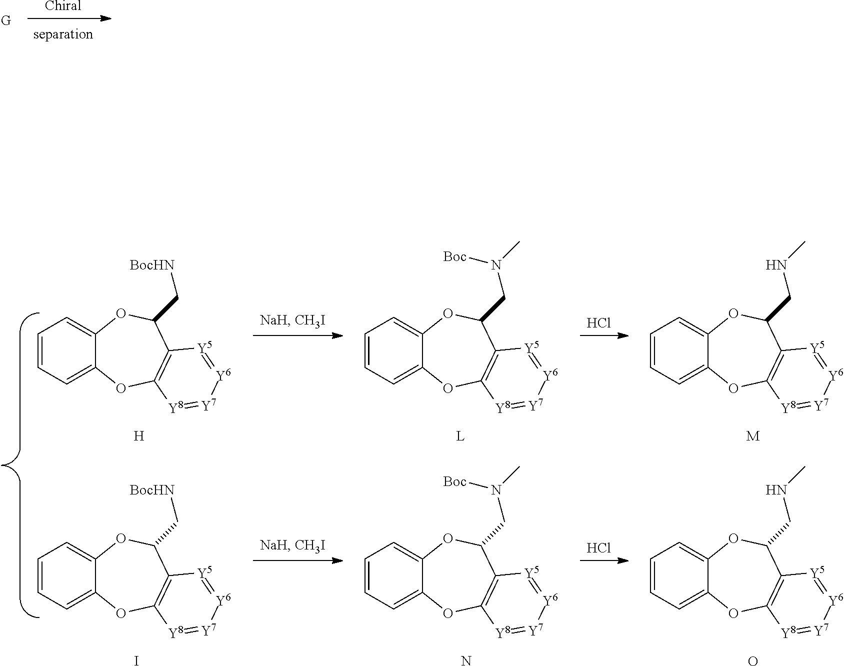

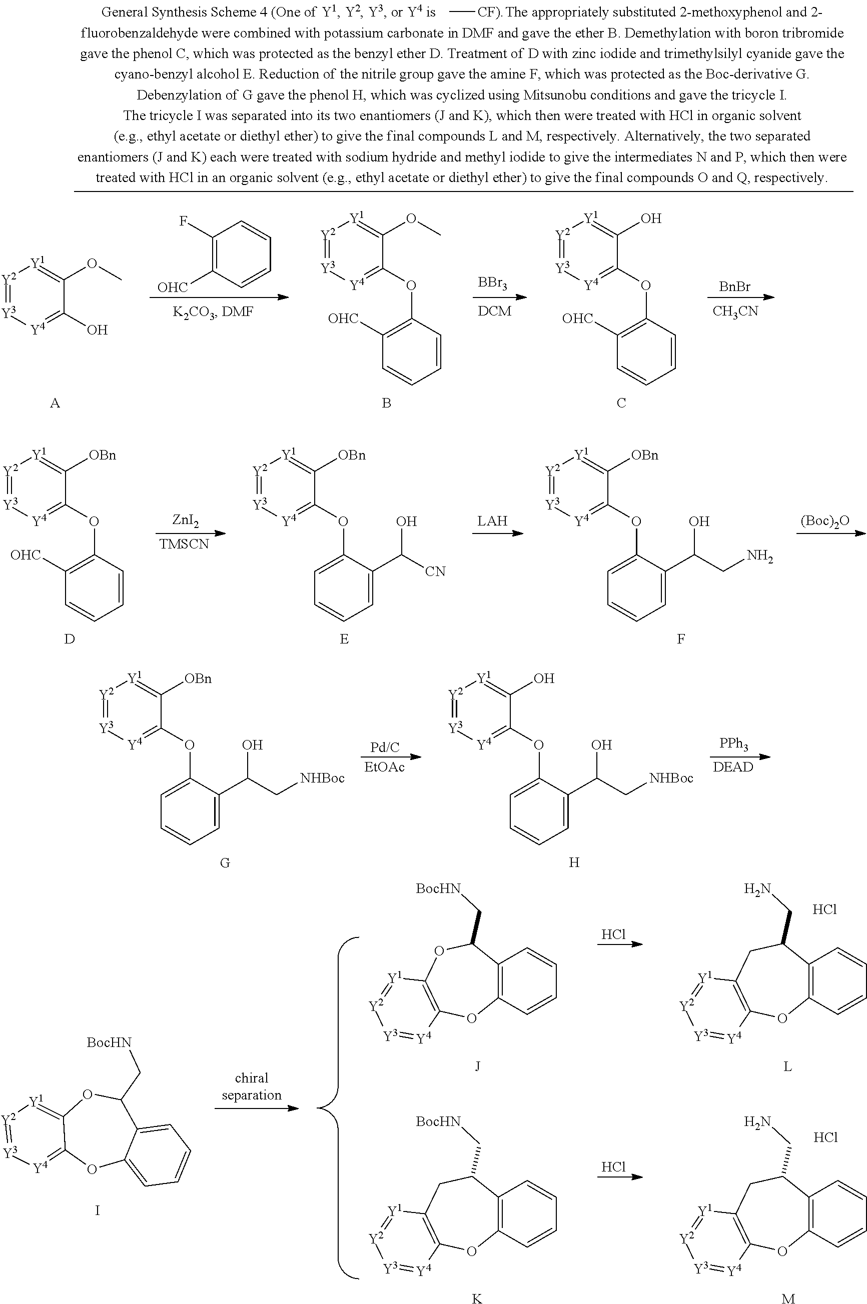

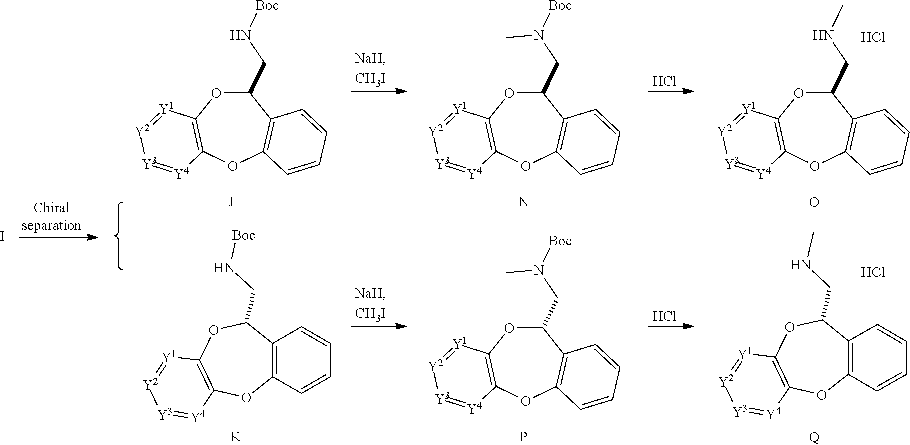

### Example 1. Synthesis of (R*)-(7-fluoro-10,11-dihydrodibenzo[b,f]oxepin-10-yl)methanamine (Compound 1) and (S*)-(7-fluoro-10,11-dihydrodibenzo[b,f]oxepin-10-yl)methanamine (Compound 2)

### a. Synthesis of 1-(2-(3-fluorophenoxy)phenyl)ethan-1-one

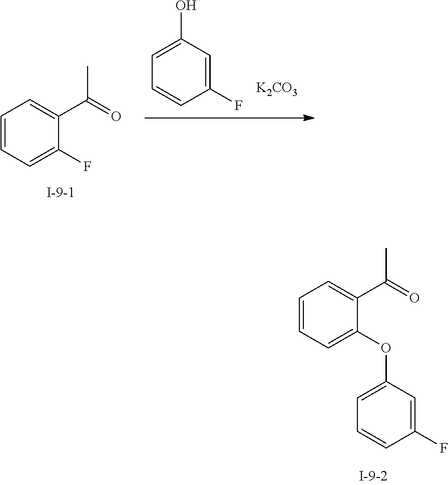

To a solution of 1-(2-fluorophenyl)ethan-1-one (13.8 g, 99.8 mmol) in DMF (50 mL) was added potassium carbonate (27.5 g, 199 mmol) and 3-fluorophenol (11.1 g, 99.8 mmol). The reaction was stirred overnight at about 90° C. Upon completion of the reaction, water (100 mL) and ethyl acetate (100 mL) were added to the reaction vessel and the resulting biphasic mixture was transferred to a separatory funnel. The layers were separated, and the aqueous phase was washed with ethyl acetate (2×100 mL). The combined organic layers were dried over anhydrous Na₂SO₄, filtered, and concentrated in vacuo. The resulting substance was purified by silica gel column chromatography with an isocratic elution of ethyl acetate (5%) and petroleum ether (95%) to provide 1-[2-(3-fluorophenoxy)phenyl]ethan-1-one.

### b. Synthesis of 2-(2-(3-fluorophenoxy)phenyl)acetic acid

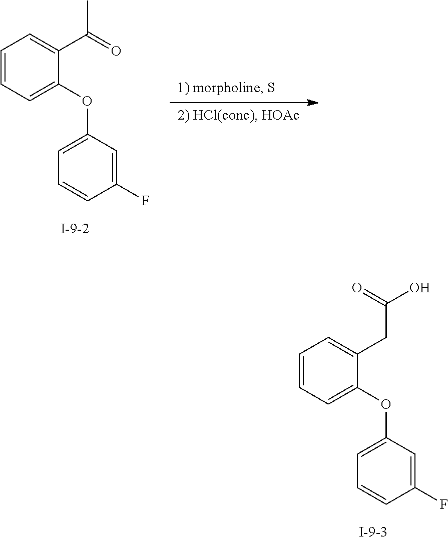

To a solution of 1-[2-(3-fluorophenoxy)phenyl]ethan-1-one (21 g, 91.2 mmol) was added morpholine (15.8 g, 182 mmol) and sulfur (5.82 g, 182 mmol). The mixture was heated to about 120° C. for about 5 hours. After cooling to about room temperature, conc. HCl (50 mL) and HOAc (100 mL) were added and the resulting mixture was stirred at reflux for about 2 hours. Upon completion of the reaction, the solvent was evaporated in vacuo. Then 200 mL of 4 N aq. NaOH was added, followed by ethyl acetate (200 mL). The water phase was separated, 6 N aq. HCl was added till pH ˜1. The mixture was extracted with ethyl acetate (200 mL), dried over Na₂SO₄ and evaporated in vacuo to give the crude product. After recrystallization in petroleum ether, 2-(2-(3-fluorophenoxy)phenyl)acetic acid was obtained. MS (ESI): m/z=247 [M+H]⁺.

### c. Synthesis of 7-fluorodibenzo[b,f]oxepin-10(11H)-one

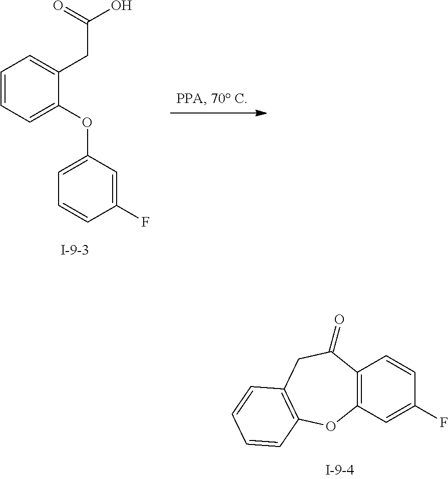 [SMILES: O=C(O)Cc1ccccc1Oc1cccc(F)c1 ; "PPA, 70° C." ; O=C1Cc2ccccc2Oc2cc(F)ccc21]

A solution of 2-[2-(3-fluorophenoxy)phenyl]acetic acid (7.5 g, 30.4 mmol) in polyphosphoric acid (60 mL) was heated at about 70° C. for about 8 h. Upon completion of the reaction, the mixture was quenched with water (200 mL), extracted with ethyl acetate (150 mL×2), dried and evaporated in vacuo and gave a residue which was purified by silica gel column chromatography eluted with petroleum ether/ethyl acetate=4:1 to yield 7-fluorodibenzo[b,f]oxepin-10(11H)-one. MS (ESI) m/z: 229 [M+H]⁺.

### d. Synthesis of ethyl (E)-2-(7-fluorodibenzo[b,f]oxepin-10(11H)-ylidene)acetate

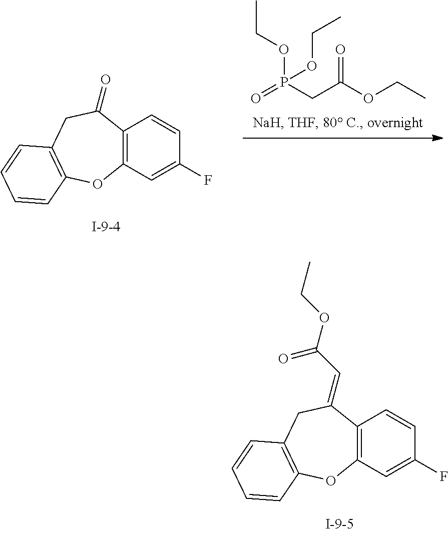

To a solution of 7-fluorodibenzo[b,f]oxepin-10(11H)-one (3 g, 13.1 mmol) in THF (40 mL) was added ethyl 2-(diethoxy phosphoryl)acetate (5.87 g, 26.2 mmol) and NaH (60% in mineral oil) (1.04 g, 26.2 mmol) under nitrogen at about room temperature. After addition, the mixture was heated overnight at reflux. Upon completion of the reaction, the mixture was diluted with ice water (200 mL), extracted with ethyl acetate (100 mL), dried and concentrated under reduced pressure. The resulting substance was purified by silica gel column chromatography using petroleum ether/ethyl acetate=5:1 to obtain ethyl (E)-2-(7-fluorodibenzo[b,f]oxepin-10(11H)-ylidene)acetate. MS (ESI) m/z: 299 [M+H]⁺.

### e. Synthesis of ethyl 2-(7-fluoro-10,11-dihydrodibenzo[b,f]oxepin-10-yl)acetate

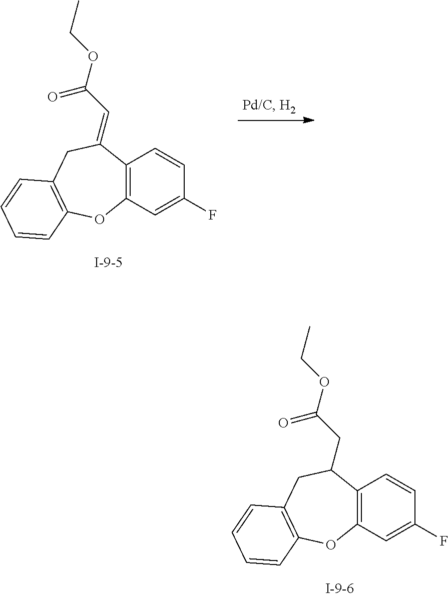 [SMILES: CCOC(=O)/C=C1\Cc2ccccc2Oc2cc(F)ccc21 ; "Pd/C, H2" ; CCOC(=O)CC1Cc2ccccc2Oc2cc(F)ccc21]

To a solution of ethyl (E)-2-(7-fluorodibenzo[b,f]oxepin-10(11H)-ylidene)acetate (1.25 g, 4.19 mmol) in EtOH (20 mL) was added Pd/C (10% wet) (222 mg). The mixture was hydrogenated at about room temperature for about 18 hours. Upon completion of the reaction, the mixture was filtered over diatomite and the filtrate was concentrated under reduced pressure to give ethyl 2-(7-fluoro-10,11-dihydrodibenzo[b,f]oxepin-10-yl)acetate. MS (ESI) m/z: 301 [M+H]⁺.

### f. Synthesis of 2-(7-fluoro-10,11-dihydrodibenzo[b,f]oxepin-10-yl)acetic acid

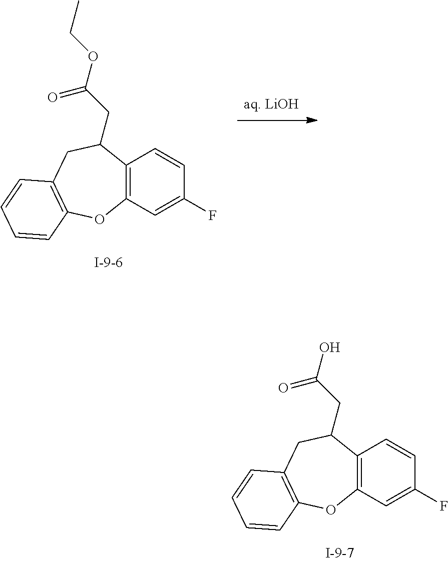 [SMILES: CCOC(=O)CC1Cc2ccccc2Oc2cc(F)ccc21 ; O=C(O)CC1Cc2ccccc2Oc2cc(F)ccc21]

To a solution of ethyl 2-(7-fluoro-10,11-dihydrodibenzo[b,f]oxepin-10-yl)acetate (1.25 g, 4.16 mmol) in EtOH/H₂O=1:1 (15 mL) was added LiOH·H₂O (520 mg, 12.4 mmol). The mixture was stirred at about room temperature for about 18 hours. Upon completion of the reaction, the mixture was acidified with 2 N aq. HCl till pH-3, extracted with ethyl acetate (40 mL×2), dried and concentrated under reduced pressure to give 2-(7-fluoro-10,11-dihydrodibenzo[b,f]oxepin-10-yl)acetic acid. MS (ESI) m/z: 295 [M+Na]⁺.

### g. Synthesis of 2-(7-fluoro-10,11-dihydrodibenzo[b,f]oxepin-10-yl)acetamide

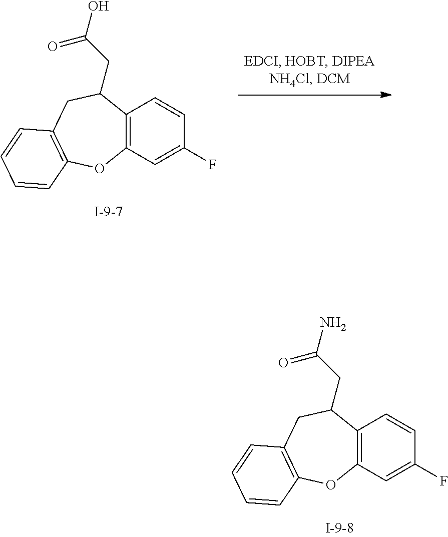 [SMILES: O=C(O)CC1Cc2ccccc2Oc2cc(F)ccc21 ; NC(=O)CC1Cc2ccccc2Oc2cc(F)ccc21]

To a solution of 2-(7-fluoro-10,11-dihydrodibenzo[b,f]oxepin-10-yl)acetic acid (1.15 g, 4.22 mmol) in DCM (30 mL) was added 3-(((ethylimino)methylene)amino)-N,N-dimethylpropan-1-amine hydrochloride (1.61 g, 8.44 mmol), 1-hydroxybenzotriazole (569 mg, 4.22 mmol) and N, N-diisopropylethylamine (2.72 g, 21.1 mmol). After stirring for about 30 min, NH₄Cl (674 mg 12.6 mmol) was added. The mixture was stirred at about room temperature for about 18 hours. Upon completion of the reaction, the mixture was quenched with 150 mL of water, extracted with DCM (80 mL×2), dried and concentrated under reduced pressure to give the crude product which was triturated in ether to yield 2-(7-fluoro-10,11-dihydrodibenzo[b,f]oxepin-10-yl)acetamide. MS (ESI) m/z: 272 [M+H]⁺.

### h. Synthesis of tert-butyl ((7-fluoro-10,11-dihydrodibenzo[b,f]oxepin-10-yl)methyl)carbamate

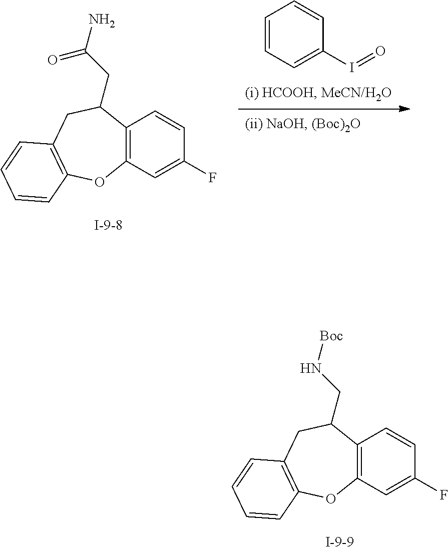 [SMILES: NC(=O)CC1Cc2ccccc2Oc2cc(F)ccc21 ; O=Ic1ccccc1 ; CC(C)(C)OC(=O)NCC1Cc2ccccc2Oc2cc(F)ccc21]

To a solution of formic acid (85% aq. 3.12 g, 58.0 mmol) in acetonitrile/water=3:1 (20 mL) was added iodosylbenzene (2.55 g, 11.6 mmol) at about room temperature. The mixture was stirred for about 15 min, then 2-(7-fluoro-10,11-dihydrodibenzo[b,f]oxepin-10-yl)acetamide (1.05 g, 3.87 mmol) in acetonitrile (3 mL) was added and the mixture was stirred for about 18 hours. The mixture was basified with 2 N aq. NaOH till pH>12 and di-tert-butyl dicarbonate (1.68 g, 7.74 mmol) was added. The mixture was stirred at about room temperature for about 2 hours. Upon completion of the reaction, the mixture was quenched with 100 mL of water, extracted with ethyl acetate (75 mL×2), dried and concentrated. The crude product was purified by silica gel column chromatography eluted with petroleum ether/ethyl acetate=8:1 to yield tert-butyl ((7-fluoro-10,11-dihydrodibenzo[b,f]oxepin-10-yl)methyl)carbamate. MS (ESI) m/z: 366 [M+Na]⁺.

### i. Chiral column separation of tert-butyl ((7-fluoro-10,11-dihydrodibenzo[b,f]oxepin-10-yl)methyl)carbamate

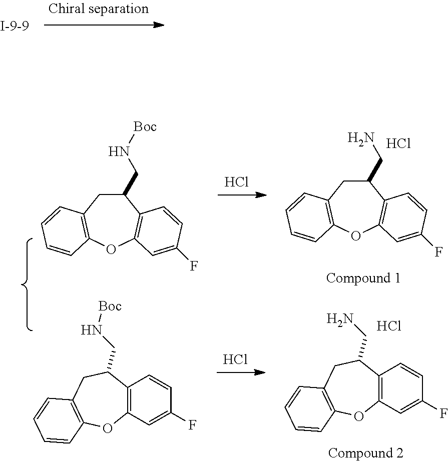

The tert-butyl ((7-fluoro-10,11-dihydrodibenzo[b,f]oxepin-10-yl)methyl)carbamate was separated by chiral column separation using:

| | |
|---|---|
| Instrument: SFC-80 (Thar, Waters) | Column: AD 20*250 |
| Column temperature: 35° C. | mm, 10 μm (Daicel) |
| Mobile phase: CO₂/MeOH (0.2% | |
| methanol in ammonia) = 87/13 | |
| Flow rate: 80 g/min | Back pressure: 100 bar |
| Detection wavelength: 214 nm | Cycle time: 3.6 min |
| Sample solution: 520 mg dissolved in | |
| 25 mL methanol | |
| Injection volume: 0.6 mL | |

After removal of the solvents, the first eluting isomer (I-9-9-P1) (180 mg, retention time=1.26 min) and the second eluting isomer (I-9-9-P2) (180 mg, retention time=1.56 min) were obtained.

### Synthesis of (R*)-(7-fluoro-10,11-dihydrodibenzo[b,f]oxepin-10-yl)methanamine (Compound 1) and (S*)-(7-fluoro-10,11-dihydrodibenzo[b,f]oxepin-10-yl)methanamine (Compound 2)

Independently, for each individual isomer I-9-9-P1 and I-9-9-P2, to a solution of each compound (80 mg, 0.23 mmol) in ethyl acetate (2 mL) was added HCl/ethyl acetate (3 M, 0.8 mL, 2.4 mmol). The mixture was stirred at about room temperature for about 18 hours. Upon completion of the reaction, the mixture was concentrated under reduced pressure to give Compound 1. MS (ESI) m/z: 244 [M+H]⁺. ¹H-NMR (500 MHz, CD₃OD) δ 7.33-7.30 (dd, J=6.5, 8.5 Hz, 1H), 7.28-7.26 (m, 2H), 7.22-7.20 (m, 1H), 7.15-7.12 (m, 1H), 7.05-7.03 (dd, J=2.5, 9.5 Hz, 1H), 6.97-6.93 (m, 1H), 3.56-3.53 (m, 1H), 3.46-3.42 (dd, J=4.0, 16.0 Hz, 1H), 3.32-3.28 (m, 1H), 3.23-3.15 (m, 2H). Chiral analysis column: AD-3 4.6*100 mm 3 μm, Acq. Method Set: AD 20% B1, Co-solvent: MeOH[0.2% NH₃ (7M in MeOH)], Run Time: 6.0 Minutes, Flow rate: 3.0 mL/min, Back pressure: 2000 psi, Column temperature: 40° C., retention time: 1.970 min.; and Compound 2. MS (ESI) m/z: 244 [M+H]⁺. ¹H-NMR (500 MHz, CD₃OD) δ 7.33-7.30 (dd, J=6.5, 8.5 Hz, 1H), 7.28-7.26 (m, 2H), 7.22-7.20 (m, 1H), 7.15-7.12 (m, 1H), 7.05-7.03 (dd, J=2.5, 9.5 Hz, 1H), 6.97-6.93 (m, 1H), 3.56-3.53 (m, 1H), 3.46-3.42 (dd, J=4.0, 16.0 Hz, 1H), 3.32-3.28 (m, 1H), 3.23-3.15 (m, 2H). Chiral analysis Column: AD-3 4.6*100 mm 3 μm, Acq. Method Set: AD 20% B1, Co-solvent: MeOH[0.2% NH₃ (7M in MeOH)], Run time: 6.0 Minutes, Flow rate: 3.0 mL/min, Back pressure: 2000 psi, Column temperature: 40° C., retention time: 1.558 min.

### Example 2. Synthesis of (R*)-1-(7-fluoro-10,11-dihydrodibenzo[b,f]oxepin-10-yl)-N-methylmethanamine (Compound 3) and (S*)-1-(7-fluoro-10,11-dihydrodibenzo[b,f]oxepin-10-yl)-N-methylmethanamine (Compound 4)

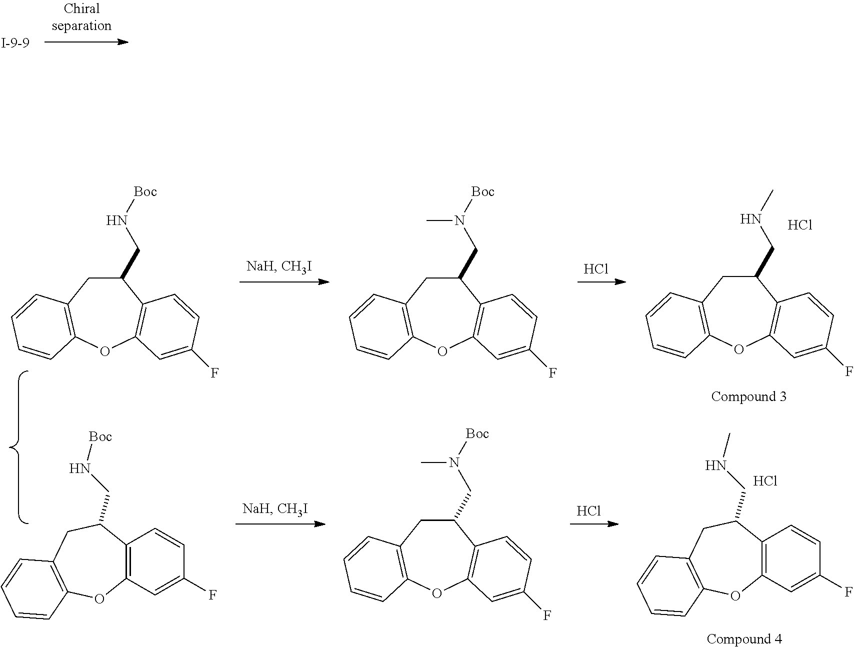

### Synthesis of (R*)-1-(7fluoro-10,1-dihydrodibenzo[b,f]oxepin-10-yl)-N-methylmethanamine (Compound 3) and (S*)-1-(7-fluoro-10,11-dihydrodibenzo[b,f]oxepin-10-yl)-N-methylmethanamine (Compound 4)

Independently, for each compound I-9-9-P1 and I-9-9-P2, to a solution of each compound (100 mg, 0.29 mmol) in DMF (3 mL) was added NaH (60% in mineral oil) (23.2 mg, 582 μmol) at about 0° C. After stirring for about 15 min, Mel (61.9 mg, 436 μmol) was added. The mixture was stirred at this temperature for 1 hour and then allowed to warm to room temperature. Upon completion of the reaction, the mixture was quenched with 50 mL of water, then extracted with ethyl acetate (20 mL×2), dried and evaporated. The substance was purified by prep-HPLC in 10 mmol/L aq. NH₄HCO₃ to give the N-methylated intermediate. MS (ESI) m/z: 358 [M+Na]⁺.

To a solution of each N-methylated intermediate (88 mg, 0.25 mmol) in ethyl acetate (3 mL) was added HCl in ethyl acetate (3M, 820 uL, 2.46 mmol). The mixture was stirred at about room temperature for about 18 hours. Upon completion of the reaction, the mixture was concentrated under reduced pressure to provide Compound 3. MS (ESI) m/z: 258 [M+H]⁺. ¹H-NMR (500 MHz, CD₃OD) δ 7.36 (dd, J=6.5, 9.0 Hz, 1H), 7.28-7.26 (m, 2H), 7.23-7.21 (m, 1 H), 7.14-7.11 (m, 1H), 7.06-7.04 (dd, J=2.5, 9.5 Hz, 1H), 6.98-6.94 (m, 1H), 3.64-3.60 (m, 1H), 3.45-3.41 (m, 2H), 3.32-3.29 (m, 1H), 3.21-3.17 (dd, J=6.0, 16.0 Hz, 1H), 2.73 (s, 3H). Chiral analysis Column: AY-H (250*4.6 mm 5 μm), Mobile phase: n-Hexane (0.1% DEA): EtOH (0.1% DEA)=90:10, Temperature: 40° C., Flow: 1.0 mL/min, Wavelength: 254 nm, Instrument: SHIMADZU, retention time: 4.365 min; and Compound 4. MS (ESI) m/z: 258 [M+H]⁺. ¹H-NMR (500 MHz, CD₃OD) δ 7.36 (dd, J=6.5, 9.0 Hz, 1H), 7.28-7.26 (m, 2H), 7.23-7.21 (m, 1H), 7.14-7.11 (m, 1H), 7.06-7.04 (dd, J=2.5, 9.5 Hz, 1H), 6.98-6.94 (m, 1H), 3.64-3.60 (m, 1H), 3.45-3.41 (m, 2H), 3.32-3.29 (m, 1H), 3.21-3.17 (dd, J=6.0, 16.0 Hz, 1H), 2.73 (s, 3H). Chiral analysis column: AY-H (250*4.6 mm 5 μm), Mobile Phase: n-Hexane (0.1% DEA):EtOH (0.1% DEA)=90:10, Temperature: 40° C., Flow: 1.0 mL/min, Wavelength: 254 nm, Instrument: SHIMADZU, retention time: 4.635 min.

### Example 5. Synthesis of (R*)-(1-fluoro-10,11-dihydrodibenzo[b,f]oxepin-10-yl)methanamine (Compound 80) and (S*)-(1-fluoro-10,11-dihydrodibenzo[b,f]oxepin-10-yl)methanamine (Compound 81)

### a. Preparation of ethyl 1-fluorodibenzo[b,f]oxepine-10-carboxylate

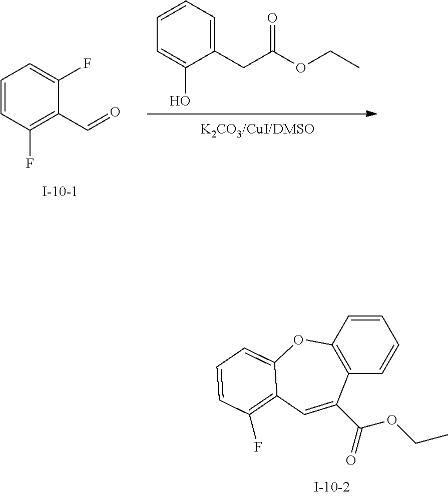 [SMILES: O=Cc1c(F)cccc1F ; CCOC(=O)Cc1ccccc1O ; CCOC(=O)C1=Cc2c(F)cccc2Oc2ccccc21]

To a solution of 2,6-difluorobenzaldehyde (4.76 g, 33.5 mmol) in DMSO (50 mL) was added ethyl 2-(2-hydroxyphenyl)acetate (5.5 g, 30.5 mmol), copper(I) iodide (1.16 g, 6.10 mmol) and potassium carbonate (8.43 g, 61.0 mmol) at about room temperature under N₂ atmosphere. The mixture was stirred at about 110° C. for about 4 hours. LCMS showed the reaction was completed. Water (50 mL) was added to the reaction vessel and the resulting mixture was extracted with ethyl acetate (3×100 mL). The combined organic phase was washed with saturated aqueous NaCl (3×50 mL). The combined organic layers were dried over anhydrous Na₂SO₄, filtered, and concentrated in vacuo. The resulting mixture was purified by silica gel column chromatography with a gradient elution of petroleum ether (100%) to petroleum ether (80%) and ethyl acetate (20%) to provide ethyl 1-fluorodibenzo[b,f]oxepine-10-carboxylate. MS (ESI): m/z 285 [M+H]⁺.

### b. Preparation of ethyl 1-fluoro-10, 11-dihydrodibenzo[b, f]oxepine-10-carboxylate

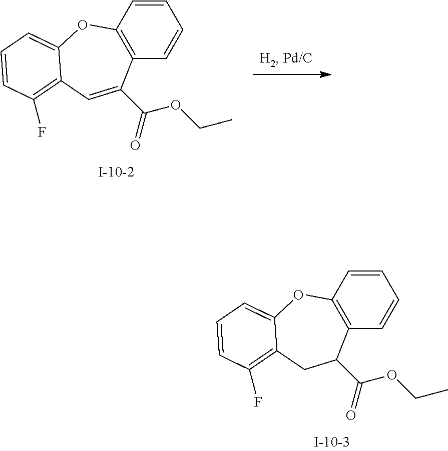 [SMILES: CCOC(=O)C1=Cc2c(F)cccc2Oc2ccccc21 ; CCOC(=O)C1Cc2c(F)cccc2Oc2ccccc21]

To a solution of ethyl 1-fluorodibenzo[b,f]oxepine-10-carboxylate (3.0 g, 10.56 mmol) in ethyl acetate/CH₃COOH (5:1, 30 mL) was added Pd/C (300 mg, 10% w/w). Then the mixture was stirred at about 45° C. for about 16 h under a hydrogen atmosphere. After filtration, the filtrate was concentrated to give a substance which was used for next step without further purification. MS (ESI): m/z 287 [M+H]⁺.

### c. Preparation of (1-fluoro-10, 11-dihydrodibenzo[b, f]oxepin-10-yl)methanol

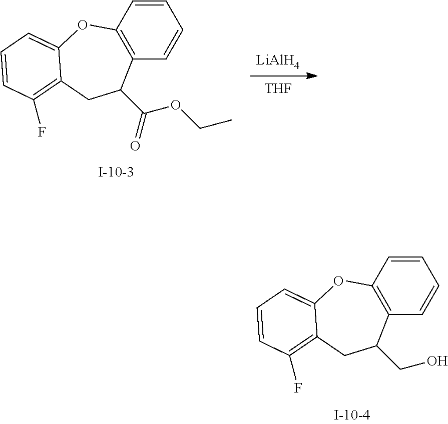 [SMILES: CCOC(=O)C1Cc2c(F)cccc2Oc2ccccc21 ; "LiAlH4" ; "THF" ; OCC1Cc2c(F)cccc2Oc2ccccc21]

To a solution of ethyl 1-fluoro-10, 11-dihydrodibenzo[b,f] oxepine-10-carboxylate (2.9 g, 10.12 mmol) in THE (30 mL) was added lithium aluminum hydride (770 mg, 20.24 mmol) at about 0° C. Then the mixture was stirred at about 0° C. for about 2 hours. The reaction was quenched with water (3.0 mL) and the mixture was filtrated. The filtrate was concentrated to give a substance which was used for the next step without further purification. MS (ESI): m/z 245 [M+H]⁺.

### d. Preparation of 2-((1-fluoro-10, 11-dihydrodibenzo[b,f]oxepin-10-yl)methyl)isoindoline-1,3-dione

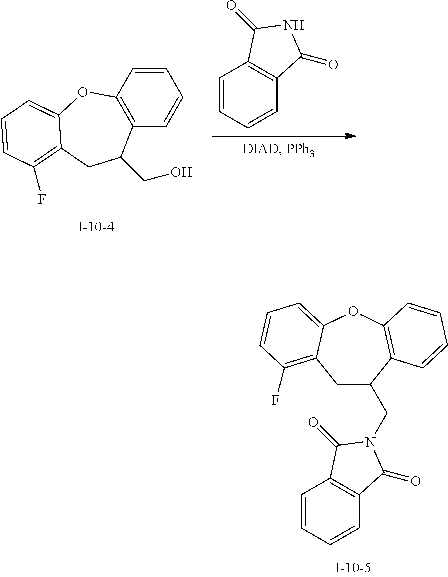

To a solution of (1-fluoro-10, 11-dihydrodibenzo[b,f]oxepin-10-yl)methanol (2.2 g, 9.0 mmol) in toluene (50 mL) was added isoindoline-1,3-dione (1.58 g, 10.8 mmol), triphenylphosphine (3.53 g, 13.49 mmol) and diisopropyl azodicarboxylate (2.35 g, 11.65 mmol) at about 0° C. under N₂ atmosphere. The mixture was stirred for about 2 hours, concentrated, and dissolved in MeOH (30 mL). The mixture was filtered, collected, and dried. MS (ESI): m/z 374 [M+H]⁺.

### e. Preparation of (1-fluoro-10, 11-dihydrodibenzo[b, f]oxepin-10-yl)methanamine

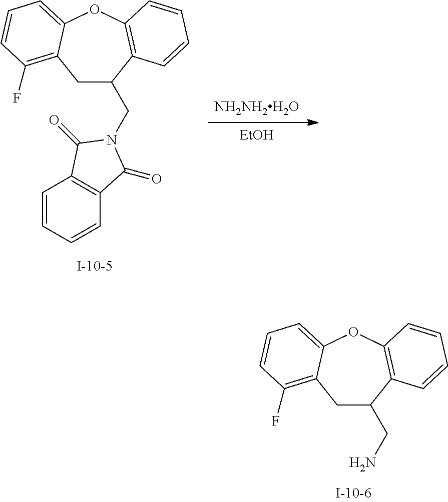 [SMILES: "EtOH" ; O=C1c2ccccc2C(=O)N1CC1Cc2c(F)cccc2Oc2ccccc21 ; "NH2NH2·H2O" ; NCC1Cc2c(F)cccc2Oc2ccccc21]

To a solution of 2-((1-fluoro-10, 11-dihydrodibenzo[b,f]oxepin-10-yl)methyl)isoindoline-1,3-dione (3.0 g, 8.04 mmol) in EtOH (100 mL) was added hydrazine hydrate (2.33 g, 46.7 mmol). The mixture was stirred at about 90° C. for about 4 hours. After cooling to about room temperature, the mixture was filtered, and the filtrate concentrated. MS (ESI): m/z 244 [M+H]⁺.

### f. Preparation of tert-butyl ((1-fluoro-10, 11-dihydrodibenzo[b, f]oxepin-10-yl)methyl)carbamate

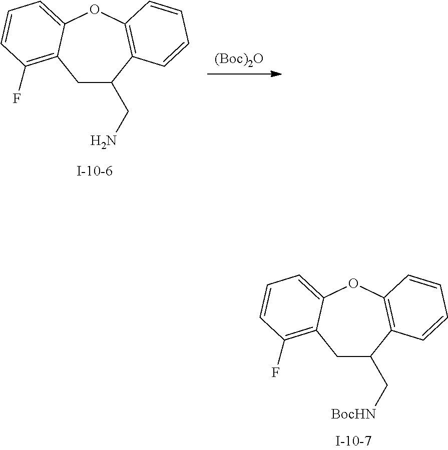 [SMILES: "(Boc)2O" ; NCC1Cc2c(F)cccc2Oc2ccccc21 ; CC(C)(C)OC(=O)NCC1Cc2c(F)cccc2Oc2ccccc21]

To a solution of (1-fluoro-10, 11-dihydrodibenzo[b,f]oxepin-10-yl)methanamine (1.5 g, 6.16 mmol) in DCM (50 mL) was added triethylamine (934 mg, 9.24 mmol) and di-tert-butyl dicarbonate (2.01 g, 9.24 mmol) at about room temperature and stirred at about room temperature for about 2 hours. Water (50 mL) was added to the reaction vessel and the resulting biphasic mixture was transferred to a separatory funnel. The layers were separated, and the aqueous phase was extracted with DCM (3×50 mL). The combined organic layers were dried over anhydrous Na₂SO₄, filtered, and concentrated in vacuo. The resulting mixture was purified by silica gel column chromatography with a gradient elution of petroleum ether (100%) to petroleum ether (90%) and ethyl acetate (10%) to provide tert-butyl ((1-fluoro-10, 11-dihydrodibenzo[b,f]oxepin-10-yl)methyl)carbamate. MS (ESI): m/z 288 [M-55]⁺.

### g. Chiral column separation of tert-butyl ((1-fluoro-10, 11-dihydrodibenzo[b, f]oxepin-10-yl)methyl)carbamate

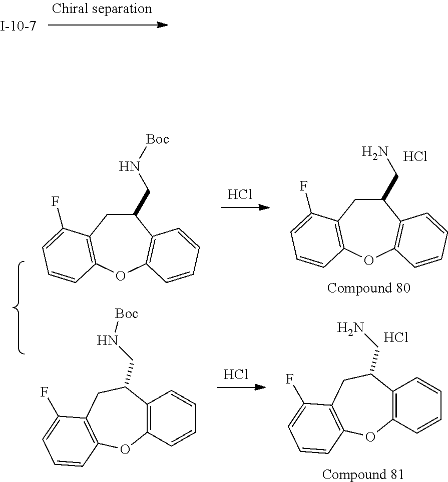

tert-butyl ((1-fluoro-10, 11-dihydrodibenzo[b, f]oxepin-10-yl)methyl)carbamate was separated by chiral column separation using the following conditions:

| | |
|---|---|
| Instrument: SFC-80 (Thar, Waters) | Column: AD 20 * 250 |
| Column temperature: 35° C. | mm, 10 μm (Daicel) |
| Mobile phase: CO₂/IPA (0.2% | |
| Methanol Ammonia) = 85/15 | |
| Flow rate: 80 g/min | Back pressure: 100 bar |
| Detection wavelength: 214 nm | Cycle time: 5.0 min |
| Sample solution: 2000 mg dissolved in | |
| 30 mL methanol | |
| Injection volume: 1.0 mL | |

After removal of the solvents, the first eluting isomer (I-10-7-P1) (800 mg, retention time=1.77 min) and the second eluting isomer (I-10-7-P2) (820 mg, retention time=2.16 min) were obtained.

### Synthesis of (R*)-(1-fluoro-10,11-dihydrodibenzo[b,f]oxepin-10-yl)methanamine (Compound 80) and (S*)-(1-fluoro-10,11-dihydrodibenzo[b,f]oxepin-10-yl)methanamine (Compound 81)

Independently, for each individual isomer I-10-7-P1 and I-10-7-P2, to a solution of each compound (350 mg, 1.01 mmol) in 3 M HCl/ethyl acetate (10 mL) was stirred at about room temperature for about 16 hours. After concentration, the substance was triturated with ethyl acetate (3×10 mL) and dried in vacuo to give Compound 80. MS (ESI): m/z 244 [M+H]⁺. ¹H NMR (500 MHz, CD₃OD) δ: 7.33 (t, J=7.8 Hz, 2H), 7.23 (dd, J=13.6, 7.1 Hz, 3H), 7.05 (d, J=8.3 Hz, 1H), 6.88 (t, J=8.9 Hz, 1H), 3.68-3.59 (m, 1H), 3.54 (dd, J=12.7, 8.1 Hz, 1H), 3.38 (dd, J=12.7, 7.0 Hz, 1H), 3.19 (qd, J=17.2, 5.2 Hz, 2H). Chiral analysis column: AD-H (250*4.6 mm 5 μm); Mobile Phase: n-Hexane (0.1% DEA):EtOH (0.1% DEA)=80:20; Temperature: 40° C.; Flow: 1.0 mL/min; Wavelength: 214 nm & 254 nm; Instrument: SHIMADZU; retention time=6.314 min.; and Compound 81. MS (ESI): m/z 244 [M+H]⁺. ¹H NMR (500 MHz, CD₃OD) δ: 7.33 (t, J=7.8 Hz, 2H), 7.23 (dd, J=13.6, 7.1 Hz, 3H), 7.05 (d, J=8.3 Hz, 1H), 6.88 (t, J=8.9 Hz, 1H), 3.68-3.59 (m, 1H), 3.54 (dd, J=12.7, 8.1 Hz, 1H), 3.38 (dd, J=12.7, 7.0 Hz, 1H), 3.19 (qd, J=17.2, 5.2 Hz, 2H). Chiral analysis column: AD-H (250*4.6 mm 5 μm); Mobile Phase: n-Hexane (0.1% DEA):EtOH (0.1% DEA)=80:20; Temperature: 40° C.; Flow: 1.0 mL/min; Wavelength: 214 nm & 254 nm; Instrument: SHIMADZU; retention time=7.225 min.

### Example 6. Synthesis of (R*)-1-(1-fluoro-10,11-dihydrodibenzo[b,f]oxepin-10-yl)-N-methylmethanamine (Compound 82) and (S*)-1-(1-fluoro-10,11-dihydrodibenzo[b,f]oxepin-10-yl)-N-methylmethanamine (Compound 83)

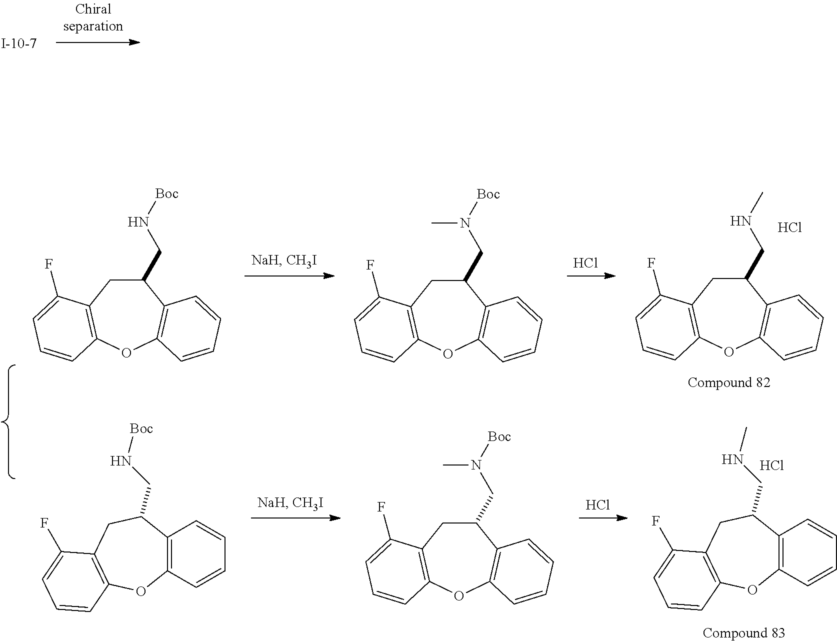

### Synthesis of (R*)-1-(1-fluoro-10,11-dihydrodibenzo[b,f]oxepin-10-yl)-N-methylmethanamine (Compound 82) and (S*)-1-(1-fluoro-10,11-dihydrodibenzo[b,f]oxepin-10-yl)-N-methylmethanamine (Compound 83)

Independently, for each compound I-10-7-P1 and I-10-7-P2, to a solution of each compound (450 mg, 1.31 mmol) in DMF (10 mL) was added sodium hydride (78.3 mg, 1.96 mmol) and iodomethane (278 mg, 1.96 mmol). Then the mixture was stirred at about room temperature for about 2 hours. Water (20 mL) was added to the reaction vessel and the mixture was extracted with ethyl acetate (3×20 mL). The organic layers were washed with saturated NaCl solution (3×20 mL). The combined organic layers were dried over anhydrous Na₂SO₄, filtered, and concentrated in vacuo. The resulting mixture was purified by silica gel column chromatography with a gradient elution of petroleum ether (100%) to petroleum ether (90%) and ethyl acetate (10%) to provide each N-methylated intermediate. MS (ESI): m/z 302 [M-55]⁺.

A solution of each N-methylated intermediate (400 mg, 1.11 mmol) in 3M HCl/ethyl acetate (10 mL) was stirred at about room temperature for about 16 hours. After concentration, the residue was washed with ethyl acetate (3×10 mL) and the solid was dried in vacuo and gave Compound 82. MS (ESI): m/z 258 [M+H]⁺. ¹H NMR (500 MHz, CD₃OD) δ: 7.37-7.31 (m, 2H), 7.27-7.20 (m, 3H), 7.06 (d, J=8.3 Hz, 1H), 6.92-6.84 (m, 1H), 3.71-3.64 (m, 2H), 3.45 (q, J=10.1 Hz, 1H), 3.20 (d, J=2.2 Hz, 2H), 2.72 (s, 3H). Chiral analysis column: OJ-H (250*4.6 mm 5 μm); Mobile Phase: n-Hexane (0.1% DEA):EtOH (0.1% DEA)=80:20; Temperature: 40° C.; Flow: 1.0 mL/min; Wavelength: 214 nm & 254 nm; Instrument: SHIMADZU; retention time=5.344 min.; and Compound 83. MS (ESI): m/z 258 [M+H]⁺. ¹H NMR (500 MHz, CD₃OD) δ: 7.42-7.32 (m, 2H), 7.29-7.16 (m, 3H), 7.06 (d, J=8.3 Hz, 1H), 6.97-6.81 (m, 1H), 3.73-3.61 (m, 2H), 3.45 (dd, J=16.1, 10.0 Hz, 1H), 3.24-3.15 (m, 2H), 2.72 (s, 3H). Chiral analysis column: OJ-H (250*4.6 mm 5 μm); Mobile Phase: n-Hexane (0.1% DEA):EtOH (0.1% DEA)=80:20; Temperature: 40° C.; Flow: 1.0 mL/min; Wavelength: 214 nm & 254 nm; Instrument: SHIMADZU; retention time=4.338 min.

### Example 13. Synthesis of (R*)-1-(5,6-dihydrobenzo[6,7]oxepino[2,3-b]pyridin-5-yl)-N-methylmethanamine (Compound 9) and (S*)-1-(5,6-dihydrobenzo[6,7]oxepino[2,3-b]pyridin-5-yl)-N-methylmethanamine (Compound 10)

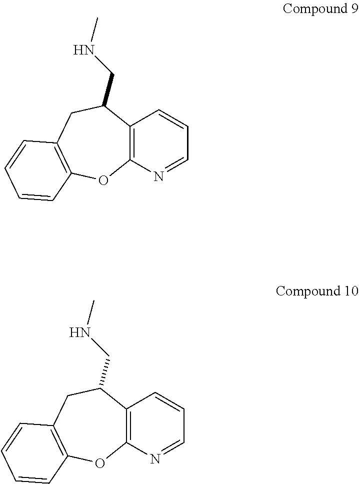 [SMILES: CNC[C@@H]1Cc2ccccc2Oc2ncccc21 ; CNC[C@H]1Cc2ccccc2Oc2ncccc21]

Compounds 9 and 10 are prepared similar to General Synthesis Scheme 2.

### Example 14. Synthesis of (R*)-(5,6-dihydrobenzo[6,7]oxepino[2,3-b]pyridin-5-yl)methanamine (Compound 11) and (S*)-(5,6-dihydrobenzo[6,7]oxepino[2,3-b]pyridin-5-yl)methanamine (Compound 12)

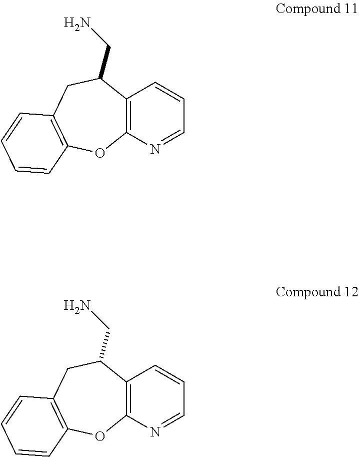 [SMILES: NC[C@@H]1Cc2ccccc2Oc2ncccc21 ; NC[C@H]1Cc2ccccc2Oc2ncccc21]

Compounds 11 and 12 are prepared similar to General Synthesis Scheme 2.

### Example 15. Synthesis of (R*)-(5,6-dihydrobenzo[6,7]oxepino[2,3-c]pyridin-5-yl)methanamine (Compound 13) and (S*)-(5,6-dihydrobenzo[6,7]oxepino[2,3-c]pyridin-5-yl)methanamine (Compound 14)

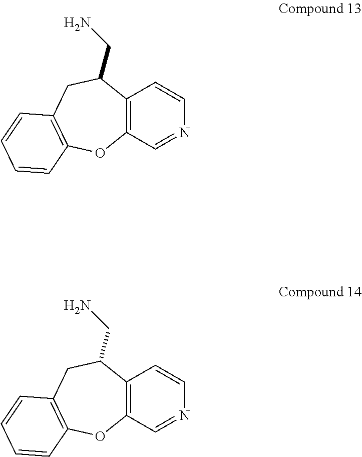 [SMILES: NC[C@@H]1Cc2ccccc2Oc2cnccc21 ; NC[C@H]1Cc2ccccc2Oc2cnccc21]

Compounds 13 and 14 are prepared similar to General Synthesis Scheme 2.

### Example 16. Synthesis of (R*)-1-(5,6-dihydrobenzo[6,7]oxepino[2,3-c]pyridin-5-yl)-N-methylmethanamine (Compound 15) and (s*)-1-(5,6-dihydrobenzo[6,7]oxepino[2,3-c]pyridin-5-yl)-N-methylmethanamine (Compound 16)

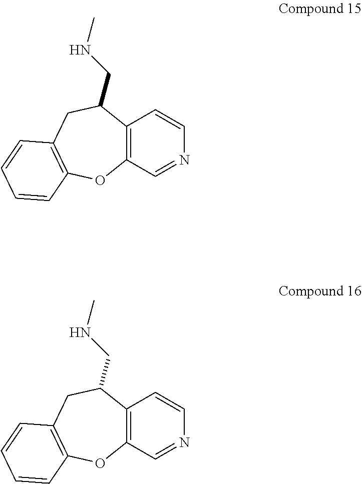 [SMILES: CNC[C@@H]1Cc2ccccc2Oc2cnccc21 ; CNC[C@H]1Cc2ccccc2Oc2cnccc21]

Compounds 15 and 16 are prepared similar to General Synthesis Scheme 2.

### Example 17. Synthesis of (R*)-(5,6-dihydrobenzo[6,7]oxepino[2,3-b]pyridin-6-yl)methanamine (Compound 17) and (S*)-(5,6-dihydrobenzo[6,7]oxepino[2,3-b]pyridin-6-yl)methanamine (Compound 18)

### a. Synthesis of ethyl benzo[6,7]oxepino[2,3-b]pyridine-6-carboxylate

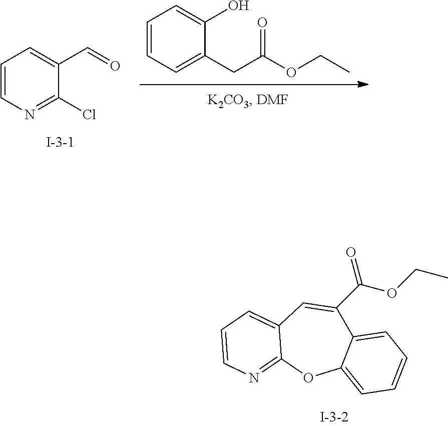

To a solution of 2-chloropyridine-3-carbaldehyde (5 g, 35.3 mmol) in DMF (100 mL) was added K₂CO₃ (9.74 g, 70.6 mmol) and ethyl 2-(2-hydroxyphenyl)acetate (6.36 g, 35.3 mmol). The mixture was heated to about 100° C. for about 18 hours. The mixture was quenched with water (300 mL), extracted with tert-butyl methyl ether (150 mL×2), dried and evaporated in vacuo to give the crude product which was purified by silica gel column chromatography, eluted with petroleum ether/ethyl acetate=3:1 to give ethyl benzo[6,7]oxepino[2,3-b]pyridine-6-carboxylate. MS (ESI) m/z: 268 [M+H]⁺.

### b. Synthesis of ethyl 5,6-dihydrobenzo[6,7]oxepino[2,3-b]pyridine-6-carboxylate

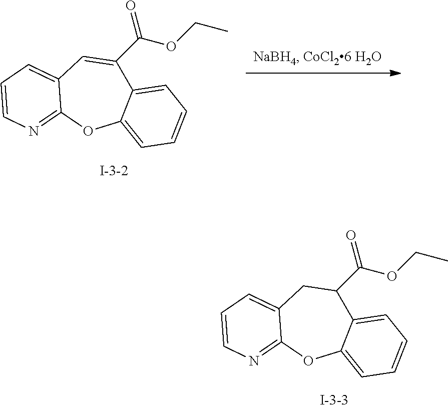 [SMILES: CCOC(=O)C1=Cc2cccnc2Oc2ccccc21 ; CCOC(=O)C1Cc2cccnc2Oc2ccccc21]

To a solution of ethyl benzo[6,7]oxepino[2,3-b]pyridine-6-carboxylate (3.1 g, 11.5 mmol) and cobalt chloride hexahydrate (1.36 g, 5.75 mmol) in MeOH (50 mL) was added NaBH₄ (4.33 g, 114 mmol) in portions. The mixture was stirred at about room temperature for 18 hours. NaBH₄ (1 g) was added to the reaction mixture. Upon completion of the reaction, the mixture was evaporated in vacuo to give a residue which was diluted with ethyl acetate (200 mL), washed with water (150 mL×2), dried and concentrated under reduced pressure to give ethyl 5,6-dihydrobenzo[6,7]oxepino[2,3-b]pyridine-6-carboxylate. MS (ESI) m/z: 270 [M+H]⁺.

### c. Synthesis of (5,6-dihydrobenzo[6,7]oxepino[2,3-b]pyridin-6-yl)methanol

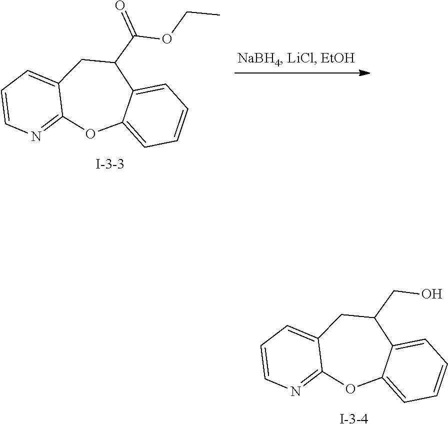 [SMILES: CCOC(=O)C1Cc2cccnc2Oc2ccccc21 ; OCC1Cc2cccnc2Oc2ccccc21]

To a solution of ethyl 5,6-dihydrobenzo[6,7]oxepino[2,3-b]pyridine-6-carboxylate (1.9 g, 7.05 mmol) and lithium chloride (597 mg, 14.1 mmol) in EtOH (30 mL) was added NaBH₄ (2.67 g, 70.4 mmol) in portions. The mixture was stirred at about room temperature for 18 hours. Additional NaBH₄ (0.5 g) was added until no starting material was left. The mixture was evaporated in vacuo and gave the residue which was diluted with ethyl acetate (200 mL), washed with water (150 mL×2), dried and concentrated under reduced pressure to give the crude product which was purified by silica gel column chromatography eluted with DCM/MeOH (20:1) to yield (5,6-dihydrobenzo[6,7]oxepino[2,3-b]pyridin-6-yl)methanol. MS (ESI) m/z: 229 [M+H]⁺.

### d. Synthesis of 2-((5,6-dihydrobenzo[6,7]oxepino[2,3-b]pyridin-6-yl)methyl)isoindoline-1,3-dione

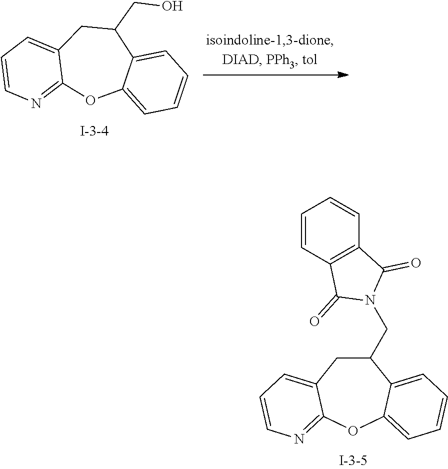 [SMILES: OCC1Cc2cccnc2Oc2ccccc21 ; O=C1c2ccccc2C(=O)N1CC1Cc2cccnc2Oc2ccccc21]

To a solution of (5,6-dihydrobenzo[6,7]oxepino[2,3-b]pyridin-6-yl)methanol (1 g, 5.72 mmol) in toluene (50 mL) was added PPh₃ (2.98 mg, 11.4 mmol) and DIAD (2.3 mg, 11.4 mmol) at about 0° C. under nitrogen. The mixture was stirred at this temperature for about 2 hours. The mixture was quenched with water (100 mL), extracted with ethyl acetate (80 mL), dried and concentrated to give the residue which was recrystallized in methanol and then filtered to afford 2-((5,6-dihydrobenzo[6,7]oxepino[2,3-b]pyridin-6-yl)methyl)isoindoline-1,3-dione. MS (ESI) m/z: 357 [M+H]⁺.

### e. Synthesis of tert-butyl ((5,6-dihydrobenzo[6,7]oxepino[2,3-b]pyridin-6-yl)methyl)carbamate

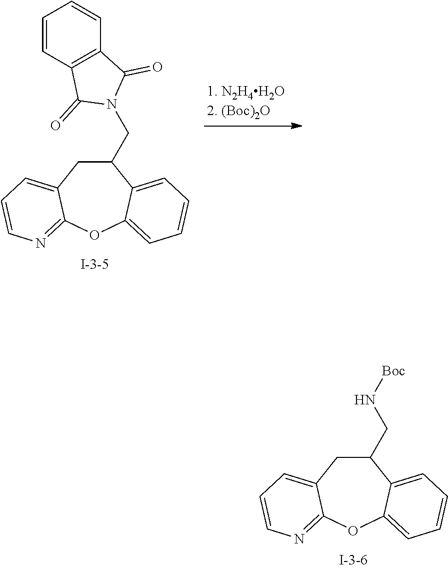 [SMILES: O=C1c2ccccc2C(=O)N1CC1Cc2cccnc2Oc2ccccc21 ; CC(C)(C)OC(=O)NCC1Cc2cccnc2Oc2ccccc21]

To a solution of 2-((5,6-dihydrobenzo[6,7]oxepino[2,3-b]pyridin-6-yl)methyl)isoindoline-1,3-dione (1.5 g, 4.2 mmol) in EtOH (50 mL) was added hydrazine hydrate (85% aq. 1.21 g, 21 mmol). The mixture was stirred at about 85° C. for about 2 hours. Then the mixture was cooled to room temperature, filtered to remove the solid, and the filtrate was evaporated in vacuo to dryness. To the resulting mixture in DCM (30 mL) was added di-tert-butyl dicarbonate (674 mg, 3.09 mmol) and triethylamine (1.05 g, 9.27 mmol) at about room temperature. The mixture was stirred at this temperature for about 2 hours. Upon the completion of the reaction, the mixture was washed with water (50 mL), dried and evaporated in vacuo to give the crude product which was purified by silica gel column chromatography with a gradient elution of petroleum ether (100%) to petroleum ether (90%) and ethyl acetate (10%) to afford tert-butyl ((5,6-dihydrobenzo[6,7]oxepino[2,3-b]pyridin-6-yl)methyl)carbamate. MS (ESI) m/z: 327 [M+H]⁺.

### f. Chiral column separation of tert-butyl ((5,6-dihydrobenzo[6,7]oxepino[2,3-b]pyridin-6-yl)methyl)carbamate

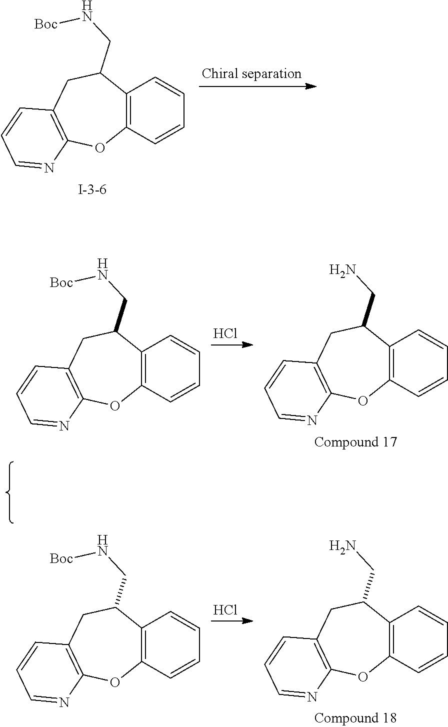

500 mg of tert-butyl ((5,6-dihydrobenzo[6,7]oxepino[2,3-b]pyridin-6-yl)methyl)carbamate was separated by chiral column and the following conditions:

| | |
|---|---|
| Instrument: SFC-150 (Waters) | Column: AD 20 × 250 |
| Column temperature: 35° C. | mm, 10 μm (Regis) |
| Mobile phase: CO₂/IPA(0.5% | |
| Methanol Ammonia) = 85/15 | |
| Flow rate: 100 g/min | Back pressure: 100 bar |
| Detection wavelength: 214 nm | Cycle time: 3.6 min |
| Sample solution: 500 mg dissolved in | |
| 30 mL methanol | |
| Injection volume: 1 mL | |

After removing solvents, the first eluting isomer I-3-6-P1 (180 mg, retention time=3.36 min) and I-3-6-P2 (180 mg, retention time=3.95 min) were obtained.

### Synthesis of (R*)-(5,6-dihydrobenzo[6,7]oxepino[2,3-b]pyridin-6-yl)methanamine (Compound 17) and (S*)-(5,6-dihydrobenzo[6,7]oxepino[2, 3-b]pyridin-6-yl)methanamine (Compound 18)

Independently, for each individual isomer I-3-6-P1 and I-3-6-P2, to a solution of each compound (90 mg, 0.28 mmol) in ethyl acetate (1 mL) was added HCl/ethyl acetate (3 M, 4 mL, 12 mmol) at about room temperature. The mixture was stirred at this temperature overnight. Upon completion of the reaction, the mixture was evaporated in vacuo to dryness and then freeze-dried on lyophilizer to yield Compound 17. MS (ESI): m/z 227 [M+H]⁺. ¹H NMR (400 MHz, CD₃OD): δ 8.46 (d, J=4.4 Hz, 1H), 8.36 (d, J=7.2 Hz, 1H), 7.64-7.61 (dd, J=5.6, 7.6 Hz, 1H), 7.51-7.37 (m, 4H), 3.76-3.70 (m, 1H), 3.56-3.37 (m, 4H). Chiral analysis Column: OZ-3 4.6*100 mm 3 μm, Acq. Method Set: OZ 25% B1, Cosolvent: MeOH [0.2% NH₃ (7M in MeOH)], Flow rate: 3.0 mL/min, Back Pressure: 2000 psi, Column Temperature: 40° C., retention time=1.764 min; and Compound 18. MS (ESI): m/z 227 [M+H]⁺. ¹H NMR (400 MHz, CD₃OD): δ 8.46 (d, J=4.4 Hz, 1H), 8.36 (d, J=7.2 Hz, 1H), 7.64-7.61 (dd, J=5.6, 7.6 Hz, 1H), 7.51-7.37 (m, 4H), 3.76-3.70 (m, 1H), 3.56-3.37 (m, 4H). Chiral analysis Column: OZ-3 4.6*100 mm 3 μm, Acq. Method Set: OZ 25% B1, Cosolvent: MeOH [0.2% NH₃ (7M in MeOH)], Flow rate: 3.0 mL/min, Back Pressure: 2000 psi, Column Temperature: 40° C., retention time=2.297 min.

### Example 18. Synthesis of (R*)-1-(5,6-dihydrobenzo[6,7]oxepino[2,3-b]pyridin-6-yl)-N-methylmethanamine (Compound 19) and (S*)-1-(5,6-dihydrobenzo[6,7]oxepino[2,3-b]pyridin-6-yl)-N-methylmethanamine (Compound 20)

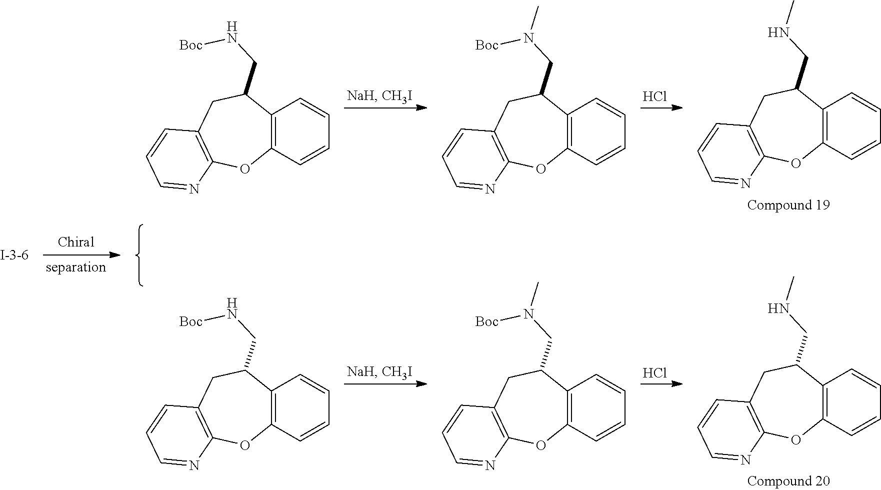

### Synthesis of (R*)-1-(5,6-dihydrobenzo[6,7]oxepino[2,3-b]pyridin-6-yl)-N-methylmethanamine (Compound 19) and (S*)-1-(5,6-dihydrobenzo[6,7]oxepino[2,3-b]pyridin-6-yl)-N-methylmethanamine (Compound 20)

Independently, for each individual isomer I-3-6-P1 and I-3-6-P2, to a solution of each compound (40 mg, 0.12 mmol) in DMF (2 mL) was added NaH (60% in mineral oil) (9.79 mg, 245 μmol) at about 0° C. After stirring for about 15 minutes, Mel (25.9 mg, 183 μmol) was added. The mixture was stirred at this temperature for about 30 minutes and then allowed to warm to about room temperature. Upon completion of the reaction, the mixture was quenched with 50 mL of water, extracted with ethyl acetate (20 mL×2), dried and evaporated in vacuo and gave the crude product which was purified by prep-HPLC in 10 mmol/L aq. NH₄HCO₃ to yield the N-methylated intermediate. MS (ESI) m/z: 363 [M+Na]⁺.

To a solution of each N-methylated intermediate (28 mg, 0.08 mmol) in ethyl acetate (1 mL) was added HCl/ethyl acetate (3M, 3 mL, 9 mmol) at about room temperature. The mixture was stirred at this temperature overnight. Upon completion of the reaction, the mixture was concentrated under reduced pressure to yield Compound 19. MS (ESI) m/z: 241 [M+H]⁺. ¹H-NMR (400 MHz, CD₃OD) δ 8.52-8.48 (m, 2H), 7.71-7.70 (m, 1H), 7.52-7.40 (m, 4H), 3.85 (m, 1H), 3.58-3.47 (m, 4H), 2.74 (s, 3H). Chiral analysis Column: AY-H (250*4.6 mm 5 μm), Mobile Phase: n-Hexane (0.1% DEA):EtOH (0.1% DEA)=80:20, Temperature: 40° C., Flow:1.0 mL/min, Wavelength: 254 nm, retention time=8.538 min; and Compound 20. MS (ESI) m/z: 241 [M+H]⁺. ¹H-NMR (400 MHz, CD₃OD) δ 8.52-8.48 (m, 2H), 7.71-7.70 (m, 1H), 7.52-7.40 (m, 4H), 3.85 (m, 1H), 3.58-3.47 (m, 4H), 2.74 (s, 3H). Chiral analysis Column: AY-H (250*4.6 mm 5 μm), Mobile Phase: n-Hexane (0.1% DEA):EtOH (0.1% DEA)=80:20, Temperature: 40° C., Flow:1.0 mL/min, Wavelength: 254 nm, retention time=6.646 min.

### Example 19. Synthesis of (R*)-(5,6-dihydrobenzo[6,7]oxepino[2,3-c]pyridin-6-yl)methanamine (Compound 21) and (S*)-(5,6-dihydrobenzo[6,7]oxepino[2,3-c]pyridin-6-yl)methanamine (Compound 22)

### a. Synthesis of methyl 5,6-dihydrobenzo[6,7]oxepino[2,3-c]pyridine-6-carboxylate

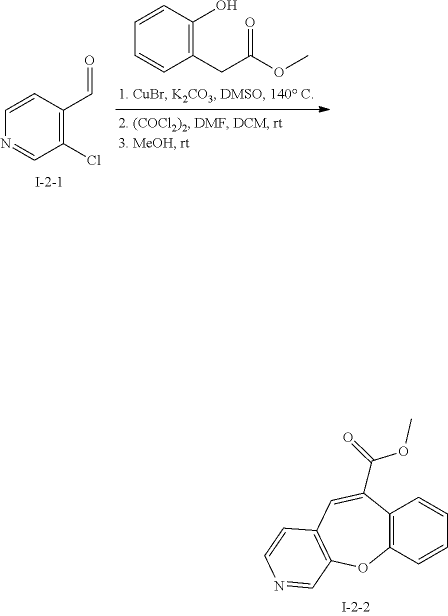

To a solution of 3-chloroisonicotinaldehyde (10.0 g, 70.9 mmol) in DMSO (150 mL) was added methyl 2-(2-hydroxyphenyl)acetate (11.8 g, 70.9 mmol), CuBr (10.2 g, 70.9 mmol) and K₂CO₃ (29.4 g, 212.8 mmol). The mixture was stirred at about 140° C. under Ar for about 100 hours. The reaction was cooled to room temperature. Water (800 mL) was added to the reaction vessel and the pH of the mixture was adjusted to about 6 using 3 M HCl aqueous solution. The mixture was extracted with ethyl acetate (3×300 mL). The combined organic layers were dried over anhydrous Na₂SO₄, filtered, and concentrated in vacuo. To the residue was added DCM (300 mL) and DMF (1 mL). Oxalyl chloride (27.0 g, 212.8 mmol) was added dropwise to above suspension. The reaction was stirred at about room temperature for about 2 hours. The mixture was concentrated, and to the residue was added MeOH (100 mL). The solution was stirred at about room temperature for about 20 minutes. The mixture was concentrated in vacuo. The resulting substance was purified by silica gel column chromatography with a gradient elution of petroleum ether (100%) to petroleum ether (70%) and ethyl acetate (30%) to provide methyl 5,6-dihydrobenzo[6,7]oxepino[2,3-c]pyridine-6-carboxylate. MS (ESI) m/z: 254 [M+H]⁺.

### b. Synthesis of methyl benzo[6,7]oxepino[2,3-c]pyridine-6-carboxylate

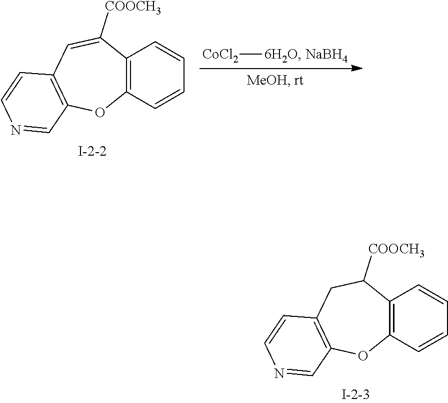

To a solution of methyl 5,6-dihydrobenzo[6,7]oxepino[2,3-c]pyridine-6-carboxylate (3.1 g, 12.3 mmol) in methanol (50 mL) was added COCl₂-6H₂O (2.92 g, 12.3 mmol) and NaBH₄ (1.4 g, 36.9 mmol). The reaction was stirred at about room temperature for about 2 hours. Water (50 mL) was added and the mixture was concentrated in vacuo to remove methanol. To the residual mixture was added DCM (50 mL). The mixture was filtered, and the filtrate was transferred to a separatory funnel. The layers were separated, and the aqueous phase was extracted with DCM (2×50 mL). The combined organic layers were dried over anhydrous Na₂SO₄, filtered, and concentrated in vacuo. The resulting substance was purified by silica gel column chromatography with a gradient elution of petroleum ether (100%) to petroleum ether (70%) and ethyl acetate (30%) to provide methyl benzo[6,7]oxepino[2,3-c]pyridine-6-carboxylate. MS (ESI) m/z: 256 [M+H]⁺. ¹H NMR (400 MHz, CDCl₃) δ 8.47 (s, 1H), 8.22 (d, J=4.8 Hz, 1H), 7.30-7.13 (m, 4H), 7.05 (d, J=4.8 Hz, 1H), 4.21-4.18 (m, 1H), 3.69 (s, 3H), 3.55-3.49 (m, 1H), 3.28-3.23 (m, 1H).

### c. Synthesis of (5,6-dihydrobenzo[6,7]oxepino[2,3-c]pyridin-6-yl)methanol

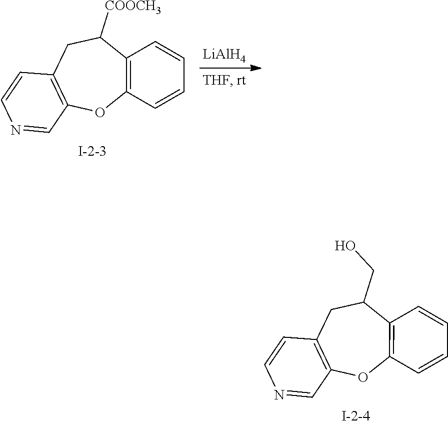

To a solution of methyl benzo[6,7]oxepino[2,3-c]pyridine-6-carboxylate (2.8 g, 11.0 mmol) in THF (40 mL) was added LiAlH₄ (418 mg, 11.0 mmol). The reaction was stirred at about room temperature for about 1 hour. Water (0.42 mL) was added slowly to quench the reaction. Then 15% NaOH (aq) (0.84 mL) and water (1.26 mL) were added. The mixture was filtered, and the filtrate was concentrated. Ethyl acetate (50 mL) was added to the residue and the mixture was washed with brine (2×20 mL). The organic layer was dried over anhydrous Na₂SO₄, filtered, and concentrated in vacuo. The resulting substance was purified by silica gel column chromatography with a gradient elution of petroleum ether (100%) to petroleum ether (70%) and ethyl acetate (30%) to provide (5,6-dihydrobenzo[6,7]oxepino[2,3-c]pyridin-6-yl)methanol. MS (ESI) m/z: 228 [M+H]⁺.

### d. Synthesis of 2-((5,6-dihydrobenzo[6,7]oxepino[2,3-c]pyridin-6-yl)methyl)isoindoline-1,3-dione

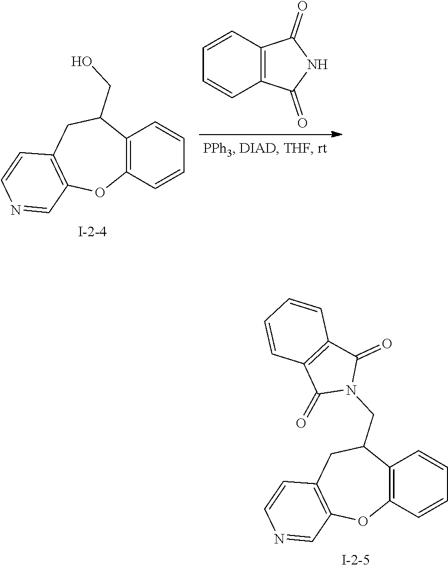 [SMILES: OCC1Cc2ccncc2Oc2ccccc21 ; O=C1NC(=O)c2ccccc21 ; O=C1c2ccccc2C(=O)N1CC1Cc2ccncc2Oc2ccccc21]

To a solution of (5,6-dihydrobenzo[6,7]oxepino[2,3-c]pyridin-6-yl)methanol (2.1 g, 9.25 mmol) in THF (30 mL) was added PPh₃ (4.84 g, 18.5 mmol) and DIAD (3.73 g, 18.5 mmol). The reaction was stirred at about room temperature for about 1 hour. Then the mixture was concentrated in vacuo. Methanol (10 mL) was added to the resulting substance and the mixture was stirred at about room temperature for about 30 minutes. The mixture was filtered and the solid was collected to provide 2-((5,6-dihydrobenzo[6,7]oxepino[2,3-c]pyridin-6-yl)methyl)isoindoline-1,3-dione. MS (ESI) m/z: 357 [M+H]⁺.

### e. Synthesis of tert-butyl ((5,6-dihydrobenzo[6,7]oxepino[2,3-c]pyridin-6-yl)methyl)carbamate

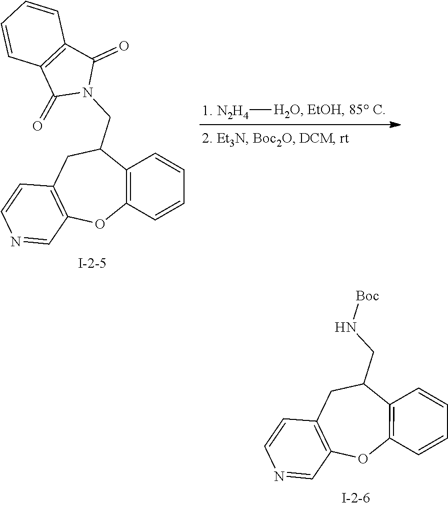

To a solution of 2-((5,6-dihydrobenzo[6,7]oxepino[2,3-c]pyridin-6-yl)methyl)isoindoline-1,3-dione (2.6 g, 7.30 mmol) in ethanol (30 mL) was added hydrazine hydrate (1.82 g, 36.5 mmol). The mixture was stirred at about 85° C. for about 3 hours. The reaction was cooled to room temperature and filtered. The filtrate was concentrated in vacuo. DCM (20 mL) was added to the residue and then Et₃N (1.47 g, 14.6 mmol) and Boc₂O (3.18 g, 14.6 mmol) were added. The mixture was stirred at about room temperature for about 1 hour. Saturated NaCl aqueous solution (15 mL) was added and the mixture was transferred to a separatory funnel. The layers were separated, and the aqueous phase was extracted with DCM (2×15 mL). The combined organic layers were dried over anhydrous Na₂SO₄, filtered, and concentrated in vacuo. The resulting substance was purified by silica gel column chromatography with a gradient elution of petroleum ether (100%) to petroleum ether (70%) and ethyl acetate (30%) to provide tert-butyl ((5,6-dihydrobenzo[6,7]oxepino[2,3-c]pyridin-6-yl)methyl)carbamate. MS (ESI) m/z: 327 [M+H]⁺. ¹H NMR (500 MHz, CDCl₃) δ 8.48 (s, 1H), 8.22 (d, J=4.5 Hz, 1H), 7.25-7.21 (m, 3H), 7.13-7.04 (m, 2H), 4.64 (bs, 1H), 3.52-3.29 (m, 4H), 3.02-2.98 (m, 1H), 1.44 (s, 9H).

### f. Chiral column separation of tert-butyl ((5,6-dihydrobenzo[6,7]oxepino[2,3-c]pyridin-6-yl)methyl)carbamate

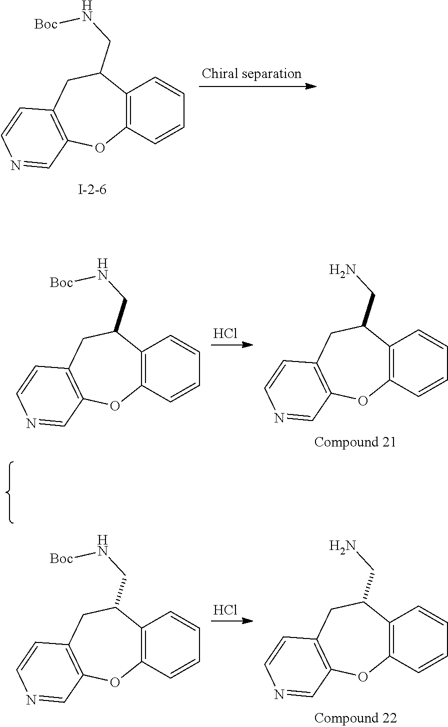

1.8 g of tert-butyl ((5,6-dihydrobenzo[6,7]oxepino[2,3-c]pyridin-6-yl)methyl)carbamate was separated by the following conditions:

| | |
|---|---|
| Instrument: SFC-150 (Waters) | Column: AD 20 × 250 |
| Column temperature: 35° C. | mm, 10 μm (Regis) |
| Mobile phase: CO₂/IPA(0.5% | |
| Methanol Ammonia) = 75/25 | |
| Flow rate: 100 g/min | Back pressure: 100 bar |
| Detection wavelength: 214 nm | Cycle time: 2.7 min |
| Sample solution: 1800 mg dissolved in | |
| 80 mL methanol | |
| Injection volume: 1 mL | |

After removing solvents, the first eluting isomer I-2-6-P1 (800 mg, retention time=1.456 min) and second eluting isomer I-2-6-P2 (700 mg, retention time=1.718 min) were obtained.

### Synthesis of (R*)-(5,6-dihydrobenzo[6,7]oxepino[2,3-c]pyridin-6-yl)methanamine (Compound 21) and (S*)-(5,6-dihydrobenzo[6,7]oxepino[2,3-c]pyridin-6-yl)methanamine (Compound 22)

Independently, for each individual isomer I-2-6-P1 and I-2-6-P2, a solution of each compound (100 mg, 306 μmol) in HCl/MeOH (3 M, 8 mL) was stirred at about room temperature for about 2 hours. The mixture was concentrated to dryness and to the residue was added ethyl acetate (10 mL). The mixture was stirred at about room temperature for about 10 minutes and then filtered. The substance was collected and dissolved in water (10 mL). The mixture was freeze-dried on lyophilizer to provide Compound 21. MS (ESI) m/z: 227 [M+H]⁺. ¹H NMR (400 MHz, CD₃OD) δ 8.94 (s, 1H), 8.53 (d, J=6.0 Hz, 1H), 7.96 (d, J=5.6 Hz, 1H), 7.47-7.42 (m, 3H), 7.38-7.33 (m, 1H), 3.79-3.68 (m, 2H), 3.58-3.44 (m, 3H). Chiral analysis: method info: AD-H 10% MeOH [0.2% NH₃ (7M in MeOH)]; flow: 4 mL/min; temperature: 40° C.; retention time=2.79 min; and Compound 22. MS (ESI) m/z: 227 [M+H]⁺. ¹H NMR (400 MHz, CD₃OD) δ 8.93 (s, 1H), 8.53 (d, J=6.0 Hz, 1H), 7.96 (d, J=5.6 Hz, 1H), 7.47-7.42 (m, 3H), 7.37-7.33 (m, 1H), 3.79-3.67 (m, 2H), 3.58-3.44 (m, 3H). Chiral analysis: method info: AD-H 10% MeOH [0.2% NH₃ (7M in MeOH)]; flow: 4 mL/min; temperature: 40° C.; retention time=3.28 min.

### Example 20. Synthesis of (R*)-1-(5,6-dihydrobenzo[6,7]oxepino[2,3-c]pyridin-6-yl)-N-methylmethanamine (Compound 23) and (S*)-1-(5,6-dihydrobenzo[6,7]oxepino[2,3-c]pyridin-6-yl)-N-methylmethanamine (Compound 24)

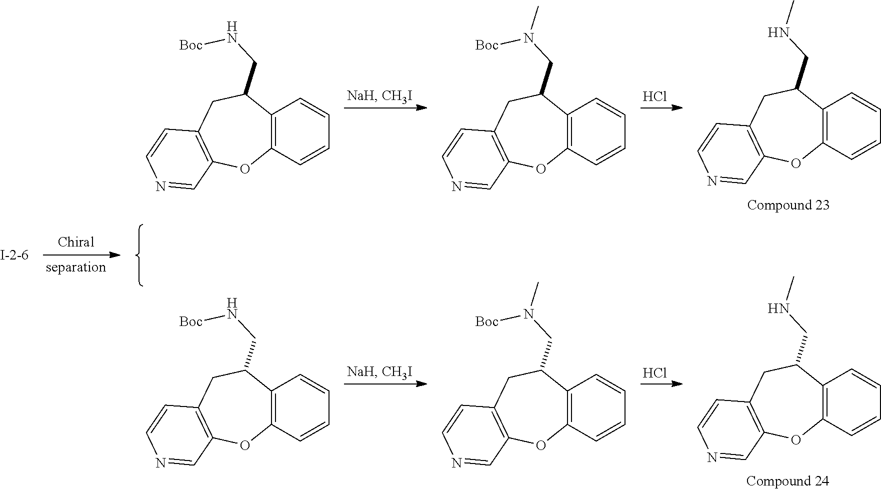

### Synthesis of (R*)-1-(5,6-dihydrobenzo[6,7]oxepino[2,3-c]pyridin-6-yl)-N-methylmethanamine (Compound 23) and (S*)-1-(5,6-dihydrobenzo[6,7]oxepino[2,3-c]pyridin-6-yl)-N-methylmethanamine (Compound 24)

Independently, for each individual isomer I-2-6-P1 and I-2-6-P2, to a solution of each compound (500 mg, 1.53 mmol) in DMF (8 mL) was added NaH (60%) (183 mg, 4.59 mmol) at about 0° C. The mixture was stirred at about 0° C. for about 10 minutes. Then CH₃I (651 mg, 4.59 mmol) was added. The reaction was stirred at about 0° C. for about 2 hours. Saturated aqueous NH₄Cl (30 mL) was added to the reaction vessel and the mixture was extracted with ethyl acetate (3×20 mL). The combined organic layers were dried over anhydrous Na₂SO₄, filtered, and concentrated in vacuo. The resulting substance was purified by silica gel column chromatography with a gradient elution of DCM (100%) to DCM (98%) and MeOH (2%) to provide the N-methylated intermediate (MS (ESI) m/z: 341 [M+H]⁺).

A solution of the N-methylated intermediate (350 mg, 1.02 mmol) in HCl/MeOH (3M, 30 mL) was stirred at about room temperature for about 2 hours. The mixture was concentrated and to the residue was added ethyl acetate/MeOH (20/1) (15 mL). The mixture was stirred at about room temperature for about 10 minutes and then filtered. The substance was collected and dissolved in water (30 mL). The mixture was freeze-dried on lyophilizer to provide Compound 23. MS (ESI) m/z: 241 [M+H]⁺. ¹H NMR (500 MHz, CD₃OD) δ 8.94 (s, 1H), 8.54 (dd, J=1.0, 6.0 Hz, 1H), 7.97 (d, J=6.0 Hz, 1H), 7.49-7.43 (m, 3H), 7.38-7.35 (m, 1H), 3.87-3.84 (m, 1H), 3.73-3.51 (m, 4H), 2.76 (s, 3H). Chiral analysis column: IC (4.6×250 mm 5 μm); mobile phase: n-Hexane (0.1% DEA):EtOH (0.1% DEA)=80:20; wavelength: 220 nm; flow rate: 1 mL/min; temperature: 40° C.; retention time=12.233 min; and Compound 24. MS (ESI) m/z: 241 [M+H]⁺. ¹H NMR (500 MHz, CD₃OD) δ 8.94 (s, 1H), 8.54 (d, J=6.0 Hz, 1H), 7.97 (d, J=5.5 Hz, 1H), 7.49-7.43 (m, 3H), 7.38-7.35 (m, 1H), 3.87-3.84 (m, 1H), 3.73-3.51 (m, 4H), 2.76 (s, 3H). Chiral analysis column: IC (4.6×250 mm 5 μm); mobile phase: n-Hexane (0.1% DEA):EtOH (0.1% DEA)=80:20; wavelength: 220 nm; flow rate: 1 mL/min; temperature: 40° C.; retention time=10.280 min.

### Example 21. Synthesis of 2-((10,11-dihydrobenzo[6,7]oxepino[3,2-c]pyridin-10-yl)methyl)isoindoline-1,3-dione (I-4-5)

### a. Synthesis of ethyl benzo[6,7]oxepino[3,2-c]pyridine-10-carboxylate

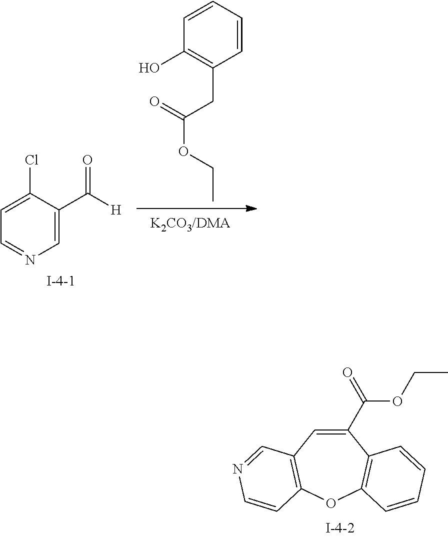 [SMILES: O=Cc1cnccc1Cl ; CCOC(=O)Cc1ccccc1O ; CCOC(=O)C1=Cc2cnccc2Oc2ccccc21]

To a solution of 4-chloronicotinaldehyde (8.3 g, 55.6 mmol) and ethyl 2-(2-hydroxyphenyl)acetate (10.5 g, 55.6 mmol) in dimethylacetamide (5 mL) was added potassium carbonate (15.6 g, 111 mmol), The reaction was stirred at about room temperature for about 2 hours. Water (100 mL) and ethyl acetate (100 mL) were added to the reaction vessel and the resulting biphasic mixture was transferred to a separatory funnel. The layers were separated, and the aqueous phase was extracted with ethyl acetate (3×100 mL). The combined organic layers were dried over anhydrous Na₂SO₄, filtered, and concentrated in vacuo. The resulting substance was purified by silica gel column chromatography with an isocratic elution of petroleum ether (60%) and ethyl acetate (40%) to provide ethyl benzo[6,7]oxepino[3,2-c]pyridine-10-carboxylate. MS (ESI): m/z=267.9. [M+H]⁺. ¹H NMR (400 MHz, CDCl₃) δ 8.55-8.43 (m, 2H), 7.76 (s, 1H), 7.46-7.41 (m, 1H), 7.30 (td, J=7.7, 1.6 Hz, 1H), 7.14 (dd, J=12.1, 4.5 Hz, 2H), 7.05 (d, J=5.5 Hz, 1H), 4.43-4.20 (m, 2H), 1.39-1.25 (m, 3H).

### b. Synthesis of ethyl 10,11-dihydrobenzo[6,7]oxepino[3,2-c]pyridine-10-carboxylate

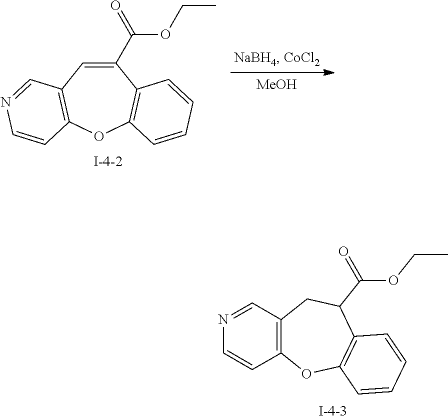 [SMILES: "MeOH" ; CCOC(=O)C1=Cc2cnccc2Oc2ccccc21 ; CCOC(=O)C1Cc2cnccc2Oc2ccccc21]

To a solution of ethyl benzo[6,7]oxepino[3,2-c]pyridine-10-carboxylate (3.8 g, 13.9 mmol) in methanol (40 mL) was added cobalt chloride hexahydrate (674 mg, 2.78 mmol) and NaBH₄ (1.60 g, 41.7 mmol) was added in some portions. The reaction was stirred at about room temperature for about 24 hours. To the mixture was added 1M HCl to adjust the pH to 6. The mixture was concentrated to remove solvent. Water (30 mL) and ethyl acetate (50 mL) were added to the reaction vessel and the resulting biphasic mixture was transferred to a separatory funnel. The layers were separated, and the aqueous phase was washed with ethyl acetate (3×30 mL). The combined organic layers were dried over anhydrous Na₂SO₄, filtered, and concentrated in vacuo. The resulting substance was purified by silica gel column chromatography with an isocratic elution of DCM (80%) and MeOH (20%) to provide ethyl 10,11-dihydrobenzo[6,7]oxepino[3,2-c]pyridine-10-carboxylate. MS (ESI): m/z=270.0 [M+H]⁺.

### c. Synthesis of (10,11-dihydrobenzo[6,7]oxepino[3,2-c]pyridin-10-yl)methanol

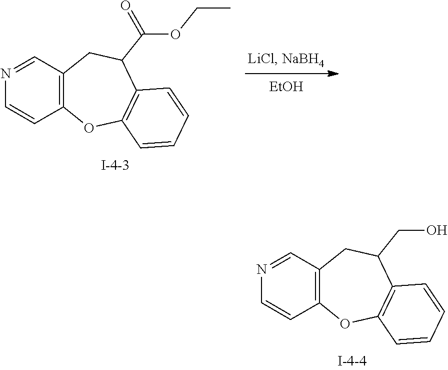 [SMILES: "EtOH" ; CCOC(=O)C1Cc2cnccc2Oc2ccccc21 ; OCC1Cc2cnccc2Oc2ccccc21]

To a solution of ethyl 10,11-dihydrobenzo[6,7]oxepino[3,2-c]pyridine-10-carboxylate (2.238 g, 8.30 mmol) in EtOH (30 mL) was added lithium chloride (504 mg, 11.9 mmol) and NaBH₄ (1.35 g, 35.7 mmol). The reaction was stirred at about room temperature for about 24 hours. To the mixture was added 1M HCl to adjust the pH to 6, the mixture was concentrated to remove solvent. Water (25 mL) was added to the reaction vessel and the resulting biphasic mixture was transferred to a separatory funnel. The layers were separated, and the aqueous phase was washed with ethyl acetate (3×30 mL). The combined organic layers were dried over anhydrous Na₂SO₄, filtered, and concentrated in vacuo. The resulting substance was purified by silica gel column chromatography with an isocratic elution of DCM (85%) and ethyl acetate (15%) to provide (10,11-dihydrobenzo[6,7]oxepino[3,2-c]pyridin-10-yl)methanol. MS (ESI): m/z=228.1 [M+H]⁺.

### d. Synthesis of 2-((10,11-dihydrobenzo[6,7]oxepino[3,2-c]pyridin-10-yl)methyl)isoindoline-1,3-dione

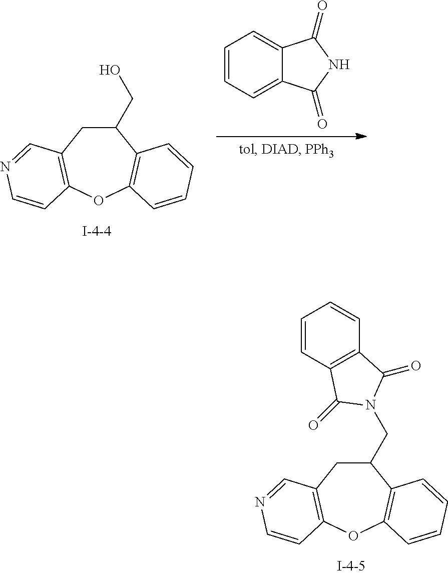

To a solution of (10,11-dihydrobenzo[6,7]oxepino[3,2-c]pyridin-10-yl)methanol (1.626 g, 6.55 mmol) in toluene (50 mL) was added phthalimide (1.95 g, 13.1 mmol), triphenylphosphine (3.50 g, 13.1 mmol) and followed by DIAD (2.77 g, 13.1 mmol). The reaction was stirred at about room temperature for about 16 hours. Additional amounts of triphenylphosphine (237 mg, 889 μmol), phthalimide (126 mg, 849 μmol) and DIAD (197 mg, 930 μmol) were added. The reaction was stirred at about room temperature for about 16 hours. Water (10 mL) and ethyl acetate (20 mL) were added to the reaction vessel and the resulting biphasic mixture was transferred to a separatory funnel. The layers were separated, and the aqueous phase was washed with ethyl acetate (3×15 mL). The combined organic layers were dried over anhydrous Na₂SO₄, filtered, and concentrated in vacuo. The resulting substance was purified by silica gel column chromatography with an isocratic elution of petroleum ether (50%) and ethyl acetate (50%) to provide crude 2-((10,11-dihydrobenzo[6,7]oxepino[3,2-c]pyridin-10-yl)methyl)isoindoline-1,3-dione. MS (ESI): m/z=357.0 [M+H]⁺.

### Example 22. Synthesis of (R*)-1-(10,11-dihydrobenzo[6,7]oxepino[3,2-c]pyridin-10-yl)-N-methylmethanamine (Compound 27) and (S*)-1-(10,11-dihydrobenzo[6,7]oxepino[3,2-c]pyridin-10-yl)-N-methylmethanamine (Compound 28)

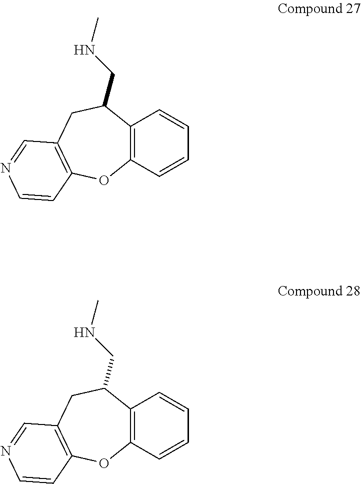 [SMILES: CNC[C@@H]1Cc2cnccc2Oc2ccccc21 ; CNC[C@H]1Cc2cnccc2Oc2ccccc21]

Compounds 27 and 28 are prepared similar to General Synthesis Scheme 1.

### Example 23. Synthesis of (R*)-(10,11-dihydrobenzo[6,7]oxepino[3,2-b]pyridin-10-yl)methanamine (Compound 29) and (S*)-(10,11-dihydrobenzo[6,7]oxepino[3,2-b]pyridin-10-yl)methanamine (Compound 30)

### a. Synthesis of 1-(2-(2-bromopyridin-3-yloxy)phenyl)ethenone

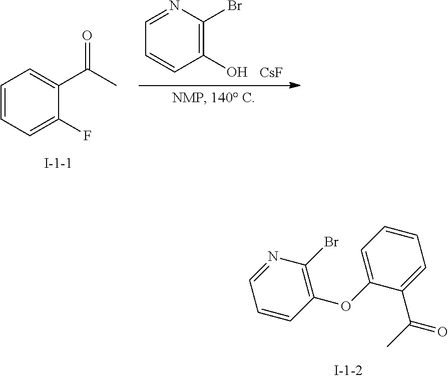

To a solution of 1-(2-fluorophenyl)ethanone (41.6 g, 301 mmol) in NMP (500 mL) was added 2-bromopyridin-3-ol (78.4 g, 451 mmol) and caesium fluoride (68.5 g, 451 mmol). The reaction mixture was heated to about 140° C. and stirred at that temperature for about 2 days. Water (2 L) was added to the reaction vessel and the mixture was extracted with ethyl acetate (3×800 mL). The combined organic layers were dried over anhydrous Na₂SO₄, filtered and concentrated in vacuo. The resulting substance was purified by silica gel column chromatography with a gradient elution of petroleum ether (100%) to petroleum ether (80%) and ethyl acetate (20%) to provide 1-(2-(2-bromopyridin-3-yloxy)phenyl)ethanone. MS (ESI) m/z: 292, 294 [M+H]⁺. ¹H NMR (400 MHz, CDCl₃) δ 8.22 (dd, J=1.6, 4.4 Hz, 1H), 7.88 (dd, J=1.6, 8.0 Hz, 1H), 7.50-7.46 (m, 1H), 7.29-7.20 (m, 3H), 6.81 (dd, J=0.8, 8.0 Hz, 1H), 2.69 (s, 3H).

### b. Synthesis of benzo[6,7]oxepino[3,2-b]pyridin-10(11H)-one

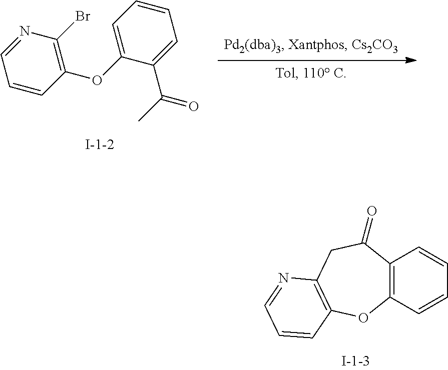 [SMILES: CC(=O)c1ccccc1Oc1cccnc1Br ; "Pd2(dba)3, Xantphos, Cs2CO3" ; "Tol, 110° C." ; O=C1Cc2ncccc2Oc2ccccc21]

To a solution of 1-(2-(2-bromopyridin-3-yloxy)phenyl)ethanone (11 g, 37.6 mmol) in toluene (150 mL) was added tris(dibenzylideneacetone)dipalladium(0) (Pd₂(dba)₃) (1.72 g, 1.88 mmol), Xantphos (2.17 g, 3.76 mmol) and Cs₂CO₃ (36.5 g, 112 mmol). The reaction mixture was heated to about 110° C. and stirred at that temperature for about 2 hours. Saturated aqueous NaCl (100 mL) was added to the reaction vessel and the resulting biphasic mixture was transferred to a separatory funnel. The layers were separated, and the aqueous phase was extracted with ethyl acetate (2×100 mL). The combined organic layers were dried over anhydrous Na₂SO₄, filtered and concentrated in vacuo. The resulting substance was purified by silica gel column chromatography with a gradient elution of petroleum ether (100%) to petroleum ether (70%) and ethyl acetate (30%) to provide benzo[6,7]oxepino[3,2-b]pyridin-10(11H)-one. MS (ESI) m/z: 212 [M+H]⁺. ¹H NMR (400 MHz, CDCl₃) δ 8.44 (dd, J=1.2, 4.4 Hz, 1H), 8.10 (dd, J=1.6, 8.0 Hz, 1H), 7.60-7.56 (m, 2H), 7.40-7.38 (m, 1H), 7.26-7.21 (m, 2H), 4.41 (s, 2H).

### c. Synthesis of benzo[6,7]oxepino[3,2-b]pyridin-10-yl trifluoromethanesulfonate

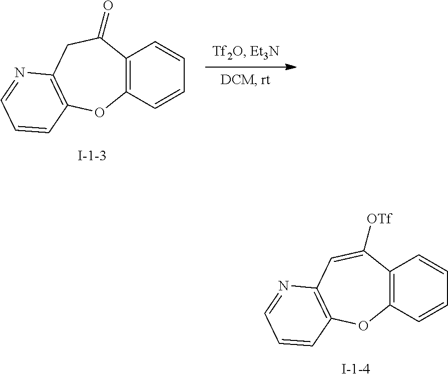

To a solution of benzo[6,7]oxepino[3,2-b]pyridin-10(11H)-one (3.0 g, 14.2 mmol) in DCM (50 mL) was added triethylamine (4.31 g, 42.6 mmol) and trifluoromethanesulfonic anhydride (9.98 g, 35.4 mmol). The reaction mixture was stirred at about room temperature for about 1 hour. Water (50 mL) was added to the reaction vessel and the resulting biphasic mixture was transferred to a separatory funnel. The layers were separated, and the aqueous phase was extracted with DCM (2×50 mL). The combined organic layers were dried over anhydrous Na₂SO₄, filtered, and concentrated in vacuo. The resulting substance was purified by silica gel column chromatography with a gradient elution of petroleum ether (100%) to petroleum ether (80%) and ethyl acetate (20%) to provide benzo[6,7]oxepino[3,2-b]pyridin-10-yl trifluoromethanesulfonate. MS (ESI) m/z: 344 [M+H]⁺. ¹H NMR (400 MHz, CDCl₃) δ 8.45 (dd, J=1.2, 4.4 Hz, 1H), 7.56-7.48 (m, 3H), 7.34-7.26 (m, 3H), 7.08 (s, 1H).

### d. Synthesis of benzo[6,7]oxepino[3,2-b]pyridine-10-carbonitrile

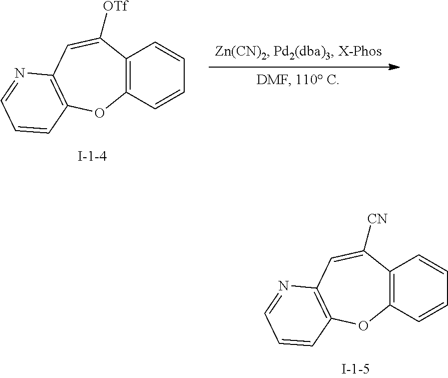 [SMILES: O=S(=O)(OC1=Cc2ncccc2Oc2ccccc21)C(F)(F)F ; "DMF, 110° C." ; "Zn(CN)2, Pd2(dba)3, X-Phos" ; N#CC1=Cc2ncccc2Oc2ccccc21]

To a solution of benzo[6,7]oxepino[3,2-b]pyridin-10-yl trifluoromethanesulfonate (2.5 g, 7.28 mmol) in DMF (40 mL) was added zinc cyanide (1.70 g, 14.5 mmol), Pd₂(dba)₃ (1.32 g, 1.45 mmol) and X-Phos (1.38 g, 2.91 mmol) under N₂. The mixture was stirred under N₂ at about 110° C. for about 1 hour. Water (150 mL) was added to the reaction vessel and the mixture was extracted with ethyl acetate (3×60 mL). The combined organic layers were dried over anhydrous Na₂SO₄, filtered and concentrated in vacuo. The resulting substance was purified by silica gel column chromatography with a gradient elution of petroleum ether (100%) to petroleum ether (65%) and ethyl acetate (35%) to provide benzo[6,7]oxepino[3,2-b]pyridine-10-carbonitrile. MS (ESI) m/z: 221 [M+H]⁺. ¹H NMR (400 MHz, CDCl₃) δ 8.48 (dd, J=1.2, 4.4 Hz, 1H), 7.65-7.62 (m, 2H), 7.55-7.53 (m, 1H), 7.49-7.45 (m, 1H), 7.39-7.36 (m, 1H), 7.32-7.24 (m, 2H).

### e. Synthesis of tert-butyl ((10,11-dihydrobenzo[6,7]oxepino[3,2-b]pyridin-10-yl)methyl)carbamate

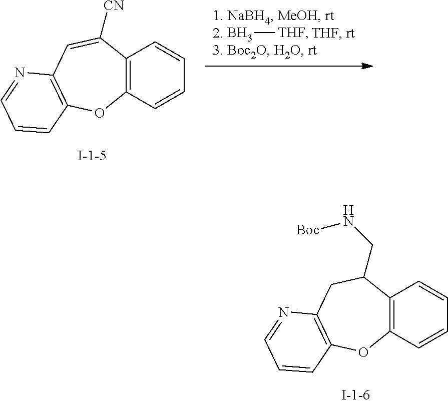

To a solution of benzo[6,7]oxepino[3,2-b]pyridine-10-carbonitrile (1.2 g, 5.44 mmol) in MeOH (20 mL) was added NaBH₄ (619 mg, 16.3 mmol). The reaction was stirred at about room temperature for about 2 hours. The mixture was concentrated in vacuo. The resulting substance was dissolved in THF (10 mL), BH₃-THF (1M in THF) (16.3 mL, 16.3 mmol) was added. The reaction was stirred at about room temperature for about 2 hours. Water (10 mL) was added slowly to quench the reaction, then 2M HCl aqueous solution (30 mL) was added. The mixture was stirred at about room temperature for about 16 hours. The pH of the mixture was adjusted to 8 using saturated NaHCO₃ aqueous solution. Then di-tert-butyl dicarbonate (2.35 g, 10.8 mmol) was added to above solution. The reaction was stirred at about room temperature for about 1 hour. The mixture was extracted with DCM (3×50 mL). The combined organic layers were dried over anhydrous Na₂SO₄, filtered and concentrated in vacuo. The resulting substance was purified by silica gel column chromatography with a gradient elution of petroleum ether (100%) to petroleum ether (66%) and ethyl acetate (34%) to provide tert-butyl ((10,11-dihydrobenzo[6,7]oxepino[3,2-b]pyridin-10-yl)methyl)carbamate. MS (ESI) m/z: 327 [M+H]⁺. ¹H NMR (400 MHz, CDCl₃) δ 8.30 (dd, J=0.8, 4.4 Hz, 1H), 7.48 (dd, J=0.6, 4.0 Hz, 1H), 7.30-7.26 (m, 1H), 7.24-7.08 (m, 4H), 4.98 (bs, 1H), 3.63-3.52 (m, 3H), 3.26-3.21 (m, 1H), 2.92-2.87 (m, 1H), 1.45 (s, 9H).

### f. Chiral column separation of tert-butyl ((10,11-dihydrobenzo[6,7]oxepino[3,2-b]pyridin-10-yl)methyl)carbamate

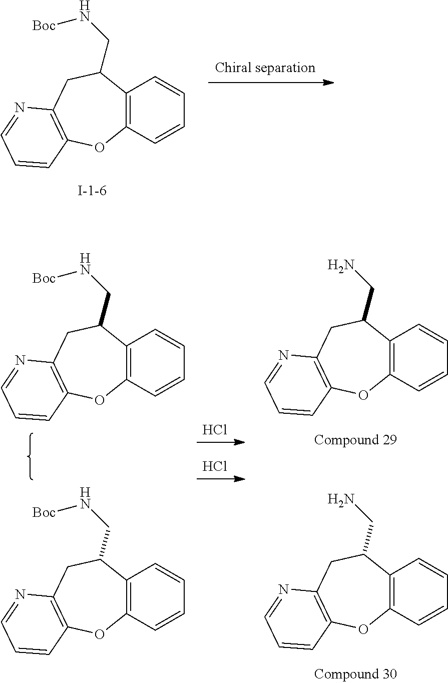

300 mg of tert-butyl ((10,11-dihydrobenzo[6,7]oxepino[3,2-b]pyridin-10-yl)methyl)carbamate was separated by chiral HPLC under following conditions:

| | |
|---|---|
| Instrument: SFC-150 (Waters) | Column: AD 20 × 250 |
| Column temperature: 35° C. | mm, 10 μm (Regis) |
| Mobile phase: CO₂/IPA (0.5% | |
| Methanol Ammonia) = 85/15 | |
| Flow rate: 100 g/min | Back pressure: 100 bar |
| Detection wavelength: 214 nm | Cycle time: 3.37 min |
| Sample solution: 300 mg dissolved in | |
| 30 mL methanol | |
| Injection volume: 1 mL | |

After removal of solvents, the first eluting isomer (I-1-6-P1) (100 mg, retention time=2.58 min) and the second eluting isomer (I-1-6-P2) (100 mg, retention time=3.11 min) were obtained.

### Synthesis of (R*)-(10,11-dihydrobenzo[6,7]oxepino[3,2-b]pyridin-10-yl)methanamine (Compound 29) and (S*)-(10,11-dihydrobenzo[6,7]oxepino[3,2-b]pyridin-10-yl)methanamine (Compound 30)

Independently, for each individual isomer I-1-6-P1 and I-1-6-P2, a solution of compound (50 mg, 153 μmol) in HCl/MeOH (3 M, 10 mL) was stirred at about room temperature for about 16 hours. The mixture was concentrated in vacuo. To the residue was added ethyl acetate (10 mL). The mixture was stirred at about room temperature for about 10 min and then filtered. The substance was collected and dissolved in water (10 mL). The mixture was freeze-dried on a lyophilizer to provide Compound 29. MS (ESI) m/z: 227 [M+H]⁺. (¹H NMR (400 MHz, CD₃OD) δ 8.61 (dd, J=1.2, 5.6 Hz, 1H), 8.47 (d, J=8.4 Hz, 1H), 7.96 (dd, J=5.6, 8.4 Hz, 1H), 7.49-7.35 (m, 4H), 3.82-3.74 (m, 2H), 3.68-3.50 (m, 3H).) (Chiral analysis column: AD-3 4.6×100 mm 3 μm; co-solvent: MeOH [0.2% NH₃ (7M in MeOH)]; Acq. method Set: AD 15% B1; flow rate: 3.0 mL/min; column temperature: 40° C.; retention time=3.118 min.); and Compound 30. MS (ESI) m/z: 227 [M+H]⁺. (¹H NMR (400 MHz, CD₃OD) δ 8.61 (dd, J=1.2, 5.6 Hz, 1H), 8.48 (d, J=8.4 Hz, 1H), 7.96 (dd, J=5.6, 8.4 Hz, 1H), 7.49-7.35 (m, 4H), 3.85-3.75 (m, 2H), 3.67-3.50 (m, 3H).) (Chiral analysis column: AD-3 4.6×100 mm 3 μm; co-solvent: MeOH [0.2% NH₃ (7M in MeOH)]; Acq. method Set: AD 15% B1; flow rate: 3.0 mL/min; column temperature: 40° C.; retention time=2.485 min.)

### Example 24. Synthesis of (R*)-1-(10,11-dihydrobenzo[6,7]oxepino[3,2-b]pyridin-10-yl)-N-methylmethanamine (Compound 31) and (S*)-1-(10,11-dihydrobenzo[6,7]oxepino[3,2-b]pyridin-10-yl)-N-methylmethanamine (Compound 32)

### a. Synthesis of tert-butyl ((10,11-dihydrobenzo[6,7]oxepino[3,2-b]pyridin-10-yl)methyl)(methyl)carbamate

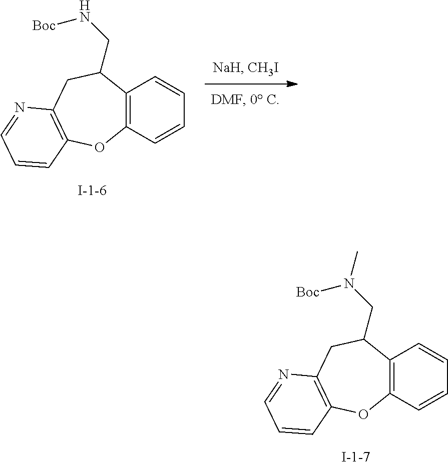 [SMILES: CC(C)(C)OC(=O)NCC1Cc2ncccc2Oc2ccccc21 ; CN(CC1Cc2ncccc2Oc2ccccc21)C(=O)OC(C)(C)C]

A solution of tert-butyl ((10,11-dihydrobenzo[6,7]oxepino[3,2-b]pyridin-10-yl)methyl)carbamate (300 mg, 0.92 mmol) in DMF (6 mL) was cooled to 0° C. Sodium hydride (60% in mineral oil) (110 mg, 2.76 mmol) was added. The mixture was stirred at about room temperature for about 10 minutes. Then iodomethane (392 mg, 2.76 mmol) was added. The reaction was stirred at about room temperature for about 3 hours. Saturated aqueous NH₄Cl (30 mL) was added to the reaction vessel and the mixture was extracted with ethyl acetate (3×20 mL). The combined organic layers were dried over anhydrous Na₂SO₄, filtered, and concentrated in vacuo. The resulting substance was purified by silica gel column chromatography with a gradient elution of DCM (100%) to DCM (98%) and MeOH (2%) to provide tert-butyl ((10,11-dihydrobenzo[6,7]oxepino[3,2-b]pyridin-10-yl)methyl)(methyl)carbamate. MS (ESI) m/z: 341 [M+H]⁺.

### b. Chiral column separation of tert-butyl ((10,11-dihydrobenzo[6,7]oxepino[3,2-b]pyridin-10-yl)methyl)(methyl)carbamate

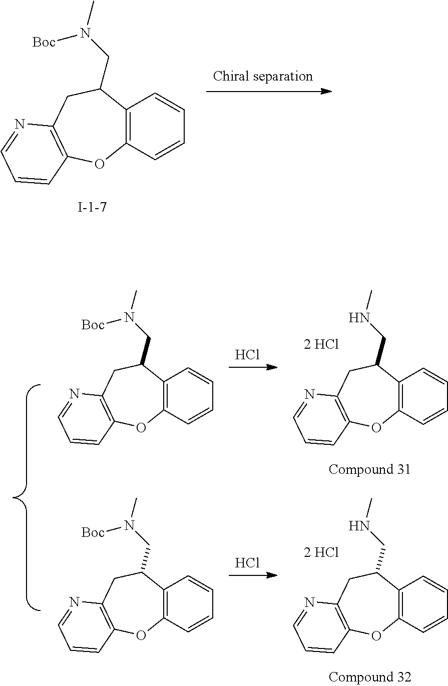

Compound I-1-7 was separated by the following conditions:

| | |
|---|---|
| Instrument: SFC-150 (Waters) | Column: IC 20 × 250 |
| Column temperature: 35° C. | mm, 10 μm (Daicel) |
| Mobile phase: CO₂/MeOH(0.2% | |
| Methanol Ammonia) = 85/15 | |
| Flow rate: 100 g/min | Back pressure: 100 bar |
| Detection wavelength: 214 nm | Cycle time: 2.23 min |
| Sample solution: 300 mg dissolved in | |
| 20 mL Methanol and Dichloromethane | |
| Injection volume: 1 mL | |

After removing solvents, I-1-7-P1 (100 mg, retention time: 1.626 min) and I-1-7-P2 (100 mg, retention time: 1.884 min) were obtained.

### Synthesis of (R*)-1-(10,11-dihydrobenzo[6,7]oxepino[3,2-b]pyridin-10-yl)-N-methylmethanamine (Compound 31) and (S*)-1-(10,11-dihydrobenzo[6,7]oxepino[3,2-b]pyridin-10-yl)-N-methylmethanamine (Compound 32)

Independently, for each individual isomer I-1-7-P1 and I-1-7-P2, a solution of each compound (100 mg, 294 μmol) in 3M HCl in MeOH (10 mL) was stirred at about room temperature for about 16 hours, and the mixture was concentrated in vacuo. To the residue was added ethyl acetate (10 mL). The mixture was stirred at about room temperature for about 10 minutes and then filtered. The substance was collected and dissolved in water (10 mL). The substance was freeze-dried on lyophilizer to provide Compound 31. MS (ESI) m/z: 241 [M+H]⁺. ¹H NMR (400 MHz, CD₃OD) δ 8.62 (dd, J=0.8, 5.6 Hz, 1H), 8.48 (d, J=8.4 Hz, 1H), 7.99-7.95 (m, 1H), 7.51-7.35 (m, 4H), 3.92-3.88 (m, 1H), 3.81-3.76 (m, 1H) 3.72-3.64 (m, 2H), 3.61-3.56 (m, 1H), 2.75 (s, 3H). Chiral analysis column: IG-3 4.6×100 mm 3 μm; co-solvent: MeOH [0.2% NH₃ (7M in MeOH)]; Acq. method Set: IG 20% B1; flow rate: 3.0 mL/min; column temperature: 40° C.; retention time=2.917 min; and Compound 32. MS (ESI) m/z: 241 [M+H]⁺. ¹H NMR (400 MHz, CD₃OD) δ 8.62 (dd, J=0.8, 5.6 Hz, 1H), 8.48 (d, J=8.4 Hz, 1H), 7.99-7.95 (m, 1H), 7.52-7.36 (m, 4H), 3.92-3.88 (m, 1H), 3.81-3.76 (m, 1H) 3.72-3.64 (m, 2H), 3.61-3.56 (m, 1H), 2.76 (s, 3H). Chiral analysis column: IG-3 4.6×100 mm 3 μm; co-solvent: MeOH [0.2% NH₃ (7M in MeOH)]; Acq. method Set: IG 20% B1; flow rate: 3.0 mL/min; column temperature: 40° C.; retention time=2.352 min.

### Example 27. Synthesis of (R*)-1-(3-fluoro-10,11-dihydrobenzo[6,7]oxepino[3,2-b]pyridin-10-yl)-N-methylmethanamine (Compound 37) and (S*)-1-(3-fluoro-10,11-dihydrobenzo[6,7]oxepino[3,2-b]pyridin-10-yl)-N-methylmethanamine (Compound 38)

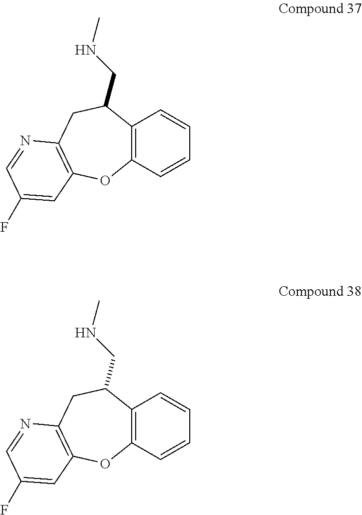 [SMILES: CNC[C@@H]1Cc2ncc(F)cc2Oc2ccccc21 ; CNC[C@H]1Cc2ncc(F)cc2Oc2ccccc21]

Compounds 37 and 38 are prepared similar to General Synthesis Scheme 1.

### Example 29. Synthesis of (R*)-(7-fluoro-11H-dibenzo[b,e][1,4]dioxepin-11-yl)methanamine (Compound 41) and (S*)-(7-fluoro-11H-dibenzo[b,e][1,4]dioxepin-11-yl)methanamine (Compound 42)

### a. Preparation of 2-(2-(benzyloxy)phenoxy)nicotinaldehyde

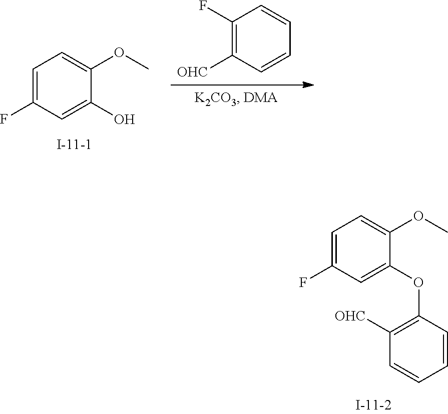

To a solution of 5-fluoro-2-methoxyphenol (1.42 g, 9.99 mmol) in DMA (20 mL) was added 2-fluorobenzaldehyde (1.23 g, 9.99 mmol) and potassium carbonate (2.75 g, 19.9 mmol). The reaction was stirred at about 80° C. for about 6 hours. Water (100 mL) and ethyl acetate (100 mL) were added to the reaction vessel and the resulting biphasic mixture was transferred to a separatory funnel. The layers were separated, and the organic phase was washed with ethyl acetate (2×50 mL) and saturated aqueous NaCl (2×50 mL). The combined organics were dried over anhydrous Na₂SO₄, filtered, and concentrated in vacuo. The resulting oil was purified by flash column chromatography with an isocratic elution of ethyl acetate (10%) and petroleum ether (90%) to provide 2-(5-fluoro-2-hydroxyphenoxy)benzaldehyde. MS (ESI): m/z 247 [M+H]⁺.

### b. Preparation of 2-(5-fluoro-2-hydroxyphenoxy)benzaldehyde

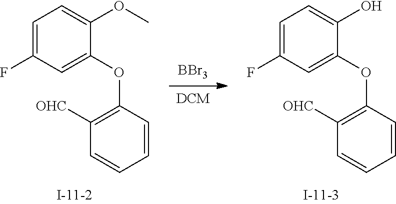

To a solution of 2-(5-fluoro-2-methoxyphenoxy)benzaldehyde (5.1 g, 20.7 mmol) in DCM (50 mL) was added tribromoborane (10.3 g, 41.4 mmol) at about −50° C. The mixture was stirred at about room temperature for about 2 hours and quenched with ice-water (30 mL). The organic layer was concentrated to give crude compound. The crude compound was dissolved in THE (10 mL)/HCl (6M in water, 10 mL). The mixture was stirred at about 70° C. for about 4 hours and cooled to room temperature. The pH of the mixture was adjusted to 8 using NaHCO₃ solution. Water (100 mL) was added to the reaction vessel and the mixture was extracted with ethyl acetate (3×100 mL). The combined organic layers were dried over anhydrous Na₂SO₄, filtered, and concentrated in vacuo. The resulting oil was purified by silica gel chromatography (petroleum ether/ethyl acetate=3/1) to give 2-(5-fluoro-2-hydroxyphenoxy)benzaldehyde. MS (ESI): m/z 233 [M+H]⁺.

### c. Preparation of 2-(2-(benzyloxy)-5-fluorophenoxy)benzaldehyde

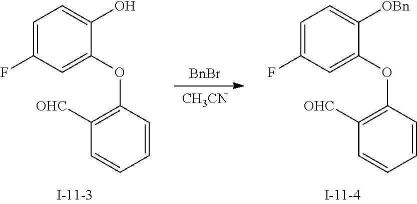

To a solution of 2-(5-fluoro-2-hydroxyphenoxy)benzaldehyde (2.1 g, 9.04 mmol) in CH₃CN (20 mL) was added potassium carbonate (2.48 g, 18.0 mmol) and (bromomethyl)benzene (1.54 g, 9.04 mmol). The mixture was stirred at about 60° C. for about 2 hours. Water (100 mL) was added to the reaction vessel and the mixture was extracted with ethyl acetate (3×100 mL). The combined organic layers were dried over anhydrous Na₂SO₄, filtered, and concentrated in vacuo. The resulting oil purified by silica gel chromatography (petroleum ether/ethyl acetate=8/1) to give 2-(2-(benzyloxy)-5-fluorophenoxy)benzaldehyde. MS (ESI): m/z 323 [M+H]⁺.

### d. Preparation of 2-(2-(2-(benzyloxy)-5-fluorophenoxy)phenyl)-2-hydroxyacetonitrile

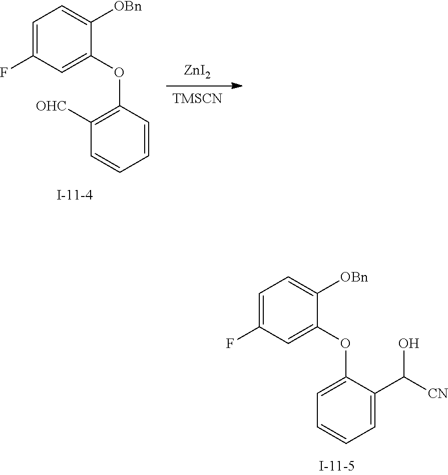 [SMILES: "TMSCN" ; O=Cc1ccccc1Oc1cc(F)ccc1OCc1ccccc1 ; "ZnI2" ; N#CC(O)c1ccccc1Oc1cc(F)ccc1OCc1ccccc1]

To a solution of 2-(2-(benzyloxy)-5-fluorophenoxy)benzaldehyde (2.1 g, 6.51 mmol) in methylene chloride (20 mL) was added trimethylsilanecarbonitrile (1.28 g, 13.0 mmol) and iodozinc (207 mg, 651 μmol). The reaction was stirred at about room temperature for about 2 hours and filtered. The filtrate was concentrated to afford 2-(2-(2-(benzyloxy)-5-fluorophenoxy) phenyl)-2-hydroxyacetonitrile. MS (ESI): m/z 350 [M+H]⁺.

### e. Preparation of 2-amino-1-(2-(2-(benzyloxy)-5-fluorophenoxy)phenyl)ethan-1-ol

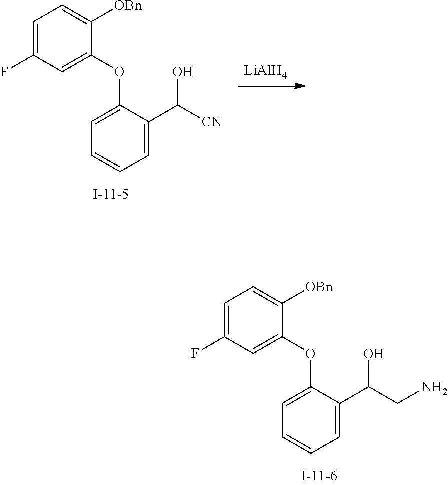 [SMILES: N#CC(O)c1ccccc1Oc1cc(F)ccc1OCc1ccccc1 ; "LiAlH4" ; NCC(O)c1ccccc1Oc1cc(F)ccc1OCc1ccccc1]

To a solution of 2-(2-(2-(benzyloxy)-5-fluorophenoxy)phenyl)-2-hydroxyacetonitrile (crude product) in tetrahydrofuran (15 mL) was added LiAlH₄ (432 mg, 11.4 mmol) at about 0° C. The mixture was stirred at about room temperature for about 1 hour and quenched with Na₂SO₄·10H₂O (2.0 g). The mixture was stirred at about room temperature for about 20 min and filtered. The filtrate was dried and concentrated to give 2-amino-1-(2-(2-(benzyloxy)-5-fluorophenoxy)phenyl)ethan-1-ol. MS (ESI): m/z 354 [M+H]⁺.

### f. Preparation of tert-butyl (2-(2-(2-(benzyloxy)-5-fluorophenoxy)phenyl)-2-hydroxyethyl)carbamate

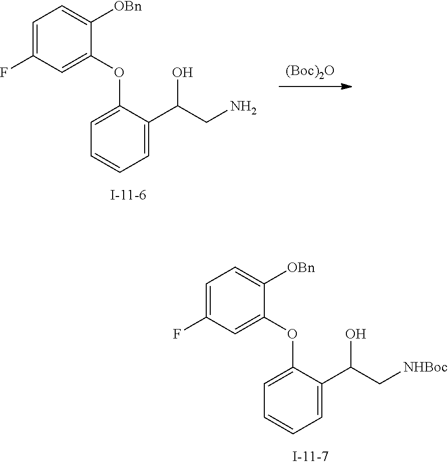 [SMILES: "(Boc)2O" ; NCC(O)c1ccccc1Oc1cc(F)ccc1OCc1ccccc1 ; CC(C)(C)OC(=O)NCC(O)c1ccccc1Oc1cc(F)ccc1OCc1ccccc1]

To a solution of 2-amino-1-{2-[2-(benzyloxy)-5-fluorophenoxy]phenyl}ethan-1-ol (1.8 g, 5.09 mmol) in DCM (30 mL) and NaHCO₃ solution (30 mL) was added di-tert-butyl dicarbonate (1.11 g, 5.09 mmol). The mixture was stirred at about room temperature for about 3 hours. Water (100 mL) was added to the reaction vessel and the mixture was extracted with ethyl acetate (3×100 mL). The combined organic layers were dried over anhydrous Na₂SO₄, filtered, and concentrated in vacuo. The resulting substance was purified by silica gel chromatography (petroleum ether/ethyl acetate=2/1) to give tert-butyl (2-(2-(2-(benzyloxy)-5-fluorophenoxy)phenyl)-2-hydroxyethyl)carbamate. MS (ESI): m/z 476 [M+Na]⁺.

### g. Preparation of tert-butyl (2-(2-(5-fluoro-2-hydroxyphenoxy)phenyl)-2-hydroxyethyl)carbamate

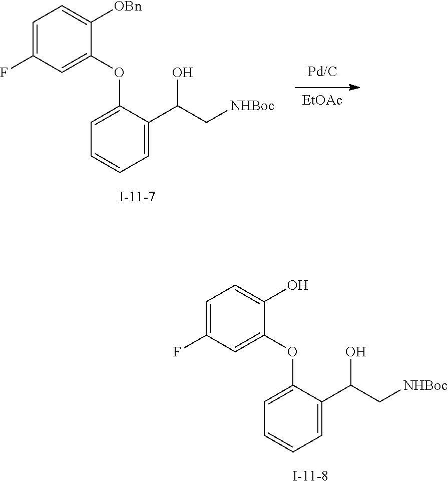 [SMILES: "EtOAc" ; CC(C)(C)OC(=O)NCC(O)c1ccccc1Oc1cc(F)ccc1OCc1ccccc1 ; CC(C)(C)OC(=O)NCC(O)c1ccccc1Oc1cc(F)ccc1O]

To a solution of tert-butyl (2-(2-(2-(benzyloxy)-5-fluorophenoxy)phenyl)-2-hydroxyethyl)carbamate (600 mg, 1.32 mmol) in methanol (10 mL) was added Pd/C (200 mg, 10%). The mixture was stirred at about room temperature for about 3 hours under a hydrogen atmosphere (1 atm) and filtered. The filtrate was concentrated to give tert-butyl (2-(2-(5-fluoro-2-hydroxyphenoxy)phenyl)-2-hydroxyethyl)carbamate. MS (ESI): m/z 386 [M+H]⁺.

### h. Preparation of tert-butyl ((7-fluoro-11H-dibenzo[b,e][1,4]dioxepin-11-yl)methyl)carbamate

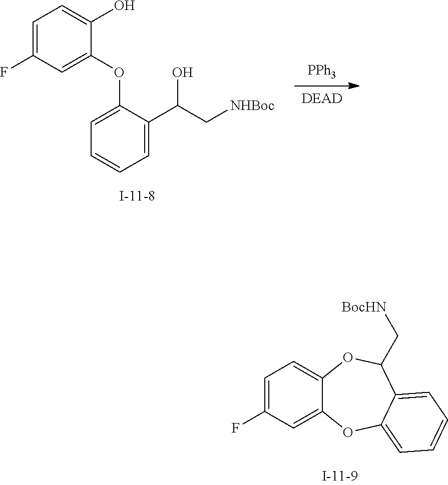 [SMILES: CC(C)(C)OC(=O)NCC(O)c1ccccc1Oc1cc(F)ccc1O ; "DEAD" ; CC(C)(C)OC(=O)NCC1Oc2ccc(F)cc2Oc2ccccc21 ; "PPh3"]

To a solution of tert-butyl (2-(2-(5-fluoro-2-hydroxyphenoxy)phenyl)-2-hydroxyethyl)carbamate (500 mg, 1.37 mmol) in tetrahydrofuran (6 mL) was added triphenylphosphine (430 mg, 1.64 mmol) and N-[(ethoxycarbonyl)imino]ethoxyformamide (285 mg, 1.64 mmol) at about 0° C. The reaction was stirred at about room temperature for about 1 hour and quenched with H₂O (10 mL). The mixture was extracted with ethyl acetate (30 mL). The organic layer was evaporated and purified by silica gel chromatography (petroleum ether/ethyl acetate=5/1) to give tert-butyl ((7-fluoro-11H-dibenzo[b,e][1,4]dioxepin-11-yl)methyl)carbamate. MS (ESI): m/z 368[M+Na]⁺

### i. Chiral column separation of tert-butyl ((7-fluoro-11H-dibenzo[b,e],[1,4]dioxepin-11-yl)methyl)carbamate

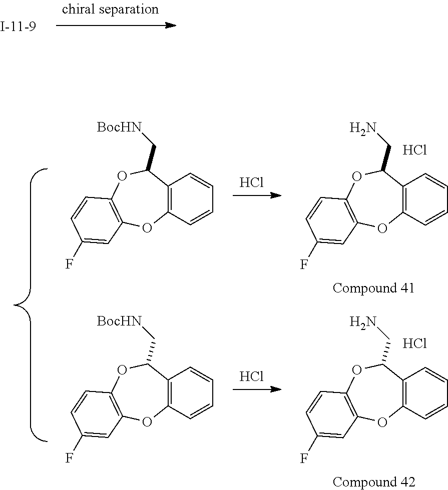

Racemic tert-butyl ((7-fluoro-11H-dibenzo[b,e][1,4]dioxepin-11-yl)methyl)carbamate was purified by chiral column separation using the following conditions:

| | |
|---|---|
| Instrument: SFC-150 (Waters) | Column: IG 20*250 |
| Column temperature: 35° C. | mm, 10 μm (Daicel) |
| Mobile phase: CO2/MeOH (0.2% | |
| Methanol Ammonia) = 88/12 | |
| Flow rate: 100 g/min | Back pressure: 100 bar |
| Detection wavelength: 214 nm | Cycle time: 8 min |
| Sample solution: 170 mg dissolved in | |
| 30 mL methanol | |
| Injection volume: 2 mL | |

After removal of the solvents, the first eluting isomer (I-11-9-P1) (80 mg, retention time=2.10 min) and the second eluting isomer (I-11-9-P2) (80 mg, retention time=2.52 min) were obtained.

### Synthesis of (R*)-(7-fluoro-11H-dibenzo[b,e],[1,4]dioxepin-11-yl)methanamine (Compound 41) and (S*)-(7-fluoro-11H-dibenzo[b,e],[1,4]dioxepin-11-yl)methanamine (Compound 42)

Independently, for each individual isomer I-11-9-P1 and I-11-9-P2, to a solution of each compound (40 mg, 115 μmol) in MeOH (5 mL) was added HCl/MeOH (3M, 3 mL, 9 mmol). The reaction was stirred at about room temperature for about 16 hours and concentrated under vacuum. The residue was dissolved in water (5 mL) and extracted with ethyl acetate (5 mL). The aqueous phase was freeze-dried in a lyophilizer to give Compound 41. MS (ESI): m/z 246 [M+H]⁺. ¹H NMR (400 MHz, CD₃OD) δ 7.51-7.19 (m, 4H), 7.08 (dd, J=9.1, 5.6 Hz, 1H), 7.00 (dd, J=9.1, 3.0 Hz, 1H), 6.90-6.72 (m, 1H), 5.79 (dd, J=10.2, 3.3 Hz, 1H), 3.86-3.61 (m, 2H). Chiral analysis column: AD-3 4.6*100 mm 3 μm, co-solvent: MeOH [0.2% NH3(7M in MeOH)], Acq. Method Set: AD 25% B1, flow rate: 3.0 mL/min, column temperature: 40° C., retention time=1.48 min.; and Compound 42. MS (ESI): m/z 246 [M+H]⁺. ¹H NMR (400 MHz, CD₃OD) δ 7.50-7.38 (m, 1H), 7.37-7.27 (m, 3H), 7.07 (dd, J=9.1, 5.6 Hz, 1H), 7.01 (dd, J=9.2, 3.0 Hz, 1H), 6.80 (ddd, J=9.1, 7.6, 3.0 Hz, 1H), 5.78 (dd, J=10.2, 3.3 Hz, 1H), 3.72 (ddd, J=16.5, 13.3, 6.8 Hz, 2H). Chiral analysis column: AD-3 4.6*100 mm 3 μm, co-solvent: MeOH [0.2% NH3(7M in MeOH)], Acq. Method Set: AD 25% B1, flow rate: 3.0 mL/min, column temperature: 40° C., retention time=2.00 min.

### Example 30. Synthesis of (R*)-1-(7-fluoro-11H-dibenzo[b,e][1,4]dioxepin-11-yl)-N-methylmethanamine (Compound 43) and (S*)-1-(7-fluoro-11H-dibenzo[b,e][1,4]dioxepin-11-yl)-N-methylmethanamine (Compound 44)

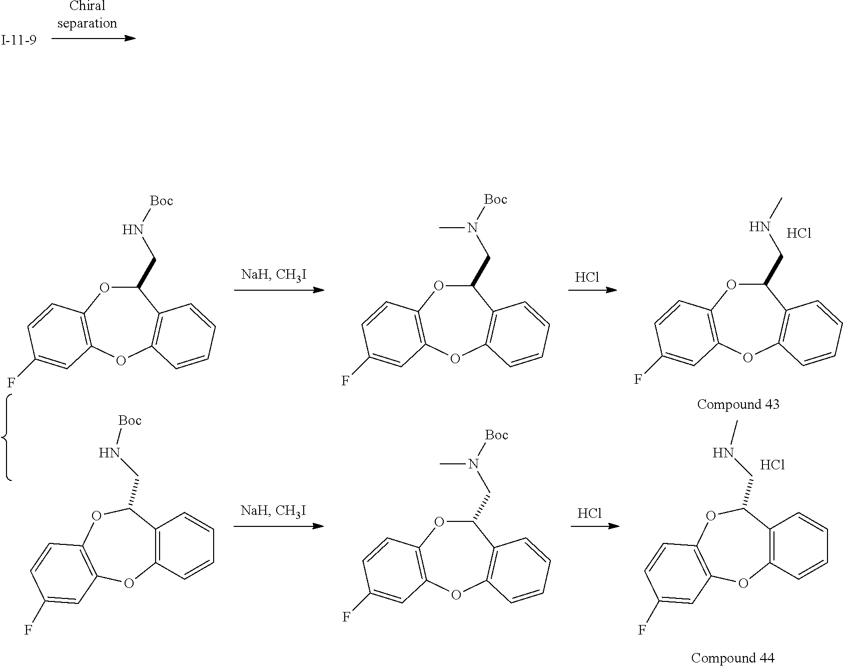

### Synthesis of (R*)-1-(7-fluoro-11H-dibenzo[b,e][1,4]dioxepin-11-yl)-N-methylmethanamine (Compound 43) and (S*)-1-(7-fluoro-11H-dibenzo[b,e],[1,4]dioxepin-11-yl)-N-methylmethanamine (Compound 44)

Independently, for each compound I-11-9-P1 and I-11-9-P2, to a solution of each compound (40 mg, 115 μmol) in tetrahydrofuran (5 mL) was added sodium hydride (11.0 mg, 460 μmol) and iodomethane (48.9 mg, 3345 μmol) at about 0° C. The reaction was stirred at about room temperature for about 3 hours and quenched with ice-water (3 mL). The mixture was extracted with ethyl acetate (15 mL×2). The combined organic layers were dried over anhydrous Na₂SO₄, filtered, and concentrated in vacuo. The resulting substance was purified by silica gel chromatography (petroleum ether/ethyl acetate=4/1) to provide each N-methylated intermediate. MS (ESI): m/z 382 [M+Na]⁺

To a solution of each N-methylated intermediate (40 mg, 111 μmol) in MeOH (5 mL) was added HCl/MeOH (5 mL, 10 mmol). The reaction was stirred at about room temperature for about 16 hours and concentrated under vacuum. The residue was dissolved in water (5 mL) and extracted with ethyl acetate (5 mL). The aqueous phase was freeze-dried to give Compound 43. MS (ESI): m/z 260 [M+H]⁺. ¹H NMR (400 MHz, CD₃OD) δ 7.55-7.18 (m, 4H), 7.08 (dd, J=9.0, 5.6 Hz, 1H), 7.01 (dd, J=9.1, 3.0 Hz, 1H), 6.89-6.70 (m, 1H), 5.87 (dd, J=10.6, 2.5 Hz, 1H), 4.02-3.83 (m, 1H), 3.75 (d, J=12.7 Hz, 1H), 2.88 (s, 3H). Chiral analysis column: AD-3 4.6*100 mm 3 μm, co-solvent: MeOH [0.2% NH3(7M in MeOH)], Acq. Method Set: AD 25% B1, flow rate: 3.0 mL/min, column temperature: 40° C., retention time=1.10 min.; and Compound 44. MS (ESI): m/z 260 [M+H]⁺. ¹H NMR (400 MHz, CD₃OD) δ 7.52-7.18 (m, 4H), 7.08 (dd, J=9.1, 5.6 Hz, 1H), 7.02 (dd, J=9.1, 3.0 Hz, 1H), 6.81 (ddd, J=9.0, 7.7, 3.0 Hz, 1H), 5.86 (dd, J=10.7, 2.7 Hz, 1H), 3.96-3.82 (m, 1H), 3.74 (dd, J=13.1, 2.5 Hz, 1H), 2.88 (s, 3H). Chiral analysis column: AD-3 4.6*100 mm 3 μm, co-solvent: MeOH [0.2% NH3(7M in MeOH)], Acq. Method Set: AD 25% B1, flow rate: 3.0 mL/min, column temperature: 40° C., retention time=1.89 min.

### Example 31. Synthesis of (R*)-(5H-benzo[2,3][1,4]dioxepino[5,6-b]pyridin-5-yl)methanamine (Compound 47) and (S*)-(5H-benzo[2,3][1,4]dioxepino[5,6-b]pyridin-5-yl)methanamine (Compound 48)

### a. Preparation of 2-(2-(benzyloxy)phenoxy)nicotinaldehyde

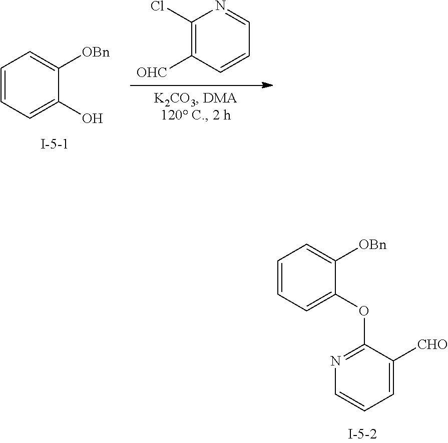

To a solution of 2-(benzyloxy)phenol (4 g, 19.9 mmol) in dimethylacetamide (5 mL) was added 2-chloronicotinaldehyde (2.81 g, 19.9 mmol) and K₂CO₃ (5.5 g, 39.8 mmol). The reaction mixture was heated to about 120° C. and stirred at that temperature for about 2 hours. Water (40 mL) and ethyl acetate (100 mL) were added to the reaction vessel and the resulting biphasic mixture was transferred to a separatory funnel. The layers were separated, and the organic phase was washed with saturated aqueous NaCl (30 mL). The combined organics were dried over anhydrous Na₂SO₄, filtered, and concentrated in vacuo. The resulting substance was purified by flash column chromatography with a gradient elution of petroleum ether (100%) and ethyl acetate (0%) to petroleum ether (60%) and ethyl acetate (40%) to provide 2-(2-(benzyloxy)phenoxy)nicotinaldehyde. MS (ESI): m/z 306 [M+H]⁺.

### b. Preparation of 2-(2-(2-(benzyloxy)phenoxy)pyridin-3-yl)-2-hydroxyacetonitrile

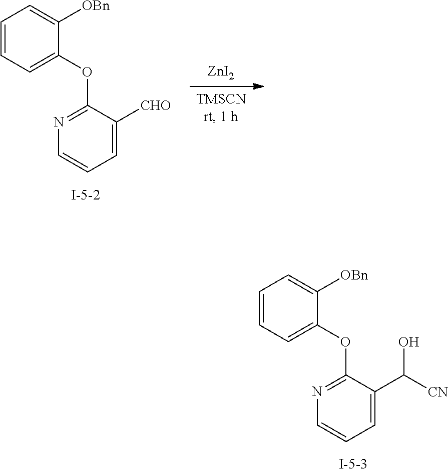 [SMILES: O=Cc1cccnc1Oc1ccccc1OCc1ccccc1 ; "ZnI2" ; "TMSCNrt, 1 h" ; N#CC(O)c1cccnc1Oc1ccccc1OCc1ccccc1]

To a solution of 2-(2-(benzyloxy)phenoxy)nicotinaldehyde (4.8 g, 15.7 mmol) in methylene chloride (40 mL) was added trimethylsilanecarbonitrile (3.10 g, 31.3 mmol) and iodozinc (1 g, 3.14 mmol). The reaction was stirred at about room temperature for about 2 hours and filtered. The filtrate was concentrated to afford 2-(2-(2-(benzyloxy)phenoxy)pyridin-3-yl)-2-hydroxyacetonitrile. MS (ESI): m/z 333 [M+H]⁺.

### c. Preparation of 2-amino-1-(2-(2-(benzyloxy)phenoxy)pyridin-3-yl)ethan-1-ol

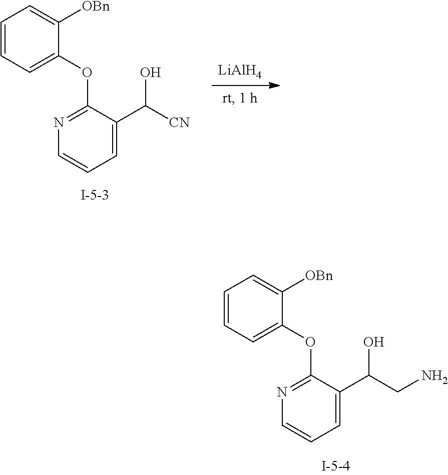 [SMILES: N#CC(O)c1cccnc1Oc1ccccc1OCc1ccccc1 ; "LiAlH4" ; "rt, 1 h" ; NCC(O)c1cccnc1Oc1ccccc1OCc1ccccc1]

To a solution of 2-(2-(2-(benzyloxy)phenoxy)pyridin-3-yl)-2-hydroxyacetonitrile (4.7 g, 14.1 mmol) in tetrahydrofuran (50 mL) was added LiAH₄ (1.07 g, 28.2 mmol) at about 0° C. The reaction was stirred at about room temperature for about 1 hours and quenched with Na₂SO₄.10H₂O (1.0 g). The mixture was stirred at about room temperature for about 20 minutes and filtered. The filtrate was dried and concentrated to give 2-amino-1-(2-(2-(benzyloxy)phenoxy)pyridin-3-yl)ethan-1-ol. MS (ESI): m/z 337 [M+H]⁺.

### d. Preparation of tert-butyl (2-(2-(2-(benzyloxy)phenoxy)pyridin-3-yl)-2-hydroxyethyl)carbamate

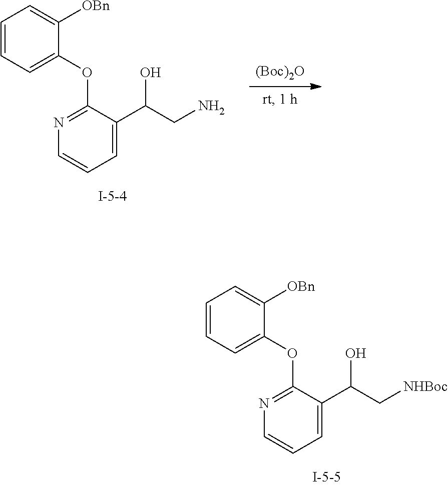 [SMILES: "(Boc)2O" ; NCC(O)c1cccnc1Oc1ccccc1OCc1ccccc1 ; "rt, 1 h" ; CC(C)(C)OC(=O)NCC(O)c1cccnc1Oc1ccccc1OCc1ccccc1]

To a solution of 2-amino-1-(2-(2-(benzyloxy)phenoxy)pyridin-3-yl)ethan-1-ol (4.5 g, 13.3 mmol) in methylene chloride (50 mL) and Na₂CO₃ solution (50 mL) was added di-tert-butyl dicarbonate (2.90 g, 13.3 mmol). The reaction was stirred at about room temperature for about 1 hour. Water (100 mL) was added to the reaction vessel and the mixture was extracted with methylene chloride (2×100 mL). The combined organic layers were dried over anhydrous Na₂SO₄, filtered, and concentrated in vacuo. The resulting substance was purified by silica gel chromatography (petroleum ether/ethyl acetate=3/1) to give tert-butyl (2-(2-(2-(benzyloxy)phenoxy)pyridin-3-yl)-2-hydroxyethyl)carbamate. MS (ESI): m/z 437 [M+H]⁺.

### e. Preparation of tert-butyl (2-hydroxy-2-(2-(2-hydroxyphenoxy)pyridin-3-yl)ethyl)carbamate

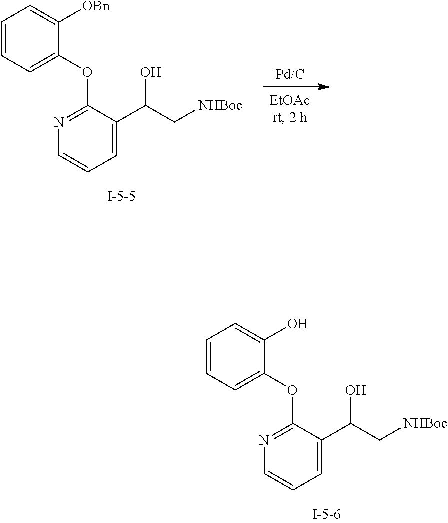 [SMILES: CC(C)(C)OC(=O)NCC(O)c1cccnc1Oc1ccccc1OCc1ccccc1 ; "EtOAcrt, 2 h" ; "Pd/C" ; CC(C)(C)OC(=O)NCC(O)c1cccnc1Oc1ccccc1O]

To a solution of tert-butyl (2-(2-(2-(benzyloxy)phenoxy)pyridin-3-yl)-2-hydroxyethyl)carbamate (5.5 g, 12.5 mmol) in ethyl acetate (5 mL) was added Pd/C (600 mg, 10%). The reaction was stirred at about room temperature for about 1 hour and filtered. The filtrate was concentrated to give crude compound. MS (ESI): m/z 347 [M+H]⁺.

### f. Preparation of tert-butyl ((5H-benzo[2,3][1,4]dioxepino[5,6-b]pyridin-5-yl)methyl)carbamate

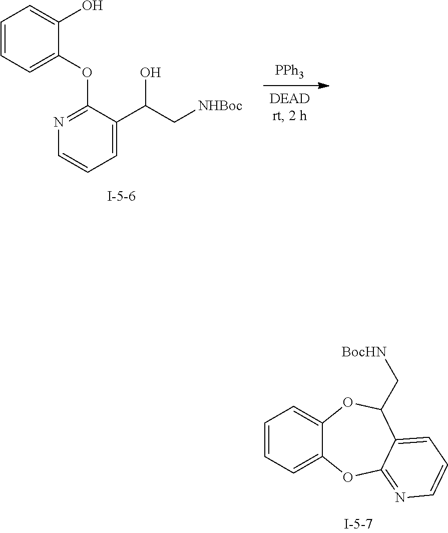 [SMILES: CC(C)(C)OC(=O)NCC(O)c1cccnc1Oc1ccccc1O ; "DEADrt, 2 h" ; CC(C)(C)OC(=O)NCC1Oc2ccccc2Oc2ncccc21 ; "PPh3"]

To a solution of tert-butyl (2-hydroxy-2-(2-(2-hydroxyphenoxy)pyridin-3-yl)ethyl)carbamate (4 g, 11.5 mmol) in tetrahydrofuran (50 mL) was added triphenylphosphine (3.01 g, 11.5 mmol) and N-(ethoxycarbonyl-imino-ethoxyformamide) (2.00 g, 11.5 mmol) at about 0° C. The reaction was stirred at about room temperature for about 2 hours and concentrated under vacuum, the residue was purified by silica gel chromatography (petroleum ether/ethyl acetate=3/1) to give tert-butyl ((5H-benzo[2,3][1,4]dioxepino[5,6-b]pyridin-5-yl)methyl)carbamate. MS (ESI): m/z 329 [M+H]⁺

### g. Chiral column separation of tert-butyl ((5H-benzo[2,3][1,4]dioxepino[5,6-b]pyridin-5-yl)methyl)carbamate

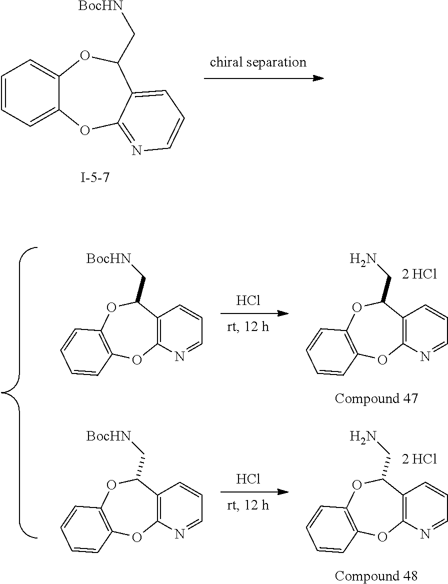

The racemic tert-butyl ((5H-benzo[2,3][1,4]dioxepino[5,6-b]pyridin-5-yl)methyl)carbamate was separated by chiral HPLC under following condition:

| | |
|---|---|
| Instrument: SFC-150 (Waters) | Column: OJ 20*250 mm, 10 μm (Daicel) |
| Column temperature: 35° C. | Mobile phase: CO2/IPA(0.5MEA) = 70/30 |
| Flow rate: 100 g/min | Back pressure: 100 bar |
| Detection wavelength: 214 nm | Cycle time: 2.01 min |
| Sample solution: 1000 mg | |
| dissolved in 35 mL Methanol | |
| Injection volume: 1 mL | |

After removal of solvents, the first eluting isomer (I-5-7-P1) (400 mg, retention time=1.50 min) and the second eluting isomer (I-5-7-P2) (400 mg, retention time=1.86 min) were obtained. MS (ESI): m/z 329 [M+H]⁺

### Synthesis of (R*)-(5H-benzo[2,3][1,4]dioxepino[5,6-b]pyridin-5-yl)methanamine (Compound 47) and (S*)-(5H-benzo[2,3][1,4]dioxepino[5,6-b]pyridin-5-yl)methanamine (Compound 48)

Independently, for each individual isomer I-5-7-P1 and I-5-7-P2, a solution of each compound (100 mg, 304 μmol) in MeOH (5 mL) was added HCl/MeOH (3 M, 3 mL). The reaction was stirred at about room temperature for about 16 hours and concentrated under vacuum. The residue was dissolved in water (5 mL) and extracted with ethyl acetate (5 mL). The aqueous phase was freeze-dried to give Compound 47. MS (ESI): m/z 229 [M+H]⁺. ¹H NMR (400 MHz, CD₃OD) δ 8.50 (d, J=5.2 Hz, 1H), 8.35 (d, J=7.6 Hz, 1H), 7.59 (dd, J=20.7, 13.9 Hz, 1H), 7.38 (dd, J=19.1, 7.7 Hz, 2H), 7.30-7.14 (m, 2H), 5.72 (d, J=9.2 Hz, 1H), 3.89 (d, J=13.3 Hz, 1H), 3.78-3.60 (m, 1H). Chiral analysis column: OJ-H (250*4.6 mm 5 μm), Mobile Phase: n-Hexane (0.1% DEA):EtOH (0.1% DEA)=80:20, flowrate: 1.0 mL/min, column Temperature: 40° C., retention time=9.48 min.; and Compound 48. MS (ESI): m/z 229 [M+H]⁺. ¹H NMR (400 MHz, CD₃OD) δ 8.43 (d, J=5.0 Hz, 1H), 8.17 (d, J=7.5 Hz, 1H), 7.61-7.44 (m, 1H), 7.36 (d, J=7.5 Hz, 1H), 7.27 (d, J=7.7 Hz, 1H), 7.22-7.09 (m, 2H), 5.70 (d, J=9.6 Hz, 1H), 3.83 (d, J=13.1 Hz, 1H), 3.78-3.63 (m, 1H). Chiral analysis column: OJ-H (250*4.6 mm 5 μm), Mobile Phase: n-Hexane (0.1% DEA):EtOH (0.1% DEA)=80:20, flowrate: 1.0 mL/min, column Temperature: 40° C., retention time=8.48 min.

### Example 32. Synthesis of (R*)-1-(5H-benzo[2,3][1,4]dioxepino[5,6-b]pyridin-5-yl)-N-methylmethanamine (Compound 45) and (S*)-1-(5H-benzo[2,3][1,4]dioxepino[5,6-b]pyridin-5-yl)-N-methylmethanamine (Compound 46)

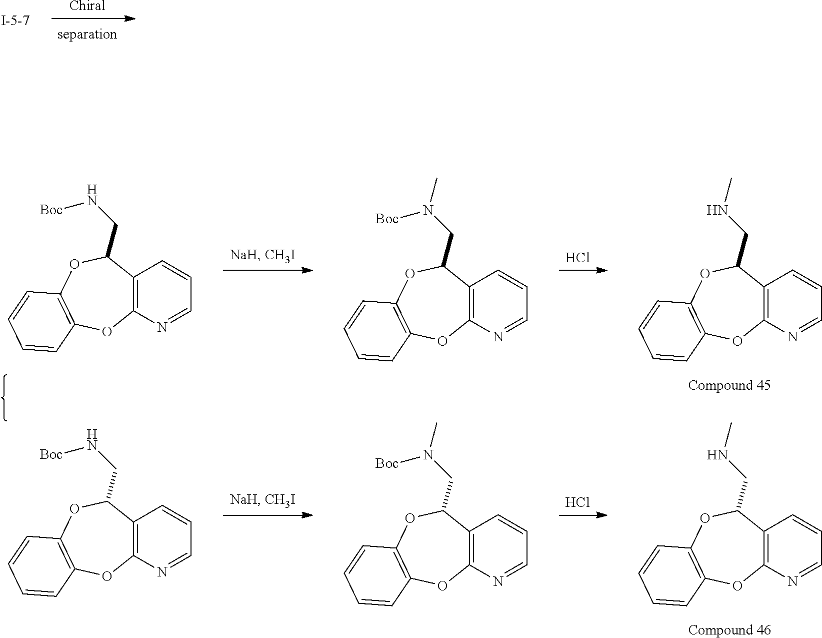

### Synthesis of (R*)-1-(5H-benzo[2,3][1,4]dioxepino[5,6-b]pyridin-5-yl)-N-methylmethanamine (Compound 45) and (S*)-1-(5H-benzo[2,3][1,4]dioxepino[5,6-b]pyridin-5-yl)-N-methylmethanamine (Compound 46)

Independently, for each individual isomer I-5-7-P1 and I-5-7-P2, to a solution of each compound (130 mg, 395 μmol) in tetrahydrofuran (5 mL) was added sodium hydride (28.3 mg, 1.18 mmol) and iodomethane (84.0 mg, 592 μmol) at about 0° C. The reaction was stirred at about room temperature for about 3 hours and quenched with ice-water (3 mL). The mixture was extracted with ethyl acetate (15 mL). The organic layer was evaporated and purified by silica gel chromatography (petroleum ether/ethyl acetate=4/1) to give the N-methylated intermediate MS (ESI): m/z 343[M+H]⁺

To a solution of each N-methylated intermediate (140 mg, 408 μmol) in MeOH (5 mL) was added HCl/MeOH (2M, 2 mL, 6 mmol). The reaction was stirred at about room temperature for about 16 hours and concentrated under vacuum. The residue was dissolved in water (5 mL) and extracted with ethyl acetate (5 mL). The aqueous phase was freeze-dried on a lyophilizer to give Compound 45. MS (ESI): m/z 243 [M+H]⁺. ¹H NMR (400 MHz, CD₃OD) δ 8.64-8.46 (m, 1H), 8.40 (d, J=7.4 Hz, 1H), 7.65 (dd, J=7.7, 5.6 Hz, 1H), 7.47-7.36 (m, 2H), 7.25 (pd, J=7.4, 1.9 Hz, 2H), 5.91-5.69 (m, 1H), 3.99 (dd, J=13.1, 2.5 Hz, 1H), 3.93-3.79 (m, 1H), 2.92 (s, 3H). Chiral analysis column: IC-3 4.6*100 mm 3 μm; co-solvent: MeOH [0.2% NH3(7M in MeOH)]; Acq. method Set: IC 35% B1; Flow rate: 3.0 mL/min; column temperature: 40° C.; retention time=1.36 min.; and Compound 46. MS (ESI): m/z 243 [M+H]⁺. ¹H NMR (400 MHz, CD3OD) δ 8.44 (dd, J=5.3, 1.5 Hz, 1H), 8.19 (d, J=7.7 Hz, 1H), 7.66-7.45 (m, 1H), 7.37 (dt, J=14.8, 4.6 Hz, 1H), 7.29 (dt, J=8.9, 4.6 Hz, 1H), 7.19 (pd, J=7.4, 1.9 Hz, 2H), 5.77 (dt, J=39.0, 19.4 Hz, 1H), 3.95-3.75 (m, 2H), 2.90 (s, 3H). Chiral analysis column: IC-3 4.6*100 mm 3 μm; co-solvent: MeOH [0.2% NH3(7M in MeOH)]; Acq. method Set: IC 35% B1; Flow rate: 3.0 mL/min; column temperature: 40° C.; retention time=2.84 min.

### Example 33. Synthesis of (R*)-(5H-benzo[2,3][1,4]dioxepino[5,6-c]pyridin-5-yl)methanamine (Compound 49) and (S*)-(5H-benzo[2,3][1,4]dioxepino[5,6-c]pyridin-5-yl)methanamine (Compound 50)

### a. Preparation of 3-(2-(benzyloxy)phenoxy)isonicotinaldehyde

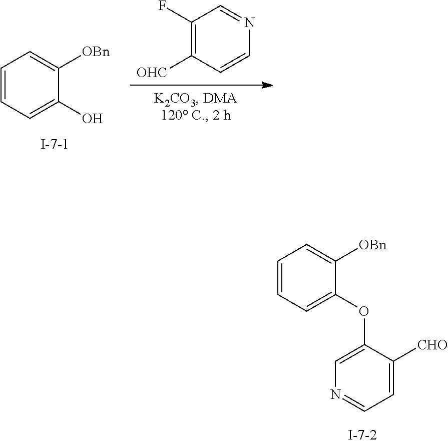

To a solution of 2-(benzyloxy)phenol (5 g, 24.9 mmol) in dimethylacetamide (30 mL) was added 3-fluoroisonicotinaldehyde (3.1 g, 24.9 mmol) and K₂CO₃ (5.5 g, 39.8 mmol). The reaction mixture was heated to about 120° C. and stirred at that temperature for about 2 hours. Water (40 mL) was added to the reaction vessel and the resulting biphasic mixture was transferred to a separatory funnel. The layers were separated, and the organic phase was washed with saturated aqueous NaCl (30 mL). The combined organics were dried over anhydrous Na₂SO₄, filtered, and concentrated in vacuo. The resulting substance was purified by flash column chromatography with a gradient elution of petroleum ether (100%) and ethyl acetate (0%) to petroleum ether (60%) and ethyl acetate (40%) to provide 3-(2-(benzyloxy)phenoxy)isonicotinaldehyde. MS (ESI): m/z 306 [M+H]⁺.

### b. Preparation of 2-(3-(2-(benzyloxy)phenoxy)pyridin-4-yl)-2-hydroxyacetonitrile

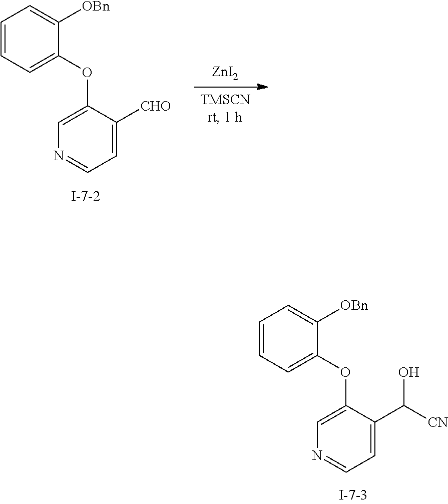 [SMILES: O=Cc1ccncc1Oc1ccccc1OCc1ccccc1 ; "ZnI2" ; "TMSCNrt, 1 h" ; N#CC(O)c1ccncc1Oc1ccccc1OCc1ccccc1]

To a solution of 3-(2-(benzyloxy)phenoxy)picolinaldehyde (6.5 g, 21.2 mmol) in methylene chloride (40 mL) was added trimethylsilanecarbonitrile (4.2 g, 42.4 mmol) and diiodozinc (673 mg, 2.11 mmol). The reaction was stirred at about room temperature for about 2 hours and filtered. The filtrate was concentrated to afford 2-(3-(2-(benzyloxy)phenoxy)pyridin-4-yl)-2-hydroxyacetonitrile. MS (ESI): m/z 333 [M+H]⁺.

### c. Preparation of 2-amino-1-(3-(2-(benzyloxy)phenoxy)pyridin-4-yl)ethan-1-ol

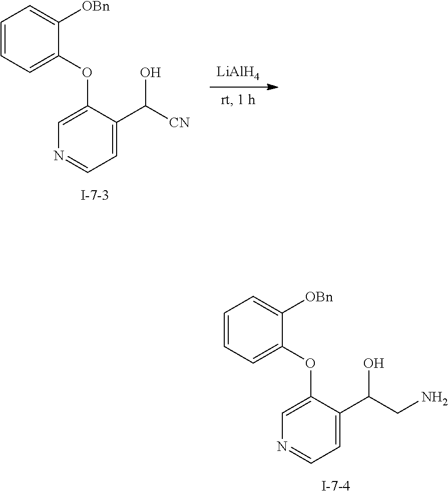 [SMILES: N#CC(O)c1ccncc1Oc1ccccc1OCc1ccccc1 ; "LiAlH4" ; "rt, 1 h" ; NCC(O)c1ccncc1Oc1ccccc1OCc1ccccc1]

To a solution of 2-(3-(2-(benzyloxy)phenoxy)pyridin-4-yl)-2-hydroxyacetonitrile (6.0 g, 18.0 mmol) in tetrahydrofuran (50 mL) was added LiAlH₄ (1.36 g, 36.0 mmol) at about 0° C. The reaction was stirred at about room temperature for about 1 hour and quenched with Na₂SO₄.10H₂O (10.0 g). The mixture was stirred at about room temperature for about 20 minutes and filtered. The filtrate was dried and concentrated to give 2-amino-1-(3-(2-(benzyloxy)phenoxy)pyridin-4-yl)ethan-1-ol. MS (ESI): m/z 337 [M+H]⁺.

### d. Preparation of tert-butyl (2-(3-(2-(benzyloxy)phenoxy)pyridin-4-yl)-2-hydroxyethyl)carbamate

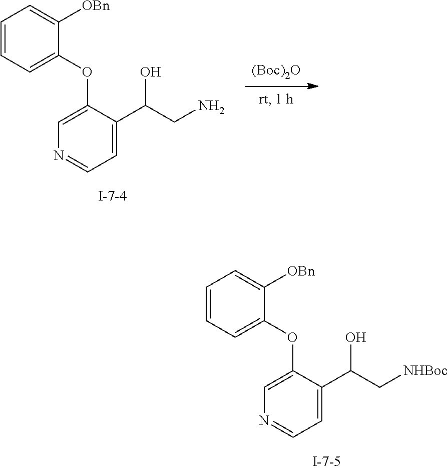 [SMILES: "(Boc)2O" ; NCC(O)c1ccncc1Oc1ccccc1OCc1ccccc1 ; "rt, 1 h" ; CC(C)(C)OC(=O)NCC(O)c1ccncc1Oc1ccccc1OCc1ccccc1]

To a solution of 2-amino-1-(3-(2-(benzyloxy)phenoxy)pyridin-4-yl)ethan-1-ol (5.0 g, 14.8 mmol) in methylene chloride (50 mL) and Na₂CO₃ solution (50 mL) was added di-tert-butyl dicarbonate (3.2 g, 14.8 mmol). The reaction was stirred at about room temperature for about 1 hour. Water (100 mL) was added to the reaction vessel and the mixture was extracted with methylene chloride (2×100 mL). The combined organic layers were dried over anhydrous Na₂SO₄, filtered, and concentrated in vacuo. The resulting substance was purified by silica gel chromatography (petroleum ether/ethyl acetate=3/1) to give tert-butyl (2-(3-(2-(benzyloxy)phenoxy)pyridin-4-yl)-2-hydroxyethyl)carbamate. MS (ESI): m/z 437 [M+H]⁺.

### e. Preparation of tert-butyl (2-hydroxy-2-(3-(2-hydroxyphenoxy)pyridin-4-yl)ethyl)carbamate

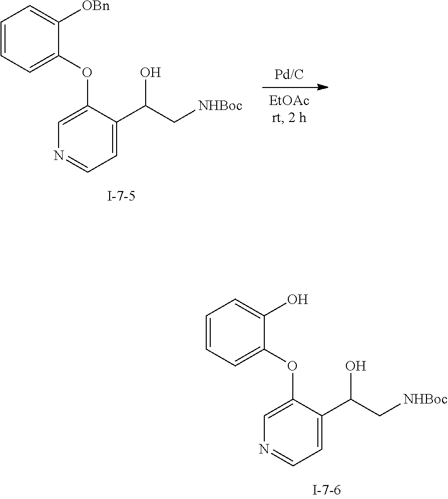 [SMILES: CC(C)(C)OC(=O)NCC(O)c1ccncc1Oc1ccccc1OCc1ccccc1 ; "EtOAcrt, 2 h" ; "Pd/C" ; CC(C)(C)OC(=O)NCC(O)c1ccncc1Oc1ccccc1O]

To a solution of tert-butyl (2-(3-(2-(benzyloxy)phenoxy)pyridin-4-yl)-2-hydroxyethyl)carbamate (2.5 g, 5.72 mmol) in ethyl acetate (30 mL) was added Pd/C (500 mg, 10%). The mixture was stirred at about room temperature for about 2 hours and filtered. The filtrate was concentrated under vacuum to give tert-butyl (2-hydroxy-2-(3-(2-hydroxyphenoxy)pyridin-4-yl)ethyl)carbamate. MS (ESI): m/z 347 [M+H]⁺.

### f. Preparation of tert-butyl ((5H-benzo[2,3][1,4]dioxepino[5,6-c]pyridin-5-yl)methyl)carbamate

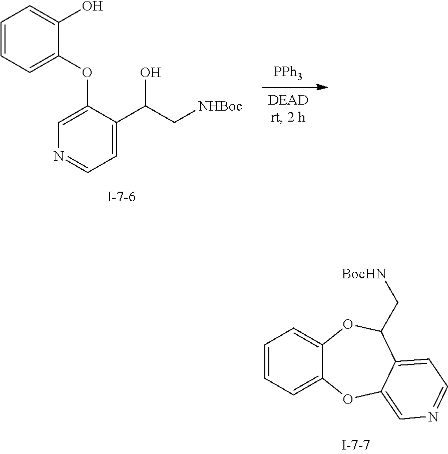 [SMILES: CC(C)(C)OC(=O)NCC(O)c1ccncc1Oc1ccccc1O ; "DEADrt, 2 h" ; CC(C)(C)OC(=O)NCC1Oc2ccccc2Oc2cnccc21 ; "PPh3"]

To a solution of tert-butyl (2-hydroxy-2-(3-(2-hydroxyphenoxy)pyridin-4-yl)ethyl)carbamate (2 g, 5.77 mmol) in tetrahydrofuran (20 mL) was added triphenylphosphine (1.81 g, 6.92 mmol) and N[(ethoxycarbonyl)imino]ethoxyformamide (1.20 g, 6.92 mmol) at about 0° C. The reaction was stirred at about room temperature for about 10 minutes and concentrated under vacuum, the resulting substance was purified by silica gel chromatography (petroleum ether/ethyl acetate=3/1) to give tert-butyl ((5H-benzo[2,3][1,4]dioxepino[5,6-c]pyridin-5-yl)methyl)carbamate. MS (ESI): m/z 329 [M+H]⁺

### g. Chiral column separation of tert-butyl ((5H-benzo[2,3][1,4]dioxepino[5,6-c]pyridin-5-yl)methyl)carbamate

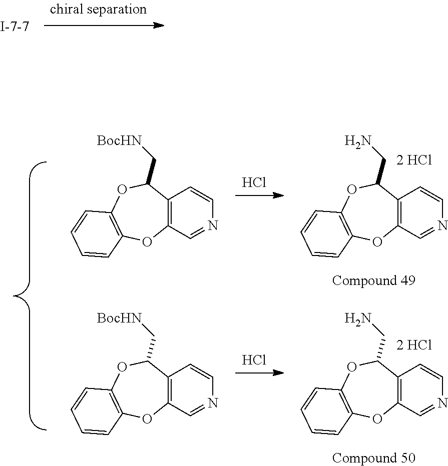

The racemic tert-butyl ((5H-benzo[2,3][1,4]dioxepino[5,6-c]pyridin-5-yl)methyl)carbamate was purified by chiral column using:

| | |
|---|---|
| Instrument: SFC-150 (Waters) | Column: IG 20*250 |
| Column temperature: 35° C. | mm, 10 μm (Daicel), |
| Mobile phase: CO2/MeOH(0.2% | |
| Methanol Ammonia) = 70/30, | |
| Flow rate: 100 g/min | Back pressure: 100 bar |
| Detection wavelength: 214 nm | Cycle time: 4 min |
| Sample solution: 1200 mg dissolved in | |
| 50 mL methanol | |
| Injection volume: 1.5 mL | |

After removal of solvents, the first eluting isomer (I-7-7-P1) (500 mg, retention time=1.79 min) and the second eluting isomer (I-7-7-P2) (500 mg, retention time=2.38 min) were obtained. MS (ESI): m/z 329 [M+H]⁺

### Synthesis of (R*)-(5H-benzo[2,3][1,4]dioxepino[5,6-c]pyridin-5-yl)methanamine (Compound 49) and (S*)-(5H-benzo[2,3][1,4]dioxepino[5,6-c]pyridin-5-yl)methanamine (Compound 50)

Independently, for each individual isomer I-7-7-P1 and I-7-7-P2, to a solution of each compound (170 mg, 517 μmol) in MeOH (5 mL) was added HCl/MeOH (3M, 3 mL, 9 mmol). The reaction was stirred at about room temperature for about 16 hours and concentrated under vacuum. The residue was dissolved in water (5 mL) and extracted with ethyl acetate (5 mL). The aqueous phase was freeze-dried on a lyophilizer to give Compound 49. MS (ESI): m/z 229 [M+H]⁺. ¹H NMR (400 MHz, CD₃OD) δ 8.98 (d, J=3.6 Hz, 1H), 8.70-8.42 (m, 1H), 7.99 (t, J=7.0 Hz, 1H), 7.50-7.29 (m, 2H), 7.29-7.08 (m, 2H), 5.83 (dd, J=9.7, 2.6 Hz, 1H), 3.91 (d, J=13.6 Hz, 1H), 3.71 (dd, J=13.4, 9.8 Hz, 1H). Chiral analysis column: OJ-H (250*4.6 mm 5 μm); Mobile Phase: n-Hexane (0.1% DEA):EtOH (0.1% DEA)=90:10; Flow rate: 1.0 mL/min; column temperature: 40° C.; retention time=15.11 min.; and Compound 50. MS (ESI): m/z 229 [M+H]⁺. ¹H NMR (400 MHz, CD₃OD) δ 8.98 (d, J=4.0 Hz, 1H), 8.63 (dd, J=5.9, 2.3 Hz, 1H), 8.01 (t, J=6.6 Hz, 1H), 7.45-7.32 (m, 2H), 7.29-7.10 (m, 2H), 5.93-5.68 (m, 1H), 3.92 (dd, J=13.4, 2.0 Hz, 1H), 3.71 (dd, J=13.4, 9.8 Hz, 1H). Chiral analysis column: OJ-H (250*4.6 mm 5 μm); Mobile Phase: n-Hexane (0.1% DEA):EtOH (0.1% DEA)=90:10; Flow rate: 1.0 mL/min; column temperature: 40° C.; retention time=16.29 min.

### Example 34. Synthesis of (R*)-1-(5H-benzo[2,3][1,4]dioxepino[5,6-c]pyridin-5-yl)-N-methylmethanamine (Compound 51) and (S*)-1-(5H-benzo[2,3][1,4]dioxepino[5,6-c]pyridin-5-yl)-N-methylmethanamine (Compound 52)

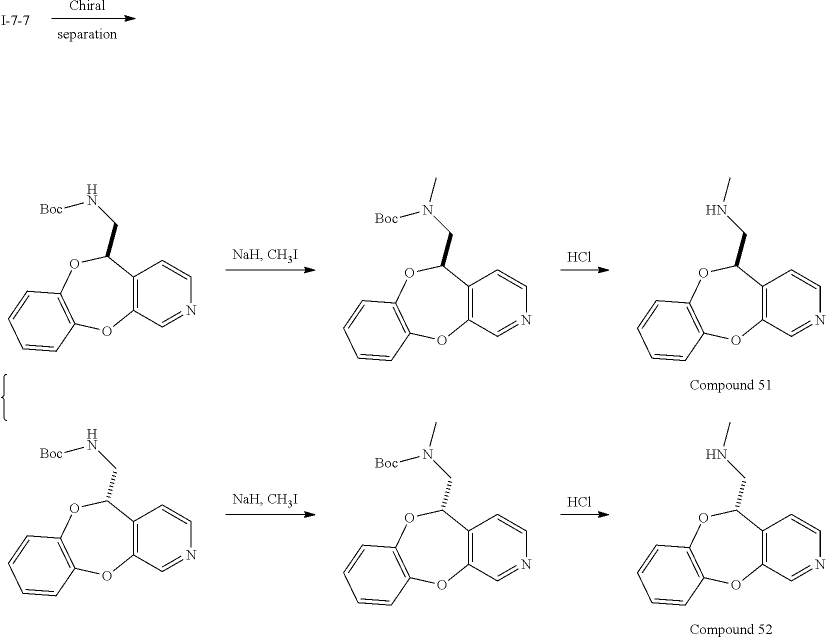

### Synthesis of (R*)-1-(5H-benzo[2,3][1,4]dioxepino[5,6-c]pyridin-5-yl)-N-methylmethanamine (Compound 51) and (S*)-1-(5H-benzo[2,3][1,4]dioxepino[5,6-c]pyridin-5-yl)-N-methylmethanamine (Compound 52)

Independently, for each individual compound I-7-7-P1 and I-7-7-P2, to a solution of each compound (120 mg, 365 μmol) in tetrahydrofuran (5 mL) was added sodium hydride (103 mg, 730 μmol) and methyl iodide (103 mg, 730 μmol) at about 0° C. The reaction was stirred at about room temperature for about 3 hours and quenched with ice-water (3 mL). The mixture was extracted with ethyl acetate (15 mL×2). The combined organic layers were dried over anhydrous Na₂SO₄, filtered, and concentrated in vacuo. The resulting substance was purified by silica gel chromatography (petroleum ether/ethyl acetate=4/1) to give the N-methylated intermediate. MS (ESI): m/z 343[M+H]⁺

To a solution of the N-methylated intermediate (120 mg, 350 μmol) in MeOH (5 mL) was added HCl/MeOH (5 mL, 10 mmol). The reaction was stirred at about room temperature for about 16 hours and concentrated. under vacuum. The residue was partitioned with ethyl acetate (5 mL)/H₂O (5 mL). The residue was dissolved in water (5 mL) and freeze-dried on a lyophilizer to give Compound 51. MS (ESI): m/z 243 [M+H]⁺. ¹H NMR (400 MHz, CD₃OD) δ 9.01 (s, 1H), 8.65 (d, J=5.9 Hz, 1H), 8.03 (d, J=5.9 Hz, 1H), 7.53-7.32 (m, 2H), 7.30-7.06 (m, 2H), 5.93 (dd, J=9.8, 2.9 Hz, 1H), 4.00 (dd, J=13.1, 2.9 Hz, 1H), 3.85 (dd, J=13.0, 10.0 Hz, 1H), 2.92 (s, 3H). Chiral analysis column: AY-H (250*4.6 mm 5 μm); Mobile Phase: n-Hexane (0.1% DEA):EtOH (0.1% DEA)=90:10; Flow rate: 1.0 mL/min; column temperature: 40° C.; retention time=10.36 min.; and Compound 52. MS (ESI): m/z 243 [M+H]⁺. ¹H NMR (400 MHz, CD₃OD) δ 8.97 (s, 1H), 8.62 (d, J=5.9 Hz, 1H), 7.95 (d, J=5.9 Hz, 1H), 7.47-7.31 (m, 2H), 7.30-7.09 (m, 2H), 5.90 (dd, J=9.9, 2.9 Hz, 1H), 3.98 (dd, J=13.2, 3.0 Hz, 1H), 3.85 (dd, J=13.0, 10.1 Hz, 1H), 2.92 (s, 3H). Chiral analysis column: AY-H (250*4.6 mm 5 μm); Mobile Phase: n-Hexane (0.1% DEA):EtOH (0.1% DEA)=90:10; Flow rate: 1.0 mL/min; column temperature: 40° C.; retention time=13.58 min.

### Example 35. Synthesis of (R*)-(11H-benzo[2,3][1,4]dioxepino[6,5-c]pyridin-11-yl)methanamine (Compound 53) and (S*)-(11H-benzo[2,3][1,4]dioxepino[6,5-c]pyridin-11-yl)methanamine (Compound 54)

### a. Preparation of 4-(2-(benzyloxy)phenoxy)nicotinaldehyde

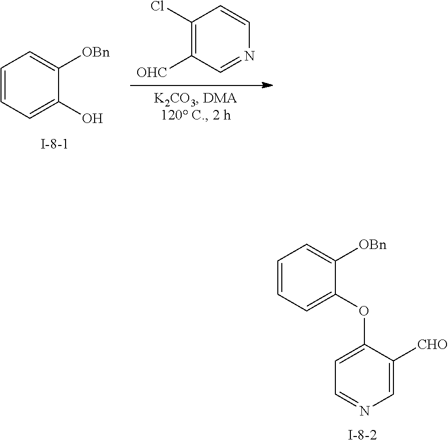

To a solution of 2-(benzyloxy)phenol (2 g, 9.98 mmol) in DMF (50 mL) was added 4-chloropyridine-3-carbaldehyde (1.41 g, 9.98 mmol) and K₂CO₃ (2.75 g, 19.9 mmol). The reaction mixture was heated to about 120° C. and stirred at that temperature for about 2 hours. Water (40 mL) and ethyl acetate (100 mL) were added to the reaction vessel and the resulting biphasic mixture was transferred to a separatory funnel. The layers were separated, and the organic phase was washed with saturated aqueous NaCl (30 mL). The combined organics were dried over anhydrous Na₂SO₄, filtered, and concentrated in vacuo. The resulting substance was purified by flash column chromatography with a gradient elution of petroleum ether (100%) to petroleum ether (60%) and ethyl acetate (40%) to provide 4-(2-(benzyloxy)phenoxy)nicotinaldehyde. MS (ESI): m/z 306 [M+H]⁺.

### b. Preparation of 2-(4-(2-(benzyloxy)phenoxy)pyridin-3-yl)-2-hydroxyacetonitrile

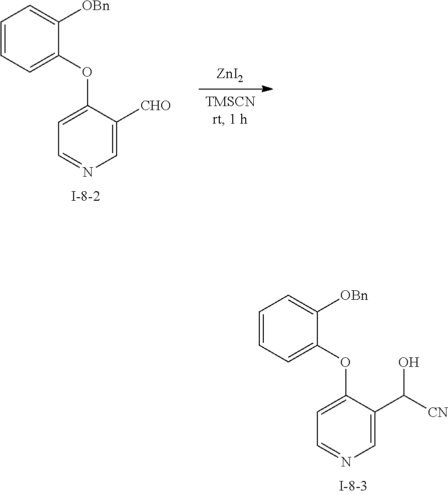 [SMILES: O=Cc1cnccc1Oc1ccccc1OCc1ccccc1 ; "ZnI2" ; "TMSCNrt, 1 h" ; N#CC(O)c1cnccc1Oc1ccccc1OCc1ccccc1]

To a solution of 4-(2-(benzyloxy)phenoxy)nicotinaldehyde (8.5 g, 27.8 mmol) in methylene chloride (40 mL) was added trimethylsilanecarbonitrile (5.51 g, 55.6 mmol) and diiodozinc (887 mg, 2.78 mmol). The reaction was stirred at about room temperature for about 2 hours and filtered. The filtrate was concentrated to afford 2-(4-(2-(benzyloxy)phenoxy)pyridin-3-yl)-2-hydroxyacetonitrile. MS (ESI): m/z 333 [M+H]⁺.

### c. Preparation of 2-amino-1-(4-(2-(benzyloxy)phenoxy)pyridin-3-yl)ethan-1-ol

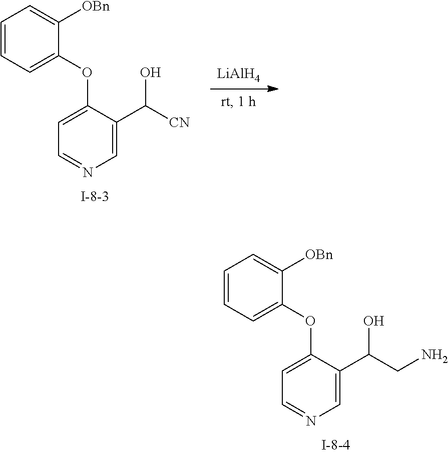 [SMILES: N#CC(O)c1cnccc1Oc1ccccc1OCc1ccccc1 ; "LiAlH4" ; "rt, 1 h" ; NCC(O)c1cnccc1Oc1ccccc1OCc1ccccc1]

To a solution of 2-(4-(2-(benzyloxy)phenoxy)pyridin-3-yl)-2-hydroxyacetonitrile (6.5 g, 19.5 mmol) in tetrahydrofuran (50 mL) was added LiAlH₄ (1.48 g, 39.0 mmol) at about 0° C. The reaction was stirred at about room temperature for about 1 hour and quenched with Na₂SO₄.10H₂O (10.0 g). The mixture was stirred at about room temperature for about 20 minutes and filtered. The filtrate was dried and concentrated to give 2-amino-1-(4-(2-(benzyloxy)phenoxy)pyridin-3-yl)ethan-1-ol. MS (ESI): m/z 337 [M+H]⁺.

### d. Preparation of tert-butyl (2-(4-(2-(benzyloxy)phenoxy)pyridin-3-yl)-2-hydroxyethyl)carbamate

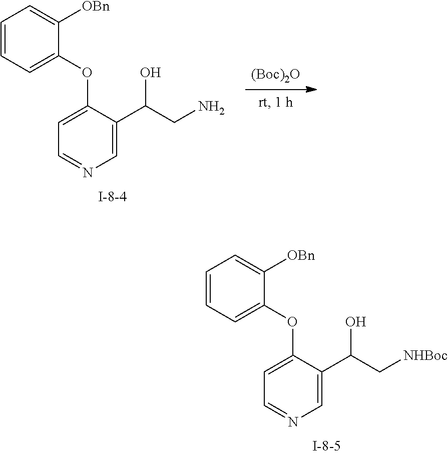 [SMILES: "(Boc)2O" ; NCC(O)c1cnccc1Oc1ccccc1OCc1ccccc1 ; "rt, 1 h" ; CC(C)(C)OC(=O)NCC(O)c1cnccc1Oc1ccccc1OCc1ccccc1]

To a solution of 2-amino-1-(4-(2-(benzyloxy)phenoxy)pyridin-3-yl)ethan-1-ol (6.0 g, 17.8 mmol) in methylene chloride (50 mL) and Na₂CO₃ solution (50 mL) was added di-tert-butyl dicarbonate (3.9 g, 17.8 mmol). The reaction was stirred at about room temperature for about 1 hour. The organic layer was purified by silica gel chromatography (petroleum ether/ethyl acetate=3/1) to give tert-butyl (2-(4-(2-(benzyloxy)phenoxy)pyridin-3-yl)-2-hydroxyethyl)carbamate. MS (ESI): m/z 437 [M+H]⁺.

### e. Preparation of tert-butyl (2-hydroxy-2-(4-(2-hydroxyphenoxy)pyridin-3-yl)ethyl)carbamate

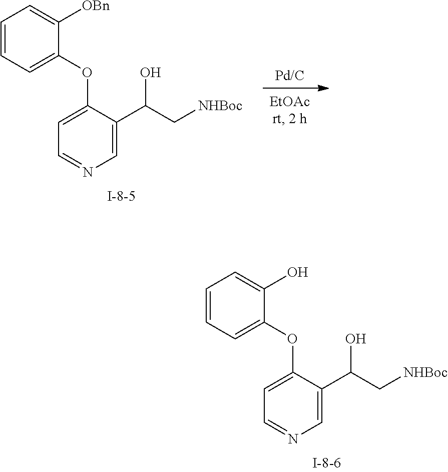 [SMILES: CC(C)(C)OC(=O)NCC(O)c1cnccc1Oc1ccccc1OCc1ccccc1 ; "EtOAcrt, 2 h" ; "Pd/C" ; CC(C)(C)OC(=O)NCC(O)c1cnccc1Oc1ccccc1O]

To a solution of tert-butyl (2-(4-(2-(benzyloxy)phenoxy)pyridin-3-yl)-2-hydroxyethyl)carbamate (4.6 g, 10.5 mmol) in ethyl acetate (30 mL) was added Pd/C (500 mg, 10%). The mixture was stirred at about room temperature for about 2 hours and filtered. The filtrate was concentrated under vacuum to give tert-butyl (2-hydroxy-2-(4-(2-hydroxyphenoxy)pyridin-3-yl)ethyl)carbamate. MS (ESI): m/z 347 [M+H]⁺.

### f. Preparation of tert-butyl ((5H-benzo[2,3][1,4]dioxepino[5,6-c]pyridin-5-yl)methyl)carbamate

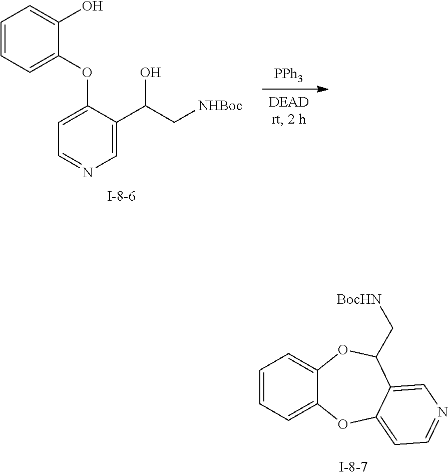 [SMILES: CC(C)(C)OC(=O)NCC(O)c1cnccc1Oc1ccccc1O ; "DEADrt, 2 h" ; CC(C)(C)OC(=O)NCC1Oc2ccccc2Oc2ccncc21 ; "PPh3"]

To a solution of tert-butyl (2-hydroxy-2-(4-(2-hydroxyphenoxy)pyridin-3-yl)ethyl)carbamate (2.35 g, 6.78 mmol) in tetrahydrofuran (20 mL) was added triphenylphosphine (2.13 g, 8.13 mmol) and N[(ethoxycarbonyl)imino]ethoxyformamide (1.41 g, 8.13 mmol) at about 0° C. The reaction was stirred at about room temperature for about 10 minutes and concentrated under vacuum to give the crude compound, which was purified by silica gel chromatography (petroleum ether/ethyl acetate=3/1) to give tert-butyl ((11H-benzo[2,3][1,4]dioxepino[6,5-c]pyridin-11-yl)methyl)carbamate. MS (ESI): m/z 329 [M+H]⁺

### g. Chiral column separation of tert-butyl ((11H-benzo[2,3][1,4]dioxepino[6,5-c]pyridin-11-yl)methyl)carbamate

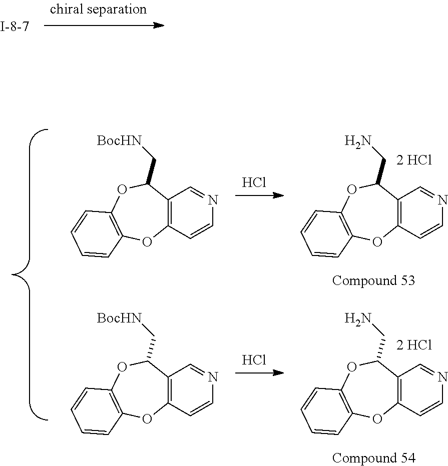

The racemic tert-butyl ((11H-benzo[2,3][1,4]dioxepino[6,5-c]pyridin-11-yl)methyl)carbamate was purified by chiral column using:

| | |
|---|---|
| Instrument: SFC-80 (Thar, Waters), | Column: AD 20*250 |
| Column temperature: 35° C. | mm, 10 μm (Daicel) |
| Mobile phase: CO2/MeOH (0.2% | |
| Methanol Ammonia) = 70/30 | |
| Flow rate: 80 g/min | Back pressure: 100 bar |
| Detection wavelength: 214 nm | Cycle time: 6.7 min |
| Sample solution: 650 mg dissolved in | |
| 25 mL methanol | |
| Injection volume: 0.6 mL | |

After removal of the solvents, the first eluting isomer (I-8-7-P1) (300 mg, retention time=1.63 min) and the second eluting isomer (I-8-7-P2) (250 mg, retention time=2.26 min) were obtained. MS (ESI): m/z 329 [M+H]⁺

### Synthesis of (R*)-(11H-benzo[2,3][1,4]dioxepino[6,5-c]pyridin-11-yl)methanamine (Compound 53) and (S*)-(11H-benzo[2,3][1,4]dioxepino[6,5-c]pyridin-11-yl)methanamine (Compound 54)

Independently, for each individual isomer I-8-7-P1 and I-8-7-P2, to a solution of each isomer (100 mg, 304 μmol) in MeOH (5 mL) was added HCl/MeOH (3 M, 3 mL, 9 mmol). The reaction was stirred at about room temperature for about 16 hours and concentrated under vacuum. The residue was dissolved in water (5 mL) and extracted with ethyl acetate (5 mL). The aqueous phase was freeze-dried on lyophilizer to give Compound 53. MS (ESI): m/z 229 [M+H]⁺. ¹H NMR (400 MHz, CD₃OD) δ 8.87 (s, 1H), 8.78 (d, J=6.6 Hz, 1H), 7.84 (d, J=6.7 Hz, 1H), 7.42 (ddd, J=10.7, 6.8, 3.6 Hz, 2H), 7.36-7.18 (m, 2H), 5.76-5.56 (m, 1H), 3.97 (dd, J=13.3, 2.8 Hz, 1H), 3.67 (dd, J=13.3, 10.0 Hz, 1H). Chiral analysis column: OJ-H (250*4.6 mm 5 μm); Mobile Phase: n-Hexane (0.1% DEA):EtOH (0.1% DEA)=90:10; Flow rate: 1.0 mL/min; column temperature: 40° C.; retention time=14.38 min.; and Compound 54. MS (ESI): m/z 229 [M+H]⁺. ¹H NMR (400 MHz, CD₃OD) δ 8.88 (s, 1H), 8.79 (d, J=6.6 Hz, 1H), 7.84 (d, J=6.7 Hz, 1H), 7.53-7.36 (m, 2H), 7.36-7.14 (m, 2H), 5.69 (dd, J=9.8, 2.6 Hz, 1H), 3.97 (dd, J=13.3, 2.5 Hz, 1H), 3.67 (dd, J=13.3, 10.1 Hz, 1H). Chiral analysis column: OJ-H (250*4.6 mm 5 μm); Mobile Phase: n-Hexane (0.1% DEA):EtOH (0.1% DEA)=90:10; Flow rate: 1.0 mL/min; column temperature: 40° C.; retention time=17.35 min.

### Example 36. Synthesis of (R*)-1-(11H-benzo[2,3][1,4]dioxepino[6,5-c]pyridin-11-yl)-N-methylmethanamine (Compound 55) and (S*)-1-(11H-benzo[2,3][1,4]dioxepino[6,5-c]pyridin-11-yl)-N-methylmethanamine (Compound 56)

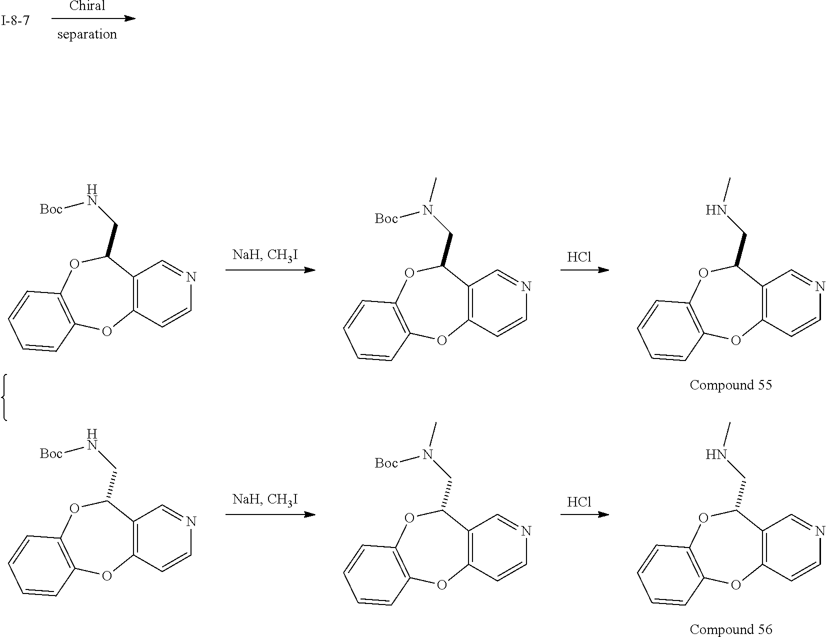

### (R)-1-(11H-benzo[2,3][1,4]dioxepino[6,5-c]pyridin-11-yl)-N-methylmethanamine (Compound 55) and (S)-1-(11H-benzo[2,3][1,4]dioxepino[6,5-c]pyridin-11-yl)-N-methylmethanamine (Compound 56)

Independently, for each compound I-8-7-P1 and I-8-7-P2, to a solution of each compound (100 mg, 304 μmol) in tetrahydrofuran (5 mL) was added sodium hydride (26.1 mg, 1.09 mmol) and iodomethane (77.6 mg, 547 μmol) at about 0° C. The reaction was stirred at about room temperature for about 3 hours and quenched with ice-water (3 mL). The mixture was extracted with ethyl acetate (15×2 mL). The combined organic layers were dried over anhydrous Na₂SO₄, filtered, and concentrated in vacuo. The resulting substance was purified by silica gel chromatography (petroleum ether/ethyl acetate=4/1) to give each N-methylated intermediate. MS (ESI): m/z 343[M+H]⁺

To a solution of each N-methylated intermediate (50 mg, 146 μmol) in MeOH (5 mL) was added HCl/MeOH (3 M, 3 mL, 9 mmol). The reaction was stirred at about room temperature for about 16 hours and concentrated under vacuum. The residue was dissolved in water (5 mL) and extracted with ethyl acetate (5 mL). The aqueous phase was freeze-dried on lyophilizer to give Compound 55. MS (ESI): m/z 243 [M+H]⁺. ¹H NMR (400 MHz, CD₃OD) δ 8.84 (s, 1H), 8.79 (d, J=6.4 Hz, 1H), 7.83 (d, J=6.6 Hz, 1H), 7.42 (tt, J=7.0, 3.6 Hz, 2H), 7.35-7.15 (m, 2H), 5.75 (dd, J=10.0, 2.7 Hz, 1H), 4.03 (d, J=10.5 Hz, 1H), 3.92-3.74 (m, 1H), 2.92 (s, 3H). Chiral analysis column: AY-H (250*4.6 mm 5 μm); Mobile Phase: n-Hexane (0.1% DEA):EtOH (0.1% DEA)=70:30; Flow rate: 1.0 mL/min; column temperature: 40° C.; retention time=8.31 min.; and Compound 56. MS (ESI): m/z 243 [M+H]⁺. ¹H NMR (400 MHz, CD₃OD) δ 8.85 (d, J=33.4 Hz, 2H), 7.86 (s, 1H), 7.45 (s, 2H), 7.30 (s, 2H), 5.80 (s, 1H), 4.06 (s, 1H), 3.85 (s, 1H), 2.93 (s, 3H). Chiral analysis column: AY-H (250*4.6 mm 5 μm); Mobile Phase: n-Hexane (0.1% DEA):EtOH (0.1% DEA)=70:30; Flow rate: 1.0 mL/min; column temperature: 40° C.; retention time=13.44 min.

### Example 37. Synthesis of (S*)-(11H-benzo[2,3][1,4]dioxepino[6,5-b]pyridin-11-yl)methanamine (Compound 59) and (R*)-(11H-benzo[2,3][1,4]dioxepino[6,5-b]pyridin-11-yl)methanamine (Compound 60)

### a. Preparation of 3-(2-(benzyloxy)phenoxy)picolinaldehyde

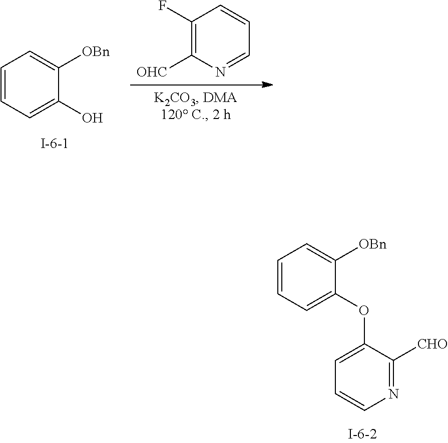

To a solution of 2-(benzyloxy)phenol (4 g, 19.9 mmol) in dimethylacetamide (5 mL) was added 3-fluoropicolinaldehyde (2.48 g, 19.9 mmol) and K₂CO₃ (5.5 g, 39.8 mmol). The reaction mixture was heated to about 120° C. and stirred at that temperature for about 2 hours. Water (40 mL) and ethyl acetate (100 mL) was added to the reaction vessel and the resulting biphasic mixture was transferred to a separatory funnel. The layers were separated, and the organic phase was washed with saturated aqueous NaCl (30 mL). The combined organics were dried over anhydrous Na₂SO₄, filtered, and concentrated in vacuo. The resulting substance was purified by flash column chromatography with a gradient elution of petroleum ether (100%) and ethyl acetate (0%) to petroleum ether (60%) and ethyl acetate (40%) to provide 3-(2-(benzyloxy)phenoxy)picolinaldehyde. MS (ESI): m/z 306 [M+H]⁺. b. Preparation of 2-(3-(2-(benzyloxy)phenoxy)pyridin-2-yl)-2-hydroxyacetonitrile

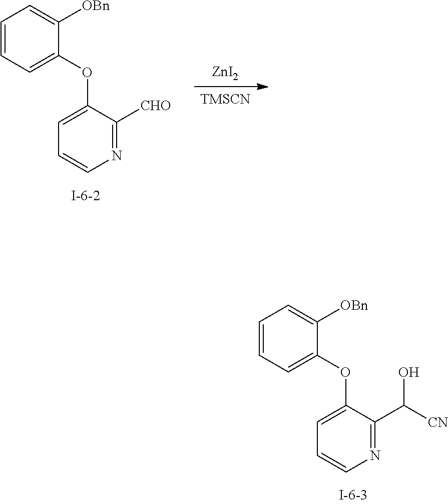 [SMILES: "TMSCN" ; O=Cc1ncccc1Oc1ccccc1OCc1ccccc1 ; "ZnI2" ; N#CC(O)c1ncccc1Oc1ccccc1OCc1ccccc1]

To a solution of 3-(2-(benzyloxy)phenoxy)picolinaldehyde (5.3 g, 17.3 mmol) in methylene chloride (40 mL) was added trimethylsilanecarbonitrile (3.43 g, 34.6 mmol) and iodozinc (552 mg, 1.73 mmol). The reaction was stirred at about room temperature for about 2 hours and filtered. The filtrate was concentrated to afford 2-(3-(2-(benzyloxy)phenoxy)pyridin-2-yl)-2-hydroxyacetonitrile. MS (ESI): m/z 333 [M+H]⁺.

### c. Preparation of 2-amino-1-(3-(2-(benzyloxy)phenoxy)pyridin-2-yl)ethan-1-ol

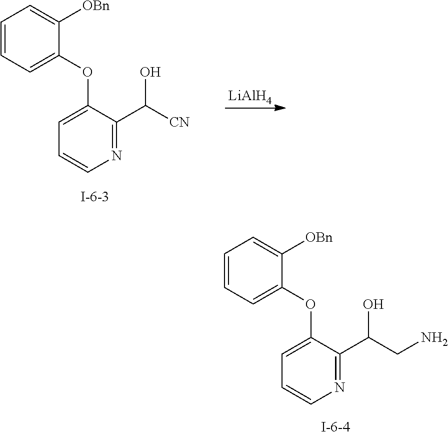 [SMILES: N#CC(O)c1ncccc1Oc1ccccc1OCc1ccccc1 ; "LiAlH4" ; NCC(O)c1ncccc1Oc1ccccc1OCc1ccccc1]

To a solution of 2-(3-(2-(benzyloxy)phenoxy)pyridin-2-yl)-2-hydroxyacetonitrile (4.7 g, 14.1 mmol) in tetrahydrofuran (50 mL) was added LiAlH₄ (1.07 g, 28.2 mmol) at about 0° C. The reaction was stirred at about room temperature for about 1 hour and quenched with Na₂SO₄.10H₂O (1.0 g). The mixture was stirred at about room temperature for 20 minutes and filtered. The filtrate was dried and concentrated to give 2-amino-1-(3-(2-(benzyloxy)phenoxy)pyridin-2-yl)ethan-1-ol. MS (ESI): m/z 337 [M+H]⁺.

### d. Preparation of tert-butyl (2-(3-(2-(benzyloxy)phenoxy)pyridin-2-yl)-2-hydroxyethyl)carbamate

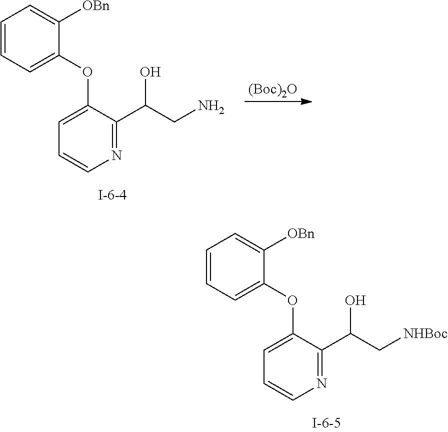 [SMILES: "(Boc)2O" ; NCC(O)c1ncccc1Oc1ccccc1OCc1ccccc1 ; CC(C)(C)OC(=O)NCC(O)c1ncccc1Oc1ccccc1OCc1ccccc1]

To a solution of 2-amino-1-(3-(2-(benzyloxy)phenoxy)pyridin-2-yl)ethan-1-ol (4.5 g, 13.3 mmol) in methylene chloride (50 mL) and Na₂CO₃ solution (50 mL) was added di-tert-butyl dicarbonate (2.90 g, 13.3 mmol). The reaction was stirred at about room temperature for 1 hour. Water (100 mL) was added to the reaction vessel and the mixture was extracted with ethyl acetate (3×100 mL). The combined organic layers were dried over anhydrous Na₂SO₄, filtered, and concentrated in vacuo. The resulting substance was purified by silica gel chromatography (petroleum ether/Ethyl acetate=3/1) to give tert-butyl (2-(3-(2-(benzyloxy)phenoxy)pyridin-2-yl)-2-hydroxyethyl)carbamate. MS (ESI): m/z 437 [M+H]⁺.

### e. Preparation of tert-butyl (2-hydroxy-2-(2-(2-hydroxyphenoxy)pyridin-3-yl)ethyl)carbamate

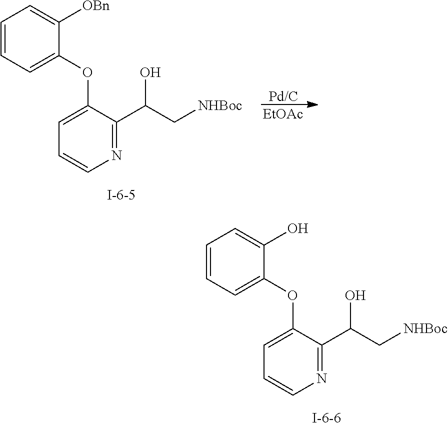 [SMILES: "EtOAc" ; CC(C)(C)OC(=O)NCC(O)c1ncccc1Oc1ccccc1OCc1ccccc1 ; "Pd/C" ; CC(C)(C)OC(=O)NCC(O)c1ncccc1Oc1ccccc1O]

To a solution of tert-butyl (2-(3-(2-(benzyloxy)phenoxy)pyridin-2-yl)-2-hydroxyethyl)carbamate (3.9 g, 8.93 mmol) in ethyl acetate (30 mL) was added Pd/C (500 mg, 10%). The mixture was stirred at about room temperature for about 2 hours and filtered. The filtrate was concentrated under vacuum to give tert-butyl (2-hydroxy-2-(2-(2-hydroxyphenoxy)pyridin-3-yl)ethyl)carbamate. MS (ESI): m/z 347 [M+H]⁺.

### f. Preparation of tert-butyl ((11H-benzo[2,3][1,4]dioxepino[6,5-b]pyridin-11-yl)methyl)carbamate

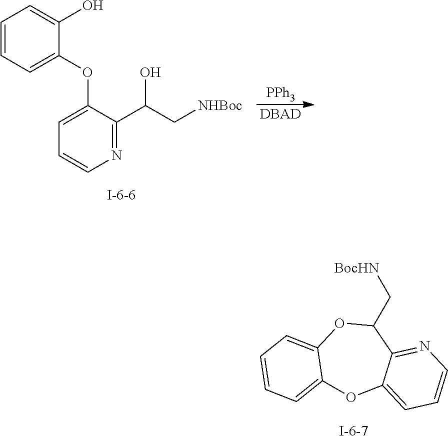

To a solution of tert-butyl (2-hydroxy-2-(2-(2-hydroxyphenoxy)pyridin-3-yl)ethyl)carbamate (2000 mg, 5.77 mmol) in tetrahydrofuran (20 mL) was added triphenylphosphine (1.81 g, 6.92 mmol) and N-{[(tert-butoxy)carbonyl]imino}(tert-butoxy)formamide (1.59 g, 6.92 mmol) at about 0° C. The reaction was stirred at about room temperature for about 10 minutes and concentrated under vacuum, the residue was purified by silica gel chromatography (petroleum ether/ethyl acetate=3/1) to give tert-butyl ((11H-benzo[2,3][1,4]dioxepino[6,5-b]pyridin-11-yl)methyl)carbamate. MS (ESI): m/z 329 [M+H]⁺.

### g. Chiral column separation of tert-butyl ((11H-benzo[2,3][1,4]dioxepino[6,5-b]pyridin-11-yl)methyl)carbamate

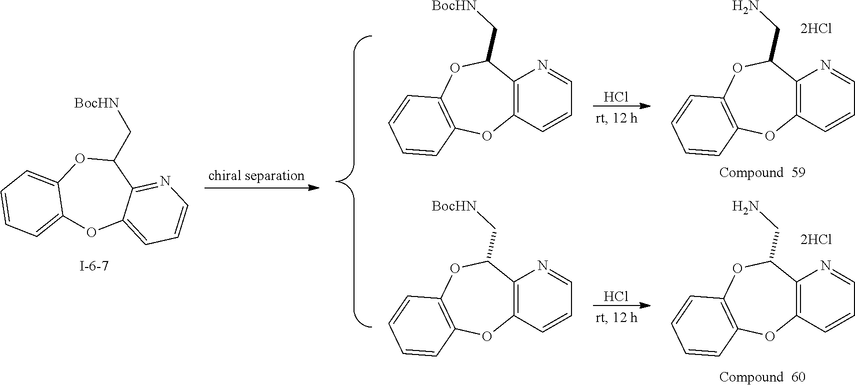

The racemic tert-butyl ((11H-benzo[2,3][1,4]dioxepino[6,5-b]pyridin-11-yl)methyl)carbamate was purified by chiral column separation using:

| | |
|---|---|
| Instrument: SFC-150 (Waters) | Column: OX 20*250 |
| Column temperature: 35° C. | mm, 10 μm (Daicel) |
| Mobile phase: CO₂/MeOH (0.2% | |
| Methanol Ammonia) = 65/35 | |
| Flow rate: 120 g/min | Back pressure: 100 bar |
| Detection wavelength: 214 nm | Cycle time: 2.4 min, |
| Sample solution: 600 mg dissolved in | |
| 60 mL Methanol | |
| Injection volume: 2 mL | |

After removal of solvents, the first eluting isomer (I-6-7-P1) (290 mg, retention time=1.49 min) and the second eluting isomer (I-6-7-P2) (290 mg, retention time=1.92 min) were obtained. MS (ESI): m/z 329 [M+H]⁺

### Synthesis of (R*)-(11H-benzo[2,3][1,4]dioxepino[6,5-b]pyridin-11-yl)methanamine (Compound 59) and (S*)-(11H-benzo[2,3][1,4]dioxepino[6,5-b]pyridin-11-yl)methanamine (Compound 60)

Independently, for each individual isomer I-6-7-P1 and I-6-7-P2, to a solution of each isomer (100 mg, 304 μmol) in MeOH (5 mL) was added HCl/MeOH (3M, 3 mL, 9 mmol). The reaction was stirred at about room temperature for about 16 hours and concentrated under vacuum. The residue was dissolved in water (5 mL) and extracted with ethyl acetate (5 mL). The aqueous phase was freeze-dried on lyophilizer to give Compound 59. MS (ESI): m/z 229 [M+H]⁺. ¹H NMR (400 MHz, CD₃OD) δ 8.37 (d, J=4.6 Hz, 1H), 7.72 (dd, J=8.2, 1.4 Hz, 1H), 7.47 (dd, J=8.2, 4.5 Hz, 1H), 7.30-7.22 (m, 1H), 7.18 (d, J=7.7 Hz, 1H), 7.15-6.95 (m, 2H), 5.96-5.65 (m, 1H), 3.82 (qd, J=13.6, 5.5 Hz, 2H). Chiral analysis column: AY-H (250*4.6 mm 5 μm); Mobile Phase: n-Hexane (0.1% DEA):EtOH (0.1% DEA)=90:10; Flow rate: 1.0 mL/min; column temperature: 40° C.; retention time=12.51 min.; and Compound 60. MS (ESI): m/z 229 [M+H]⁺. ¹H NMR (400 MHz, CD₃OD) δ 8.36 (d, J=4.4 Hz, 1H), 7.72 (d, J=8.2 Hz, 1H), 7.48 (d, J=4.7 Hz, 1H), 7.32-7.23 (m, 1H), 7.18 (d, J=7.3 Hz, 1H), 7.15-6.98 (m, 2H), 5.80 (d, J=3.9 Hz, 1H), 4.01-3.65 (m, 2H). Chiral analysis column: AY-H (250*4.6 mm 5 μm); Mobile Phase: n-Hexane (0.1% DEA):EtOH (0.1% DEA)=90:10; Flow rate: 1.0 mL/min; column temperature: 40° C.; retention time=18.73 min.

### Example 38. Synthesis of (S*)-1-(11H-benzo[2,3][1,4]dioxepino[6,5-b]pyridin-11-yl)-N-methylmethanamine (Compound 57) and (R*)-1-(11H-benzo[2,3][1,4]dioxepino[6,5-b]pyridin-11-yl)-N-methylmethanamine (Compound 58)

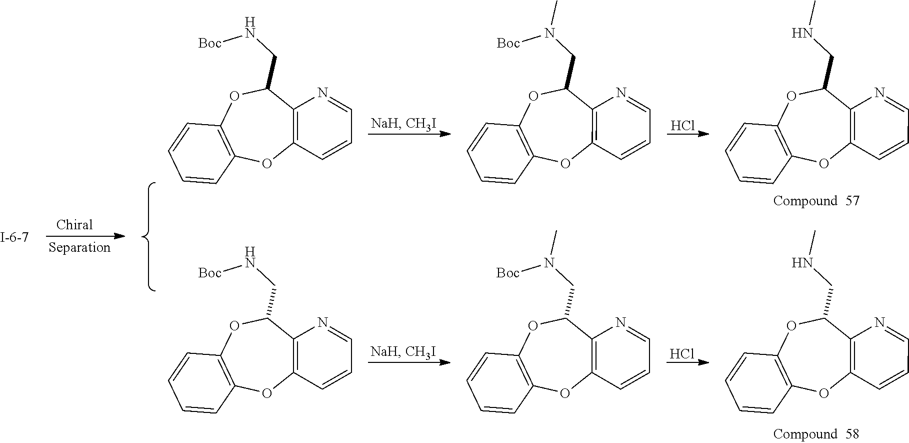

### Synthesis of (R*)-1-(11H-benzo[2,3][1,4]dioxepino[6,5-b]pyridin-11-yl)-N-methylmethanamine (Compound 57) and (S*)-1-(11H-benzo[2,3][1,4]dioxepino[6,5-b]pyridin-11-yl)-N-methylmethanamine (Compound 58)

Independently, for each individual isomer I-6-7-P1 and I-6-7-P2, to a solution of each (120 mg, 365 μmol) in tetrahydrofuran (5 mL) was added sodium hydride (103 mg, 730 μmol) and methyl iodide (103 mg, 730 μmol) at about 0° C. The reaction was stirred at about room temperature for about 3 hours and quenched with ice-water (3 mL). The mixture was extracted with ethyl acetate (15 mL×2). The combined organic layers were dried over anhydrous Na₂SO₄, filtered, and concentrated in vacuo. The resulting substance was purified by silica gel chromatography (petroleum ether/ethyl acetate=4/1) to give the N-methylated intermediate. MS (ESI): m/z 343[M+H]⁺

To a solution of each N-methylated intermediate (120 mg, 350 μmol) in MeOH (5 mL) was added HCl/MeOH (5 mL, 10 mmol). The reaction was stirred at about room temperature for about 16 hours and concentrated under vacuum. The residue was dissolved in water (5 mL) and extracted with ethyl acetate (5 mL). The aqueous phase was freeze-dried on a lyophilizer to give Compound 57. MS (ESI): m/z 243 [M+H]⁺. ¹H NMR (400 MHz, CD₃OD) δ 8.39 (s, 1H), 7.78 (s, 1H), 7.52 (d, J=4.5 Hz, 1H), 7.33-7.25 (m, 1H), 7.21 (s, 1H), 7.10 (pd, J=7.5, 3.5 Hz, 2H), 5.89 (d, J=4.1 Hz, 1H), 3.91 (t, J=9.8 Hz, 2H), 2.91 (s, 3H). Chiral analysis column: AY-H (250*4.6 mm 5 μm); Mobile Phase: n-Hexane (0.1% DEA):EtOH (0.1% DEA)=70:30; Flow rate: 1.0 mL/min; column temperature: 40° C.; retention time=8.96 min.; and Compound 58. MS (ESI): m/z 243 [M+H]⁺. ¹H NMR (400 MHz, CD₃OD) δ 8.37 (dd, J=4.7, 1.3 Hz, 1H), 7.73 (dd, J=8.3, 1.4 Hz, 1H), 7.48 (dd, J=8.3, 4.7 Hz, 1H), 7.34-7.24 (m, 1H), 7.18 (ddd, J=7.2, 4.5, 2.9 Hz, 1H), 7.14-6.95 (m, 2H), 5.86 (t, J=5.5 Hz, 1H), 3.92 (d, J=5.6 Hz, 2H), 2.90 (s, 3H). Chiral analysis column: AY-H (250*4.6 mm 5 μm); Mobile Phase: n-Hexane (0.1% DEA):EtOH (0.1% DEA)=70:30; Flow rate: 1.0 mL/min; column temperature: 40° C.; retention time=15.65 min.

### Example 39. Synthesis of (S*)-1-(7-fluoro-11H-benzo[2,3][1,4]dioxepino[6,5-b]pyridin-11-yl)-N-methylmethanamine (Compound 61) and (R*)-1-(7-fluoro-11H-benzo[2,3][1,4]dioxepino[6,5-b]pyridin-11-yl)-N-methylmethanamine (Compound 62)

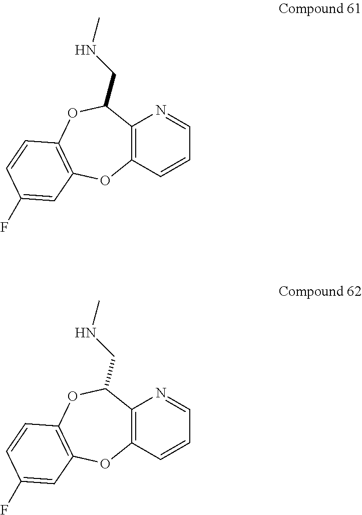 [SMILES: CNC[C@@H]1Oc2ccc(F)cc2Oc2cccnc21 ; CNC[C@H]1Oc2ccc(F)cc2Oc2cccnc21]

Compounds 61 and 62 are prepared similar to General Synthesis Scheme 3.

### Example 40. Synthesis of (S*)-(7-fluoro-11H-benzo[2,3][1,4]dioxepino[6,5-b]pyridin-11-yl)methanamine (Compound 63) and (R*)-(7-fluoro-11H-benzo[2,3][1,4]dioxepino[6,5-b]pyridin-11-yl)methanamine (Compound 64)

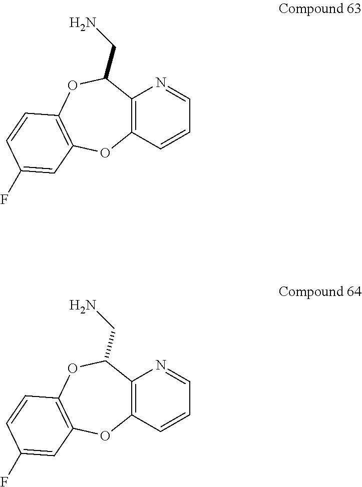 [SMILES: NC[C@@H]1Oc2ccc(F)cc2Oc2cccnc21 ; NC[C@H]1Oc2ccc(F)cc2Oc2cccnc21]

Compounds 63 and 64 are prepared similar to General Synthesis Scheme 3.

### Example 41. Synthesis of (S*)-1-(3-fluoro-11H-benzo[2,3][1,4]dioxepino[6,5-b]pyridin-11-yl)-N-methylmethanamine (Compound 65) and (R*)-1-(3-fluoro-11H-benzo[2,3][1,4]dioxepino[6,5-b]pyridin-11-yl)-N-methylmethanamine (Compound 66)

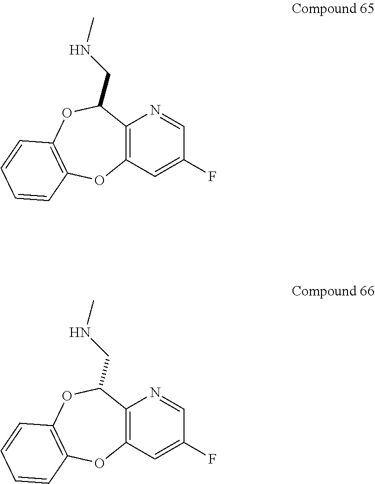 [SMILES: CNC[C@@H]1Oc2ccccc2Oc2cc(F)cnc21 ; CNC[C@H]1Oc2ccccc2Oc2cc(F)cnc21]

Compounds 65 and 66 are prepared similar to General Synthesis Scheme 3.

### Example 42. Synthesis of (S*)-(3-fluoro-11H-benzo[2,3][1,4]dioxepino[6,5-b]pyridin-11-yl)methanamine (Compound 67) and (R*)-(3-fluoro-11H-benzo[2,3][1,4]dioxepino[6,5-b]pyridin-11-yl)methanamine (Compound 68)

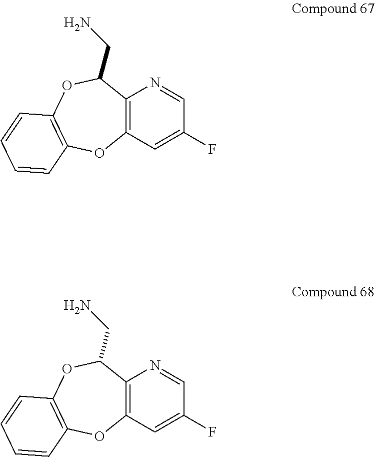 [SMILES: NC[C@@H]1Oc2ccccc2Oc2cc(F)cnc21 ; NC[C@H]1Oc2ccccc2Oc2cc(F)cnc21]

Compounds 67 and 68 are prepared similar to General Synthesis Scheme 3.

### Example 43. (R*)-1-(3-fluoro-10H-benzo[5,6][1,4]dioxepino[2,3-b]pyridin-10-yl)-N-methylmethanamine (Compound 69) and (S*)-1-(3-fluoro-10H-benzo[5,6][1,4]dioxepino[2,3-b]pyridin-10-yl)-N-methylmethanamine (Compound 70)

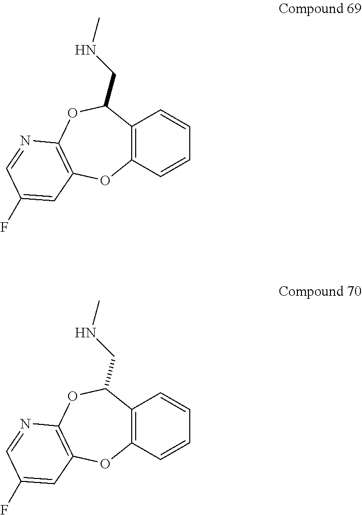 [SMILES: CNC[C@@H]1Oc2ncc(F)cc2Oc2ccccc21 ; CNC[C@H]1Oc2ncc(F)cc2Oc2ccccc21]

Compounds 69 and 70 are prepared similar to General Synthesis Scheme 4.

### Example 44. (R*)-(3-fluoro-10H-benzo[5,6][1,4]dioxepino[2,3-b]pyridin-10-yl)methanamine (Compound 71) and (S*)-(3-fluoro-10H-benzo[5,6][1,4]dioxepino[2,3-b]pyridin-10-yl)methanamine (Compound 72)

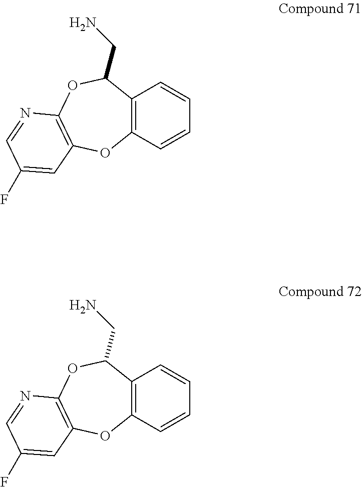 [SMILES: NC[C@@H]1Oc2ncc(F)cc2Oc2ccccc21 ; NC[C@H]1Oc2ncc(F)cc2Oc2ccccc21]

Compounds 71 and 72 are prepared similar to General Synthesis Scheme 4.

### Example 45. (R*)-1-(7-fluoro-10H-benzo[5,6][1,4]dioxepino[2,3-b]pyridin-10-yl)-N-methylmethanamine (Compound 73) and (S*)-1-(7-fluoro-10H-benzo[5,6][1,4]dioxepino[2,3-b]pyridin-10-yl)-N-methylmethanamine (Compound 74)

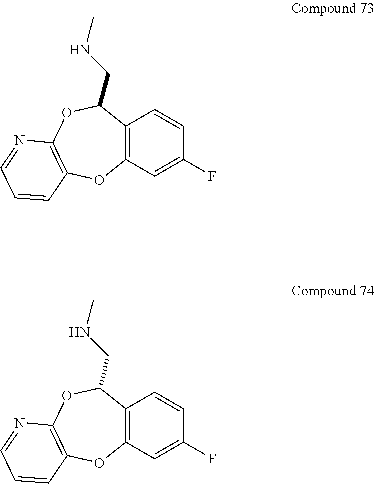 [SMILES: CNC[C@@H]1Oc2ncccc2Oc2cc(F)ccc21 ; CNC[C@H]1Oc2ncccc2Oc2cc(F)ccc21]

Compounds 73 and 74 are prepared similar to General Synthesis Scheme 3.

### Example 46. (R*)-(7-fluoro-10H-benzo[5,6][1,4]dioxepino[2,3-b]pyridin-10-yl)methanamine (Compound 75) and (S*)-(7-fluoro-10H-benzo[5,6][1,4]dioxepino[2,3-b]pyridin-10-yl)methanamine (Compound 76)

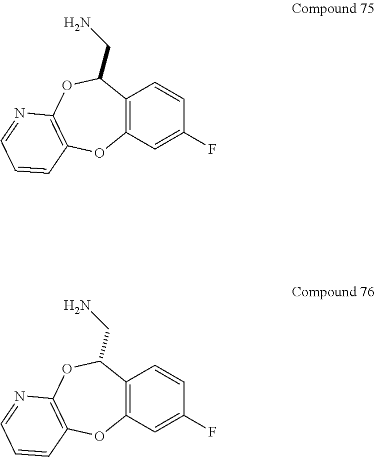 [SMILES: NC[C@@H]1Oc2ncccc2Oc2cc(F)ccc21 ; NC[C@H]1Oc2ncccc2Oc2cc(F)ccc21]

Compounds 75 and 76 are prepared similar to General Synthesis Scheme 3.

### Example 47. (6,11-dihydrodibenzo[b,e]oxepin-6-yl)methanamine (Compound

77), (R*)-(6,11-dihydrodibenzo[b,e]oxepin-6-yl)methanamine (Compound 78), and (S*)-(6,11-dihydrodibenzo[b,e]oxepin-6-yl)methanamine (Compound 79)

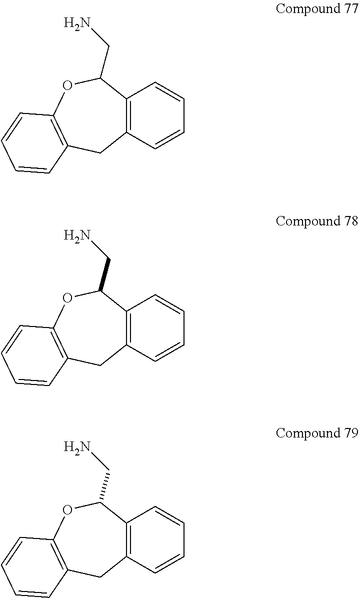 [SMILES: NCC1Oc2ccccc2Cc2ccccc21 ; NC[C@@H]1Oc2ccccc2Cc2ccccc21 ; NC[C@H]1Oc2ccccc2Cc2ccccc21]

### a. Preparation of (2-bromophenyl)(2-methoxyphenyl)methanol

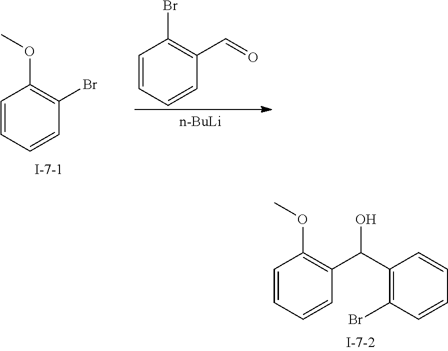

To a solution of 1-bromo-2-methoxybenzene (9.33 g, 49.88 mmol) in tetrahydrofuran (130 mL) was added n-butyllithium (2.5 M in tetrahydrofuran, 25 mL, 62.5 mmol) at −78° C., After stirring at this temperature for 1 h, 2-bromobenzaldehyde (12 g, 64.84 mmol) was added and the mixture was stirred at this temperature for 3 h. Upon completion, saturated ammonium chloride aqueous solution (50 mL) was added, and the mixture was extracted with ethyl acetate (50 mL×3), dried and concentrated. The residue was purified by flash column chromatography with a gradient elution of petroleum ether (100%) and ethyl acetate (0%) to petroleum ether (90%) and ethyl acetate (10%) to provide the desired compound. MS (ESI): m/z=275, 277[M-OH]⁺.

### b. Preparation of I-bromo-2-(2-methoxybenzyl) benzene

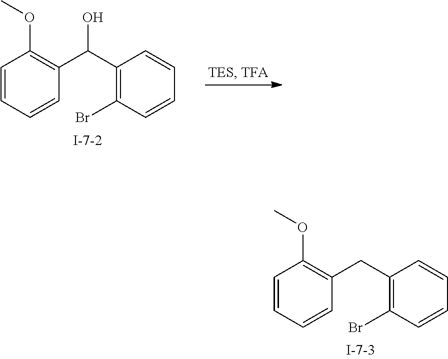 [SMILES: COc1ccccc1C(O)c1ccccc1Br ; COc1ccccc1Cc1ccccc1Br]

To a solution of (2-bromophenyl)(2-methoxyphenyl)methanol (7.9 g, 26.95 mmol) in dichloromethane (200 mL) was added triethylsilane (TES) (18.67 g, 161.7 mmol) and 2,2,2-trifluoroacetic acid (9.22 g, 80.85 mmol) at 0° C. The reaction was stirred at ambient temperature for 3 h. Upon completion, the mixture was washed with water (30 mL×3), dried and concentrated. The crude was used in the next step without further purification. MS (ESI): m/z=277, 279[M+H]⁺.

### c. Preparation of 2-(2-bromobenzyl) phenol

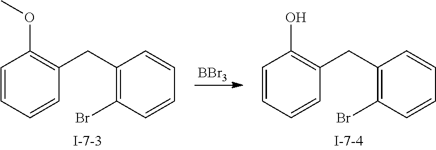

To a solution of 1-bromo-2-(2-methoxybenzyl)benzene (7 g, 25.26 mmol) in dichloromethane (100 mL) was added tribromoborane (18.98 g, 75.78 mmol) at −20° C. The reaction was stirred at ambient temperature for 3 h. Upon completion, water was added to the reaction and the mixture was washed with sodium bicarbonate solution (100 mL×2). The organic phase was washed with brine, dried and concentrated. The residue was purified by flash column chromatography with a gradient elution of petroleum ether (100%) and ethyl acetate (0%) to petroleum ether (80%) and ethyl acetate (20%) to provide the desired compound. MS (ESI): m/z=263, 265[M+H]⁺.

### d. Preparation of (2-(2-bromobenzyl) phenoxy) (tert-butyl)dimethylsilane

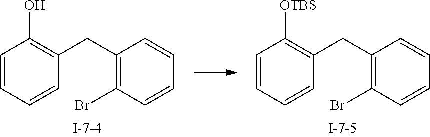 [SMILES: Oc1ccccc1Cc1ccccc1Br ; CC(C)(C)[Si](C)(C)Oc1ccccc1Cc1ccccc1Br]

To a solution of 2-(2-bromobenzyl)phenol (2.89 g, 10.98 mmol) in dichloromethane (50 mL) was added tert-butylchlorodimethylsilane (1.99 g, 13.27 mmol) and 1H-imidazole (1.53 g, 22.57 mmol). The reaction was stirred at ambient temperature for 1 h. Upon completion, the mixture was washed with water (30 mL×3), dried and concentrated. The residue was purified by flash column chromatography with petroleum ether to provide I-7-5. e. Preparation of 2-(2-(tert-butyldimethylsilyloxy)benzyl)benzonitrile

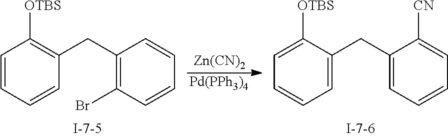

To a solution of (2-(2-bromobenzyl) phenoxy) (tert-butyl)dimethylsilane (3.69 g, 9.78 mmol) in N,N-dimethylformamide (15 mL) was added dicyanozinc (2.3 g, 19.56 mmol) and tetrakis(triphenylphosphine)palladium (1.13 g, 0.98 mmol). The mixture was heated to 130° C. under nitrogen for overnight. Upon completion, water (30 mL) was added and the mixture was extracted with ethyl acetate (50 mL×2), the combined organic layers were washed with brine, dried and concentrated. The residue was purified by flash column chromatography with petroleum ether to provide the desired compound. MS (ESI): m/z=324[M+H]⁺.

### f. Preparation of 2-(2-(tert-butyldimethylsilyloxy) benzyl) benzaldehyde

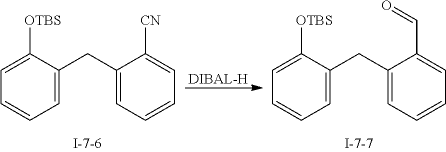

To a solution of 2-(2-((tert-butyldimethylsilyl)oxy)benzyl)benzonitrile (1.7 g, 5.26 mmol) in dichloromethane (15 mL) was added diisobutylaluminium hydride (1M in THF solution 10.5 mL, 10.5 mmol) at −20° C. Then the mixture was stirred at this temperature for 2 h. Upon completion, 2.4 mL of water was added to the mixture and the mixture was filtered. The filtration was dried and concentrated to get the crude product which was purified by flash column chromatography with petroleum ether to provide the desired compound. MS (ESI): m/z=327[M+H]⁺.

### g. Preparation of 2-(2-(2-(tert-butyldimethylsilyloxy) benzyl) phenyl)-2-hydroxyacetonitrile

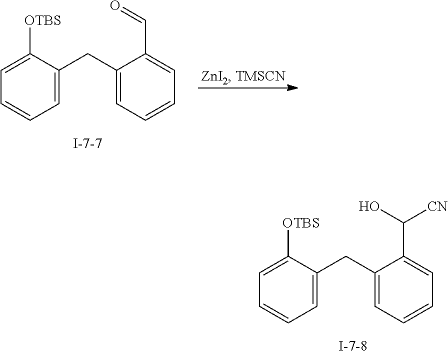

To a solution of 2-(2-((tert-butyldimethylsilyl) oxy) benzyl) benzaldehyde (1.38 g, 4.23 mmol) in dichloromethane (20 mL) was added trimethylsilanecarbonitrile (0.84 g, 8.46 mmol) and zinc iodide (0.27 g, 0.85 mmol). The reaction was stirred at ambient temperature for 4 h. Upon completion, the mixture was filtered and the filtrate was concentrated. The crude was used for next step without further purification. MS (ESI): m/z=376[M+Na]⁺.

### h. Preparation of 2-amino-1-(2-(2-(tert-butyldimethylsilyloxy) benzyl) phenyl) ethanol

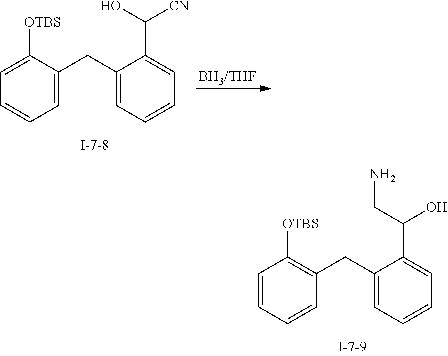

To a solution of 2-(2-(2-((tert-butyldimethylsilyl) oxy) benzyl) phenyl)-2-hydroxyacetonitrile (1.48 g, 4.19 mmol) in tetrahydrofuran (4 mL) was added borane/tetrahydrofuran (1M) (13 mL, 13 mmol). The mixture was heated to 40° C. for 2 h. Upon completion, water (20 mL) was added to quench the reaction, and the aqueous phase was extracted with ethyl acetate (20 mL×3), dried and concentrated. The crude was used for next step without further purification. MS (ESI): m/z=358[M+H]⁺.

### i. Preparation of tert-butyl 2-(2-(2-(tert-butyldimethylsilyloxy) benzyl) phenyl)-2-hydroxyethylcarbamate

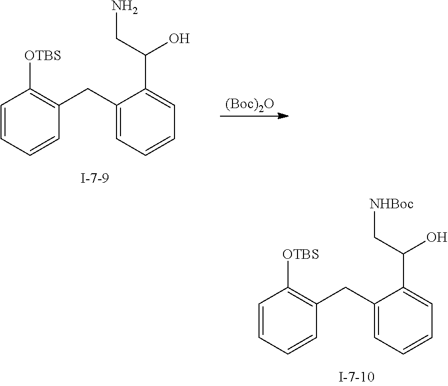 [SMILES: "(Boc)2O" ; CC(C)(C)[Si](C)(C)Oc1ccccc1Cc1ccccc1C(O)CN ; CC(C)(C)OC(=O)NCC(O)c1ccccc1Cc1ccccc1O[Si](C)(C)C(C)(C)C]

To a solution of 2-amino-1-(2-(2-((tert-butyldimethylsilyl) oxy) benzyl) phenyl) ethanol in dichloromethane (40 mL) was added di-tert-butyl dicarbonate (1.37 g, 6.3 mmol) and triethylamine (1.7 g, 16.8 mmol). The reaction was stirred at ambient temperature for 16 h. Upon completion, the mixture was washed with water (40 mL×2), dried and concentrated. The residue was purified by flash column chromatography with a gradient elution of petroleum ether (100%) and ethyl acetate (0%) to petroleum ether (75%) and ethyl acetate (25%) to provide I-7-10. MS (ESI): m/z=480[M+Na]⁺.

### j. Preparation of tert-butyl 2-hydroxy-2-(2-(2-hydroxybenzyl) phenyl) ethylcarbamate

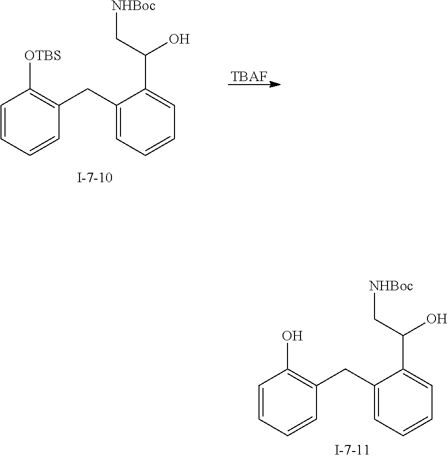

To a solution of tert-butyl (2-(2-(2-((tert-butyldimethylsilyl) oxy) benzyl) phenyl)-2-hydroxyethyl)carbamate (560 mg, 1.22 mmol) in tetrahydrofuran (10 mL) was added tetrabutylammonium fluoride (TBAF) (0.64 g, 2.45 mmol). The reaction was stirred at ambient temperature for 1 h. Upon completion, the mixture was washed with water (20 mL×2), dried and concentrated. The residue was purified by flash column chromatography with a gradient elution of petroleum ether (100%) and ethyl acetate (0%) to petroleum ether (75%) and ethyl acetate (25%) to provide the desired compound.

### k. Preparation of tert-butyl ((6,11-dihydrodibenzo[b,e]oxepin-6-yl) methyl)carbamate

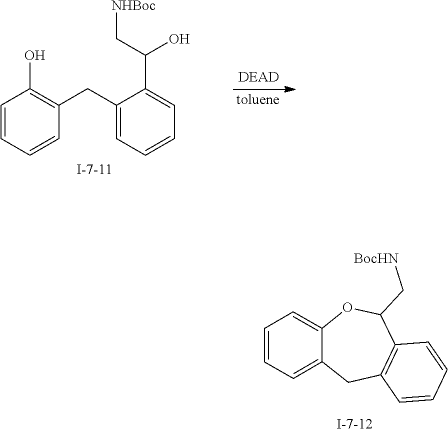 [SMILES: CC(C)(C)OC(=O)NCC(O)c1ccccc1Cc1ccccc1O ; CC(C)(C)OC(=O)NCC1Oc2ccccc2Cc2ccccc21]

To a mixture of tert-butyl (2-hydroxy-2-(2-(2-hydroxybenzyl)phenyl)ethyl) carbamate (414 mg, 1.19 mmol) and triphenylphosphane (623.6 mg, 2.38 mmol) in toluene (10 mL) was added diethyl azodicarboxylate (414 mg, 2.38 mmol) at 0° C. under nitrogen. The mixture was stirred at room temperature for 30 min. Upon completion, the mixture was washed with water (20 mL×2), dried and concentrated. The residue was purified by flash column chromatography with a gradient elution of petroleum ether (100%) and ethyl acetate (0%) to petroleum ether (80%) and ethyl acetate (20%) to provide the desired compound. MS (ESI): m/z=226 [M-100+H]⁺, 270[M-55]⁺.

### l. Synthesis of (6,11-dihydrodibenzo[b,e]oxepin-6-yl)methanamine (Compound 77)

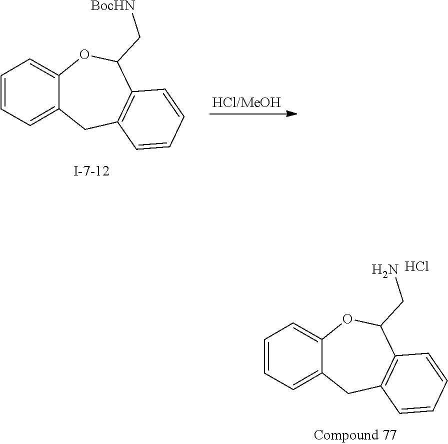 [SMILES: CC(C)(C)OC(=O)NCC1Oc2ccccc2Cc2ccccc21 ; Cl ; NCC1Oc2ccccc2Cc2ccccc21]

A solution of tert-butyl ((6,11-dihydrodibenzo[b,e]oxepin-6-yl)methyl)carbamate (0.202 g, 0.61 mmol) in hydrogen chloride/methanol (3 mL) was stirred at room temperature for 3 h. Upon completion, the solvent was removed, and the solid was dried by freeze dryer to give Compound 77 HCl salt. MS (ESI): m/z=226[M+H]⁺. ¹H-NMR (freebase, 400 MHz, CDCl₃): δ 7.28˜7.12 (m, 6H), 7.03˜6.94 (m, 2H), 5.36˜5.34 (dd, J₁=3.6 Hz, J₂=8 Hz, 1H), 4.29˜4.25 (d, J=14.4 Hz, 1H), 4.12˜4.09 (d, J=14.4 Hz, 1H), 3.42˜3.30 (m, 2H), 1.80 (brs, 2H).

Chiral chromatography separation of Compound 77 provides Compounds 78 and 79.

### Example 50. Synthesis of (R*)-(8-fluoro-10,11-dihydrodibenzo[b,f]oxepin-10-yl)methanamine (Compound 92) and (S*)-(8-fluoro-10,11-dihydrodibenzo[b,f]oxepin-10-yl)methanamine (Compound 93)

### a. Preparation of methyl 8-fluorodibenzo[b,f]oxepine-10-carboxylate

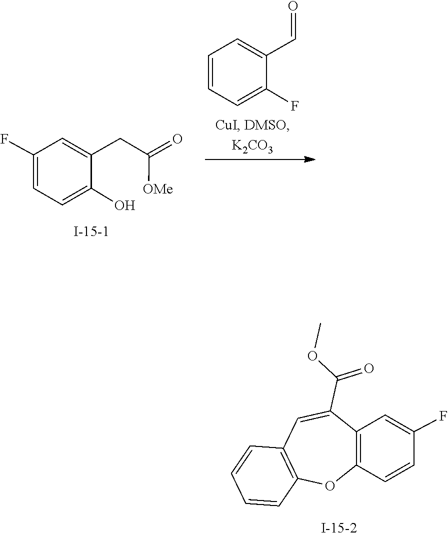 [SMILES: O=Cc1ccccc1F ; COC(=O)Cc1cc(F)ccc1O ; COC(=O)C1=Cc2ccccc2Oc2ccc(F)cc21]

To a solution of methyl 2-(5-fluoro-2-hydroxyphenyl)acetate (10.8 g, 58.6 mmol) in DMSO (150 mL) was added 2-fluorobenzaldehyde (10.9 g, 87.9 mmol), K₂CO₃ (16.1 g, 117 mmol) and copper(I) iodide (2.22 g, 11.7 mmol) under nitrogen. The mixture was heated to 110° C. with stirring for 3 h. Upon the completion, the mixture was quenched with 500 mL of water, extracted with ethyl acetate (300 mL×2), dried and evaporated in vacuo to get the crude which was purified by silica gel chromatography with a gradient elution of petroleum ether (100%) to petroleum ether (80%) and ethyl acetate (20%) to provide methyl 8-fluorodibenzo[b,f]oxepine-10-carboxylate (1.8 g, yield: 9.5%) as a yellow oil. MS (ESI) m/z: 271 [M+H]⁺.

### b. Preparation of methyl 8-fluoro-10,11-dihydrodibenzo[b,f]oxepine-10-carboxylate

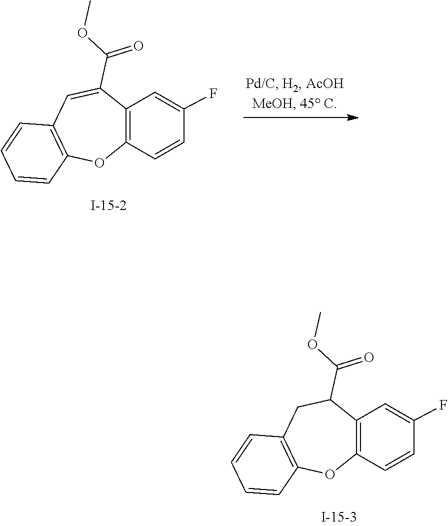 [SMILES: COC(=O)C1=Cc2ccccc2Oc2ccc(F)cc21 ; COC(=O)C1Cc2ccccc2Oc2ccc(F)cc21]

To a solution of methyl 8-fluorodibenzo[b,f]oxepine-10-carboxylate (1.8 g, 6.67 mmol) in methanol (40 mL) was added Pd/C (10% wet) (360 mg) and AcOH (4 mL). The mixture was stirred at 45° C. under hydrogen for overnight. Upon the completion, the mixture was filtered over diatomite and the filtration was evaporated in vacuo to get methyl 8-fluoro-10,11-dihydrodibenzo[b,f]oxepine-10-carboxylate (1.5 g, yield: 72.8%) as a yellow oil. MS (ESI) m/z: 273 [M+H]⁺.

### c. Preparation of (8-fluoro-10,11-dihydrodibenzo[b,f]oxepin-10-yl)methanol

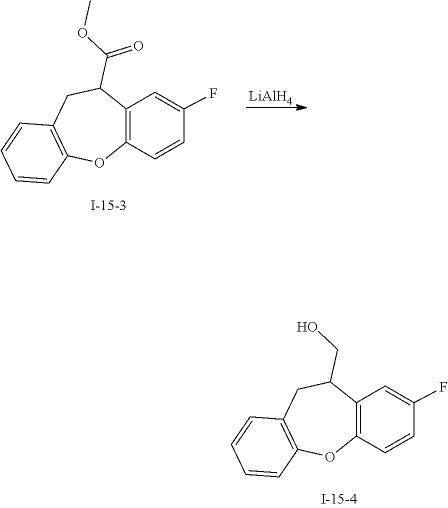

To a solution of methyl 8-fluoro-10,11-dihydrodibenzo[b,f]oxepine-10-carboxylate (1.5 g, 5.51 mmol) in tetrahydrofuran (30 mL) was added LiAlH₄ (251 mg, 6.61 mmol) by portions in ice bath. The reaction was stirred at this temperature for 1 h. Upon the completion, the mixture was carefully quenched with 1.5 g of water, filtered and the filtration was dried and concentrated under reduced pressure to get the residue which was purified by silica gel chromatography with a gradient elution of petroleum ether (100%) to petroleum ether (85%) and ethyl acetate (15%) to provide (6-fluoro-10,11-dihydrodibenzo[b,f]oxepin-10-yl)methanol (1.2 g, yield: 89.4%) as a white solid. MS (ESI) m/z: 227 [M-OH]⁺.

### d. Preparation of 2-((8-fluoro-10,11-dihydrodibenzo[b,f]oxepin-10-yl)methyl)isoindoline-1,3-dione

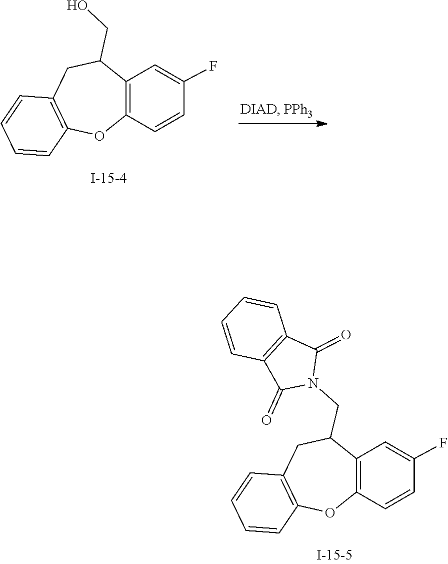 [SMILES: OCC1Cc2ccccc2Oc2ccc(F)cc21 ; O=C1c2ccccc2C(=O)N1CC1Cc2ccccc2Oc2ccc(F)cc21]

To a solution of (6-fluoro-10,11-dihydrodibenzo[b,f]oxepin-10-yl)methanol (1.2 g, 4.91 mmol) in toluene (25 mL) was added triphenylphosphane (2.58 g, 9.84 mol), diisopropyl (E)-diazene-1,2-dicarboxylate (1.99 g, 9.84 mol) and isoindoline-1,3-dioneat (1.08 g, 7.37 mmol) at 0° C. under nitrogen. The mixture was stirred at that temperature for 2 h. Upon the completion, the mixture was quenched with 100 mL of water, extracted with ethyl acetate (60 mL×2), dried and evaporated in vacuo to dryness. The crude was used directly for next step without further purification. MS (ESI) m/z: 374 [M+H]⁺.

### e. Preparation of tert-butyl ((8-fluoro-10,11-dihydrodibenzo[b,f]oxepin-10-yl)methyl)carbamate

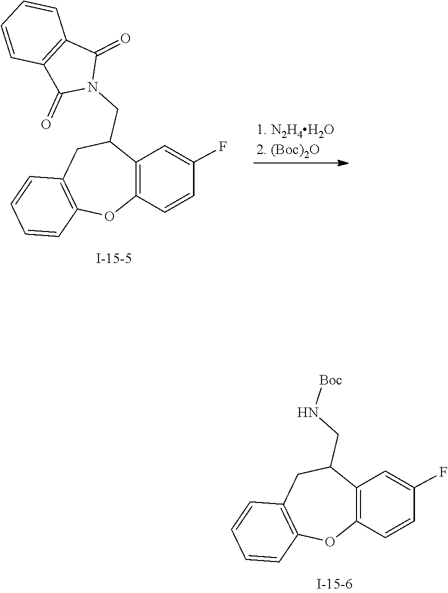 [SMILES: O=C1c2ccccc2C(=O)N1CC1Cc2ccccc2Oc2ccc(F)cc21 ; CC(C)(C)OC(=O)NCC1Cc2ccccc2Oc2ccc(F)cc21]

To a solution of 2-((8-fluoro-10,11-dihydrodibenzo[b,f]oxepin-10-yl)methyl)isoindoline-1,3-dione (crude, 4.91 mmol) in ethanol (40 mL) was added N₂H₄·H₂O (85% aq. 866 mg, 14.7 mmol). The reaction mixture was heated to 70° C. and stirred at that temperature for 3 h. After cooling to room temperature, the mixture was filtered and the filtration was concentrated under reduced pressure to get the residue which was diluted in 100 mL of dichloromethane, washed with water (50 mL×2), then extracted with 1 N aq. HCl (40 mL×2). The aqueous phase was basified with 2 N aq. NaOH till pH-12, extracted with dichloromethane (50 mL×2), dried and evaporated in vacuo to dryness. To a solution of the resulted mixture in dichloromethane (20 mL) was added triethylamine (992 mg, 9.82 mmol) and di-tert-butyl dicarbonate (1.61 g, 7.37 mmol). The reaction mixture was stirred at ambient temperature for 3 h. Upon the completion, the mixture was washed with water (30 mL), dried and concentrated under reduced pressure to get the crude which was purified by flash column chromatography with a gradient elution of petroleum ether (100%) to petroleum ether (90%) and ethyl acetate (10%) to provide tert-butyl ((8-fluoro-10,11-dihydrodibenzo[b,f]oxepin-10-yl)methyl)carbamate (660 mg, yield: 39.2%) as a white solid. MS (ESI) m/z: 244 [M-99]⁺.

### f. Preparation of (8-fluoro-10,11-dihydrodibenzo[b,f]oxepin-10-yl)methanamine

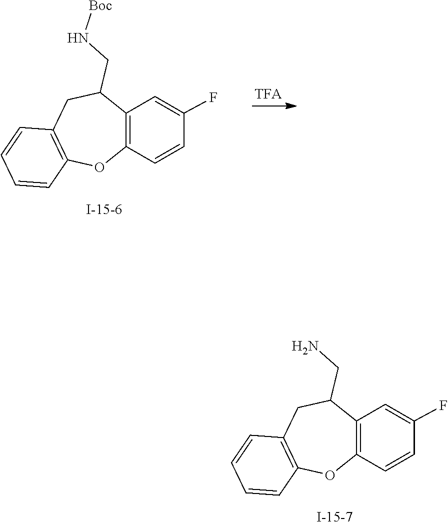 [SMILES: CC(C)(C)OC(=O)NCC1Cc2ccccc2Oc2ccc(F)cc21 ; NCC1Cc2ccccc2Oc2ccc(F)cc21]

To a solution of tert-butyl ((8-fluoro-10,11-dihydrodibenzo[b,f]oxepin-10-yl)methyl)carbamate (660 mg, 1.92 mmol) in dichloromethane (15 mL) was added TFA (8 mL). The reaction was stirred at ambient temperature for 2 h. Upon the completion, the mixture was evaporated in vacuo to dryness and the residue was basified with 2 N aq. NaOH till pH-12, extracted with dichloromethane (30 mL×3), dried and concentrated under reduced pressure to provide (8-fluoro-10,11-dihydrodibenzo[b,f]oxepin-10-yl)methanamine. MS (ESI) m/z: 244 [M+H]⁺.

### Synthesis of (R*)-(8-fluoro-10,11-dihydrodibenzo[b,f]oxepin-10-yl)methanamine (Compound 92) and (S*)-(8-fluoro-10,11-dihydrodibenzo[b,f]oxepin-10-yl)methanamine (Compound 93)

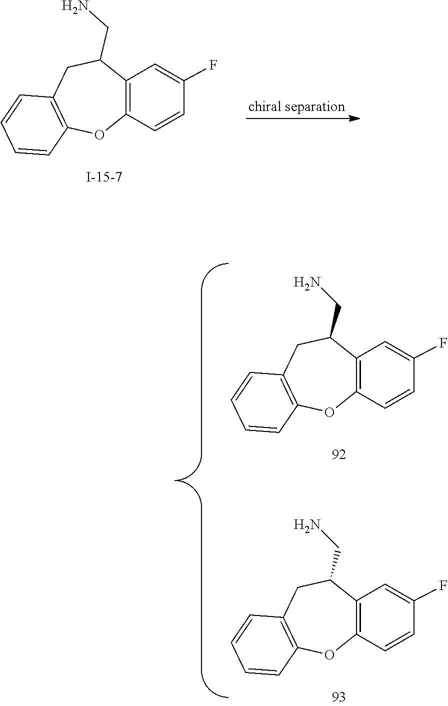 [SMILES: NCC1Cc2ccccc2Oc2ccc(F)cc21 ; NC[C@@H]1Cc2ccccc2Oc2ccc(F)cc21 ; NC[C@H]1Cc2ccccc2Oc2ccc(F)cc21]

The mixture was purified by chiral separation using Instrument: SFC-150 (Waters), Column: AD 20*250 mm, 10 μm (Daicel), Column temperature: 35° C., Mobile phase: CO2/(MEOH/ACN (0.2% Methanol Ammonia)=9:1)=75/25, Flow rate: 120 g/min, Back pressure: 100 bar, Detection wavelength: 214 nm, Cycle time: 3.4 min, Sample solution: 400 mg dissolved in 19 mL Methanol and Dichloromethane and Injection volume: 1.5 mL to yield compound 92 freebase form (170 mg, retention time=1.360 min) and compound 93 freebase form (150 mg, retention time=1.732 min).

To a solution of compound 92 freebase form from above (80 mg, 0.33 mmol) in ethyl acetate (1 mL) was added hydrogen chloride in ethyl acetate (0.2 mL, 3 mol/L, 0.6 mmol) at room temperature. The mixture was stirred at this temperature for 15 mins. Upon the completion, the mixture was evaporated in vacuo to dryness and the residue was quenched with excess water, washed with ethyl acetate (10 mL×2) and then aqueous phase was freeze-dried to yield compound 92 as a HCl salt. MS (ESI) m/z: 244 [M+H]⁺. ¹H NMR (400 MHz, CD₃OD) δ 7.28-7.19 (m, 4H), 7.13-7.05 (m, 3H), 3.45-3.43 (m, 1H), 3.42-3.39 (m, 2H), 3.33-3.26 (m, 1H), 3.27-3.19 (m, 1H). Chiral analysis Column: AD-3 4.6*100 mm 3 μm; Acq. Method Set: AD 20% B1; Co-Solvent: methanol[0.2% NH₃ (7M in methanol)]; Run Time: 6.0 Minutes; Flow_rate: 3.0 mL/min; Back_Pressure: 2000 psi; Column Temperature: 40° C.; retention time: 1.360 min.

To a solution of compound 93 freebase form from above (70 mg, 0.29 mmol) in ethyl acetate (1 mL) was added hydrogen chloride in ethyl acetate (0.2 mL, 3 mol/L, 0.6 mmol) at room temperature. The mixture was stirred at this temperature for 15 mins. Upon the completion, the mixture was evaporated in vacuo to dryness and the residue was quenched with excess water, washed with ethyl acetate (10 mL×2) and then freeze-dried to yield compound 93 as a HCl salt. MS (ESI) m/z: 244 [M+H]⁺. ¹H NMR (400 MHz, CD₃OD) δ 7.28-7.19 (m, 4H), 7.13-7.05 (m, 3H), 3.45-3.43 (m, 1H), 3.42-3.39 (m, 2H), 3.33-3.26 (m, 1H), 3.27-3.19 (m, 1H). Chiral analysis Column: AD-3 4.6*100 mm 3 μm; Acq. Method Set: AD 20% B1; Co-Solvent: methanol[0.2% NH₃ (7M in methanol)]; Run Time: 6.0 Minutes; Flow_rate: 3.0 mL/min; Back_Pressure: 2000 psi; Column Temperature: 40° C.; retention time: 1.732 min.

### Example 51. Synthesis of (R*)-(8-fluoro-10,11-dihydrodibenzo[b,f]oxepin-10-yl)-N-methylmethanamine (Compound 94) and (S*)-(8-fluoro-10,11-dihydrodibenzo[b,f]oxepin-10-yl)-N-methylmethanamine (Compound 95)

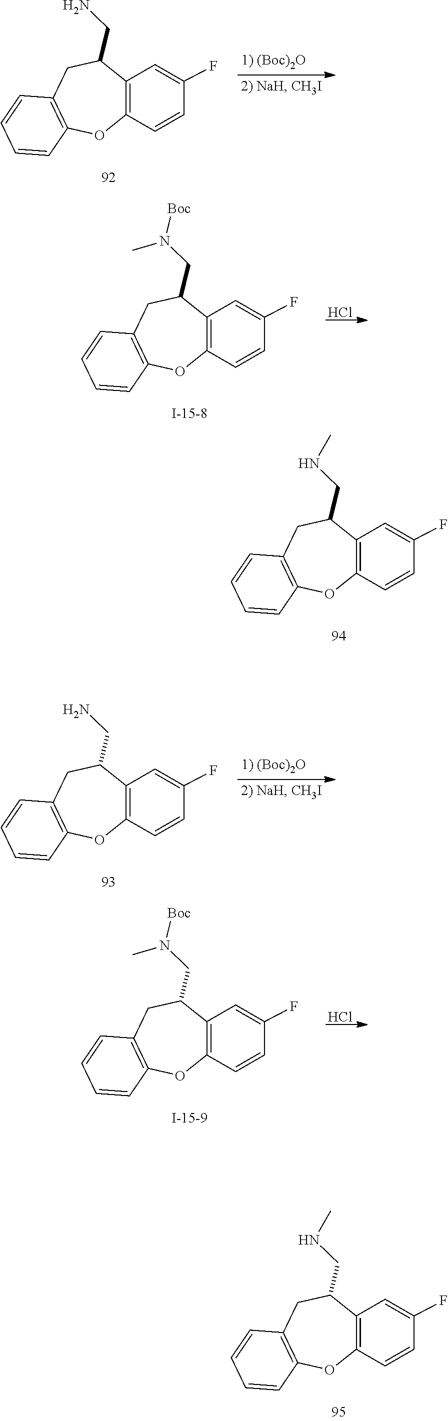

### Synthesis of (R*)-(8-fluoro-10,11-dihydrodibenzo[b,f]oxepin-10-yl)-N-methylmethanamine (Compound 94) and (S*)-(8-fluoro-10,11-dihydrodibenzo[b,f]oxepin-10-yl)-N-methylmethanamine (Compound 95)

To a solution of compound 92 (free base form, 80 mg, 0.33 mmol) in dichloromethane (10 mL) was added di-tert-butyl dicarbonate (144 mg, 660 μmol) and triethylamine (83.0 mg, 822 μmol). The mixture was stirred at ambient temperature for 2 h. Upon the completion, the mixture was evaporated in vacuo to dryness and the residue was purified by silica gel chromatography with a gradient elution of petroleum ether (100%) to petroleum ether (90%) and ethyl acetate (10%) to provide the BOC protected intermediate. To it was added N,N-dimethylformamide (3 mL) and to the resulting solution was added sodium hydride (60% in mineral oil) (13 mg, 320 μmol) at 0° C. After stirring for 15 mins, iodomethane (34 mg, 240 μmol) was added. The mixture was stirred at this temperature for 2 h. Upon the completion, the mixture was quenched with 50 mL of water, extracted with ethyl acetate (20 mL×2), dried and evaporated in vacuo to dryness. The residue was purified by silica gel chromatography with a gradient elution of petroleum ether (100%) to petroleum ether (90%) and ethyl acetate (10%) to provide compound I-15-8 (24 mg, yield: 42.0%) as a yellow oil. MS (ESI) m/z: 380 [M+Na]⁺. To a solution of compound I-15-8 (24 mg, 0.067 mmol) in ethyl acetate (1 mL) was added hydrogen chloride in ethyl acetate (2 mL, 3 mol/L, 6 mmol). The mixture was stirred at ambient temperature for overnight. Upon the completion, the mixture was evaporated in vacuo to dryness. The residue was triturated with ethyl acetate (3 mL) and freeze-dried to provide compound 94. MS (ESI) m/z: 258 [M+H]⁺. ¹H NMR (400 MHz, CD₃OD) δ 7.29-7.03 (m, 7H), 3.63-3.53 (m, 2H), 3.43-3.36 (m, 2H), 3.18 (dd, J=16.2, 5.8 Hz, 1H), 2.72 (s, 3H). Chiral analysis Column: AD-3 4.6*100 mm 3 μm; Acq. Method Set: AD 20% B1; Co-Solvent: methanol[0.2% NH₃ (7M in methanol)]; Run Time: 3.0 Minutes; Flow_rate: 3.0 mL/min; Back_Pressure: 2000 psi; Column Temperature: 40° C.; retention time: 0.970 min.

To a solution of compound 93 (freebase form, 80 mg, 0.33 mmol) in dichloromethane (10 mL) was added di-tert-butyl dicarbonate (144 mg, 660 μmol) and triethylamine (83.0 mg, 822 μmol). The mixture was stirred at ambient temperature for 2 h. Upon the completion, the mixture was evaporated in vacuo to dryness and the residue was purified by silica gel chromatography with a gradient elution of petroleum ether (100%) to petroleum ether (90%) and ethyl acetate (10%) to provide BOC protected intermediate. To it was added N,N-dimethylformamide (5 mL) and to the resulting solution was added sodium hydride (60% in mineral oil) (23 mg, 580 μmol) at 0° C. After stirring for 15 mins, iodomethane (62 mg, 435 μmol) was added. The mixture was stirred at this temperature for 2 h. Upon the completion, the mixture was quenched with 50 mL of water, extracted with ethyl acetate (20 mL×2), dried and evaporated in vacuo to dryness. The residue was purified by silica gel chromatography with a gradient elution of petroleum ether (100%) to petroleum ether (90%) and ethyl acetate (10%) to provide compound I-15-9 (88 mg, yield: 85.0%) as a yellow oil. MS (ESI) m/z: 380 [M+Na]⁺. To a solution of compound I-15-9 (88 mg, 0.25 mmol) in ethyl acetate (1 mL) was added hydrogen chloride in ethyl acetate (2 mL, 3 mol/L, 6 mmol). The mixture was stirred at ambient temperature for overnight. Upon the completion, the mixture was evaporated in vacuo to dryness. The residue was triturated with ethyl acetate (6 mL) and freeze-dried to provide compound 95. MS (ESI) m/z: 258 [M+H]⁺. ¹H NMR (400 MHz, CD₃OD) δ 7.29-7.03 (m, 7H), 3.63-3.53 (m, 2H), 3.43-3.36 (m, 2H), 3.18 (dd, J=16.2, 5.8 Hz, 1H), 2.72 (s, 3H). Chiral analysis Column: AD-3 4.6*100 mm 3 μm; Acq. Method Set: AD 20% B1; Co-Solvent: methano[0.2% NH₃ (7M in methanol)]; Run Time: 3.0 Minutes; Flow_rate: 3.0 mL/min; Back_Pressure: 2000 psi; Column Temperature: 40° C.; retention time: 1.032 min.

### Example 52. Synthesis of (R*)-(3-fluoro-10,11-dihydrodibenzo[b,f]oxepin-10-yl)methanamine (Compound 88) and (S*)-(3-fluoro-10,11-dihydrodibenzo[b,f]oxepin-10-yl)methanamine (Compound 89)

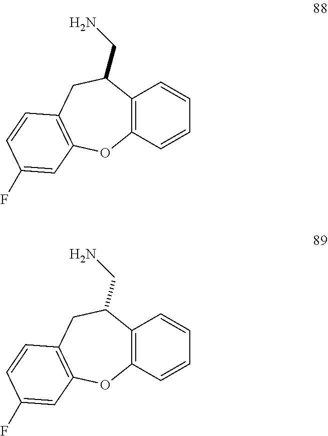 [SMILES: NC[C@@H]1Cc2ccc(F)cc2Oc2ccccc21 ; NC[C@H]1Cc2ccc(F)cc2Oc2ccccc21]

Compound 88 and compound 89 were prepared using a similar procedure described in Example 50.

Compound 88. MS (ESI) m/z: 244 [M+H]⁺. ¹H NMR (400 MHz, CD₃OD) δ 7.35-7.31 (m, 2H), 7.27-7.19 (m, 3H), 6.96 (dd, J=9.6, 24 Hz, 1H), 6.85 (td, J=8.4, 2.8 Hz, 1H), 3.55 (s, 1H), 3.40-3.36 (m, 2H), 3.27 (dd, J=12.8, 7.2 Hz, 1H), 3.14 (dd, J=16, 6.4 Hz, 1H). Chiral analysis column: AD-3 4.6*100 mm 3 μm, Method Set: AD 20% B1; Co-Solvent: methanol[0.2% NH3(7M in methanol)]Temperature: 40° C.; Flow: 3.0 mL/min. Retention time: 1.855 min.

Compound 89. MS(ESI) m/z: 244 [M+H]⁺. ¹H NMR (400 MHz, CD₃OD) δ 7.36-7.31 (m, 2H), 7.27-7.19 (m, 3H), 6.96 (dd, J=9.6, 24 Hz, 1H), 6.85 (td, J=8.4, 2.8 Hz, 1H), 3.55 (s, 1H), 3.40-3.36 (m, 2H), 3.27 (dd, J=12.8, 7.2 Hz, 1H), 3.14 (dd, J=16, 6.4 Hz, 1H). Chiral analysis column: AD-3 4.6*100 mm 3 μm, Method Set: AD 20% B1; Co-Solvent: methanol[0.2% NH3(7M in methanol)]Temperature: 40° C.; Flow: 3.0 mL/min. Retention time: 1.380 min.

### Example 53. Synthesis of (R*)-(3-fluoro-10,11-dihydrodibenzo[b,f]oxepin-10-yl)-N-methylmethanamine (Compound 90) and (S*)-(3-fluoro-10,11-dihydrodibenzo[b,f]oxepin-10-yl)-N-methylmethanamine (Compound 91)

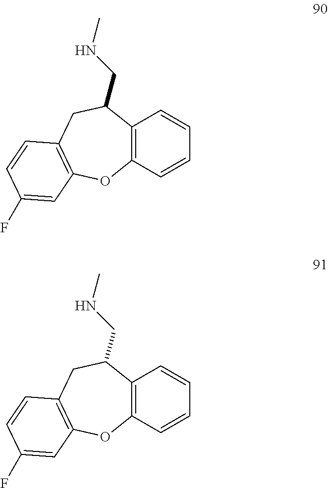 [SMILES: CNC[C@@H]1Cc2ccc(F)cc2Oc2ccccc21 ; CNC[C@H]1Cc2ccc(F)cc2Oc2ccccc21]

Compound 90 and compound 91 were prepared using a similar procedure described in Example 51.

Compound 90. MS (ESI) m/z: 258[M+H]⁺. ¹H NMR (400 MHz, CD₃OD) δ 7.37-7.33 (m, 2H), 7.28-7.20 (m, 3H), 6.99 (dd, J=9.6, 2.8 Hz, 1H), 6.87 (td, J=8.4, 2.8 Hz, 1H), 3.65-3.60 (m, 1H), 3.53-3.48 (m, 1H), 3.39-3.34 (m, 2H), 3.20 (dd, J=16.4, 6.0 Hz, 1H), 2.72 (s, 3H). Chiral analysis column: AY-H (250*4.6 mm 5 μm); Mobile Phase: n-Hexane (0.1% DEA):ethanol (0.1% DEA)=95:5; Temperature: 40° C.; Flow: 1.0 mL/min. Retention time: 4.825 min.

Compound 91. MS (ESI) m/z: 258[M+H]⁺. ¹H NMR (400 MHz, CD₃OD) δ 7.37-7.33 (m, 2H), 7.28-7.20 (m, 3H), 6.99 (dd, J=9.6, 2.8 Hz, 1H), 6.87 (td, J=8.4, 2.8 Hz, 1H), 3.66-3.60 (m, 1H), 3.53-3.48 (m, 1H), 3.39-3.34 (m, 2H), 3.20 (dd, J=16.4, 6.0 Hz, 1H), 2.72 (s, 3H). Chiral analysis column: AY-H (250*4.6 mm 5 μm); Mobile Phase: n-Hexane (0.1% DEA):ethanol (0.1% DEA)=95:5; Temperature: 40° C.; Flow: 1.0 mL/min. Retention time: 5.898 min.

### Example 54. Synthesis of (R*)-(4-fluoro-10,11-dihydrodibenzo[b,f]oxepin-10-yl)methanamine (Compound 84) and (S*)-(4-fluoro-10,11-dihydrodibenzo[b,f]oxepin-10-yl)methanamine (Compound 85)

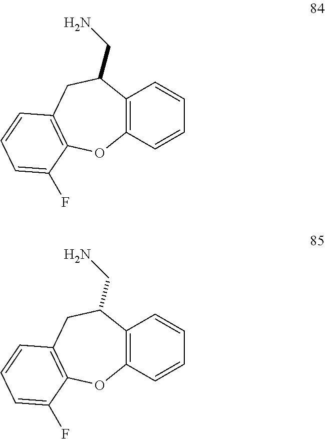 [SMILES: NC[C@@H]1Cc2cccc(F)c2Oc2ccccc21 ; NC[C@H]1Cc2cccc(F)c2Oc2ccccc21]

Compound 84 and compound 85 were prepared using a similar procedure described in Example 50.

Compound 84. MS (ESI) m/z: 244 [M+H]⁺. ¹H NMR (400 MHz, CD₃OD) δ 7.35-7.28 (m, 2H), 7.25-7.17 (m, 2H), 7.06-7.04 (m, 3H), 3.65-3.62 (m, 1H), 3.47-3.32 (m, 2H), 3.28-3.20 (m, 2H). Chiral analysis column: OJ-3 4.6*100 mm 3 μm; co-solvent: methanol [0.2% NH3(7M in methanol)]; Acq. method Set: OJ=10% B1; flow rate: 3.0 mL/min; column temperature: 40° C.; retention time=1.455 min.

Compound 85. MS (ESI) m/z: 244 [M+H]⁺. ¹H NMR (400 MHz, CD₃OD) δ 7.36-7.32 (m, 2H), 7.28-7.20 (m, 2H), 7.10-7.04 (m, 3H), 3.62-3.59 (m, 1H), 3.47-3.32 (m, 2H), 3.29-3.20 (m, 2H). Chiral analysis column: OJ-3 4.6*100 mm 3 μm; co-solvent: methanol [0.2% NH3(7M in methanol)]; Acq. method Set: OJ=10% B1; flow rate: 3.0 mL/min; column temperature: 40° C.; retention time=1.883 min.

### Example 55. Synthesis of (R*)-(4-fluoro-10,11-dihydrodibenzo[b,f]oxepin-10-yl)-N-methylmethanamine (Compound 86) and (S*)-(4-fluoro-10,11-dihydrodibenzo[b,f]oxepin-10-yl)-N-methylmethanamine (Compound 87)

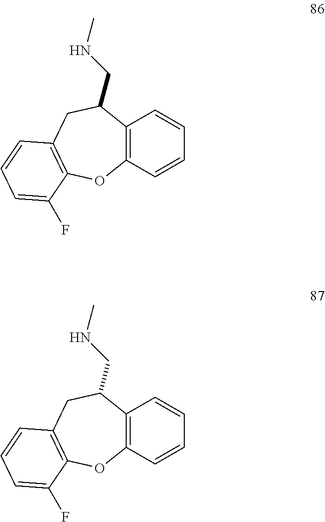 [SMILES: CNC[C@@H]1Cc2cccc(F)c2Oc2ccccc21 ; CNC[C@H]1Cc2cccc(F)c2Oc2ccccc21]

Compound 86 and compound 87 were prepared using a similar procedure described in example 51.

Compound 86. MS (ESI) m/z: 258 [M+H]⁺. ¹H NMR (400 MHz, CD₃OD) δ 7.39-7.35 (m, 2H), 7.30-7.22 (m, 2H), 7.10-7.04 (m, 3H), 3.66-3.63 (m, 1H), 3.56-3.51 (m, 1H), 3.46-3.40 (m, 2H), 3.24 (dd, J=16.4, 6.4 Hz, 2H), 2.736 (s, 3H). Chiral analysis column: AY-H (250*4.6 mm 5 μm); Moblie Phase: n-Hexane (0.1% DEA):ethanol (0.1% DEA)=95:5; Temperature: 40° C.; Flow:1.0 mL/min; Wavelength:214 nm&254 nm; Instrument: SHIMADZU; Inject Volume: 0.8 μl; Vial: 48; retention time=5.325 min.

Compound 87. MS (ESI) m/z: 258 [M+H]⁺. ¹H NMR (400 MHz, CD₃OD) δ 7.39-7.35 (m, 2H), 7.30-7.22 (m, 2H), 7.12-7.02 (m, 3H), 3.66-3.63 (m, 1H), 3.56-3.51 (m, 1H), 3.46-3.40 (m, 2H), 3.24 (dd, J=16.4, 6.4 Hz, 2H), 2.736 (s, 3H). Chiral analysis column: AY-H (250*4.6 mm 5 μm); Moblie Phase: n-Hexane (0.1% DEA):ethanol (0.1% DEA)=95:5; Temperature: 40° C.; Flow:1.0 mL/min; Wavelength:214 nm&254 nm; Instrument: SHIMADZU; Inject Volume: 0.8 μl; Vial: 49; retention time=6.078 min.

### Example 56. Synthesis of (R*)-(2-fluoro-10,11-dihydrodibenzo[b,f]oxepin-10-yl)methanamine (Compound 96) and (S*)-(2-fluoro-10,11-dihydrodibenzo[b,f]oxepin-10-yl)methanamine (Compound 97)

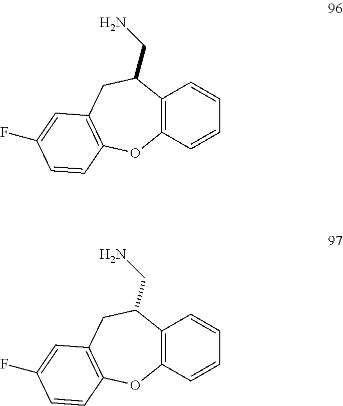 [SMILES: NC[C@@H]1Cc2cc(F)ccc2Oc2ccccc21 ; NC[C@H]1Cc2cc(F)ccc2Oc2ccccc21]

Compound 96 and compound 97 were prepared using a similar procedure described in Example 50.

Compound 96. MS (ESI) m/z: 244 [M+H]⁺. ¹H NMR (400 MHz, CD₃OD) δ 7.35-7.30 (m, 2H), 7.26-7.18 (m, 3H), 7.03-6.96 (m, 2H), 3.56-3.53 (m, 1H), 3.44-3.39 (m, 2H), 3.30-3.25 (m, 1H), 3.13 (dd, J=16.1, 6.4 Hz, 1H). Chiral analysis column Name: AD-3 4.6*100 mm 3 μm; Acq. Method Set: AD 20% B1; Co-Solvent: methanol[0.2% NH3(7M in methanol)]; Injection Volume: 5.00 μl; Run Time: 3.0 Minutes; Flow_rate: 3.0 mL/min; Back_Pressure: 2000 psi; Column Temperature: 40° C.; retention time: 1.457 min.

Compound 97. MS (ESI) m/z: 244 [M+H]⁺. ¹H NMR (400 MHz, CD₃OD) δ 7.35-7.30 (m, 2H), 7.26-7.18 (m, 3H), 7.03-6.96 (m, 2H), 3.56-3.53 (m, 1H), 3.44-3.39 (m, 2H), 3.30-3.25 (m, 1H), 3.13 (dd, J=16.1, 6.4 Hz, 1H). Chiral analysis column Name: AD-3 4.6*100 mm 3 μm; Acq. Method Set: AD 20% B1; Co-Solvent: methanol[0.2% NH3(7M in methanol)]; Injection Volume: 5.00 μl; Run Time: 3.0 Minutes; Flow_rate: 3.0 mL/min; Back_Pressure: 2000 psi; Column Temperature: 40° C.; retention time: 1.800 min.

### Example 57. Synthesis of (R*)-(2-fluoro-10,11-dihydrodibenzo[b,f]oxepin-10-yl)-N-methylmethanamine (Compound 98) and (S*)-(2-fluoro-10,11-dihydrodibenzo[b,f]oxepin-10-yl)-N-methylmethanamine (Compound 99)

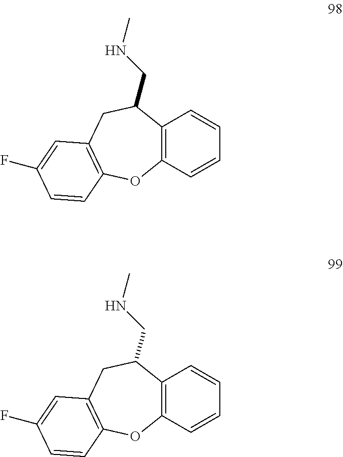 [SMILES: CNC[C@@H]1Cc2cc(F)ccc2Oc2ccccc21 ; CNC[C@H]1Cc2cc(F)ccc2Oc2ccccc21]

Compound 98 and compound 99 were prepared using a similar procedure described in example 51.

Compound 98. MS (ESI) m/z: 258 [M+H]⁺. ¹H NMR (CD₃OD, 500 MHz) δ 7.36-7.33 (m, 2H), 7.27-7.20 (m, 3H), 7.02-6.96 (m, 2H), 3.64-3.60 (m, 1H), 3.57-3.52 (dd, J=3.0, 12.5 Hz, 1H), 3.42-3.37 (m, 2H), 3.21-3.16 (dd, J=6.0, 16.5 Hz, 1H), 2.74 (s, 3H). Chiral analysis column: AY-H (250*4.6 mm 5 μm); mobile phase: n-Hexane (0.1% DEA):ethanol (0.1% DEA)=95:5; wavelength: 275 nm; flow rate: 1 mL/min; temperature: 40° C.; Instrument: SHIMADZU; retention time=6.09 min.

Compound 99. MS (ESI) m/z: 258 [M+H]⁺. ¹H NMR (CD₃OD, 500 MHz) δ 7.36-7.33 (m, 2H), 7.27-7.20 (m, 3H), 7.02-6.96 (m, 2H), 3.64-3.60 (m, 1H), 3.57-3.52 (dd, J=3.0, 12.5 Hz, 1H), 3.42-3.37 (m, 2H), 3.21-3.16 (dd, J=6.0, 16.5 Hz, 1H), 2.74 (s, 3H). Chiral analysis column: AY-H (250*4.6 mm 5 μm); mobile phase: n-Hexane (0.1% DEA):ethanol (0.1% DEA)=95:5; wavelength: 275 nm; flow rate: 1 mL/min; temperature: 40° C.; Instrument: SHIMADZU; retention time=5.58 min.

### Example 58. Synthesis of (R*)-(6-fluoro-10,11-dihydrodibenzo[b,f]oxepin-10-yl)methanamine (Compound 100) and (S*)-(6-fluoro-10,11-dihydrodibenzo[b,f]oxepin-10-yl)methanamine (Compound 101)

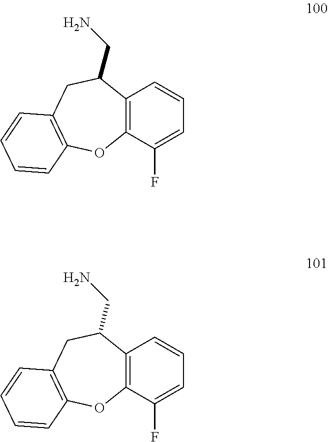 [SMILES: NC[C@@H]1Cc2ccccc2Oc2c(F)cccc21 ; NC[C@H]1Cc2ccccc2Oc2c(F)cccc21]

Compound 100 and compound 101 were prepared using a similar procedure described in Example 50.

Compound 100. MS (ESI) m/z: 244 [M+H]⁺. ¹H NMR (500 MHz, CD₃OD) δ 7.27-7.24 (m, 3H), 7.19-7.10 (m, 4H), 3.63-3.60 (m, 1H), 3.46-3.41 (m, 2H), 3.31-3.28 (m, 1H), 3.18 (dd, J=16.0, 6.5 Hz, 1H). Chiral analysis chiral analysis column: AD-H (250*4.6 mm 5 μm); Moblie Phase: n-Hexane (0.11% DEA):ethanol (0.1% DEA)=95:5; flow rate: 1.0 mL/min; column temperature: 40° C.; Wavelength: 270 nm; Instrument: SHIMADZU; retention time=11.513 min.

Compound 101. MS (ESI) m/z: 244 [M+H]⁺. ¹H NMR (500 MHz, CD₃OD) δ 7.27-7.24 (m, 3H), 7.19-7.10 (m, 4H), 3.63-3.60 (m, 1H), 3.46-3.41 (m, 2H), 3.31-3.28 (m, 1H), 3.18 (dd, J=16.0, 6.5 Hz, 1H). Chiral analysis column: AD-H (250*4.6 mm 5 μm); Moblie Phase: n-Hexane (0.11% DEA):ethanol (0.1% DEA)=95:5; flow rate: 1.0 mL/min; column temperature: 40° C.; Wavelength: 270 nm; Instrument: SHIMADZU; retention time=14.803 min.

### Example 59. Synthesis of (R*)-(6-fluoro-10,11-dihydrodibenzo[b,f]oxepin-10-yl)-N-methylmethanamine (Compound 102) and (S*)-(6-fluoro-10,11-dihydrodibenzo[b,f]oxepin-10-yl)-N-methylmethanamine (Compound 103)

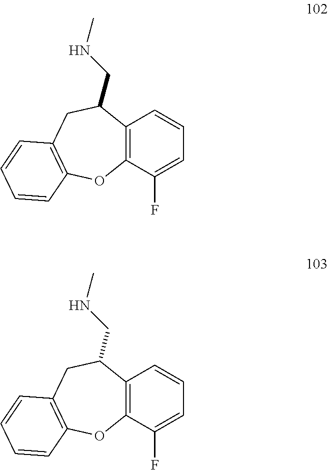 [SMILES: CNC[C@@H]1Cc2ccccc2Oc2c(F)cccc21 ; CNC[C@H]1Cc2ccccc2Oc2c(F)cccc21]

Compound 102 and compound 103 were prepared using a similar procedure described in example 51.

Compound 102. MS (ESI) m/z: 258[M+H]⁺. ¹H NMR (CD₃OD, 500 MHz) δ 7.29-7.24 (m, 3H), 7.20-7.12 (m, 4H), 3.71-3.67 (m, 1H), 3.56 (dd, J=8.0, 12.5 Hz, 1H), 3.45-3.37 (m, 2H), 3.22 (dd, J=6.0, 16.5 Hz, 1H), 2.74 (s, 3H). Chiral analysis column: AY-H (250*4.6 mm, 5 μm); Mobile Phase: n-Hexane (0.11% DEA):ethanol (0.1% DEA)=90:10; flow_rate: 1.0 mL/min; column temperature: 40° C.; Wavelength: 254 nm; Instrument: SHIMADZU; retention time=5.292 min.

Compound 103. MS (ESI) m/z: 258[M+H]⁺. ¹H NMR (CD₃OD, 500 MHz) δ 7.29-7.24 (m, 3H), 7.20-7.12 (m, 4H), 3.71-3.67 (m, 1H), 3.56 (dd, J=8.0, 12.5 Hz, 1H), 3.45-3.37 (m, 2H), 3.22 (dd, J=6.0, 16.5 Hz, 1H), 2.74 (s, 3H). Chiral analysis column: AY-H (250*4.6 mm, 5 μm); Mobile Phase: n-Hexane (0.11% DEA):ethanol (0.1% DEA)=90:10; flow_rate: 1.0 mL/min; column temperature: 40° C.; Wavelength: 254 nm; Instrument: SHIMADZU; retention time=4.612 min.

### Example 60. Synthesis of (R*)-(9-fluoro-10,11-dihydrodibenzo[b,f]oxepin-10-yl)methanamine (Compound 104) and (S*)-(9-fluoro-10,11-dihydrodibenzo[b,f]oxepin-10-yl)methanamine (Compound 105)

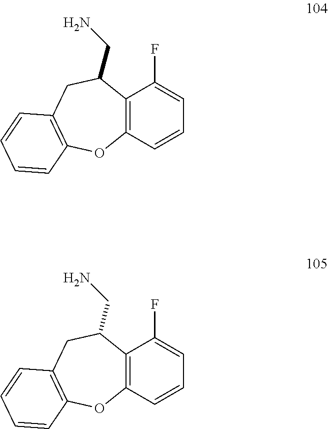 [SMILES: NC[C@@H]1Cc2ccccc2Oc2cccc(F)c21 ; NC[C@H]1Cc2ccccc2Oc2cccc(F)c21]

Compound 104 and compound 105 were prepared using a similar procedure described in Example 50.

Compound 104. MS (ESI) m/z: 244 [M+H]⁺. ¹H NMR (500 MHz, CD₃OD) δ 7.36-7.29 (m, 3H), 7.24-7.11 (m, 3H), 7.00-6.96 (m, 1H), 3.83 (m, 1H), 3.52 (dd, J=15.0, 4.0 Hz, 1H), 3.22-3.08 (m, 3H). Chiral analysis Column: AY-H (250*4.6 mm 5 μm); Mobile Phase: n-Hexane (0.10% DEA):ethanol (0.10% DEA)=70:30; Temperature: 40° C.; Flow:1.0 mL/min; Wavelength:214 nm&254 nm; Instrument: SHIMADZU; retention time: 5.86 min.

Compound 105. MS (ESI) m/z: 244 [M+H]⁺. ¹H NMR (500 MHz, CD₃OD) δ 7.36-7.29 (m, 3H), 7.24-7.11 (m, 3H), 7.00-6.96 (m, 1H), 3.83 (m, 1H), 3.52 (dd, J=15.0, 4.0 Hz, 1H), 3.22-3.08 (m, 3H). Chiral analysis Column: AY-H (250*4.6 mm 5 μm); Mobile Phase: n-Hexane (0.10% DEA):ethanol (0.10% DEA)=70:30; Temperature: 40° C.; Flow:1.0 mL/min; Wavelength:214 nm&254 nm; Instrument: SHIMADZU; retention time: 4.326 min.

### Example 61. Synthesis of (R*)-(9-fluoro-10,11-dihydrodibenzo[b,f]oxepin-10-yl)-N-methylmethanamine (Compound 106) and (S*)-(9-fluoro-10,11-dihydrodibenzo[b,f]oxepin-10-yl)-N-methylmethanamine (Compound 107)

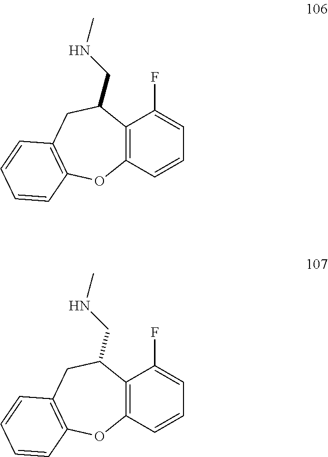 [SMILES: CNC[C@@H]1Cc2ccccc2Oc2cccc(F)c21 ; CNC[C@H]1Cc2ccccc2Oc2cccc(F)c21]

Compound 106 and compound 107 were prepared using a similar procedure described in example 51.

Compound 106. MS (ESI) m/z: 258 [M+H]⁺. ¹H NMR (CD₃OD, 500 MHz) δ 7.37-7.28 (m, 3H), 7.24-7.22 (m, 1H), 7.18-7.12 (m, 2H), 7.02-6.98 (m, 1H), 3.93-3.91 (m, 1H), 3.50 (dd, J=3.5, 15.5 Hz, 1H), 3.26-3.22 (m, 3H), 2.72 (s, 3H). Chiral analysis column: OJ-H (250*4.6 mm 5 μm); mobile phase: n-Hexane (0.11% DEA):ethanol (0.1% DEA)=90:10; wavelength: 214 nm&254 nm; flow rate: 1 mL/min; temperature: 40° C.; Instrument: SHIMADZU; retention time=4.394 min.

Compound 107. MS (ESI) m/z: 258 [M+H]⁺. ¹H NMR (CD₃OD, 500 MHz) δ 7.37-7.28 (m, 3H), 7.24-7.22 (m, 1H), 7.18-7.12 (m, 2H), 7.02-6.98 (m, 1H), 3.93-3.91 (m, 1H), 3.50 (dd, J=3.5, 15.5 Hz, 1H), 3.26-3.22 (m, 3H), 2.72 (s, 3H). Chiral analysis column: OJ-H (250*4.6 mm 5 μm); mobile phase: n-Hexane (0.11% DEA):ethanol (0.1% DEA)=90:10; wavelength: 214 nm&254 nm; flow rate: 1 mL/min; temperature: 40° C.; Instrument: SHIMADZU; retention time=5.027 min.

### Example 62. Synthesis of (R*)-(8-fluoro-10,11-dihydrobenzo[6,7]oxepino[3,2-b]pyridin-10-yl)methanamine (Compound 163) and (S*)-(8-fluoro-10,11-dihydrobenzo[6,7]oxepino[3,2-b]pyridin-10-yl)methanamine (Compound 164)

### Preparation of methyl 8-fluorobenzo[6,7]oxepino[3,2-b]pyridine-10-carboxylate

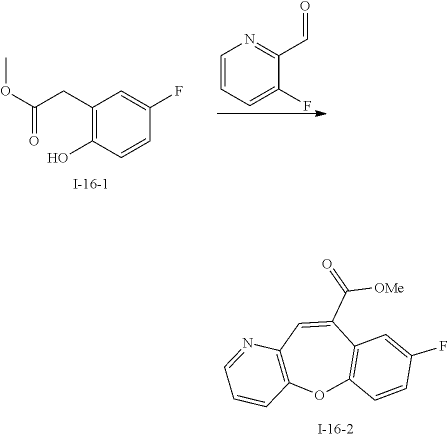

To a solution of methyl 2-(5-fluoro-2-hydroxyphenyl)acetate (2.3 g, 11.8 mmol) in dimethyl sulfoxide (25 mL) was added 3-fluoropyridine-2-carbaldehyde (1.79 g, 14.1 mmol) and caesium carbonate (7.68 g, 23.6 mmol). The reaction was stirred at 100° C. for 16 h. Water (100 mL) was added to the reaction vessel and the mixture was extracted with ethyl acetate (3×100 mL). The combined organics were washed with brine (2×100 mL) and dried over anhydrous sodium sulphate, filtered and concentrated in vacuo. The resulting mixture was purified by flash column chromatography with an isocratic elution of petroleum ether (75%) and ethyl acetate (25%) to provide methyl 8-fluorobenzo[6,7]oxepino[3,2-b]pyridine-10-carboxylate (1.16 g, yield 36.4%) as a yellow solid. MS (ESI) m/z: 272.0 [M+H]⁺.

### Preparation of methyl 8-fluoro-10,11-dihydrobenzo[6,7]oxepino[3,2-b]pyridine-10-carboxylate

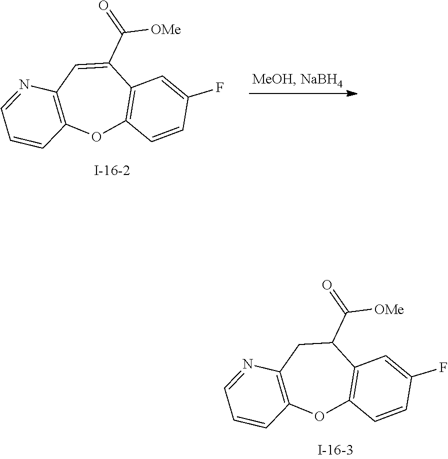 [SMILES: COC(=O)C1=Cc2ncccc2Oc2ccc(F)cc21 ; COC(=O)C1Cc2ncccc2Oc2ccc(F)cc21]

To a solution of methyl 8-fluorobenzo[6,7]oxepino[3,2-b]pyridine-10-carboxylate (1.238 g, 4.54 mmol) in methanol (15 mL) was added sodium borohydride (1.71 g, 45.3 mmol), The reaction was stirred at ambient temperature for 2 h. 1M aqueous hydrogen chloride (50 mL) was added to the reaction vessel and the resulting biphasic mixture was transferred to a separatory funnel. The layers were separated and the aqueous phase was washed with ethyl acetate (3×20 mL). The combined organics were dried over anhydrous sodium sulphate, filtered and concentrated in vacuo. The resulting mixture was purified by flash column chromatography with a gradient elution of petroleum ether (100%) and ethyl acetate (0%) to petroleum ether (0%) and ethyl acetate (100%) to provide methyl 8-fluoro-10,11-dihydrobenzo[6,7]oxepino[3,2-b]pyridine-10-carboxylate (740 mg, 3.01 mmol) as a white solid. MS (ESI) m/z: 274.0 [M+H]⁺.

### Preparation of (8-fluoro-10,11-dihydrobenzo[6,7]oxepino[3,2-b]pyridin-10-yl)methanol

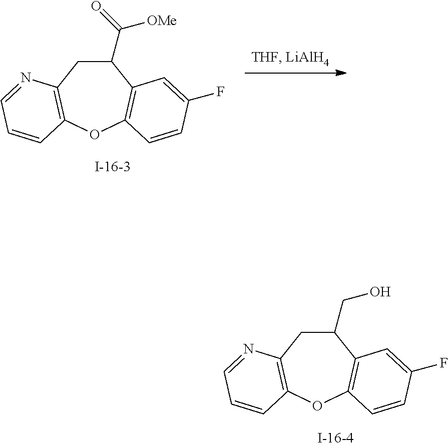

To a solution of methyl 8-fluoro-10,11-dihydrobenzo[6,7]oxepino[3,2-b]pyridine-10-carboxylate (401 mg, 1.46 mmol) in tetrahydrofuran (10 mL) was added aluminium lithium tetrahydride (110 mg, 2.92 mmol). The reaction was stirred at ambient temperature for 2 h. Water (0.66 mL) was added to the reaction vessel slowly to quench the reaction. The mixture was filtered and the filtrate was concentrated in vacuo. The resulting mixture was purified by flash column chromatography with an isocratic elution of petroleum ether (85%) and ethyl acetate (15%) to provide (8-fluoro-10,11-dihydrobenzo[6,7]oxepino[3,2-b]pyridin-10-yl)methanol (341 mg, yield: 95.2%) as a yellow solid. MS (ESI) m/z: 246.1 [M+H]⁺.

### Preparation of 2-((8-fluoro-10,11-dihydrobenzo[6,7]oxepino[3,2-b]pyridin-10-yl)methyl)isoindoline-1,3-dione

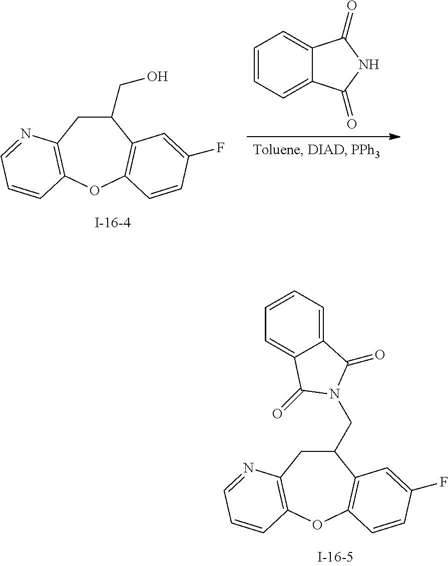

To a solution of (8-fluoro-10,11-dihydrobenzo[6,7]oxepino[3,2-b]pyridin-10-yl)methanol (971 mg, 3.95 mmol) in toluene (20 mL) was added diisopropyl azodicarboxylate (2.38 g, 11.8 mmol), isoindoline-1,3-dione (871 mg, 5.92 mmol) and triphenylphosphane (108 mg, 414 μmol). The reaction mixture was cooled to 0° C. and stirred at that temperature for 2 h. Water (20 mL) was added to the reaction vessel and the resulting biphasic mixture was transferred to a separatory funnel. The layers were separated and the aqueous phase was washed with ethyl acetate (3×20 mL). The combined organics were dried over anhydrous sodium sulphate, filtered and concentrated in vacuo. The crude product was used for next step without further purification.

### Preparation of (8-fluoro-10,11-dihydrobenzo[6,7]oxepino[3,2-b]pyridin-10-yl)methanamine

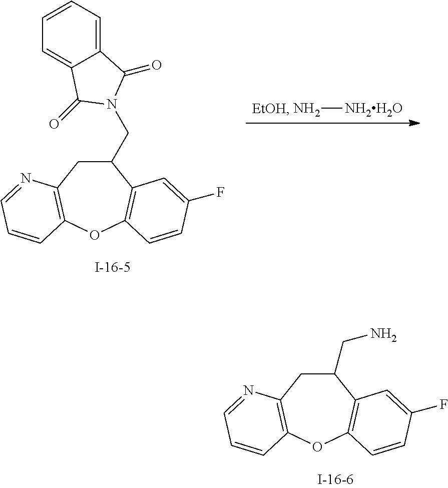

To a solution of 2-((8-fluoro-10,11-dihydrobenzo[6,7]oxepino[3,2-b]pyridin-10-yl)methyl)isoindoline-1,3-dione (crude) in ethanol (20 mL) was added hydrazine hydrate (2 mL), The reaction was stirred at ambient temperature for 3 h. The resulting biphasic mixture was transferred to a separatory funnel. The layers were separated and the organic phase was washed with ethyl acetate (3×5 mL) and water (2×5 mL). The combined organics were dried over anhydrous sodium sulphate, filtered and concentrated in vacuo to provide (8-fluoro-10,11-dihydrobenzo[6,7]oxepino[3,2-b]pyridin-10-yl)methanamine. MS (ESI): m/z=245.1[M+H]⁺.

### Preparation of tert-butyl ((8-fluoro-10,11-dihydrobenzo[6,7]oxepino[3,2-b]pyridin-10-yl)methyl)carbamate

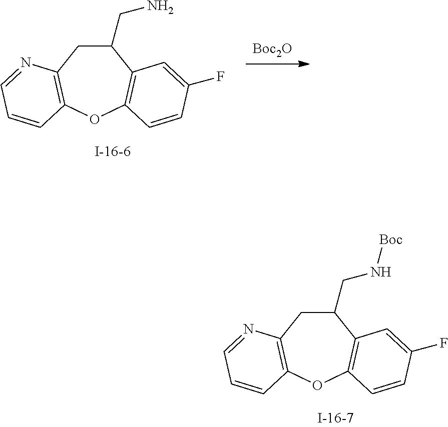 [SMILES: "Boc2O" ; NCC1Cc2ncccc2Oc2ccc(F)cc21 ; CC(C)(C)OC(=O)NCC1Cc2ncccc2Oc2ccc(F)cc21]

To a solution of (8-fluoro-10,11-dihydrobenzo[6,7]oxepino[3,2-b]pyridin-10-yl)methanamine (1.1 g, 4.50 mmol) in dichloromethane (10 mL) and water (5 mL) was added sodium hydroxide (359 mg, 9.00 mmol) and di-tert-butyl dicarbonate (1.47 g, 6.75 mmol). The resulting biphasic mixture was transferred to a separatory funnel. The layers were separated and the aqueous phase was washed with dichloromethane (3×10 mL). The combined organics were dried over anhydrous sodium sulphate, filtered and concentrated in vacuo. The resulting mixture was purified by flash column chromatography with an isocratic elution of petroleum ether (45%) and ethyl acetate (55%) to provide tert-butyl ((8-fluoro-10,11-dihydrobenzo[6,7]oxepino[3,2-b]pyridin-10-yl)methyl)carbamate (1.46 g, yield: 94%) as a yellow solid. MS (ESI) m/z: 345.1. [M+H]⁺.

### Chiral separation of tert-butyl ((8-fluoro-10,11-dihydrobenzo[6,7]oxepino[3,2-b]pyridin-10-yl)methyl)carbamate

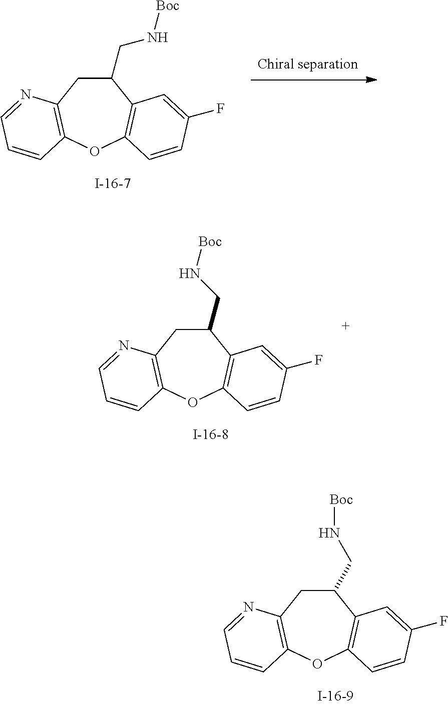 [SMILES: CC(C)(C)OC(=O)NCC1Cc2ncccc2Oc2ccc(F)cc21 ; "Chiral separation" ; CC(C)(C)OC(=O)NC[C@@H]1Cc2ncccc2Oc2ccc(F)cc21 ; CC(C)(C)OC(=O)NC[C@H]1Cc2ncccc2Oc2ccc(F)cc21]

To a solution of tert-butyl ((8-fluoro-10,11-dihydrobenzo[6,7]oxepino[3,2-b]pyridin-10-yl)methyl)carbamate (695 mg) was sent to chiral-separation by Instrument: SFC-150 (Waters) Column: AD 20*250 mm, 10 μm (Daicel) Column temperature: 35° C. Mobile phase: CO2/MEOH (0.2% Methanol Ammonia)=85/15 Flow rate: 100 g/min Back pressure: 100 bar Detection wavelength: 214 nm Cycle time: 3.5 min Sample solution: 695 mg dissolved in 70 mL Methanol Injection volume: 2 mL to provide I-16-8 (288 mg, retention time: 2.16 min) and I-16-9 (180 mg, retention time: 2.51 min).

### Synthesis of (R*)-(8-fluoro-10,11-dihydrobenzo[6,7]oxepino[3,2-b]pyridin-10-yl)methanamine (Compound 163) and (S*)-(8-fluoro-10,11-dihydrobenzo[6,7]oxepino[3,2-b]pyridin-10-yl)methanamine (Compound 164)

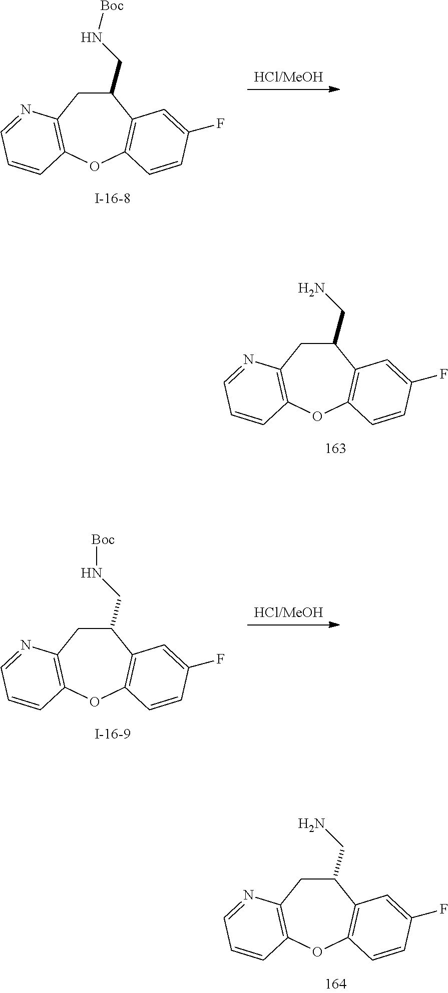 [SMILES: CC(C)(C)OC(=O)NC[C@@H]1Cc2ncccc2Oc2ccc(F)cc21 ; "HCl/MeOH" ; NC[C@@H]1Cc2ncccc2Oc2ccc(F)cc21 ; CC(C)(C)OC(=O)NC[C@H]1Cc2ncccc2Oc2ccc(F)cc21 ; "HCl/MeOH" ; NC[C@H]1Cc2ncccc2Oc2ccc(F)cc21]

To a solution of I-16-8 (157 mg, 438 μmol) in methanol (3 mL) was added 3M HCl/methanol (5 mL). The reaction was stirred at ambient temperature for 16 h. After concentration, compound 163 was obtained. MS (ESI) m/z: 245.1 ¹H NMR (500 MHz, CD₃OD) δ 8.62 (d, J=5.5 Hz, 1H), 8.47 (d, J=8.5 Hz, 1H), 7.95 (dd, J=5.5, 8.5 Hz, 1H), 7.46 (dd, J=5.0, 8.5 Hz, 1H), 7.28 (dd, J=3.0, 8.5 Hz, 1H), 7.21-7.17 (m, 1H), 3.84-3.76 (m, 2H), 3.65-3.51 (m, 3H). Chiral analysis column: IE (4.6*250 mm 5 μm), mobile phase: n-Hexane (0.1% DEA):EtOH (0.1% DEA)=90:10, flowrate: 1 mL/min, temperature: 40° C., retention time: 18.501 min.

To a solution of I-16-9 (46 mg, 129 μmol) in methanol (3 mL) was added 3M hydrogen chloride/methanol (3 mL). The reaction was stirred at ambient temperature for 16 h. Concentrated to afford compound 164. MS (ESI): m/z=245.1 ¹H NMR (500 MHz, CD₃OD) δ 8.62 (d, J=5.0 Hz, 1H), 8.46 (d, J=8.5 Hz, 1H), 7.95 (dd, J=5.0, 7.5 Hz, 1H), 7.46 (dd, J=5.0, 8.5 Hz, 1H), 7.28 (dd, J=3.0, 8.5 Hz, 1H), 7.21-7.17 (m, 1H), 3.84-3.76 (m, 2H), 3.64-3.54 (m, 3H). Chiral analysis column: IE (4.6*250 mm 5 μm), mobile phase: n-Hexane (0.1% DEA):EtOH (0.1% DEA)=90:10, flowrate: 1 mL/min, temperature: 40° C., retention time: 20.970 min.

### Example 63. Synthesis of (R*)-(8-fluoro-10,11-dihydrobenzo[6,7]oxepino[3,2-b]pyridin-10-yl)-N-methylmethanamine (Compound 165) and (S*)-(8-fluoro-10,11-dihydrobenzo[6,7]oxepino[3,2-b]pyridin-10-yl)-N-methylmethanamine (Compound 166)

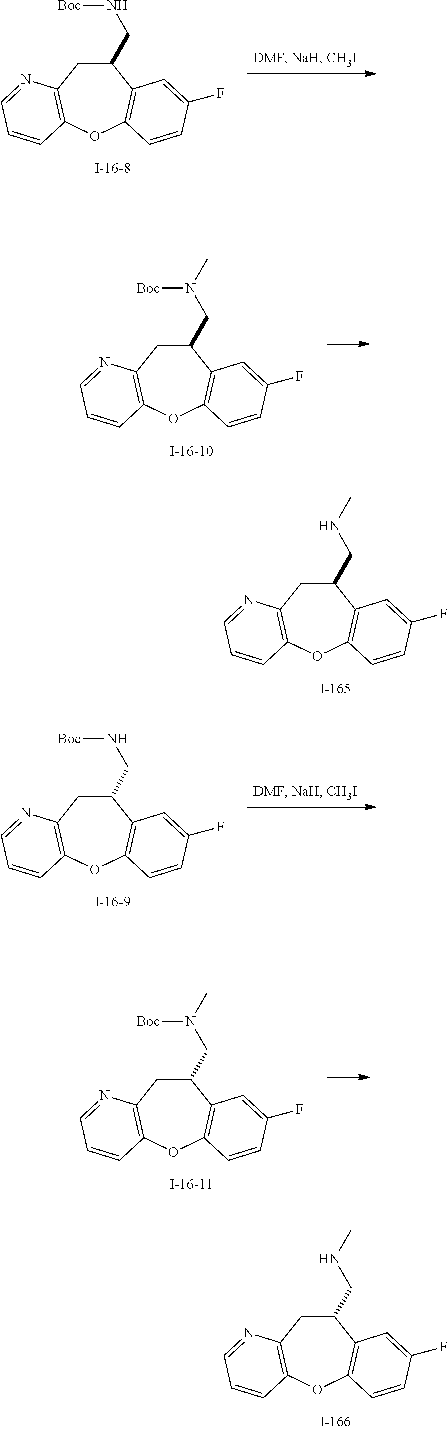

To a solution of I-16-8 (213 mg, 612 μmol) in dimethylformamide (5 mL) was added sodium hydride (60% in mineral oil, 163 mg, 4.10 mmol) and iodomethane (207 mg, 1.46 mmol). The reaction was stirred at ambient temperature for 2 h. Saturated aqueous ammonium chloride (20 mL) was added to the reaction vessel and the resulting biphasic mixture was transferred to a separatory funnel. The layers were separated and the aqueous phase was washed with ethyl acetate (3×10 mL). The combined organics were washed with brine (2×10 mL), dried over anhydrous sodium sulphate, filtered and concentrated in vacuo. The resulting mixture was purified by flash chromatography with an isocratic elution of dichloromethane (90%) and methanol (10%) to provide I-16-10 (157 mg, yield: 71.6%) as a yellow oil. MS (ESI) m/z: 359.1 [M+H]⁺. To a solution of I-16-10 (157 mg, 438 μmol) in methanol (3 mL) was added 3M HCl/Methanol (3 mL). The reaction was stirred at ambient temperature for 16 h. Concentrated to provide compound 165. MS (ESI): m/z=259.2 [M+H]⁺. ¹H NMR (500 MHz, CD₃OD) δ 8.63 (s, 1H), 8.50 (d, J=7.0 Hz, 1H), 7.98 (s, 1H), 7.47 (dd, J=4.5, 8.5 Hz, 1H), 7.32 (d, J=7.0 Hz, 1H), 7.21 (t, J=7.5 Hz, 1H), 3.94-3.79 (m, 2H), 3.70-3.61 (m, 3H), 2.78 (s, 3H). Chiral analysis column: IG (4.6*250 mm 5 μm), mobile phase: n-Hexane (0.1% DEA):EtOH (0.1% DEA)=80:20, flowrate:1.0 mL/min, temperature: 40° C., retention time: 9.777 min.

To a solution of I-16-9 (143 mg, 400 μmol) in dimethylformamide (5 mL) was added sodium hydride (60% in mineral oil, 107 mg, 2.68 mmol) and iodomethane (136 mg, 960 μmol). The reaction was stirred at ambient temperature for 2 h. Saturated aqueous ammonium chloride (20 mL) was added to the reaction vessel and the resulting biphasic mixture was transferred to a separatory funnel. The layers were separated and the aqueous phase was washed with ethyl acetate (3×10 mL). The combined organics were washed with brine (2×10 mL) dried over anhydrous sodium sulphate, filtered and concentrated in vacuo. The resulting mixture was purified by column chromatography with an isocratic elution of dichloromethane (90%) and methanol (10%) to provide I-16-11 (92.6 mg, 258 μmol, yield: 64.7%) as a yellow oil. MS (ESI): m/z=359.1 [M+H]⁺. To a solution of I-16-11 (95 mg, 256 μmol) in methanol (3 mL) was added 3M HCl/methanol (3 mL). The reaction was stirred at ambient temperature for 2 h. Concentrated to provide compound 166. MS (ESI) m/z: 259.2 [M+H]⁺¹H NMR (400 MHz, CD₃OD) δ 8.61 (d, J=2.8 Hz, 1H), 8.45 (d, J=8.0 Hz, 1H), 7.94 (s, 1H), 7.46 (dd, J=4.4, 8.4 Hz, 1H), 7.31 (d, J=6.4 Hz, 1H), 7.21 (t, J=7.6 Hz, 1H), 3.91-3.76 (m, 2H), 3.67-3.60 (m, 3H), 2.78 (s, 3H). Chiral analysis column: IG (4.6*250 mm 5 μm), mobile phase: n-Hexane (0.1% DEA):EtOH (0.1% DEA)=80:20, flowrate:1.0 mL/min, temperature: 40° C., retention time: 10.722 min.

### Example 64. Synthesis of (R*)-(9-fluoro-10,11-dihydrobenzo[6,7]oxepino[3,2-b]pyridin-10-yl)methanamine (Compound 155) and (S*)-(9-fluoro-10,11-dihydrobenzo[6,7]oxepino[3,2-b]pyridin-10-yl)methanamine (Compound 156)

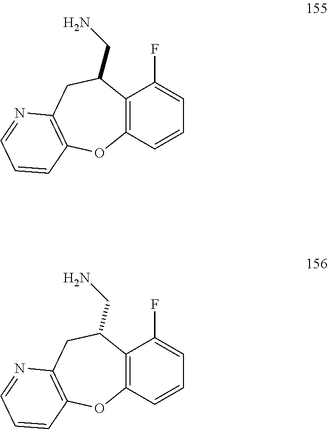 [SMILES: NC[C@@H]1Cc2ncccc2Oc2cccc(F)c21 ; NC[C@H]1Cc2ncccc2Oc2cccc(F)c21]

Compound 155 and compound 156 were prepared using a similar procedure described in example 62.

Compound 155. MS (ESI) m/z: 245 [M+H]⁺. ¹H-NMR (400 MHz, CD₃OD): δ 8.66 (d, J=5.6 Hz, 1H), 8.50 (d, J=8.4 Hz, 1H), 8.01-7.98 (m, 1H), 7.50-7.45 (m, 1H), 7.31 (d, J=8.4 Hz, 1H), 7.21 (t, J=8.8 Hz, 1H), 4.17-4.14 (m, 1H), 3.82 (m, 2H), 3.52 (m, 2H). Chiral analysis column: OX-H (4.6*100 mm 5 μm); co-solvent: methanol [0.2% NH₃ (7M in methanol)]; Co-Solvent % Values: 20%; flow rate: 4.0 mL/min; column temperature: 40° C.; Front Pressure (Bar): 146; retention time=2.14 min.

Compound 156. MS (ESI) m/z: 245 [M+H]⁺. ¹H-NMR (400 MHz, CD₃OD): δ 8.66 (d, J=5.6 Hz, 1H), 8.50 (d, J=8.4 Hz, 1H), 8.01-7.98 (m, 1H), 7.50-7.45 (m, 1H), 7.31 (d, J=8.4 Hz, 1H), 7.21 (t, J=8.8 Hz, 1H), 4.17-4.14 (m, 1H), 3.82 (m, 2H), 3.52 (m, 2H). Chiral analysis column: OX-H (4.6*100 mm 5 μm); co-solvent: methanol [0.2% NH₃ (7M in methanol)]; Co-Solvent % Values: 20%; flow rate: 4.0 mL/min; column temperature: 40° C.; Front Pressure (Bar): 146; retention time=2.65 min.

### Example 65. Synthesis of (R*)-(9-fluoro-10,11-dihydrobenzo[6,7]oxepino[3,2-b]pyridin-10-yl)-N-methylmethanamine (Compound 157) and (S*)-(9-fluoro-10,11-dihydrobenzo[6,7]oxepino[3,2-b]pyridin-10-yl)-N-methylmethanamine (Compound 158)

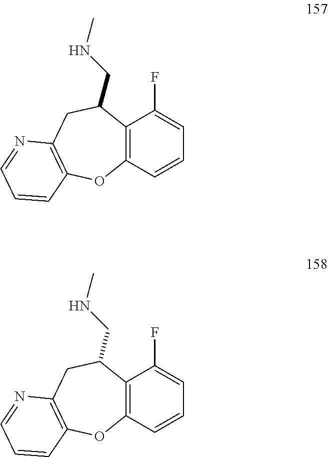 [SMILES: CNC[C@@H]1Cc2ncccc2Oc2cccc(F)c21 ; CNC[C@H]1Cc2ncccc2Oc2cccc(F)c21]

Compound 157 and compound 158 were prepared using a similar procedure described in example 63.

Compound 157. MS (ESI) m/z: 259 [M+H]⁺. ¹H-NMR (400 MHz, CD₃OD): δ 8.69 (d, J=5.2 Hz, 1H), 8.57 (d, J=8.4 Hz, 1H), 8.06-8.03 (m, 1H), 7.51-7.46 (m, 1H), 7.32 (d, J=8.4 Hz, 1H), 7.23 (t, J=8.8 Hz, 1H), 4.26 (S, 1H), 3.94-3.79 (m, 2H), 3.67-3.55 (m, 2H), 2.75 (s, 3H). Chiral analysis column: IG 4.6*100 mm, 5 μm; co-solvent: ethanol [1% NH3 (7 M in methanol)]; Acq. Method Set: 25% B2; flow_rate: 3.0 mL/min; column temperature: 40° C.; Run Time: 6.0 Minutes; Back_Pressure: 2000 psi; retention time=1.93 min.

Compound 158. MS (ESI) m/z: 259 [M+H]⁺. ¹H-NMR (400 MHz, CD₃OD): δ 8.69 (d, J=5.2 Hz, 1H), 8.57 (d, J=8.4 Hz, 1H), 8.06-8.03 (m, 1H), 7.51-7.46 (m, 1H), 7.32 (d, J=8.4 Hz, 1H), 7.23 (t, J=8.8 Hz, 1H), 4.26 (s, 1H), 3.94-3.79 (m, 2H), 3.67-3.55 (m, 2H), 2.75 (s, 3H). Chiral analysis column: IG 4.6*100 mm, 5 μm; co-solvent: ethanol[1% NH3(7 M in methanol)]; Acq. method Set: 25% B2; flow_rate: 3.0 mL/min; column temperature: 40° C.; Run Time: 6.0 Minutes; Back_Presure: 2000 psi; retention time=1.58 min.

### Example 66. Synthesis of (R*)-10-(aminomethyl)-10,11-dihydrobenzo[6,7]-oxepino[3,2-b]pyridine-8-carbonitrile (Compound 159) and (S*)-10-(aminomethyl)-10,11-dihydrobenzo[6,7]oxepino[3,2-b]pyridine-8-carbonitrile (Compound 160)

### Preparation of tert-butyl ((8-bromo-10,11-dihydrobenzo[6,7]oxepino[3,2-b]pyridin-10-yl)methyl)carbamate

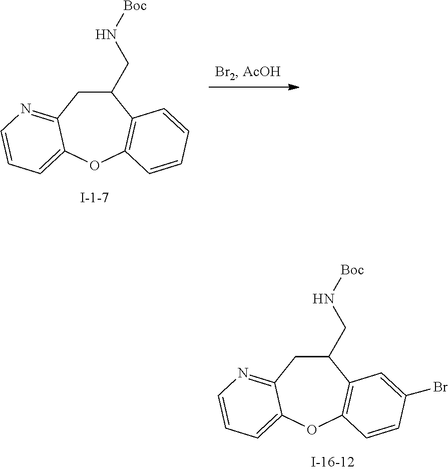

To a solution of tert-butyl ((10,11-dihydrobenzo[6,7]oxepino[3,2-b]pyridin-10-yl)methyl)carbamate (800 mg, 2.45 mmol) in acetic acid (10 mL) was added bromine (1.94 g, 12.2 mmol). The reaction was stirred at ambient temperature for 16 h. sodium thiosulfate (sat. 50 mL) and ethyl acetate (50 mL) were added to the reaction vessel and the resulting biphasic mixture was transferred to a separatory funnel. The layers were separated and the aqueous phase was washed with ethyl acetate (2×50 mL). The combined organics were dried over anhydrous sodium sulphate, filtered and concentrated in vacuo. The resulting oil was purified by flash column chromatography with a gradient elution of petroleum ether (100%) and ethyl acetate (0%) to petroleum ether (70%) and ethyl acetate (30%) to provide tert-butyl ((8-bromo-10,11-dihydrobenzo[6,7]oxepino[3,2-b]pyridin-10-yl)methyl)carbamate (800 mg, yield: 80%) as a yellow oil. MS (ESI) m/z: 405 [M+H]⁺.

### Preparation of tert-butyl ((8-cyano-10,11-dihydrobenzo[6,7]oxepino[3,2-b]pyridin-10-yl)methyl)carbamate

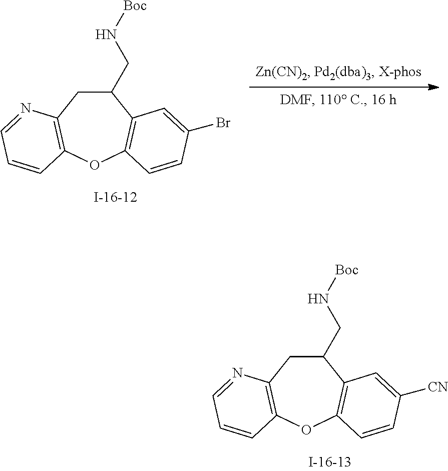 [SMILES: CC(C)(C)OC(=O)NCC1Cc2ncccc2Oc2ccc(Br)cc21 ; "DMF, 110° C., 16 h" ; "Zn(CN)2, Pd2(dba)3, X-phos" ; CC(C)(C)OC(=O)NCC1Cc2ncccc2Oc2ccc(C#N)cc21]

To a solution of tert-butyl ((8-bromo-10,11-dihydrobenzo[6,7]oxepino[3,2-b]pyridin-10-yl)methyl)carbamate (800 mg, 1.97 mmol) in N,N-dimethylformamide (10 mL) was added zincdicarbonitrile (464 mg, 3.95 mmol), Pd₂(dba)₃ (361 mg, 395 μmol) and 2-Di-tert-butylphosphino-2′,4′,6′-triisopropylbiphenyl (188 mg, 790 μmol). The reaction mixture was heated to 110° C. under Ar and stirred at that temperature for 16 h. The mixture was cooled to ambient temperature, saturated aqueous brine (50 mL) and ethyl acetate (30 mL) were added to the reaction vessel and the resulting biphasic mixture was transferred to a separatory funnel. The layers were separated and the aqueous phase was washed with ethyl acetate (2×30 mL). The combined organics were dried over anhydrous sodium sulphate, filtered and concentrated in vacuo. The resulting solid was purified by flash column chromatography (petroleum ether/ethyl acetate=3/1) to provide tert-butyl ((8-cyano-10,11-dihydrobenzo[6,7]oxepino[3,2-b]pyridin-10-yl)methyl)carbamate (500 mg, yield: 72%) as a yellow solid. MS (ESI) m/z: 352 [M+H]⁺.

### Chiral separation of tert-butyl ((8-cyano-10,11-dihydrobenzo[6,7]oxepino[3,2-b]pyridin-10-yl)methyl)carbamate

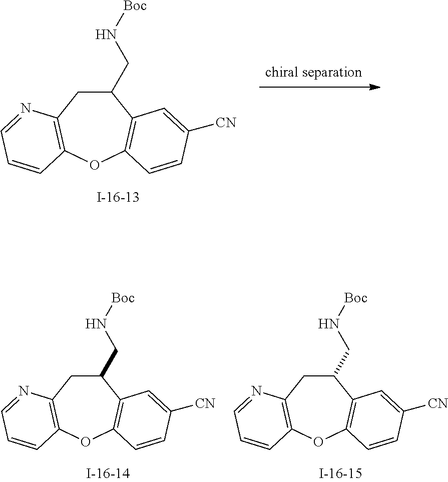

Tert-butyl ((8-cyano-10,11-dihydrobenzo[6,7]oxepino[3,2-b]pyridin-10-yl)methyl)-carbamate (500 mg, 1.42 mmol) was separated by chiral HPLC under following condition:
Instrument: SFC-150 (Thar, Waters)
Column: AD 20*250 mm, 10 μm (Daicel)
Column temperature: 35° C.
Mobile phase: CO2/ETOH (0.5% Methanol Ammonia)=100/20
Flow rate: 100 g/min
Back pressure: 100 bar
Detection wavelength: 214 nm
Cycle time: 2.5 min
Sample solution: 500 mg dissolved in 32 mL Methanol
Injection volume: 1.0 mL

After removing solvents, I-16-14 (200 mg, retention time: 1.98 min) and I-16-15 (200 mg, retention time: 2.33 min) were obtained.

### Synthesis of (R*)-10-(aminomethyl)-10,11-dihydrobenzo[6,7]-oxepino[3,2-b]pyridine-8-carbonitrile (Compound 159) and (S*)-10-(aminomethyl)-10,11-dihydrobenzo[6,7]oxepino[3,2-b]pyridine-8-carbonitrile (Compound 160)

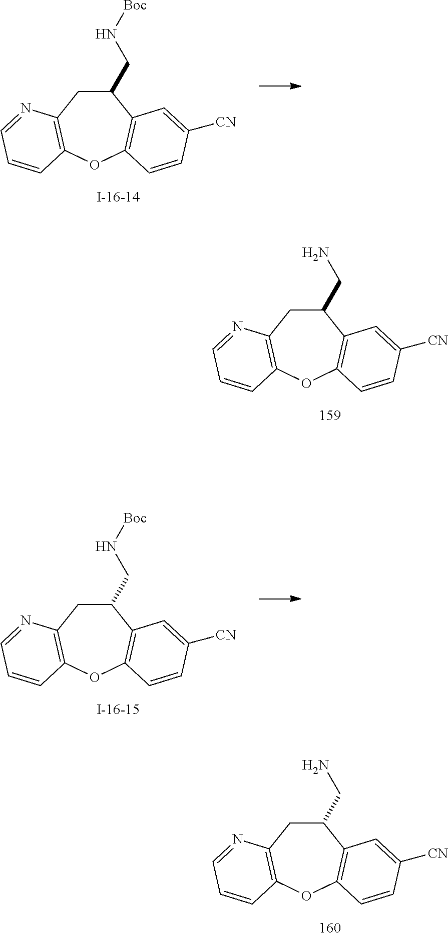 [SMILES: CC(C)(C)OC(=O)NC[C@@H]1Cc2ncccc2Oc2ccc(C#N)cc21 ; N#Cc1ccc2c(c1)[C@H](CN)Cc1ncccc1O2 ; CC(C)(C)OC(=O)NC[C@H]1Cc2ncccc2Oc2ccc(C#N)cc21 ; N#Cc1ccc2c(c1)[C@@H](CN)Cc1ncccc1O2]

A solution of I-16-14 (80 mg, 227 mol) in 3M hydrogen chloride/methanol (10 mL) was stirred at ambient temperature for 16 h. The mixture was concentrated and the residue was added ethyl acetate (3 mL). The mixture was stirred at ambient temperature for 10 min than filtered. The solid was collected to provide Compound 159. MS (ESI) m/z: 252 [M+H]⁺. ¹H NMR (500 MHz, CD₃OD) δ 8.64-8.63 (m, 1H), 8.46-8.44 (m, 1H), 7.96-7.89 (m, 2H), 7.85 (dd, J=2.0, 8.5 Hz, 1H), 7.60 (d, J=8.5 Hz, 1H), 3.89 (bs, 1H), 3.81-3.76 (m, 1H), 3.68-3.63 (m, 1H), 3.58-3.48 (m, 2H). Chiral analysis column: Column: IG (4.6*250 mm 5 μm), mobile phase: n-Hexane (0.1% DEA):ethanol (0.1% DEA)=70:30, wavelength: 254 nm, flowrate:1 mL/min, temperature: 40° C.; retention time=23.911 min.

A solution of I-16-15 (70 mg, 199 μmol) in 3M hydrogen chloride/methanol (10 mL) was stirred at ambient temperature for 16 h. The mixture was concentrated to dryness. Water (5 mL) was added and the mixture was dried by freeze-dryer to provide Compound 160. MS (ESI) m/z: 252 [M+H]⁺. ¹H NMR (500 MHz, CD₃OD) δ 8.66 (d, J=5.5 Hz, 1H), 8.51 (d, J=8.5 Hz, 1H), 8.00 (dd, J=5.5, 8.5 Hz, 1H), 7.91 (d, J=2.0, Hz, 1H), 7.86 (dd, J=2.0, 8.5 Hz, 1H), 7.61 (d, J=8.0 Hz, 1H), 3.92 (bs, 1H), 3.84-3.80 (m, 1H), 3.71-3.66 (m, 1H), 3.57-3.54 (m, 2H). Chiral analysis column: Column: IG (4.6*250 mm 5 μm), mobile phase: n-Hexane (0.1% DEA):ethanol (0.1% DEA)=70:30, wavelength: 254 nm, flowrate:1 mL/min, temperature: 40° C.; retention time=19.793 min.

### Example 67. Synthesis of (R*)-10-((methylamino)methyl)-10,11-dihydrobenzo[6,7]oxepino[3,2-b]pyridine-8-carbonitrile (Compound 161) and (S*)-10-((methylamino)methyl)-10,11-dihydrobenzo[6,7]oxepino[3,2-b]pyridine-8-carbonitrile (Compound 162)

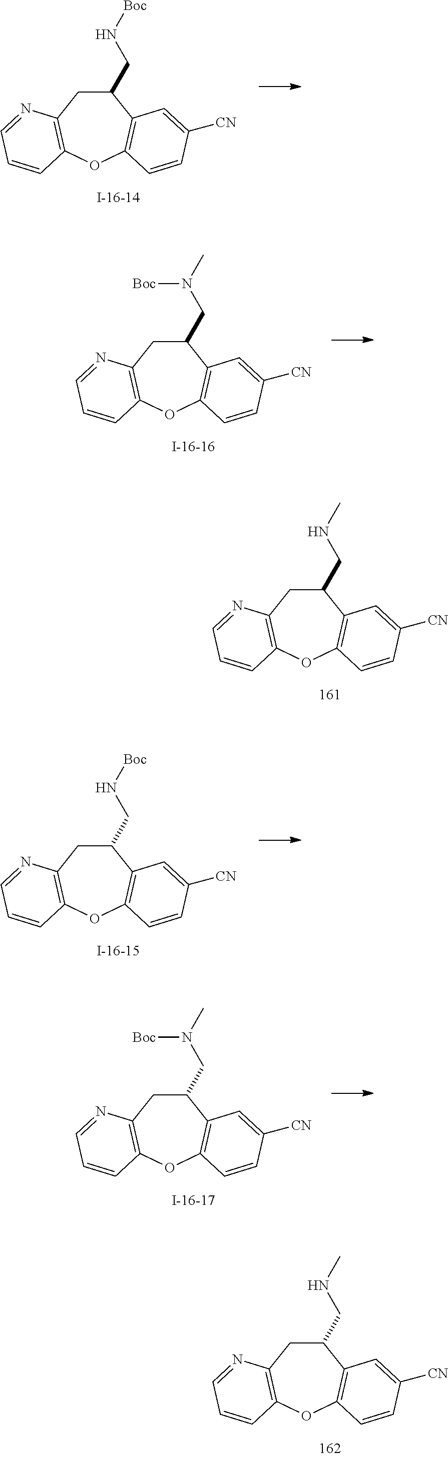 [SMILES: CC(C)(C)OC(=O)NC[C@@H]1Cc2ncccc2Oc2ccc(C#N)cc21 ; CN(C[C@@H]1Cc2ncccc2Oc2ccc(C#N)cc21)C(=O)OC(C)(C)C ; CNC[C@@H]1Cc2ncccc2Oc2ccc(C#N)cc21 ; CC(C)(C)OC(=O)NC[C@H]1Cc2ncccc2Oc2ccc(C#N)cc21 ; CN(C[C@H]1Cc2ncccc2Oc2ccc(C#N)cc21)C(=O)OC(C)(C)C ; CNC[C@H]1Cc2ncccc2Oc2ccc(C#N)cc21]

To a solution of I-16-14 (80 mg, 227 μmol) in N,N-dimethylformamide (3 mL) was added sodium hydride (60% in mineral oil, 27.1 mg, 681 μmol) and iodomethane (96.6 mg, 681 mol). The reaction was stirred at ambient temperature for 5 h. Water (15 mL) was added to the reaction vessel and the mixture was washed with ethyl acetate (3×10 mL). The combined organics were dried over anhydrous sodium sulphate, filtered and concentrated in vacuo. The resulting oil was purified by flash column chromatography (petroleum ether/ethyl acetate=3/1) to provide I-16-16 (70.0 mg, yield: 84%) as a yellow oil. MS (ESI) m/z: 366 [M+H]⁺. A solution of I-16-16 (70 mg, 191 μmol) in 3M hydrogen chloride/methanol (10 mL) was stirred at ambient temperature for 16 h. The mixture was concentrated and the residue was added ethyl acetate (3 mL). The mixture was stirred at ambient temperature for 10 min than filtered. The solid was dissolved in water (10 mL) and dried by freeze-dryer to provide Compound 161. MS (ESI) m/z: 266 [M+H]⁺. ¹H NMR (400 MHz, CD₃OD) δ 8.64 (d, J=5.2 Hz, 1H), 8.44 (d, J=8.0 Hz, 1H), 7.95-7.93 (m, 2H), 7.86 (d, J=1.8, 8.2 Hz, 1H), 7.60 (d, J=8.4 Hz, 1H), 3.99 (bs, 1H), 3.83-3.78 (m, 1H), 3.70-3.53 (m, 3H), 2.77 (s, 3H). Chiral analysis column: Method Info: IG 25% ethanol[1% NH3(7M in methanol)], flow: 4 mL/min, temperature: 40° C., PB: 120 bar, retention time=3.14 min.

To a solution of I-16-15 (80 mg, 227 μmol) in N,N-dimethylformamide (3 mL) was added sodium hydride (60% in mineral oil, 27.1 mg, 681 μmol) and iodomethane (96.6 mg, 681 mol). The reaction was stirred at ambient temperature for 5 h. water (15 mL) was added to the reaction vessel and the mixture was washed with ethyl acetate (3×10 mL). The combined organics were dried over anhydrous sodium sulphate, filtered and concentrated in vacuo. The resulting oil was purified by flash column chromatography (petroleum ether/ethyl acetate=3/1) to provide I-16-17 (70.0 mg, yield: 84%) as a yellow oil. MS (ESI) m/z: 366 [M+H]⁺. A solution of I-16-17 (70 mg, 191 μmol) in 3M hydrogen chloride/methanol (10 mL) was stirred at ambient temperature for 16 h. The mixture was concentrated in vacuo. The residue was added sodium bicarbonate (3 mL) and the mixture was extracted with dichloromethane (3×3 mL). The combined organics were dried over anhydrous sodium sulphate, filtered and concentrated in vacuo to provide Compound 162. MS(ESI) m/z: 266 [M+H]⁺. ¹H NMR (500 MHz, CD₃OD) δ 8.6 (d, J=5.5 Hz, 1H), 8.53 (d, J=9.0 Hz, 1H), 8.01 (dd, J=1.0, 8.5 Hz, 1H), 7.93 (d, J=2.0 Hz, 1H), 7.87 (d, J=2.0, 8.5 Hz, 1H), 7.62 (d, J=9.0 Hz, 1H), 4.00 (bs, 1H), 3.85-3.81 (m, 1H), 3.74-3.56 (m, 3H), 2.77 (s, 3H). Chiral analysis column: Method Info: IG 25% ethanol[1% NH3(7M in methanol)], flow: 4 mL/min, temperature: 40° C., PB: 120 bar, retention time=2.13 min.

### Example 68. Synthesis of (R*)-(8-chloro-10,11-dihydrobenzo[6,7]oxepino[3,2-b]pyridin-10-yl)methanamine (Compound 167) and (S*)-(8-chloro-10,11-dihydrobenzo[6,7]oxepino[3,2-b]pyridin-10-yl)methanamine (Compound 168)

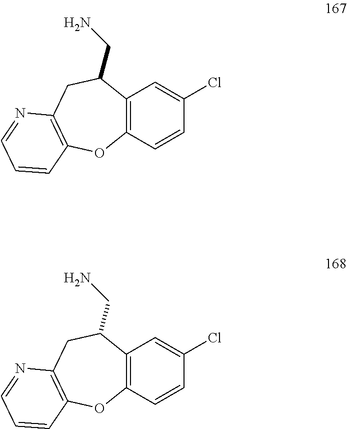 [SMILES: NC[C@@H]1Cc2ncccc2Oc2ccc(Cl)cc21 ; NC[C@H]1Cc2ncccc2Oc2ccc(Cl)cc21]

Compound 167 and Compound 168 were prepared using a similar procedure described in Example 62.

Compound 167. MS (ESI) m/z: 261[M+H]⁺. ¹H NMR (500 MHz, CD₃OD) δ 8.62 (d, J=5.5 Hz, 1H), 8.46 (d, J=8.5 Hz, 1H), 7.96 (dd, J=8.2, 5.8 Hz, 1H), 7.53 (d, J=2.4 Hz, 1H), 7.47-7.42 (m, 2H), 3.84-3.76 (m, 2H), 3.68-3.50 (m, 3H). Chiral analysis column: Method Info: OJ-H 15% methanol[0.2% NH3(7M in methanol)] Flow: 4 mL/min. Temperature: 40° C. PB: 120bar. Retention time: 1.28 min.

Compound 168. MS (ESI) m/z: 261 [M+H]⁺. ¹H NMR (400 MHz, CD₃OD) δ 8.62 (s, 1H), 8.45 (d, J=7.2 Hz, 1H), 7.95 (s, 1H), 7.54 (s, 1H), 7.54-7.41 (m, 2H), 3.83-3.76 (m, 2H), 3.67-3.54 (m, 3H). Chiral analysis column: Column Name: IC-3 4.6*100 mm 3 μm; Acq Method Set: IC 45% B1 Co-Solvent: MeOH[0.2% NH3(7M in MeOH)], Back_Pressure: 2000 psi, Column_Temperature: 40° C.; Flow: 1.0 mL/min. Retention time: 0.860 min.

### Example 69. Synthesis of (R*)-1-(8-chloro-10,11-dihydrobenzo[6,7]oxepino[3,2-b]pyridin-10-yl)-N-methylmethanamine (Compound 169) and (S*)-1-(8-chloro-10,11-dihydrobenzo[6,7]oxepino[3,2-b]pyridin-10-yl)-N-methylmethanamine (Compound 170)

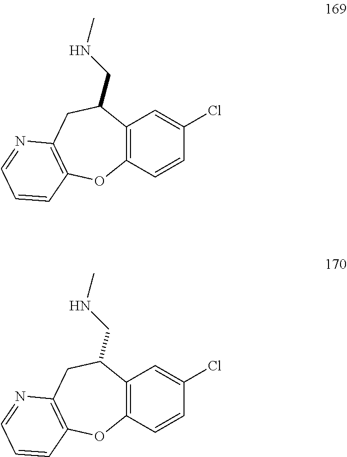 [SMILES: CNC[C@@H]1Cc2ncccc2Oc2ccc(Cl)cc21 ; CNC[C@H]1Cc2ncccc2Oc2ccc(Cl)cc21]

Compound 169 and Compound 170 were prepared using a similar procedure described in Example 63.

Compound 169. MS (ESI) m/z: 275 [M+H]⁺. ¹H NMR (500 MHz, MeOD) δ 8.63 (d, J=4.7 Hz, 1H), 8.46 (br, 1H), 7.96 (br, 1H), 7.56 (s, 1H), 7.48-7.42 (m, 2H), 3.90-3.78 (m, 2H), 2.77 (s, 3H). Chiral analysis column: OJ-H (250*4.6 mm 5 μm); Mobile Phase n-Hexane (0.1% DEA):ethanol (0.1% DEA)=90:10 Temperature: 40° C.; Flow: 1.0 mL/min. Retention time: 5.397 min.

Compound 170. (ESI): m/z=275[M+H]⁺. ¹H NMR (400 MHz, dimethylsulfoxide-d₆) δ 9.13 (s, 2H), 8.33 (dd, J=4.5, 1.4 Hz, 1H), 7.66 (dd, J=8.2, 1.4 Hz, 1H), 7.50 (d, J=2.5 Hz, 1H), 7.43-7.23 (m, 3H), 3.99-3.75 (m, 1H), 3.49 (dd, J=17.1, 3.9 Hz, 1H), 3.41-3.20 (m, 3H), 2.57 (s, 3H). Chiral analysis column: IH (250*4.6 mm 5 μm); Mobile Phase n-Hexane (0.1% DEA):ethanol (0.1% DEA)=80:20 Temperature: 40° C.; Flow: 1.0 mL/min. Retention time: 4.293 min.

### Example 70. Synthesis of (R*)-(7-fluoro-10,11-dihydrobenzo[6,7]oxepino[3,2-b]pyridin-10-yl)methanamine (Compound 35) and (S*)-(7-fluoro-10,11-dihydrobenzo[6,7]oxepino[3,2-b]pyridin-10-yl)methanamine (Compound 36)

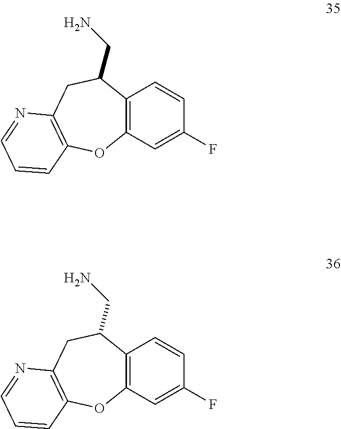 [SMILES: NC[C@@H]1Cc2ncccc2Oc2cc(F)ccc21 ; NC[C@H]1Cc2ncccc2Oc2cc(F)ccc21]

Compound 35 and Compound 36 were prepared using a similar procedure described in Example 62.

Compound 35. MS (ESI) m/z: 245 [M+H]⁺. ¹H NMR (400 MHz, CD₃OD) δ 8.65 (dd, J=5.6, 1.2 Hz, 1H), 8.52 (dd, J=8.4, 1.2 Hz, 1H), 7.01 (dd, J=8.4, 5.6 Hz, 1H), 7.51 (dd, J=8.4, 6.0 Hz, 1H), 7.28 (dd, J=9.2, 2.4 Hz, 1H), 7.14 (td, J=8.4, 2.4 Hz, 1H), 3.86-3.51 (m, 5H). Chiral analysis column: AY-H (254*4.6 mm 5 μm), Mobile Phase: n-Hexane (0.1% DEA): ethanol (0.1% DEA)=80:20, Temperature: 40° C., Flow: 1.0 mL/min, Wavelength: 214 nm&254 nm, Instrument: SHIMADZU, Inject Volume: 4 μl, Vial: 86. Retention time: 7.206 min.

Compound 36. MS (ESI) m/z: 245 [M+H]⁺. ¹H NMR (400 MHz, CD₃OD) δ 8.64 (dd, J=5.6, 1.2 Hz, 1H), 8.53 (dd, J=8.4, 1.2 Hz, 1H), 8.01 (dd, J=8.4, 5.6 Hz, 1H), 7.52 (dd, J=8.4, 6.0 Hz, 1H), 7.28 (dd, J=8.8, 2.8 Hz, 1H), 7.14 (td, J=8.4, 2.4 Hz, 1H), 3.90-3.50 (m, 5H). Chiral analysis column: AY-H (250*4.6 mm 5 μm), Mobile Phase: n-Hexane (0.1% DEA): ethanol (0.1% DEA)=80:20, Temperature: 40° C., Flow: 1.0 mL/min, Wavelength: 214 nm&254 nm, Instrument: SHIMADZU, Inject Volume: 0.5p l, Vial: 87. Retention time: 6.382 min.

### Example 71. Synthesis of (R*)-1-(7-fluoro-10,11-dihydrobenzo[6,7]oxepino[3,2-b]pyridin-10-yl)-N-methylmethanamine (Compound 33) and (S*)-1-(7-fluoro-10,11-dihydrobenzo[6,7]oxepino[3,2-b]pyridin-10-yl)-N-methylmethanamine (Compound 34)

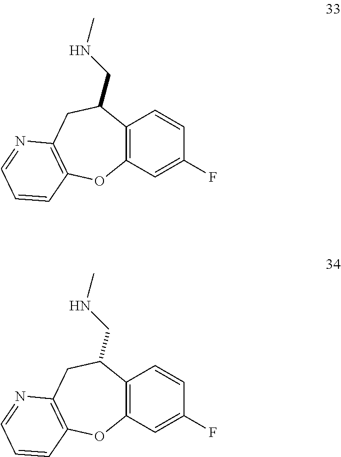 [SMILES: CNC[C@@H]1Cc2ncccc2Oc2cc(F)ccc21 ; CNC[C@H]1Cc2ncccc2Oc2cc(F)ccc21]

Compound 33 and Compound 34 were prepared using a similar procedure described in Example 63.

Compound 33. MS (ESI) m/z: 259 [M+H]⁺. ¹H NMR (400 MHz, CD₃OD) δ 8.65 (dd, J=5.6, 1.2 Hz, 1H), 8.53 (dd, J=8.8, 1.2 Hz, 1H), 8.02 (dd, J=8.4, 5.6 Hz, 1H), 7.53 (dd, J=8.8, 6.4 Hz, 1H), 7.28 (dd, J=9.2, 2.8 Hz, 1H), 7.15 (td, J=8.4, 2.8 Hz, 1H), 3.94-3.82 (m, 1H), 3.78-3.55 (m, 4H), 2.76 (s, 3H). Chiral analysis column: OJ-H (250*4.6 mm 5 μm), Mobile Phase: n-Hexane (0.1% DEA):ethanol (0.1% DEA)=95:5, Temperature: 40° C., Flow: 1.0 mL/min, Wavelength: 214 nm&254 nm, Instrument: SHIMADZU, Inject Volume: 1 μl, Vial: 28. Retention time: 6.713 min.

Compound 34. MS (ESI) m/z: 259 [M+H]⁺. ¹H NMR (400 MHz, CD₃OD) δ 8.66 (dd, J=5.6, 1.2 Hz, 1H), 8.53 (dd, J=8.8, 1.2 Hz, 1H), 8.02 (dd, J=8.4, 5.6 Hz, 1H), 7.54 (dd, J=8.8, 6.4 Hz, 1H), 7.28 (dd, J=9.2, 2.8 Hz, 1H), 7.15 (td, J=8.4, 2.8 Hz, 1H), 3.94-3.82 (m, 1H), 3.78-3.55 (m, 4H), 2.76 (s, 3H). Chiral analysis column: OJ-H (250*4.6 mm 5 μm), Mobile Phase: n-Hexane (0.1% DEA):ethanol (0.1% DEA)=95:5, Temperature: 40° C., Flow: 1.0 mL/min, Wavelength: 214 nm&254 nm, Instrument: SHIMADZU, Inject Volume: 1 μl, Vial: 29. Retention time: 7.519 min.

### Example 72. Synthesis of (R*)-(7-chloro-10,11-dihydrobenzo[6,7]oxepino[3,2-b]pyridin-10-yl)methanamine (Compound 171) and (S*)-(7-chloro-10,11-dihydrobenzo[6,7]oxepino[3,2-b]pyridin-10-yl)methanamine (Compound 172)

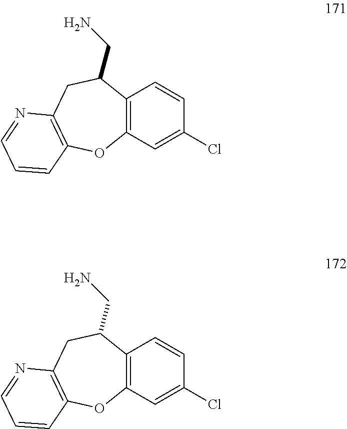 [SMILES: NC[C@@H]1Cc2ncccc2Oc2cc(Cl)ccc21 ; NC[C@H]1Cc2ncccc2Oc2cc(Cl)ccc21]

Compound 171 and Compound 172 were prepared using a similar procedure described in Example 62.

Compound 171. MS (ESI) m/z: 261[M+H]⁺. ¹H NMR (500 MHz, CD₃OD) δ 8.62 (dd, J=5.6, 1.3 Hz, 1H), 8.48 (dd, J=8.6, 1.2 Hz, 1H), 7.97 (dd, J=8.5, 5.6 Hz, 1H), 7.51 (d, J=2.1 Hz, 1H), 7.45 (d, J=8.3 Hz, 1H), 7.37 (dd, J=8.2, 2.1 Hz, 1H), 3.87-3.69 (m, 2H), 3.69-3.39 (m, 3H). Chiral analysis column: IG (4.6*250 mm 5 μm); Mobile Phase: n-Hexane (0.1% DEA):ethanol (0.1% DEA)=60:40; Temperature: 40° C.; Flow: 1.0 mL/min; Wavelength: 214 nm&254 nm; Retention time: 10.06 min.

Compound 172. MS (ESI) m/z: 261 [M+H]⁺. ¹H NMR (400 MHz, CD₃OD) δ 8.62 (d, J=4.7 Hz, 1H), 8.49 (d, J=7.8 Hz, 1H), 7.97 (dd, J=8.5, 5.6 Hz, 1H), 7.51 (d, J=2.0 Hz, 1H), 7.45 (d, J=8.2 Hz, 1H), 7.37 (dd, J=8.2, 2.0 Hz, 1H), 3.91-3.69 (m, 2H), 3.71-3.40 (m, 3H). Chiral analysis column: IG (4.6*250 mm 5 μm); Mobile Phase: n-Hexane (0.1% DEA):ethanol (0.1% DEA)=60:40; Temperature: 40° C.; Flow: 1.0 mL/min; Wavelength: 214 nm&254 nm; Retention time: 15.66 min.

### Example 73. Synthesis of (R*)-1-(7-chloro-10,11-dihydrobenzo[6,7]oxepino[3,2-b]pyridin-10-yl)-N-methylmethanamine (Compound 173) and (S*)-1-(7-chloro-10,11-dihydrobenzo[6,7]oxepino[3,2-b]pyridin-10-yl)-N-methylmethanamine (Compound 174)

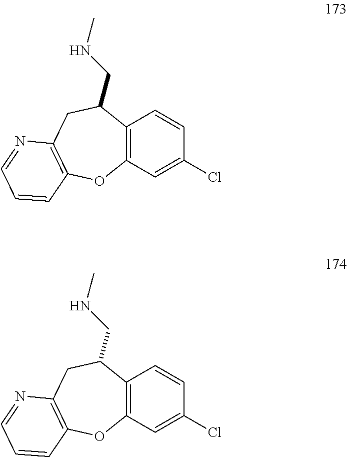 [SMILES: CNC[C@@H]1Cc2ncccc2Oc2cc(Cl)ccc21 ; CNC[C@H]1Cc2ncccc2Oc2cc(Cl)ccc21]

Compound 173 and Compound 174 were prepared using a similar procedure described in Example 63.

Compound 173. MS (ESI) m/z:275 [M+H]⁺. ¹H NMR (400 MHz, CD₃OD) δ 8.73-8.55 (m, 1H), 8.49 (d, J=7.7 Hz, 1H), 7.98 (dd, J=8.5, 5.6 Hz, 1H), 7.52 (d, J=2.0 Hz, 1H), 7.48 (d, J=8.3 Hz, 1H), 7.38 (dd, J=8.2, 2.1 Hz, 1H), 3.90 (s, 1H), 3.83-3.46 (m, 4H), 2.74 (s, 3H). Chiral analysis column: IE (4.6*250 mm 5 μm); Mobile Phase: n-Hexane (0.1% DEA):ethanol (0.1% DEA)=70:30; Temperature: 40° C.; Flow: 1.0 mL/min; Wavelength: 254 nm; Retention time: 5.17 min.

Compound 174. MS (ESI) m/z: 275 [M+H]⁺. ¹H NMR (400 MHz, CD₃OD) δ 8.63 (dd, J=5.6, 1.0 Hz, 1H), 8.55-8.43 (m, 1H), 7.99 (dd, J=8.5, 5.7 Hz, 1H), 7.52 (d, J=2.1 Hz, 1H), 7.48 (d, J=8.3 Hz, 1H), 7.38 (dd, J=8.2, 2.1 Hz, 1H), 3.94-3.57 (m, 5H), 2.74 (s, 3H). Chiral analysis column: IE (4.6*250 mm 5 μm); Mobile Phase: n-Hexane (0.1% DEA):ethanol (0.1% DEA)=70:30; Temperature: 40° C.; Flow: 1.0 mL/min; Wavelength: 254 nm; Retention time: 5.57 min.

### Example 74. Synthesis of (R*)-(6-fluoro-10,11-dihydrobenzo[6,7]oxepino[3,2-b]pyridin-10-yl)methanamine (Compound 175) and (S*)-(6-fluoro-10,11-dihydrobenzo[6,7]oxepino[3,2-b]pyridin-10-yl)methanamine (Compound 176)

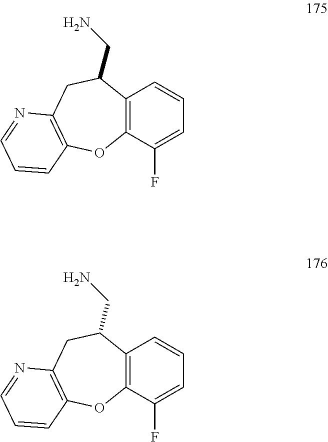 [SMILES: NC[C@@H]1Cc2ncccc2Oc2c(F)cccc21 ; NC[C@H]1Cc2ncccc2Oc2c(F)cccc21]

Compound 175 and Compound 176 were prepared using a similar procedure described in Example 62.

Compound 175. MS (ESI) m/z: 245 [M+H]⁺. ¹H NMR (400 MHz, CD₃OD) δ 8.62 (dd, J=5.6, 1.2 Hz, 1H), 8.50 (dd, J=8.6, 1.2 Hz, 1H), 7.99 (dd, J=8.5, 5.6 Hz, 1H), 7.35-7.26 (m, 3H), 3.92-3.71 (m, 2H), 3.71-3.52 (m, 3H). Chiral analysis column: AD-H (4.6*100 mm 5 μm), Co-Solvent: methanol [0.2% NH3 (7M in methanol)], Sample Well 13C, Temperature: 40° C., Flow: 4.0 mL/min, CO2% Values: 80.0, Co-Solvent % Values: 20.0, Front Pressure (Bar): 147.3 Inject Volume: 5 μl, retention time: 1.55 min.

Compound 176. MS (ESI) m/z: 245 [M+H]⁺. ¹H NMR (400 MHz, CD₃OD) δ 8.62 (dd, J=5.6, 1.2 Hz, 1H), 8.50 (dd, J=8.6, 1.2 Hz, 1H), 7.99 (dd, J=8.5, 5.6 Hz, 1H), 7.35-7.26 (m, 3H), 3.92-3.71 (m, 2H), 3.71-3.52 (m, 3H). Chiral analysis column: AD-H (4.6*100 mm 5 μm), Co-Solvent: methanol [0.2% NH3 (7M in methanol)], Sample Well 13C, Temperature: 40° C., Flow: 4.0 mL/min, CO2% Values: 80.0, Co-Solvent % Values: 20.0, Front Pressure (Bar): 147.3 Inject Volume:5 μl, retention time: 1.26 min.

### Example 75. Synthesis of (R*)-1-(6-fluoro-10,11-dihydrobenzo[6,7]oxepino[3,2-b]pyridin-10-yl)-N-methylmethanamine (Compound 177) and (S*)-1-(6-fluoro-10,11-dihydrobenzo[6,7]oxepino[3,2-b]pyridin-10-yl)-N-methylmethanamine (Compound 178)

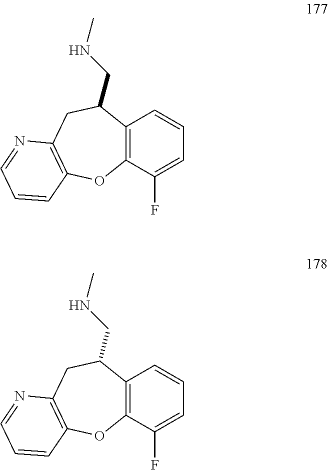 [SMILES: CNC[C@@H]1Cc2ncccc2Oc2c(F)cccc21 ; CNC[C@H]1Cc2ncccc2Oc2c(F)cccc21]

Compound 177 and Compound 178 were prepared using a similar procedure described in Example 63.

Compound 177. MS (ESI) m/z: 259 [M+H]⁺. ¹H NMR (400 MHz, CD₃OD) δ 8.62 (dd, J=5.6, 1.2 Hz, 1H), 8.41 (dd, J=8.6, 1.1 Hz, 1H), 7.90 (dd, J=8.5, 5.6 Hz, 1H), 7.35-7.27 (m, 3H), 4.00-3.58 (m, 5H), 2.77 (s, 3H). Chiral analysis column: IG (100*4.6 mm, 5 μm), Co-Solvent: methanol [0.2% NH3 (7M in methanol)], Temperature: 40° C., Flow: 3.0 mL/min, Inject Volume:5 μl, Back Pressure: 2000 psi, Run Time: 4.0 Minutes, retention time: 1.80 min.

Compound 178. MS (ESI) m/z: 259 [M+H]⁺. ¹H NMR (400 MHz, CD₃OD) δ 8.62 (dd, J=5.6, 1.2 Hz, 1H), 8.41 (dd, J=8.6, 1.1 Hz, 1H), 7.90 (dd, J=8.5, 5.6 Hz, 1H), 7.35-7.27 (m, 3H), 4.00-3.58 (m, 5H), 2.77 (s, 3H). Chiral analysis column: IG (100*4.6 mm, 5 μm), Co-Solvent: methanol [0.2% NH3 (7M in methanol)], Temperature: 40° C., Flow: 3.0 mL/min, Inject Volume: 5 μl, Back Pressure: 2000 psi, Run Time: 4.0 Minutes, retention time: 1.42 min.

### Example 76. Synthesis of (R*)-(3-chloro-10,11-dihydrobenzo[6,7]oxepino[3,2-b]pyridin-10-yl)methanamine (Compound 179) and (S*)-(3-chloro-10,11-dihydrobenzo[6,7]oxepino[3,2-b]pyridin-10-yl)methanamine_(Compound 180)

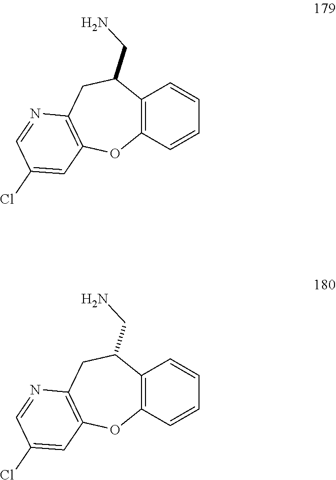 [SMILES: NC[C@@H]1Cc2ncc(Cl)cc2Oc2ccccc21 ; NC[C@H]1Cc2ncc(Cl)cc2Oc2ccccc21]

Compound 179 and Compound 180 were prepared using a similar procedure described in Example 62.

Compound 179. MS (ESI) m/z: 261 [M+H]⁺. ¹H NMR (400 MHz, CD₃OD) δ 8.56 (d, J=4.6 Hz, 1H), 8.35 (d, J=7.9 Hz, 1H), 7.86 (dd, J=8.4, 5.5 Hz, 1H), 7.50 (d, J=2.3 Hz, 1H), 7.42 (m, 2H), 3.74 (m, 2H), 3.64-3.40 (m, 3H). Chiral analysis column: AD-H (100*4.6 mm 5 μm); Mobile Phase: methanol [0.2% NH3 (7M in methanol)] Temperature: 40° C.; Flow: 4.0 mL/min, retention time: 2.76 min.

Compound 180. MS (ESI) m/z: 261 [M+H]⁺. ¹H NMR (400 MHz, CD₃OD) δ 8.56 (d, J=4.6 Hz, 1H), 8.35 (d, J=7.9 Hz, 1H), 7.86 (dd, J=8.4, 5.5 Hz, 1H), 7.50 (d, J=2.3 Hz, 1H), 7.42 (m, 2H), 3.74 (m, 2H), 3.64-3.40 (m, 3H). Chiral analysis column: AD-H (100*4.6 mm 5 μm); Mobile Phase: methanol [0.2% NH3 (7M in methanol)] Temperature: 40° C.; Flow: 4.0 mL/min, retention time: 2.15 min.

### Example 77. Synthesis of (R*)-(3-fluoro-10,11-dihydrobenzo[6,7]oxepino[3,2-b]pyridin-10-yl)methanamine (Compound 39) and (S*)-(3-fluoro-10,11-dihydrobenzo[6,7]oxepino[3,2-b]pyridin-10-yl)methanamine (Compound 40)

### Preparation of methyl 3-(3-bromo-5-fluoropyridin-2-yl)-2-(2-methoxyphenyl)propanoate

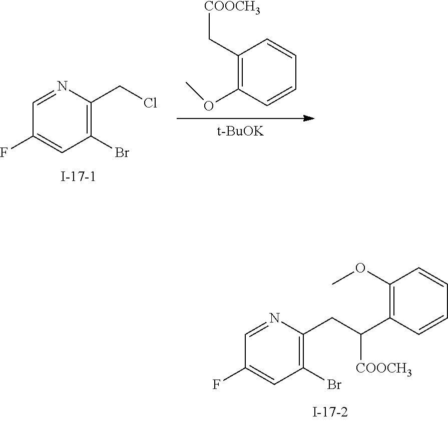

To a solution of potassium tert-butoxide (5.38 g, 48.0 mmol) in tetrahydrofuran (500 mL) was added methyl 2-(2-methoxyphenyl)acetate (7.92 g, 44.0 mmol) at 0° C. After stirred at 0° C. for 1 h, 3-bromo-2-(chloromethyl)-5-fluoropyridine (9 g, 40.0 mmol) was added. The reaction was stirred at ambient temperature for 3 h. Upon the completion, ethyl acetate (200 mL) and water (200 mL) was added. The mixture was extracted with ethyl acetate (200 mL×3), dried and concentrated. The resulting oil was purified by flash column chromatography with a gradient elution of petroleum ether (100%) to petroleum ether (90%) and ethyl acetate (10%) to provide methyl 3-(3-bromo-5-fluoropyridin-2-yl)-2-(2-methoxyphenyl)propanoate (8.09 g, yield:55%) as a colorless oil. MS (ESI) m/z: 368 [M+H]⁺.

### Preparation of 3-(3-bromo-5-fluoropyridin-2-yl)-2-(2-methoxyphenyl)propan-1-ol

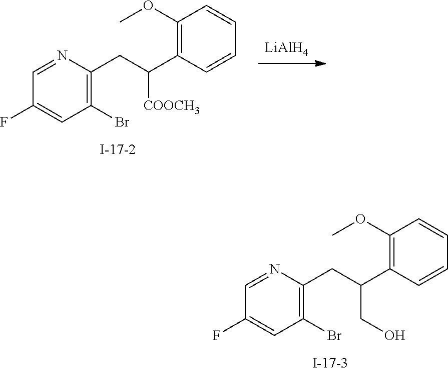 [SMILES: COC(=O)C(Cc1ncc(F)cc1Br)c1ccccc1OC ; "LiAlH4" ; COc1ccccc1C(CO)Cc1ncc(F)cc1Br]

To a solution of methyl 3-(3-bromo-5-fluoropyridin-2-yl)-2-(2-methoxyphenyl)propanoate (11.56 g, 31.2 mmol) in tetrahydrofuran (150 mL) was added lithium aluminum hydride (1.06 g, 28.0 mmol) at 0° C. The reaction was stirred at 0° C. for 1 h. Upon the completion, water (6 mL) was added quenched the reaction and then filtered. The filtrate was concentrated. The resulting oil was purified by flash column chromatography with a gradient elution of petroleum ether (100%) to petroleum ether (80%) and ethyl acetate (20%) to provide 3-(3-bromo-5-fluoropyridin-2-yl)-2-(2-methoxyphenyl)propan-1-ol (6.70 g, yield: 63%) as a colorless oil. MS (ESI) m/z: 340 [M+H]⁺.

### Preparation of 2-(1-(3-bromo-5-fluoropyridin-2-yl)-3-hydroxypropan-2-yl)phenol

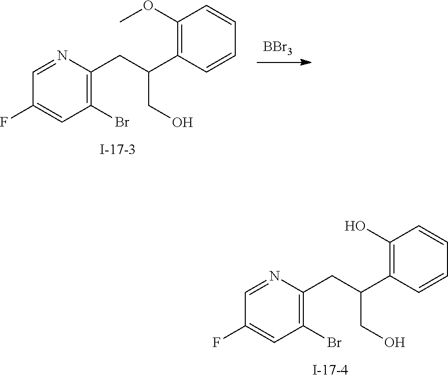

To a solution of 3-(3-bromo-5-fluoropyridin-2-yl)-2-(2-methoxyphenyl)propan-1-ol (6.7 g, 19.6 mmol) in dichloromethane (70 mL) was added boron tribromide (40 mL, 39.2 mmol) at 0° C. The reaction mixture was cooled to 0° C. and stirred at that temperature for 3 h. Upon the completion, sodium bicarbonate solution (100 mL) was added to adjust pH to 10 and then extracted with dichloromethane (50 mL×2), dried and concentrated. The resulting oil was purified by flash column chromatography with a gradient elution of petroleum ether (100%) to petroleum ether (70%) and ethyl acetate (30%) to provide 2-(1-(3-bromo-5-fluoropyridin-2-yl)-3-hydroxypropan-2-yl)phenol (4.25 g, yield: 64.5%) as a white solid. MS (ESI) m/z: 326 [M+H]⁺.

### Preparation of (3-fluoro-10,11-dihydrobenzo[6,7]oxepino[3,2-b]pyridin-10-yl)methanol

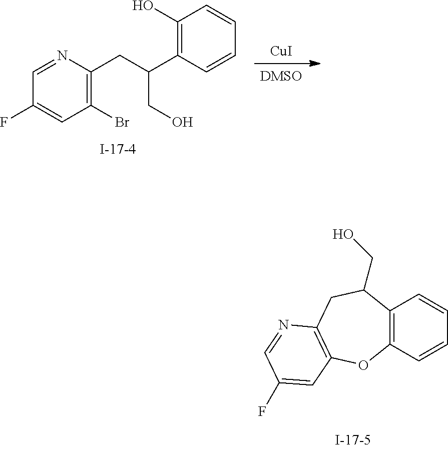

To a solution of 2-(1-(3-bromo-5-fluoropyridin-2-yl)-3-hydroxypropan-2-yl)phenol (2.11 g, 6.46 mmol) in dimethyl sulfoxide (5 mL) was added copper(I) iodide (124 mg, 646 μmol) and cesium carbonate (4.20 g, 12.9 mmol). The reaction mixture was heated to 80° C. and stirred at that temperature for 16 h. Upon the completion, ethyl acetate (50 mL) and water (50 mL) was added and the mixture was washed with brine (100 mL×2), dried and concentrated. The resulting oil was purified by flash column chromatography with a gradient elution of petroleum ether (100%) to petroleum ether (80%) and ethyl acetate (20%) to provide (3-fluoro-10,11-dihydrobenzo[6,7]oxepino[3,2-b]pyridin-10-yl)methanol (900 mg, yield: 56.9%) as a colorless oil. MS (ESI) m/z: 246 [M+H]⁺.

### Preparation of 2-((3-fluoro-10,11-dihydro-5H-benzo[4,5]cyclohepta[1,2-b]pyridin-10-yl)methyl)isoindoline-1,3-dione

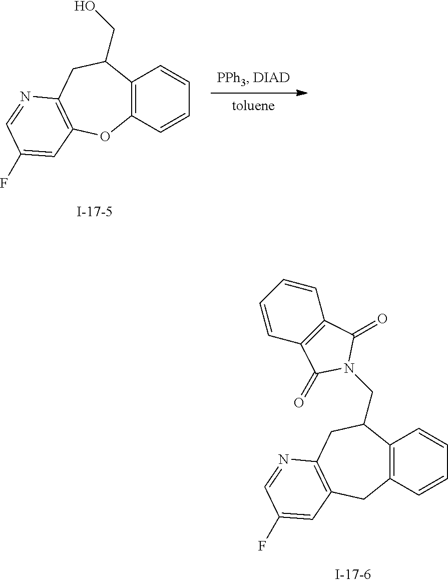

To a solution of (3-fluoro-10,11-dihydrobenzo[6,7]oxepino[3,2-b]pyridin-10-yl)methanol (0.9 g, 3.66 mmol), phthalimide (807 mg, 5.49 mmol) and triphenylphosphane (1.91 g, 7.32 mmol) in toluene (30 mL) was added diisopropyl azodicarboxylate (1.48 g, 7.32 mmol) at 0° C. The reaction was stirred at 0° C. for 2 h. Upon the completion, ethyl acetate (50 mL) was added and the mixture was washed with water (100 mL×2), dried and concentrated. The crude was dissolved with methanol (50 mL) and then filtered. The white solid was collected. MS (ESI) m/z: 375[M+H]⁺.

### Preparation of (3-fluoro-10,11-dihydrobenzo[6,7]oxepino[3,2-b]pyridin-10-yl)methanamine

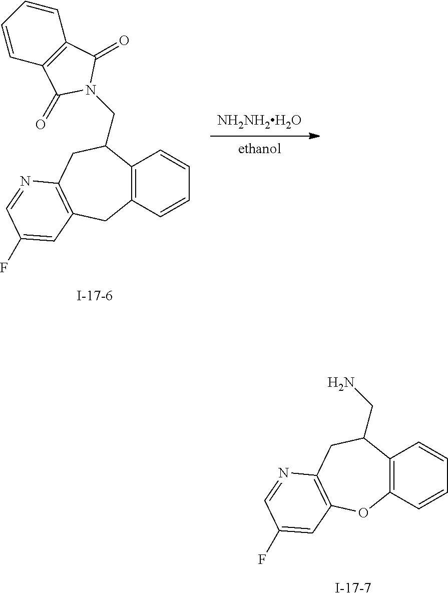 [SMILES: O=C1c2ccccc2C(=O)N1CC1Cc2ncc(F)cc2Cc2ccccc21 ; "NH2NH2·H2O" ; "ethanol" ; NCC1Cc2ncc(F)cc2Oc2ccccc21]

To a solution of 2-((3-fluoro-10,11-dihydro-5H-benzo[4,5]cyclohepta[1,2-b]pyridin-10-yl)methyl)isoindoline-1,3-dione (1.2 g, 3.20 mmol) in ethanol (50 mL) was added hydrazine hydrate (0.5 mL). The reaction mixture was heated to 90° C. and stirred at that temperature for 1 h. Upon the completion, the mixture was filtered and the filtrate was removed. The resulting residue was dissolved with dichloromethane (50 mL), washed with water (100 mL×3), then dried and concentrated. The crude was used for next step without further purification. MS (ESI) m/z: 245 [M+H]⁺.

### Preparation of tert-butyl ((3-fluoro-10,11-dihydrobenzo[6,7]oxepino[3,2-b]pyridin-10-yl)methyl)carbamate

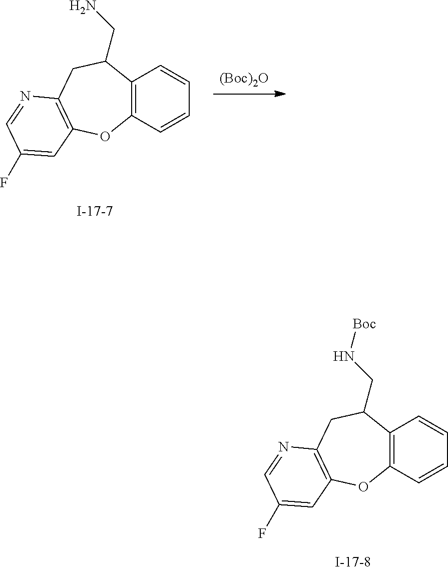 [SMILES: "(Boc)2O" ; NCC1Cc2ncc(F)cc2Oc2ccccc21 ; CC(C)(C)OC(=O)NCC1Cc2ncc(F)cc2Oc2ccccc21]

To a solution of (3-fluoro-10,11-dihydrobenzo[6,7]oxepino[3,2-b]pyridin-10-yl)methanamine (0.78 g, 3.19 mmol) in dichloromethane (50 mL) was added triethylamine (966 mg, 9.57 mmol) and di-tert-butyl dicarbonate (1.04 g, 4.78 mmol). The reaction was stirred at ambient temperature for 1 h. Upon the completion, the mixture was washed with water (50 mL×2), dried and concentrated. The resulting oil was purified by flash column chromatography with a gradient elution of petroleum ether (100%) to petroleum ether (90%) and ethyl acetate (10%) to provide tert-butyl ((3-fluoro-10,11-dihydrobenzo[6,7]oxepino[3,2-b]pyridin-10-yl)methyl)carbamate (900 mg, 2.61 mmol) as a white solid. Ms (ESI) m/z: 345 [M+H]⁺.

### Chiral separation of tert-butyl ((3-fluoro-10,11-dihydrobenzo[6,7]oxepino[3,2-b]pyridin-10-yl)methyl)carbamate

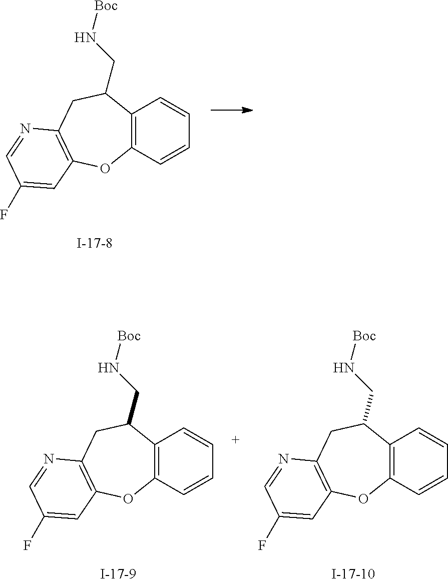 [SMILES: CC(C)(C)OC(=O)NCC1Cc2ncc(F)cc2Oc2ccccc21 ; CC(C)(C)OC(=O)NC[C@@H]1Cc2ncc(F)cc2Oc2ccccc21 ; CC(C)(C)OC(=O)NC[C@H]1Cc2ncc(F)cc2Oc2ccccc21]

The compound was purified by Chiral IPLC using the conditions listed below to get I-17-9 (410 mg, retention time: 0.627 min) and I-17-10 (374 mg, retention time: 0.740 min).
Instrument: SFC-150 (Thar, Waters)
Column: AD 20*250 mm, 10 μm (Daicel)
Column temperature: 35° C.
Mobile phase: CO2/methanol (0.2% Methanol Ammonia)=70/30
Flow rate: 100 g/min
Back pressure: 100 bar
Detection wavelength: 214 nm
Cycle time: 2.03 min
Sample solution: 900 mg dissolved in 45 mL Methanol
Injection volume: 2 mL

### Synthesis of (R*)-(3-fluoro-10,11-dihydrobenzo[6,7]oxepino[3,2-b]pyridin-10-yl)methanamine (Compound 39) and (S*)-(3-fluoro-10,11-dihydrobenzo-[6,7]oxepino[3,2-b]pyridin-10-yl)methanamine (Compound 40)

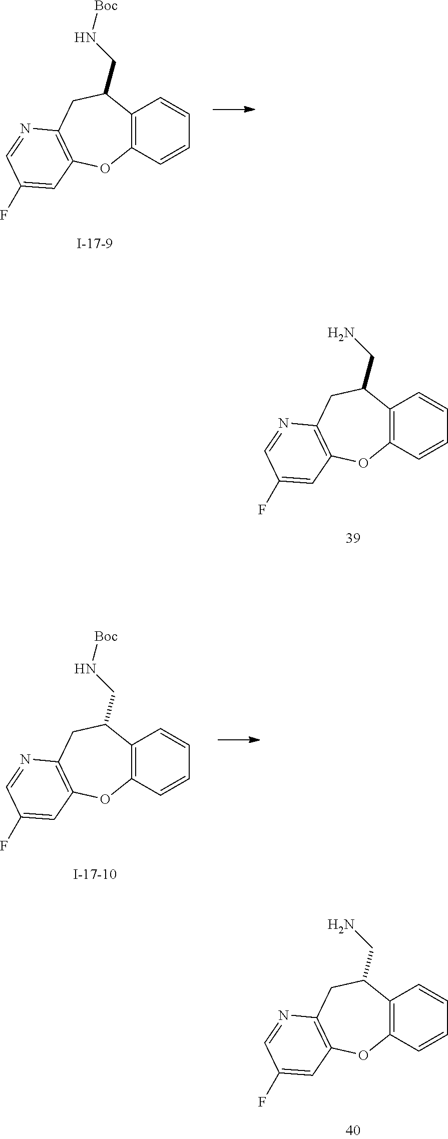 [SMILES: CC(C)(C)OC(=O)NC[C@@H]1Cc2ncc(F)cc2Oc2ccccc21 ; NC[C@@H]1Cc2ncc(F)cc2Oc2ccccc21 ; CC(C)(C)OC(=O)NC[C@H]1Cc2ncc(F)cc2Oc2ccccc21 ; NC[C@H]1Cc2ncc(F)cc2Oc2ccccc21]

A solution of I-17-9 (0.1 g, 290 μmol) in hydrogen chloride/methanol (3M, 20 mL) was stirred at ambient temperature for overnight. Upon the completion, the solvent was removed and the solid was dried by freeze dryer to provide 39. MS (ESI) m/z: 245[M+H]⁺. ¹H NMR (400 MHz, CD₃OD) δ 8.57 (d, J=2.4 Hz, 1H), 8.06 (dd, J=8.8, 2.4 Hz, 1H), 7.45-7.31 (m, 4H), 3.76-3.75 (m, 1H), 3.63 (dt, J=17.6, 2.4 Hz, 1H), 3.56-3.39 (m, 3H). Chiral analysis column: AY-H (250*4.6 mm 5 μm); Mobile Phase n-Hexane (0.11% DEA):ethanol (0.1% DEA)=90:10 Temperature: 40° C.; Flow: 1.0 mL/min. Retention time: 13.361 min.

A solution of I-17-10 (0.1 g, 290 μmol) in hydrogen chloride/ethyl acetate (3M, 20 mL) was stirred at ambient temperature for overnight. Upon the completion, the solvent was removed and the solid was dried by freeze dryer to provide 40. MS (ESI) m/z: 245 [M+H]⁺. ¹H NMR (400 MHz, CD₃OD) δ 8.60 (d, J=2.4 Hz, 1H), 8.10 (dd, J=8.8, 2.4 Hz, 1H), 7.45-7.31 (m, 4H), 3.78-3.73 (m, 1H), 3.64 (dt, J=17.6, 2.4 Hz, 1H), 3.56-3.40 (m, 3H). Chiral analysis column: AY-H (250*4.6 mm 5 μm); Mobile Phase n-Hexane (0.11% DEA):ethanol (0.1% DEA)=90:10 Temperature: 40° C.; Flow: 1.0 mL/min. Retention time: 12.325 min.

### Example 78. Synthesis of (R*)-(3-fluoro-10,11-dihydrobenzo[6,7]oxepino[3,2-b]pyridin-10-yl)-N-methylmethanamine (Compound 37) and (S*)-(3-fluoro-10,11-dihydrobenzo[6,7]oxepino[3,2-b]pyridin-10-yl)-N-methylmethanamine (Compound 38)

Compound 37 and Compound 38 are prepared using a similar procedure described in Example 63.

Compound 37. MS (ESI) m/z: 259 [M+H]⁺. ¹H NMR (400 MHz, CD₃OD) δ 8.60 (d, J=2.4 Hz, 1H), 8.10 (dd, J=8.8, 2.4 Hz, 1H), 7.48-7.32 (m, 4H), 3.83-3.82 (m, 1H), 3.68-3.60 (m, 2H), 3.55-3.47 (m, 2H), 2.75 (s, 3H). Chiral analysis column: AD-H (250*4.6 mm 5 μm); Mobile Phase n-Hexane (0.11% DEA):ethanol (0.1% DEA)=90:10 Temperature: 40° C.; Flow: 1.0 mL/min. Retention time: 9.167 min.

Compound 38. MS (ESI) m/z: 259 [M+H]⁺. ¹H NMR (400 MHz, CD₃OD) δ 8.54-8.53 (m, 1H), 8.02-7.99 (m, 1H), 7.45-7.31 (m, 4H), 3.83-3.82 (m, 1H), 3.65-3.60 (m, 2H), 3.50-3.49 (m, 2H), 2.74 (s, 3H). Chiral analysis column: AD-H (250*4.6 mm 5 μm); Mobile Phase n-Hexane (0.11% DEA):ethanol (0.1% DEA)=90:10 Temperature: 40° C.; Flow: 1.0 mL/min, retention time: 13.350 min.

### Example 79. Synthesis of (R*)-(4-fluoro-5,6-dihydrobenzo[6,7]oxepino[2,3-c]pyridin-6-yl)methanamine (Compound 153) and (S*)-(4-fluoro-5,6-dihydrobenzo[6,7]oxepino[2,3-c]pyridin-6-yl)methanamine (Compound 154)

### Preparation of methyl 4-fluorobenzo[6,7]oxepino[2,3-c]pyridine-6-carboxylate

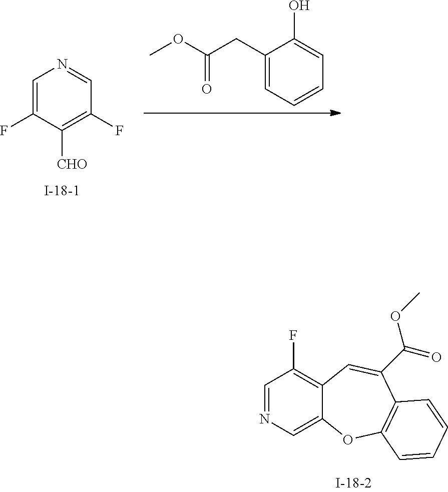 [SMILES: O=Cc1c(F)cncc1F ; COC(=O)Cc1ccccc1O ; COC(=O)C1=Cc2c(F)cncc2Oc2ccccc21]

To a solution of methyl 2-(2-hydroxyphenyl)acetate (1.3 g, 7 mmol) in dimethyl sulfoxide (10 mL) was added copper(I) iodide (150 mg, 0.8 mmol), cesium carbonate (5.2 g, 18 mmol) and 3,5-3,5-difluoroisonicotinaldehyde (1.0 g, 7 mmol) under nitrogen. The reaction mixture was heated to 100° C. and stirred at that temperature for 2 h. Upon the completion, ethyl acetate (90 mL) and water (150 mL) were added to the reaction vessel and the resulting biphasic mixture was transferred to a separatory funnel. The layers were separated and the organic phase was washed with brine (2×100 mL). The combined organics were dried over anhydrous sodium sulphate, filtered and concentrated in vacuo. The resulting solid was purified by flash column chromatography with a gradient elution of petroleum ether (100%) to petroleum ether (70%) and ethyl acetate (30%) to provide methyl 4-fluorobenzo[6,7]oxepino[2,3-c]pyridine-6-carboxylate (0.65 g, yield: 30%) as a white solid. (ESI) m/z: 272[M+H]⁺.

### Preparation of methyl 4-fluoro-5,6-dihydrobenzo[6,7]oxepino[2,3-c]pyridine-6-carboxylate

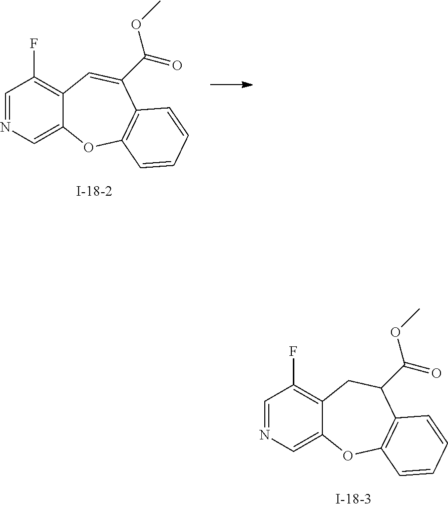 [SMILES: COC(=O)C1=Cc2c(F)cncc2Oc2ccccc21 ; COC(=O)C1Cc2c(F)cncc2Oc2ccccc21]

To a solution of methyl 4-fluorobenzo[6,7]oxepino[2,3-c]pyridine-6-carboxylate (0.65 g, 2.5 mmol) in methanol (20 mL) was added sodium borohydride (0.3 g, 8 mmol) and nickel chloride (0.35 g, 2.5 mmol). The reaction was stirred at ambient temperature for 1 h. Ice water (10 mL) was added to quench the reaction. Then ethyl acetate (60 mL) was added to the mixture and the resulting biphasic mixture was transferred to a separatory funnel. The layers were separated and the organic phase was washed with brine (50 mL). The combined organics were dried over anhydrous sodium sulphate, filtered and concentrated in vacuo. The resulting oil was purified by flash column chromatography with a gradient elution of petroleum ether (100%) to petroleum ether (70%) and ethyl acetate (30%) to provide methyl 4-fluoro-5,6-dihydrobenzo[6,7]oxepino[2,3-c]pyridine-6-carboxylate (0.45 g, yield: 68%) as a white solid. (ESI) m/z: 274[M+H]⁺.

### Preparation of (4-fluoro-5,6-dihydrobenzo[6,7]oxepino[2,3-c]pyridin-6-yl)methanol

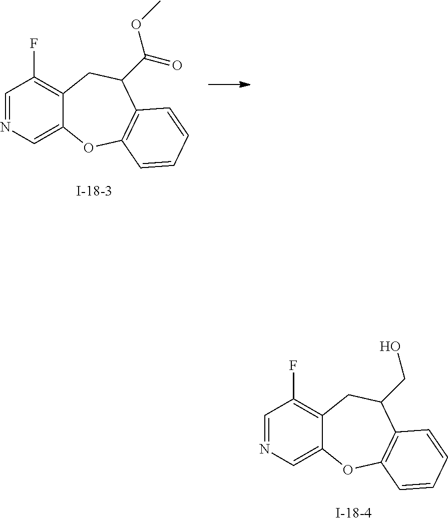 [SMILES: COC(=O)C1Cc2c(F)cncc2Oc2ccccc21 ; OCC1Cc2c(F)cncc2Oc2ccccc21]

To a solution of methyl 4-fluoro-5,6-dihydrobenzo[6,7]oxepino[2,3-c]pyridine-6-carboxylate (0.45 g, 3 mmol) in tetrahydrofuran (15 mL) was added lithium aluminum hydride (330 mg, 6 mmol). The reaction was stirred at 0° C. for 1 h. Ice water (10 mL) was added to the reaction vessel. Then ethyl acetate (30 mL) and water (30 mL) were added to the mixture. The layers were separated and the org phase was washed with brine (2×15 mL). The combined organics were dried over anhydrous sodium sulphate, filtered and concentrated in vacuo. The resulting oil was purified by flash column chromatography with a gradient elution of petroleum ether (100%) to petroleum ether (15%) and ethyl acetate (85%) to provide ((4-fluoro-5,6-dihydrobenzo[6,7]oxepino[2,3-c]pyridin-6-yl)methanol (0.25 g, yield: 62%) as a yellow oil. (ESI) m/z: 246[M+H]⁺.

### Preparation of 2-((4-fluoro-5,6-dihydrobenzo[6,7]oxepino[2,3-c]pyridin-6-yl)methyl)isoindoline-1,3-dione

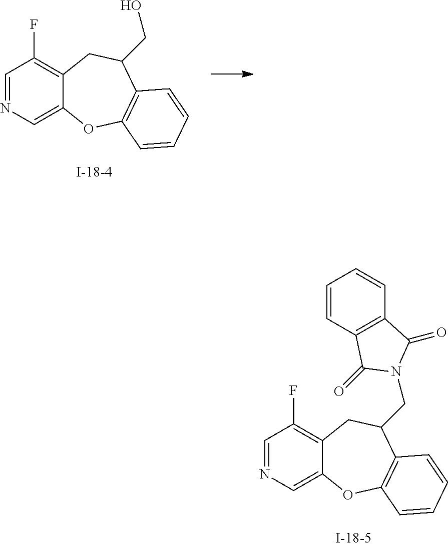 [SMILES: OCC1Cc2c(F)cncc2Oc2ccccc21 ; O=C1c2ccccc2C(=O)N1CC1Cc2c(F)cncc2Oc2ccccc21]

To a solution of (4-fluoro-5,6-dihydrobenzo[6,7]oxepino[2,3-c]pyridin-6-yl)methanol (0.25 g, 1.0 mmol), 2,3-dihydro-1H-isoindole-1,3-dione (0.3 g, 1.6 mmol) and triphenylphosphane (3.3 g, 2.1 mmol) in toluene (20 mL) was added diisopropyl azodicarboxylate (0.65 g, 3.2 mmol) under nitrogen in ice bath. The mixture was stirred at 0° C. for 2 h. Upon the completion, ethyl acetate (50 mL) and water (80 mL) were added to the reaction vessel and the resulting biphasic mixture was transferred to a separatory funnel. The layers were separated and the organic phase was washed with water (2×15 mL). The combined organics were dried over anhydrous sodium sulphate, filtered and concentrated in vacuo. The resulting mixture was used for next step without purification. (ESI) m/z: 375[M+H]⁺.

### Preparation of (4-fluoro-5,6-dihydrobenzo[6,7]oxepino[2,3-c]pyridin-6-yl)methanamine

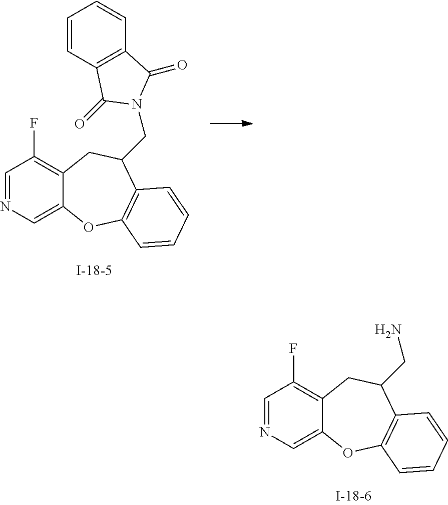 [SMILES: O=C1c2ccccc2C(=O)N1CC1Cc2c(F)cncc2Oc2ccccc21 ; NCC1Cc2c(F)cncc2Oc2ccccc21]

To a solution of 2-((4-fluoro-5,6-dihydrobenzo[6,7]oxepino[2,3-c]pyridin-6-yl)methyl)isoindoline-1,3-dione (crude, 1.0 mmol) in ethanol (30 mL) was added hydrazine hydrate (85% aq. 1.29 g, 25.8 mmol). The mixture was heated to 80° C. with stirring for 2 h. Upon the completion, the reaction was cooled to ambient temperature, filtered to remove the solid and the filtrate was concentrated in vacuo to get the resulting mixture used for next step without purification. (ESI) m/z: 245[M+H]⁺.

### Preparation of tert-butyl ((4-fluoro-5,6-dihydrobenzo[6,7]oxepino[2,3-c]pyridin-6-yl)methyl)carbamate

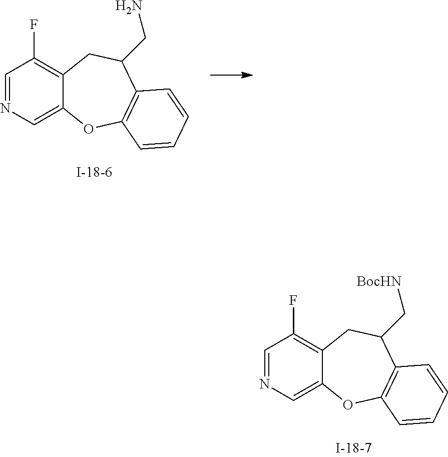 [SMILES: NCC1Cc2c(F)cncc2Oc2ccccc21 ; CC(C)(C)OC(=O)NCC1Cc2c(F)cncc2Oc2ccccc21]

To a solution of (4-fluoro-5,6-dihydrobenzo[6,7]oxepino[2,3-c]pyridin-6-yl)methanamine (crude, 1.0 mmol) in dichloromethane (50 mL) was added triethylamine (9.50 g, 93.9 mmol) and di-tert-butyl dicarbonate (7.15 g, 32.8 mmol). The reaction was stirred at ambient temperature for overnight. Water (20 mL) was added to the reaction vessel and the resulting biphasic mixture was transferred to a separatory funnel. The layers were separated and the organic phase was washed with saturated aqueous brine (2×100 mL). The combined organics were dried over anhydrous sodium sulphate, filtered and concentrated in vacuo. The resulting oil was purified by flash column chromatography with a gradient elution of petroleum ether (100%) to petroleum ether (80%) and ethyl acetate (20%) to provide tert-butyl ((4-fluoro-5,6-dihydrobenzo[6,7]oxepino[2,3-c]pyridin-6-yl)methyl)carbamate (0.16 g, yield: 46%) as a white solid. (ESI) m/z: 345[M+H]⁺.

### Chiral separation of tert-butyl ((4-fluoro-5,6-dihydrobenzo[6,7]oxepino[2,3-c]pyridin-6-yl)methyl)carbamate

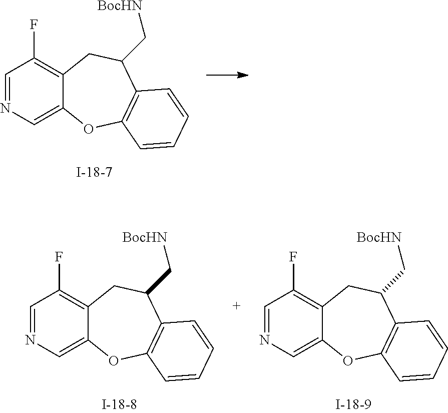 [SMILES: CC(C)(C)OC(=O)NCC1Cc2c(F)cncc2Oc2ccccc21 ; CC(C)(C)OC(=O)NC[C@@H]1Cc2c(F)cncc2Oc2ccccc21 ; CC(C)(C)OC(=O)NC[C@H]1Cc2c(F)cncc2Oc2ccccc21]

Tert-butyl ((4-fluoro-5,6-dihydrobenzo[6,7]oxepino[2,3-c]pyridin-6-yl)methyl)carbamate (0.16 g, 0.5 mmol) was purified by chiral separation using Instrument: SFC-80 (Waters), Column: AD 20*250 mm, 10 μm (Daicel), Column temperature: 35° C., Mobile phase: CO2/IPA (0.2% Methanol Ammonia)=80/20, Flow rate: 110 g/min, Back pressure: 100 bar, Detection wavelength: 214 nm, Cycle time: 4.5 min, Sample solution: 160 mg dissolved in 40 mL Methanol and Injection volume: 1 mL to provide I-18-8 (50 mg, retention time=1.2 min) as a white solid and I-18-9 (50 mg, retention time=1.4 min) as a white solid.

### Synthesis of (R*)-(4-fluoro-5,6-dihydrobenzo[6,7]oxepino[2,3-c]pyridin-6-yl)methanamine (Compound 153) and (S*)-(4-fluoro-5,6-dihydrobenzo[6,7]oxepino[2,3-c]pyridin-6-yl)methanamine (Compound 154)

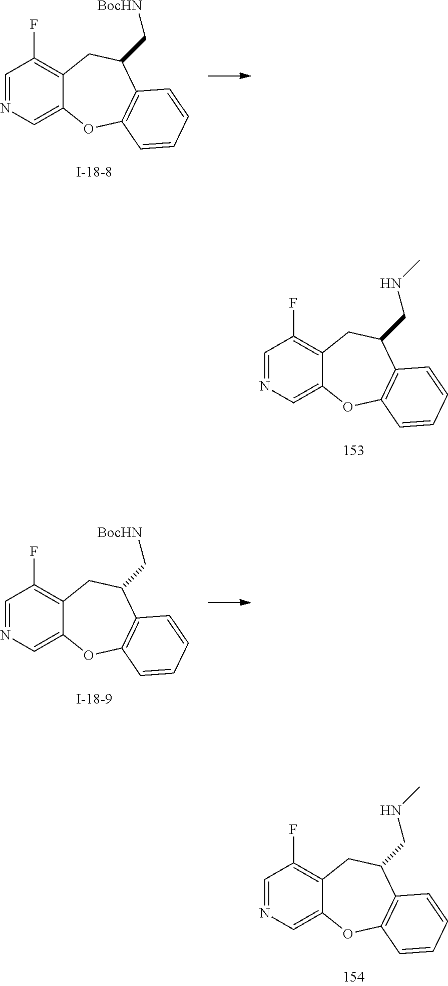 [SMILES: CC(C)(C)OC(=O)NC[C@@H]1Cc2c(F)cncc2Oc2ccccc21 ; CNC[C@@H]1Cc2c(F)cncc2Oc2ccccc21 ; CC(C)(C)OC(=O)NC[C@H]1Cc2c(F)cncc2Oc2ccccc21 ; CNC[C@H]1Cc2c(F)cncc2Oc2ccccc21]

A solution of compound I-18-1 (30 mg, 0.1 mmol) in 3M hydrogen chloride/methanol (5 mL) was stirred at ambient temperature for 2 h. Upon the completion, the mixture was evaporated in vacuo to dryness and then freeze-dried to yield Compound 153. MS (ESI) m/z: 245 [M+H]⁺. ¹H NMR (500 MHz, CD₃OD) δ 8.62-8.40 (m, 2H), 7.43-7.32 (m, 4H), 3.75-3.33 (m, 5H). Chiral analysis column: IG (100*4.6 mm, 5 μm), Co-Solvent: methanol [0.2% NH3 (7M in methanol)], Temperature: 40° C., Flow: 3.0 mL/min, Inject Volume: 5 μl, Back Pressure: 2000 psi, Run Time: 3.5 Minutes, Retention time=1.32 min.

A solution of compound I-18-9 (20 mg, 0.09 mmol) in 3M hydrogen chloride/methanol (5 mL) was stirred at ambient temperature for 2 h. Upon the completion, the mixture was evaporated in vacuo to dryness and then freeze-dried to yield Compound 154. MS (ESI) m/z: 245 [M+H]⁺. ¹H NMR (500 MHz, CD₃OD) δ 8.62-8.40 (m, 2H), 7.43-7.32 (m, 4H), 3.75-3.33 (m, 5H). Chiral analysis column: IG (100*4.6 mm, 5 μm), Co-Solvent: methanol [0.2% NH3 (7M in methanol)], Temperature: 40° C., Flow: 3.5 mL/min, Inject Volume: 5 μl, Back Pressure: 2000 psi, Run Time: 3.5 Minutes, Retention time=1.51 min.

### Example 80. Synthesis of (S*)-(10,11-dihydrobenzo[6,7]oxepino[3,2-b]pyridin-11-yl)methanamine (Compound 7) and (R*)-(10,11-dihydrobenzo[6,7]oxepino[3,2-b]pyridin-11-yl)methanamine (Compound 8)

### Preparation of methyl benzo[6,7]oxepino[3,2-b]pyridine-11-carboxylate

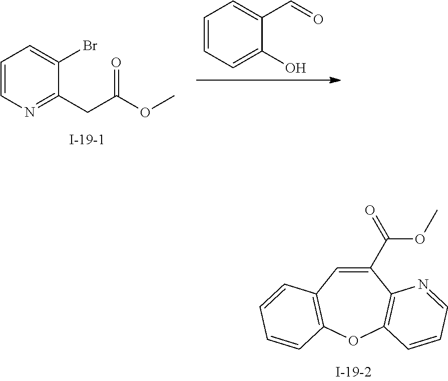 [SMILES: COC(=O)Cc1ncccc1Br ; O=Cc1ccccc1O ; COC(=O)C1=Cc2ccccc2Oc2cccnc21]

To a solution of methyl 2-(3-bromopyridin-2-yl)acetate (5.3 g, 23.0 mmol) in DMSO (50 mL) was added 2-hydroxybenzaldehyde (4.21 g, 34.5 mmol), CuI (438 mg, 2.30 mmol) and K₂CO₃ (6.34 g, 46.0 mmol). The reaction mixture was heated to 120° C. and stirred at that temperature for 2 h under N₂ atmosphere. Upon the completion, water and ethyl acetate was added and the organic phases was washed with brine (200 mL×3), dried and concentrated. The resulting oil was purified by silica gel column chromatography with a gradient elution of petroleum ether (100%) to petroleum ether (87%) and ethyl acetate (13%) to provide methyl benzo[6,7]oxepino[3,2-b]pyridine-11-carboxylate (2.1 g, yield: 36%) as a white solid. MS (ESI):: m/z=254 [M+H]⁺.

### Preparation of methyl 10, 11-dihydrobenzo[6,7]oxepino[3,2-b]pyridine-11-carboxylate

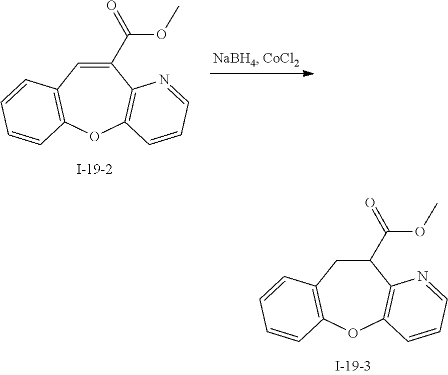 [SMILES: COC(=O)C1=Cc2ccccc2Oc2cccnc21 ; COC(=O)C1Cc2ccccc2Oc2cccnc21]

To a solution of methyl benzo[6,7]oxepino[3,2-b]pyridine-11-carboxylate (2.1 g, 8.29 mmol) in ethanol (300 mL) was added cobalt chloride hexahydrate (1.97 g, 8.29 mmol) and NaBH₄ (3.14 g, 82.8 mmol) at 0° C. The reaction was stirred at ambient temperature for 4 h. Upon the completion, water and DCM was added and the mixtures was extracted with DCM (100 mL×3), dried and concentrated. The crude product (1.72 g) was used for next step without further purification. MS (ESI): m/z=255 [M+H]⁺.

### Preparation of (10, 11 dihydrobenzo [6, 7] oxepino[3, 2-b] pyridin-11-yl) methanol

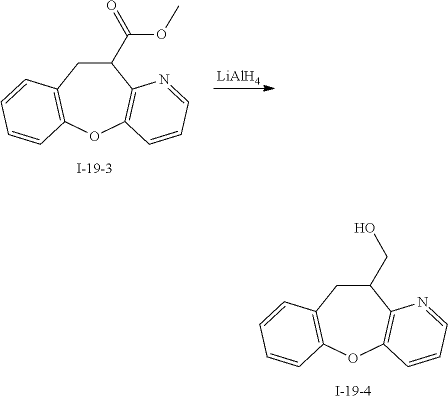

To a solution of methyl 10, 11-dihydrobenzo[6,7]oxepino[3,2-b]pyridine-11-carboxylate (1.72 g, 6.73 mmol) in THE (40 mL) was added LiAlH₄ (508 mg, 13.4 mmol) at 0° C. The reaction was stirred at ambient temperature for 2 h. Upon the completion, water (3 g) and ethyl acetate (50 mL) was added and then filtered. The filtrate was concentrated. The resulting oil was purified by silica gel column chromatography with a gradient elution of petroleum ether (100%) to petroleum ether (80%) and ethyl acetate (20%) to provide (10, 11-dihydrobenzo [6, 7] oxepino [3, 2-b] pyridin-11-yl) methanol (0.9 g, yield: 47.8% of 2 steps) as a yellow oil. MS (ESI): m/z=228 [M+H]⁺

### Preparation of 2-((10,11-dihydrobenzo[6,7]oxepino[3,2-b]pyridin-11-yl)methyl)-isoindoline-1,3-dione

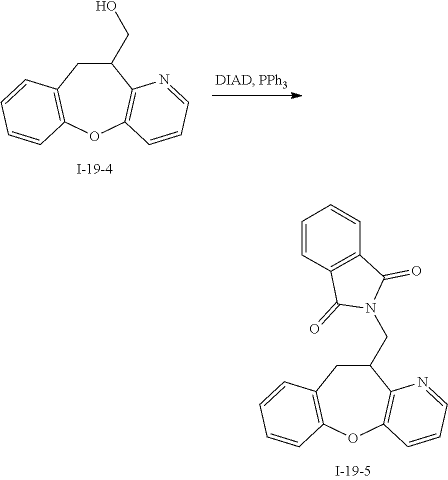 [SMILES: OCC1Cc2ccccc2Oc2cccnc21 ; O=C1c2ccccc2C(=O)N1CC1Cc2ccccc2Oc2cccnc21]

To a solution of (10, 11-dihydrobenzo [6, 7] oxepino [3, 2-b] pyridin-11-yl) methanol (0.8 g, 3.52 mmol), phthalimide (776 mg, 5.28 mmol) and triphenylphosphine (1.84 g, 7.04 mmol) in toluene (20 mL) was added diisopropyl azodicarboxylate (1.42 g, 7.04 mmol) at 0° C. The mixture was stirred at 0° C. for 2 h. Upon the completion, water (20 mL) and ethyl acetate (20 mL) were added, and the organic phase was dried and concentrated. The crude product was used for next step without further purification. MS (ESI): m/z=357 [M+H]⁺.

### Preparation of tert-butyl ((10,11-dihydrobenzo[6,7]oxepino[3,2-b]pyridin-11-yl)methyl)carbamate

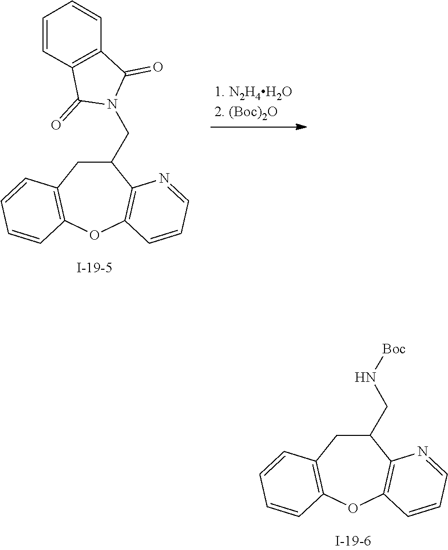 [SMILES: O=C1c2ccccc2C(=O)N1CC1Cc2ccccc2Oc2cccnc21 ; CC(C)(C)OC(=O)NCC1Cc2ccccc2Oc2cccnc21]

To a solution of 2-((10,11-dihydrobenzo[6,7]oxepino[3,2-b]pyridin-11-yl)methyl)isoindoline-1,3-dione (1.4 g, 3.92 mmol) in ethanol (50 mL) was added hydrazine hydrate (1.95 g, 39.1 mmol). The reaction mixture was heated to 100° C. and stirred at that temperature for 2 h. the mixture was filtered and the filtrate was concentrated. The residue was dissolved in DCM (20 mL), di-tert-butyl dicarbonate (693 mg, 3.18 mmol) and triethylamine (802 mg, 7.95 mmol) were added. The reaction was stirred at ambient temperature for 2 h. Water (50 mL) and DCM (50 mL) were added, and then the organic phase were dried and concentrated. The resulting oil was purified by silica gel column chromatography with a gradient elution of petroleum ether (100%) to petroleum ether (85%) and ethyl acetate (15%) to provide tert-butyl ((10,11-dihydrobenzo[6,7]oxepino[3,2-b]pyridin-11-yl)methyl)carbamate (720 mg, yield: 55.8% of 3 steps) as a colorless oil. MS (ESI): m/z=327 [M+H]⁺.

### Preparation of (10,11-dihydrobenzo[6,7]oxepino[3,2-b]pyridin-11-yl)methanamine

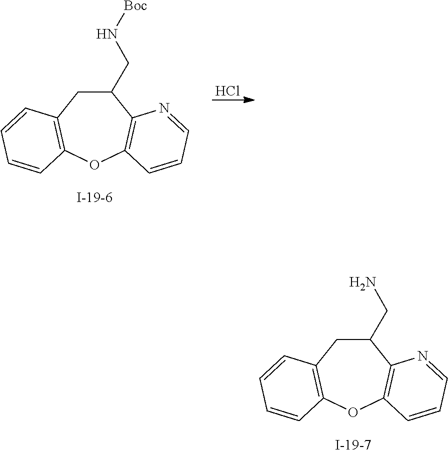 [SMILES: CC(C)(C)OC(=O)NCC1Cc2ccccc2Oc2cccnc21 ; Cl ; NCC1Cc2ccccc2Oc2cccnc21]

A solution of tert-butyl ((10,11-dihydrobenzo[6,7]oxepino[3,2-b]pyridin-11-yl)methyl)carbamate (0.76 g, 2.32 mmol) in 3 M HCl/methanol (20 mL) was stirred at ambient temperature for 16 h. Upon the completion, the solvent was removed and the residue was neutralized with 1 M NaOH aqueous solution, and then extracted with DCM (50 mL), the organic phase was dried by Na₂SO₄ and evaporated in vacuo to get (10,11-dihydrobenzo[6,7]oxepino[3,2-b]pyridin-11-yl)methanamine. MS (ESI): m/z=227 [M+H]⁺.

### Chiral separation of (10,11-dihydrobenzo[6,7]oxepino[3,2-b]pyridin-11-yl)methanamine

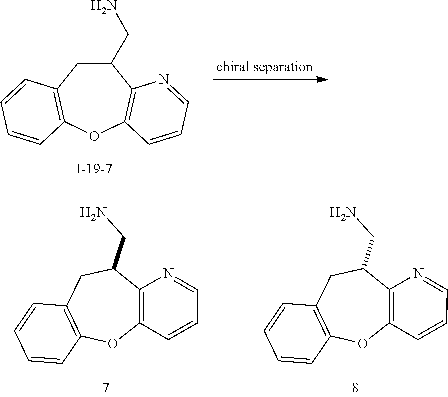 [SMILES: NCC1Cc2ccccc2Oc2cccnc21 ; NC[C@@H]1Cc2ccccc2Oc2cccnc21 ; NC[C@H]1Cc2ccccc2Oc2cccnc21]

400 mg of (10,11-dihydrobenzo[6,7]oxepino[3,2-b]pyridin-11-yl)methanamine was separated by below conditions:
Instrument: Gilson-281 Column: AY 20*250, 10 μm
Mobile Phase: n-Hexane (0.1% DEA):EtOH (0.1% DEA)=80:20
Flow Rate: 35 mL/min
Run time per injection: 14 min
Injection: 0.8 mL
Sample solution: 400 mg in 36 mL MeOH
   Compound 7 (160 mg, retention time=10.04 min) and Compound 8 (172 mg, retention time=13.00 min) were obtained in their freebase form.

### Synthesis of (S*)-(10,11-dihydrobenzo[6,7]oxepino[3,2-b]pyridin-11-yl)methanamine (Compound 7) and (R*)-(10,11-dihydrobenzo[6,7]oxepino[3,2-b]pyridin-11-yl)-methanamine (Compound 8)

Compound 7 (freebase form) was converted to its HCl salt using 3 M HCl/methanol. MS (ESI): m/z=227 [M+H]⁺. ¹H NMR (400 MHz, CD₃OD) δ 8.54 (m, 1H), 8.17-8.15 (m, 1H), 7.76 (m, 1H), 7.41-7.24 (m, 4H), 3.99-3.94 (m, 1H), 3.55-3.49 (m, 1H), 3.39-3.34 (m, 1H), 3.28-3.24 (m, 2H). Chiral analysis column: AY-H (250*4.6 mm 5 μm); mobile phase: n-Hexane (0.1% DEA):EtOH (0.1% DEA)=90:10; temperature: 40° C.; flow: 1.0 mL/min; retention time: 10.027 min.

Compound 8 (freebase form) was converted to its HCl salt using 3 M HCl/methanol. MS (ESI): m/z=227 [M+H]⁺. ¹H NMR (400 MHz, CD3OD) δ 8.65-8.39 (m, 1H), 8.36-7.97 (m, 1H), 7.92-7.53 (m, 1H), 7.48-7.12 (m, 4H), 3.99-3.94 (s, 1H), 3.55-3.49 (m, 1H), 3.39-3.32 (m, 1H), 3.29-3.15 (m, 2H). Chiral analysis column: AY-H (250*4.6 mm 5 μm); mobile phase: n-Hexane (0.1% DEA):EtOH (0.1% DEA)=90:10; temperature: 40° C.; flow:1.0 mL/min; retention time: 13.023 min.

### Example 81. Synthesis of (S*)-1-(10,11-dihydrobenzo[6,7]oxepino[3,2-b]pyridin-11-yl)-N-methylmethanamine (Compound 5) and (R*)-1-(10,11-dihydrobenzo-[6,7]oxepino[3,2-b]pyridin-11-yl)-N-methylmethanamine (Compound 6)

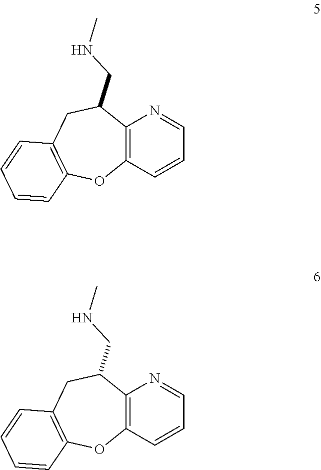 [SMILES: CNC[C@@H]1Cc2ccccc2Oc2cccnc21 ; CNC[C@H]1Cc2ccccc2Oc2cccnc21]

Compound 5 and Compound 6 were prepared using a similar procedure described in Example 63.

### Example 82. Synthesis of (S*)-1-(10,11-dihydrobenzo[6,7]oxepino[3,2-b]pyridin-11-yl)-N-ethylmethanamine (Compound 108) and (R*)-1-(10,11-dihydrobenzo-[6,7]oxepino[3,2-b]pyridin-11-yl)-N-ethylmethanamine (Compound 109)

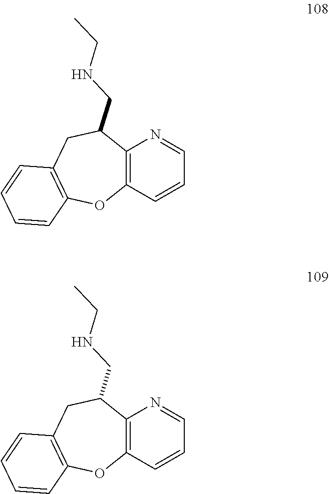 [SMILES: CCNC[C@@H]1Cc2ccccc2Oc2cccnc21 ; CCNC[C@H]1Cc2ccccc2Oc2cccnc21]

Compound 108 and Compound 109 were prepared using a similar procedure described in Example 63.

Compound 108. MS (ESI): m/z=255[M+H]⁺. ¹H NMR (400 MHz, CD₃OD) δ 8.56 (dd, J=5.2, 1.2 Hz, 1H), 8.20 (dd, J=8.4, 1.2 Hz, 1H), 7.80 (dd, J=8.4, 5.2 Hz, 1H), 7.46-7.44 (m, 1H), 7.37-7.31 (m, 2H), 7.26-7.19 (m, 1H), 4.11-4.06 (m, 1H), 3.55 (dd, J=15.1, 3.2 Hz, 1H), 3.45-3.40 (m, 1H), 3.36-3.32 (m, 2H), 3.20-3.15 (m, 2H), 1.38 (t, J=7.2 Hz, 3H). Chiral analysis column: IA (250*4.6 mm 5 μm); Mobile Phase: n-Hexane (0.1% DEA): IPA (0.1% DEA)=95:5; Temperature: 40° C.; Flow: 1.0 mL/min. Retention time: 5.870 min.

Compound 109. MS (ESI): m/z=255[M+H]⁺. ¹H NMR (400 MHz, CD₃OD) δ 8.55-8.53 (m, 1H), 8.17-8.15 (m, 1H), 7.77-7.74 (m, 1H), 7.44-7.22 (m, 4H), 4.06-4.04 (m, 1H), 3.55-3.50 (m, 1H), 3.45-3.40 (m, 1H), 3.37-3.27 (m, 2H), 3.21-3.15 (m, 2H), 1.38 (t, J=7.2 Hz, 3H). Chiral analysis: column: IA (250*4.6 mm 5 μm); Mobile Phase: n-Hexane (0.1% DEA): IPA (0.1% DEA)=95:5; Temperature: 40° C.; Flow: 1.0 mL/min. Retention time: 6.288 min.

### Example 83. Synthesis of (S*)-1-(10,11-dihydrobenzo[6,7]oxepino[3,2-b]pyridin-11-yl)-N,N-dimethylmethanamine (Compound 110) and (R*)-1-(10,11-dihydro-benzo[6,7]oxepino[3,2-b]pyridin-11-yl)-N,N-dimethylmethanamine (Compound 111)

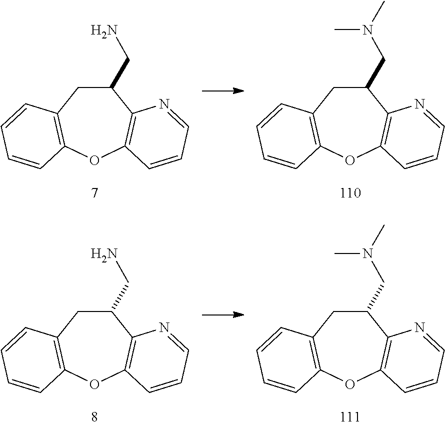 [SMILES: NC[C@@H]1Cc2ccccc2Oc2cccnc21 ; CN(C)C[C@@H]1Cc2ccccc2Oc2cccnc21 ; NC[C@H]1Cc2ccccc2Oc2cccnc21 ; CN(C)C[C@H]1Cc2ccccc2Oc2cccnc21]

To a solution of Compound 7 (1.10 mmol) in methanol (20 mL) was added formaldehyde (164 mg, 5.50 mmol) and sodium triacetoxyborohydride (699 mg, 3.30 mmol). The reaction was stirred at ambient temperature for 1 h. Upon the completion, the solvent was removed and dichloromethane (50 mL) was added, the organic phase was washed with sodium bicarbonate solution (50 mL), then dried and concentrated. The crude was purified by pre-HPLC to get 200 mg as colorless oil. The oil was dissolved in 4M hydrogen chloride/methanol (2 mL, 8 mmol). The solution was stirred at ambient temperature for 30 min. Upon the completion, the solvent was removed and the solid was dried by freeze dryer to give Compound 110. MS (ESI): m/z=255 [M+H]⁺. ¹H NMR (400 MHz, CD₃OD) δ 8.52 (dd, J=4.8, 1.2 Hz, 1H), 8.09 (dd, J=8.8, 1.6 Hz, 1H), 7.70 (dd, J=8.8, 5.2 Hz, 1H), 7.45 (d, J=7.6 Hz, 0.8 Hz, 1H), 7.36-7.29 (m, 2H), 7.25-7.21 (m, 1H), 4.10-4.05 (m, 1H), 3.65 (dd, J=13.6, 9.2 Hz, 1H), 3.46-3.39 (m, 2H), 3.32-3.29 (m, 1H), 3.02 (d, J=4 Hz, 6H). Chiral analysis: column: IG (250*4.6 mm 5 μm); mobile phase: n-Hexane (0.1% DEA): IPA (0.1% DEA)=100:2; temperature: 40° C.; flow: 1.0 mL/min, retention time: 6.477 min.

To a solution of Compound 8 (353 μmol) in methanol (15 mL) was added formaldehyde (52.8 mg, 1.76 mmol) and sodium triacetoxyborohydride (222 mg, 1.05 mmol). The reaction was stirred at ambient temperature for 1 h. Upon the completion, the solvent was removed and dichloromethane (50 mL) was added, the organic phase was washed with sodium bicarbonate solution (50 mL), then dried and concentrated. The crude was purified by pre-HPLC to get 60 mg of colorless oil. The oil was dissolved in 4M hydrogen chloride/methanol (2 mL, 8 mmol). The solution was stirred at ambient temperature for 30 min. Upon the completion, the solvent was removed and the solid was dried by freeze dryer Compound 111. MS (ESI): m/z=255 [M+H]⁺. ¹H NMR (400 MHz, CD₃OD) δ 8.46 (d, J=4.8 Hz, 1H), 7.94 (d, J=8.5 Hz, 1H), 7.56 (dd, J=8, 4.8 Hz, 1H), 7.41 (d, J=7.6 Hz, 1H), 7.34-7.23 (m, 2H), 7.21 (td, J=7.2, 1.6 Hz, 1H), 4.01-3.95 (m, 1H), 3.64 (dd, J=13.2, 9.6 Hz, 1H), 3.40 (dd, J=13.2, 4.8 Hz, 1H), 3.37-3.23 (m, 2H), 3.03 (d, J=12.8 Hz, 6H). Chiral analysis: column: IG (250*4.6 mm 5 μm); mobile phase: n-Hexane (0.1% DEA): IPA (0.1% DEA)=100:2; temperature: 40° C.; flow: 1.0 mL/min, retention time: 7.875 min.

### Example 84. Synthesis of (S*)-(8-chloro-10,11-dihydrobenzo[6,7]oxepino[3,2-b]pyridin-11-yl)methanamine (Compound 120) and (R*)-(8-chloro-10,11-dihydrobenzo[6,7]oxepino[3,2-b]pyridin-11-yl)methanamine (Compound 121)

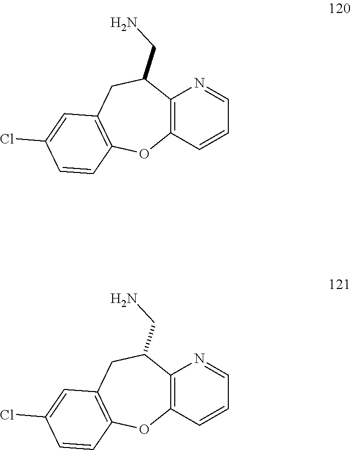 [SMILES: NC[C@@H]1Cc2cc(Cl)ccc2Oc2cccnc21 ; NC[C@H]1Cc2cc(Cl)ccc2Oc2cccnc21]

Compound 120 and Compound 121 were prepared using a similar procedure in Example 80.

Compound 120. (ESI): m/z=261[M+H]⁺. ¹H NMR (400 MHz, CD₃OD) δ 8.48 (d, J=4 Hz, 1H), 7.94 (d, J=8.0 Hz, 1H), 7.59-7.56 (m, 1H), 7.42 (s, 1H), 7.32-7.26 (m, 2H), 3.86 (s, 1H), 3.42-3.39 (m, 3H), 3.18 (dd, J=14.8, 8.4 Hz, 1H). Chiral analysis column: IG (250*4.6 mm 5 μm); Mobile Phase: n-Hexane (0.1% DEA):ethanol (0.1% DEA)=70:30; Temperature: 40° C.; Flow: 1.0 mL/min. Retention time: 11.806 min.

Compound 121. MS (ESI): m/z=261[M+H]⁺. ¹H NMR (400 MHz, CD₃OD) δ 8.49 (d, J=4.8 Hz, 1H), 7.98 (d, J=8.0 Hz, 1H), 7.63-7.59 (m, 1H), 7.43 (d, J=1.6 Hz, 1H), 7.33-7.28 (m, 2H), 3.90-3.85 (m, 1H), 3.45-3.30 (m, 3H), 3.19 (dd, J=15.2, 8.8 Hz, 1H). Chiral analysis column: IG (250*4.6 mm 5 μm); Mobile Phase: n-Hexane (0.1% DEA):ethanol (0.1% DEA)=70:30; Temperature: 40° C.; Flow: 1.0 mL/min. Retention time: 16.859 min.

### Example 85. Synthesis of (S*)-(8-chloro-10,11-dihydrobenzo[6,7]oxepino[3,2-b]pyridin-11-yl)-N-methylmethanamine (Compound 122) and (R*)-(8-chloro-10,11-dihydrobenzo[6,7]oxepino[3,2-b]pyridin-11-yl)-N-methylmethanamine (Compound 123)

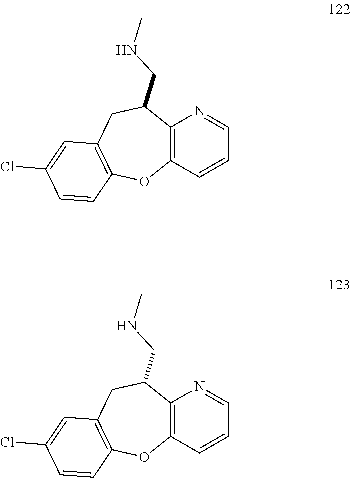 [SMILES: CNC[C@@H]1Cc2cc(Cl)ccc2Oc2cccnc21 ; CNC[C@H]1Cc2cc(Cl)ccc2Oc2cccnc21]

Compound 122 and Compound 123 were prepared using a similar methylation procedure in Example 63.

Compound 122. (ESI): m/z=261[M+H]⁺. ¹H NMR (400 MHz, CD₃OD) δ 8.58 (d, J=4.4 Hz, 1H), 8.20 (d, J=8.4 Hz, 1H), 7.80 (dd, J=8.0, 4.8 Hz, 1H), 7.52 (d, J=1.2 Hz, 1H), 7.37-7.31 (m, 2H), 4.09 (s, 1H), 3.54-3.36 (m, 3H), 3.33-3.27 (m, 1H), 2.82 (s, 3H). Chiral analysis column: IA (250*4.6 mm 5 μm); Mobile Phase: n-Hexane (0.1% DEA):ethanol (0.1% DEA)=90:10; Temperature: 40° C.; Flow: 1.0 mL/min. Retention time: 7.136 min.

Compound 123. (ESI): m/z=261[M+H]⁺. ¹H NMR (400 MHz, CD₃OD) δ 8.55 (d, J=4.4 Hz, 1H), 8.14 (d, J=8.4 Hz, 1H), 7.75 (dd, J=8.0, 4.8 Hz, 1H), 7.49 (d, J=1.6 Hz, 1H), 7.36-7.30 (m, 2H), 4.04 (s, 1H), 3.52-3.35 (m, 3H), 3.33-3.24 (m, 1H), 2.82 (s, 3H). Chiral analysis column: IA (250*4.6 mm 5 μm); Mobile Phase: n-Hexane (0.1% DEA):ethanol (0.1% DEA)=90:10; Temperature: 40° C.; Flow: 1.0 mL/min. Retention time: 6.255 min.

### Example 86. Synthesis of (S*)-(7-chloro-10,11-dihydrobenzo[6,7]oxepino[3,2-b]pyridin-11-yl)methanamine (Compound 124) and (R*)-(7-chloro-10,11-dihydrobenzo[6,7]oxepino[3,2-b]pyridin-11-yl)methanamine (Compound 125)

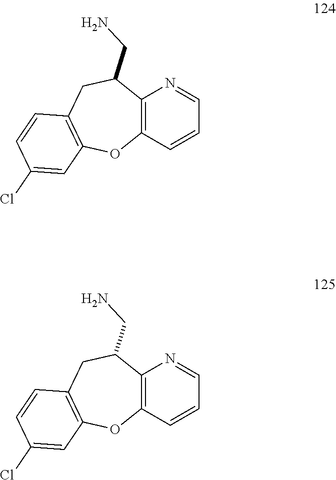 [SMILES: NC[C@@H]1Cc2ccc(Cl)cc2Oc2cccnc21 ; NC[C@H]1Cc2ccc(Cl)cc2Oc2cccnc21]

Compound 124 and Compound 125 were prepared using a similar procedure in Example 80.

Compound 124. MS (ESI): m/z=261.2 [M+H]⁺. ¹H NMR (500 MHz, DMSO-d₆) δ 8.40 (d, J=3.5 Hz, 1H), 8.22 (s, 3H), 7.74 (d, J=7.1 Hz, 1H), 7.43-7.40 (m, 3H), 7.24 (dd, J=8.2, 2.0 Hz, 1H), 3.78-3.77 (m, 1H), 3.38-3.33 (m, 2H), 3.11-3.03 (m, 2H). Chiral analysis column: AD-H (250*4.6 mm 5 μm), Mobile Phase: n-Hexane (0.11% DEA):ethanol (0.1% DEA)=70:30, Temperature: 40° C., Flow:1.0 mL/min, retention time: 8.941 min.

Compound 125. MS (ESI): m/z=261[M+H]⁺. ¹H NMR (500 MHz, DMSO-d₆) δ 8.41 (dd, J=1.5, 4.5 Hz, 1H), 8.28 (s, 3H), 7.75 (dd, J=1.0, 8.5 Hz, 1H), 7.43-7.40 (m, 3H), 7.25 (dd, J=1.5, 8.0 Hz, 1H), 3.78-3.75 (m, 1H), 3.39-3.33 (m, 2H), 3.11-3.04 (m, 2H). Chiral analysis column: AD-H (250*4.6 mm 5 μm), Mobile Phase: n-Hexane (0.11% DEA):EtOH (0.1% DEA)=70:30, Temperature: 40° C., Flow:1.0 mL/min, retention time: 13.476 min.

### Example 87. Synthesis of (S*)-(7-chloro-10,11-dihydrobenzo[6,7]oxepino[3,2-b]pyridin-11-yl)-N-methylmethanamine (Compound 126) and (R*)-(7-chloro-10,11-dihydrobenzo[6,7]oxepino[3,2-b]pyridin-11-yl)-N-methylmethanamine (Compound 127)

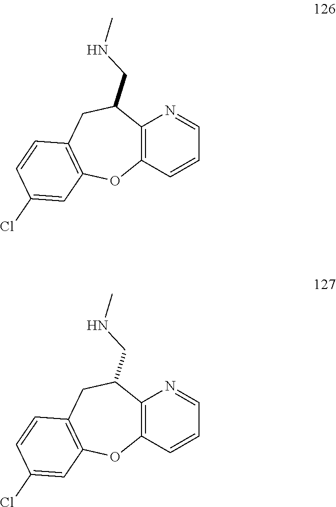 [SMILES: CNC[C@@H]1Cc2ccc(Cl)cc2Oc2cccnc21 ; CNC[C@H]1Cc2ccc(Cl)cc2Oc2cccnc21]

Compound 126 and Compound 127 were prepared using a similar methylation procedure in Example 63.

Compound 126. MS (ESI): m/z=275.1[M+H]⁺. ¹H NMR (500 MHz, DMSO-d₆) δ 9.27 (bs, 1H), 8.83 (bs, 1H), 8.40 (dd, J=1.0, 4.5 Hz, 1H), 7.76 (dd, J=1.0, 8.5 Hz, 1H), 7.44-7.40 (m, 3H), 7.25 (dd, J=2.0, 8.0 Hz, 1H), 3.88-3.83 (m, 1H), 3.45-3.37 (m, 2H), 3.22-3.06 (m, 2H), 2.61 (t, J=5.5 Hz, 3H). Chiral analysis column: AY-H (250*4.6 mm 5 μm), Mobile Phase: n-Hexane (0.1% DEA):EtOH (0.1% DEA)=70:30, Temperature: 40 C, Flow:1.0 mL/min, retention time: 6.022 min.

Compound 127. MS (ESI): m/z=275 [M+H]⁺. ¹H NMR (500 MHz, DMSO-d₆) δ 9.27 (bs, 1H), 8.83 (bs, 1H), 8.40 (dd, J=1.0, 4.5 Hz, 1H), 7.76 (dd, J=1.0, 8.5 Hz, 1H), 7.44-7.40 (m, 3H), 7.25 (dd, J=2.0, 8.0 Hz, 1H), 3.88-3.83 (m, 1H), 3.42-3.39 (m, 2H), 3.19-3.07 (m, 2H), 2.61 (t, J=5.5 Hz, 3H). Chiral analysis column: AY-H (250*4.6 mm 5 μm), Mobile Phase: n-Hexane (0.1% DEA):EtOH (0.1% DEA)=70:30, Temperature: 40 C, Flow:1.0 mL/min, retention time: 4.621 min.

### Example 88. Synthesis of (S*)-(8-methyl-10,11-dihydrobenzo[6,7]oxepino[3,2-b]pyridin-11-yl)methanamine (Compound 128) and (R*)-(8-methyl-10,11-dihydrobenzo[6,7]oxepino[3,2-b]pyridin-11-yl)methanamine (Compound 129)

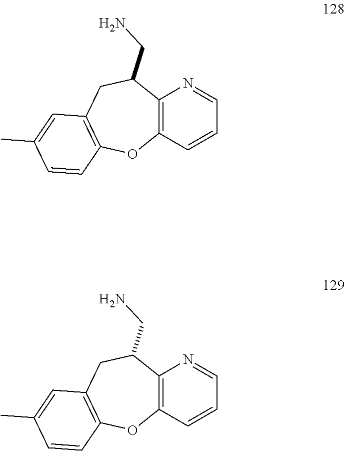 [SMILES: Cc1ccc2c(c1)C[C@@H](CN)c1ncccc1O2 ; Cc1ccc2c(c1)C[C@H](CN)c1ncccc1O2]

Compound 128 and Compound 129 were prepared using a similar procedure in Example 80.

Compound 128. Ms (ESI): m/z=241[M+H]⁺. ¹H NMR (400 MHz, CD₃OD) δ 8.49-8.46 (m, 1H), 8.01 (dd, J=19.6, 8.4 Hz, 1H), 7.67-7.60 (m, 1H), 7.20-7.11 (m, 3H), 3.89-3.86 (m, 1H), 3.46-3.25 (m, 3H), 3.20-3.13 (m, 1H), 2.34 (s, 3H). Chiral analysis column: Column: IG (4.6*250 mm 5 μm), Mobile phase: n-Hexane (0.1% DEA):ethanol (0.1% DEA)=60:40, Wavelength: 214 nm&254 nm, Flowrate:1.0 mL/min, Temperature: 40° C. Retention time=11.258 min.

Compound 129. MS (ESI): m/z=241[M+H]⁺. ¹H NMR (400 MHz, CD₃OD) δ 8.54 (bs, 1H), 8.18 (d, J=7.6 Hz, 1H), 7.79 (bs, 1H), 7.24-7.14 (m, 3H), 4.01 (bs, 1H), 3.54-3.23 (m, 4H), 2.35 (s, 3H). Chiral analysis column: Column: IG (4.6*250 mm 5 μm), Mobile phase: n-Hexane (0.1% DEA):ethanol (0.1% DEA)=60:40, Wavelength: 214 nm&254 nm, Flowrate:1.0 mL/min, Temperature: 40° C. Retention time=18.210 min.

### Example 89. Synthesis of (S*)—N-methyl-1-(8-methyl-10,11-dihydrobenzo[6,7]oxepino[3,2-b]pyridin-11-yl)methanamine (Compound 130) and (R*)—N-methyl-1-(8-methyl-10,11-dihydrobenzo[6,7]oxepino[3,2-b]pyridin-11-yl)methanamine (Compound 131)

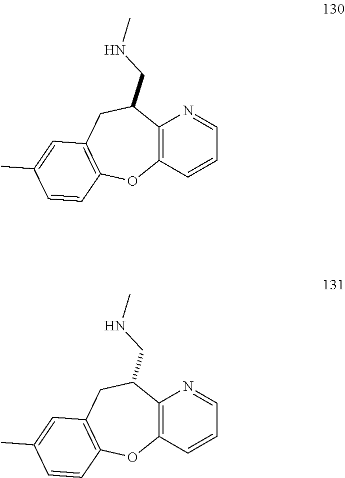 [SMILES: CNC[C@@H]1Cc2cc(C)ccc2Oc2cccnc21 ; CNC[C@H]1Cc2cc(C)ccc2Oc2cccnc21]

Compound 130 and Compound 131 were prepared using a similar methylation procedure in Example 63.

Compound 130. MS(ESI): m/z=254[M+H]⁺. ¹H NMR (400 MHz, CD₃OD) δ 8.49 (d, J=4 Hz, 1H), 8.02 (d, J=8 Hz, 1H), 7.65 (dd, J=8.4, 4.8 Hz, 1H), 7.21-7.12 (m, 3H), 4.10-3.78 (m, 1H), 3.45-3.35 (m, 3H), 3.19 (dd, J=14.8, 7.6 Hz, 1H), 2.81 (s, 3H), 2.35 (s, 3H). Chiral analysis column: AY-H (250*4.6 mm 5 μm); Moblie Phase: n-Hexane (0.1% DEA):ethanol (0.1% DEA)=70:30; Temperature: 40° C.; Flow:1.0 mL/min; Wavelength: 254 nm; Instrument: SHIMADZU; Inject Volume: 5 μl; retention time=6.236 min.

Compound 131. MS (ESI) m/z: 255[M+H]⁺. ¹H NMR (400 MHz, CD₃OD) δ 8.59 (d, J=4.8 Hz, 1H), 8.28 (d, J=8 Hz, 1H), 7.87 (dd, J=8.0, 5.2 Hz, 1H), 7.30-7.15 (m, 3H), 4.14 (s, 1H), 3.57-3.26 (m, 4H), 2.82 (s, 3H), 2.36 (s, 3H). Chiral analysis column: AY-H (250*4.6 mm 5 μm); Moblie Phase: n-Hexane (0.1% DEA):ethanol (0.1% DEA)=70:30; Temperature: 40° C.; Flow:1.0 mL/min; Wavelength:254 nm; Instrument: SHIMADZU; Inject Volume: 5 μl; retention time=4.640 min.

### Example 90. Synthesis of (S*)-(7-methyl-10,11-dihydrobenzo[6,7]oxepino[3,2-b]pyridin-11-yl)methanamine (Compound 132) and (R*)-(7-methyl-10,11-dihydrobenzo[6,7]oxepino[3,2-b]pyridin-11-yl)methanamine (Compound 133)

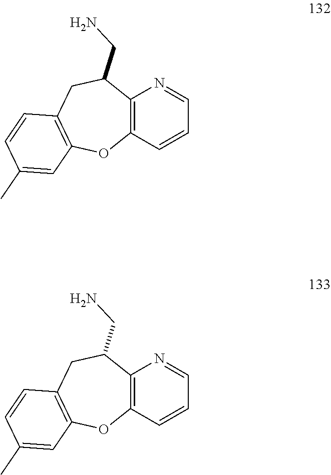 [SMILES: Cc1ccc2c(c1)Oc1cccnc1[C@H](CN)C2 ; Cc1ccc2c(c1)Oc1cccnc1[C@@H](CN)C2]

Compound 132 and Compound 133 were prepared using a similar procedure in Example 80.

Compound 132. MS (ESI) m/z: 241[M+H]⁺. ¹H NMR (400 MHz, CD₃OD) δ:8.54 (dd, J=1.2, 4.2 Hz, 1H), 8.19 (d, J=8.0 Hz, 1H), 7.78 (dd, J=4.2, 8.4 Hz, 1H), 7.27 (d, J=7.6 Hz, 1H), 7.14 (s, 1H), 7.05 (d, J=7.2 Hz, 1H), 4.01-3.98 (m, 1H), 3.50 (dd, J=2.8, 15.2 Hz, 1H), 3.36-3.33 (m, 1H), 3.24-3.17 (m, 2H), 2.34 (s, 3H). Chiral analysis column: AD-H (250*4.6 mm 5 μm), mobile phase: n-hexane (0.1% DEA):ethanol (0.1% DEA)=70:30, temperature: 40° C., flow:1.0 mL/min, retention time: 10.769 min.

Compound 133. MS (ESI): 241[M+H]⁺. ¹H NMR (400 MHz, CD₃OD) δ:8.55 (dd, J=1.6, 4.2 Hz, 1H), 8.19 (dd, J=1.2, 8.8 Hz, 1H), 7.79 (dd, J=4.2, 8.4 Hz, 1H), 7.29 (d, J=7.6 Hz, 1H), 7.16 (s, 1H), 7.07 (d, J=7.6 Hz, 1H), 4.02-4.00 (m, 1H), 3.50 (dd, J=3.2, 15.2 Hz, 1H), 3.39-3.33 (m, 1H), 3.26-3.19 (m, 2H), 2.36 (s, 3H). Chiral analysis column: AD-H (250*4.6 mm 5 μm), mobile phase: n-hexane (0.1% DEA):ethanol (0.1% DEA)=70:30, temperature: 40° C., flow:1.0 mL/min, retention time: 8.369 min.

### Example 91. Synthesis of (S*)—N-methyl-1-(7-methyl-10,11-dihydrobenzo[6,7]-oxepino[3,2-b]pyridin-11-yl)methanamine (Compound 134) and (R*)—N-methyl-1-(7-methyl-10,11-dihydrobenzo[6,7]oxepino[3,2-b]pyridin-11-yl)methanamine (Compound 135)

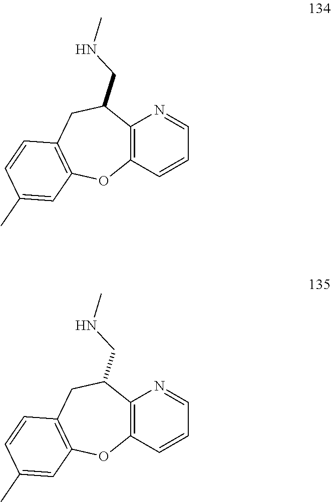 [SMILES: CNC[C@@H]1Cc2ccc(C)cc2Oc2cccnc21 ; CNC[C@H]1Cc2ccc(C)cc2Oc2cccnc21]

Compound 134 and Compound 135 were prepared using a similar methylation procedure in Example 63.

Compound 134. MS (ESI) m/z: 255[M+H]⁺. ¹H NMR (400 MHz, CD₃OD) δ:8.51 (dd, J=1.2, 4.8 Hz, 1H), 8.07 (dd, J=1.2, 8.4 Hz, 1H), 7.69 (dd, J=4.2, 8.4 Hz, 1H), 7.28 (d, J=7.6 Hz, 1H), 7.14 (s, 1H), 7.05 (d, J=7.6 Hz, 1H), 3.99-3.97 (m, 1H), 3.47-3.34 (m, 3H), 3.23-3.18 (m, 1H), 2.81 (s, 3H), 2.36 (s, 3H). Chiral analysis column: AY-H (250*4.6 mm 5 μm), mobile phase: n-hexane (0.1% DEA):ethanol (0.1% DEA)=80:20, temperature: 40° C., flow:1.0 mL/min, retention time: 5.467 min.

Compound 135. MS (ESI): m/z: 255[M+H]⁺. ¹H NMR (400 MHz, CD₃OD) δ:8.50 (dd, J=1.2, 4.8 Hz, 1H), 8.06 (dd, J=1.2, 8.0 Hz, 1H), 7.68 (dd, J=4.2, 8.4 Hz, 1H), 7.27 (d, J=7.6 Hz, 1H), 7.14 (s, 1H), 7.05 (d, J=7.6 Hz, 1H), 3.98-3.96 (m, 1H), 3.46-3.34 (m, 3H), 3.23-3.17 (m, 1H), 2.81 (s, 3H), 2.36 (s, 3H). Chiral analysis column: AY-H (250*4.6 mm 5 μm), mobile phase: n-hexane (0.1% DEA):ethanol (0.1% DEA)=80:20, temperature: 40° C., flow:1.0 mL/min, retention time: 11.709 min.

### Example 92. Synthesis of (S*)-(9-fluoro-10,11-dihydrobenzo[6,7]oxepino[3,2-b]pyridin-11-yl)methanamine (Compound 136) and (R*)-(9-fluoro-10,11-dihydrobenzo[6,7]oxepino[3,2-b]pyridin-11-yl)methanamine (Compound 137)

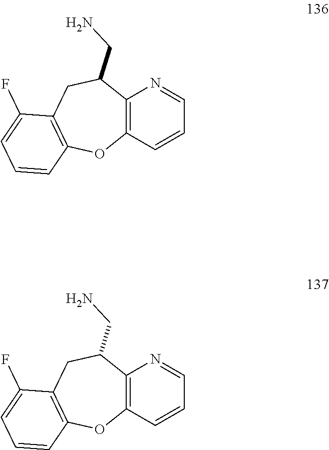 [SMILES: NC[C@@H]1Cc2c(F)cccc2Oc2cccnc21 ; NC[C@H]1Cc2c(F)cccc2Oc2cccnc21]

Compound 136 and Compound 137 were prepared using a similar procedure in Example 80.

Compound 136. MS (ESI): m/z 245 [M+H]⁺. ¹H NMR (400 MHz, CD₃OD) δ: 8.48 (d, J=4.8 Hz, 1H), 7.87 (d, J=7.2 Hz, 1H), 7.53 (d, J=2.8 Hz, 1H), 7.34-7.28 (m, 1H), 7.15 (d, J=8.4 Hz, 1H), 7.00 (t, J=8.4 Hz, 1H), 3.99-3.97 (m, 1H), 3.59-3.53 (m, 1H), 3.44-3.33 (m, 2H), 3.10-3.04 (m, 1H). Chiral analysis column: IA (4.6*250 mm 5 μm); Mobile Phase: n-Hexane (0.1% DEA):ethanol (0.1% DEA)=80:20; Temperature: 40° C.; Flow: 1.0 mL/min; Wavelength: 214 nm & 254 nm; retention time=7.496 min.

Compound 137. MS (ESI): m/z 245 [M+H]⁺. ₁H NMR (400 MHz, CD₃OD) δ: 8.49 (d, J=4.4 Hz, 1H), 7.90 (d, J=7.6 Hz, 1H), 7.56-7.53 (m, 1H), 7.34-7.28 (m, 1H), 7.15 (d, J=8.4 Hz, 1H), 7.00 (t, J=8.4 Hz, 1H), 4.02-3.97 (m, 1H), 3.59-3.53 (m, 1H), 3.44-3.33 (m, 2H), 3.12-3.06 (m, 1H). Chiral analysis column: IA (4.6*250 mm 5 μm); Mobile Phase: n-Hexane (0.1% DEA):ethanol (0.1% DEA)=80:20; Temperature: 40° C.; Flow: 1.0 mL/min; Wavelength: 214 nm & 254 nm; retention time=5.800 min.

### Example 93. Synthesis of (S*)-1-(9-fluoro-10,11-dihydrobenzo[6,7]oxepino[3,2-b]pyridin-11-yl)-N-methylmethanamine (Compound 138) and (R*)-1-(9-fluoro-10,11-dihydrobenzo[6,7]oxepino[3,2-b]pyridin-11-yl)-N-methylmethanamine (Compound 139)

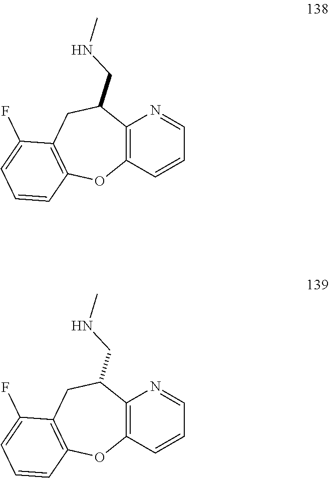 [SMILES: CNC[C@@H]1Cc2c(F)cccc2Oc2cccnc21 ; CNC[C@H]1Cc2c(F)cccc2Oc2cccnc21]

Compound 138 and Compound 139 were prepared using a similar methylation procedure in Example 63.

Compound 138. MS (ESI): m/z 259 [M+H]⁺. ¹H NMR (400 MHz, CD₃OD) δ: 8.53 (dd, J=4.8, 1.2 Hz, 1H), 7.99 (dd, J=8.4, 1.2 Hz, 1H), 7.63 (dd, J=8.4, 4.8 Hz, 1H), 7.36-7.30 (m, 1H), 7.17 (d, J=8.4 Hz, 1H), 7.05-7.00 (m, 1H), 4.11-4.05 (m, 1H), 3.67-3.61 (m, 1H), 3.49-3.39 (m, 2H), 3.20-3.14 (m, 1H), 2.83 (s, 3H). Chiral analysis column: AY-H (250*4.6 mm 5 μm); Mobile Phase: n-Hexane (0.1% DEA):ethanol (0.1% DEA)=80:20; Temperature: 40° C.; Flow: 1.0 mL/min; Wavelength: 214 nm & 254 nm; Instrument: SHIMADZU; retention time=8.156 min.

Compound 139. MS (ESI): m/z 259 [M+H]⁺. ¹H NMR (400 MHz, CD₃OD) δ: 8.50 (dd, J=4.8, 1.2 Hz, 1H), 7.93 (d, J=8.4 Hz, 1H), 7.59-7.56 (m, 1H), 7.35-7.29 (m, 1H), 7.16 (d, J=8.4 Hz, 1H), 7.04-6.99 (m, 1H), 4.07-4.02 (m, 1H), 3.66-3.61 (m, 1H), 3.49-3.34 (m, 2H), 3.16-3.09 (m, 1H), 2.83 (s, 3H). Chiral analysis column: AY-H (250*4.6 mm 5 μm); Mobile Phase: n-Hexane (0.1% DEA):ethanol (0.1% DEA)=80:20; Temperature: 40° C.; Flow: 1.0 mL/min; Wavelength: 214 nm & 254 nm; Instrument: SHIMADZU; retention time=5.123 min.

### Example 94. Synthesis of (S*)-(9-methyl-10,11-dihydrobenzo[6,7]oxepino[3,2-b]pyridin-11-yl)methanamine (Compound 140) and (R*)-(9-methyl-10,11-dihydrobenzo[6,7]oxepino[3,2-b]pyridin-11-yl)methanamine (Compound 141)

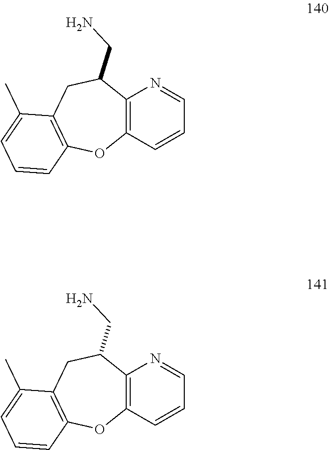 [SMILES: Cc1cccc2c1C[C@@H](CN)c1ncccc1O2 ; Cc1cccc2c1C[C@H](CN)c1ncccc1O2]

Compound 140 and Compound 141 were prepared using a similar procedure in Example 80.

Compound 140. MS (ESI): m/z 241 [M+H]⁺. ¹H NMR (400 MHz, CD₃OD) δ: 8.51-8.50 (m, 1H), 8.09-8.07 (m, 1H), 7.70-7.67 (m, 1H), 7.19-7.08 (m, 3H), 3.96-3.94 (m, 1H), 3.50-3.45 (m, 2H), 3.31-3.25 (m, 2H), 2.39 (s, 3H). Chiral analysis: column Name: OZ 4.6*100 mm 5 μm; Acq. Method Set: OZ 20% B1; Co-Solvent: MeOH[0.2% NH₃ (7M in MeOH)]; Flow_rate: 3.0 mL/min; Back_Pressure: 2000 psi; Column_Temperature: 40° C.; retention time=4.018 min.

Compound 141. MS (ESI): m/z 241 [M+H]⁺. ¹H NMR (400 MHz, CD₃OD) δ: 8.55-8.52 (m, 1H), 8.19-8.16 (m, 1H), 7.77-7.71 (m, 1H), 7.20-7.09 (m, 3H), 3.99-3.98 (m, 1H), 3.55-3.44 (m, 2H), 3.37-3.24 (m, 2H), 2.40 (s, 3H). Chiral analysis: Column Name: OZ 4.6*100 mm 5 μm; Acq. Method Set: OZ 20% B1; Co-Solvent: MeOH[0.2% NH₃ (7M in MeOH)]; Flow_rate: 3.0 mL/min; Back_Pressure: 2000 psi; Column_Temperature: 40° C.; retention time=4.635 min.

### Example 95. Synthesis of (S*)-(9-methyl-10,11-dihydrobenzo[6,7]oxepino[3,2-b]pyridin-11-yl)-N-methylmethanamine (Compound 142) and (R*)-(9-methyl-10,11-dihydrobenzo[6,7]oxepino[3,2-b]pyridin-11-yl)-N-methylmethanamine (Compound 143)

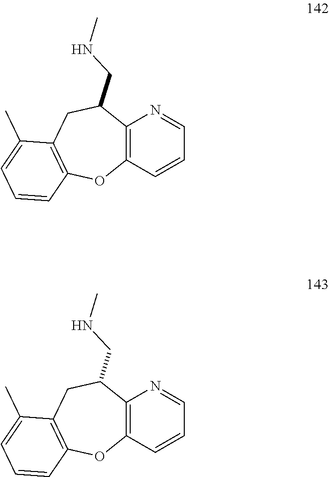 [SMILES: CNC[C@@H]1Cc2c(C)cccc2Oc2cccnc21 ; CNC[C@H]1Cc2c(C)cccc2Oc2cccnc21]

Compound 142 and Compound 143 were prepared using a similar methylation procedure in Example 63.

Compound 142. MS (ESI): m/z 255 [M+H]⁺. ¹H NMR (500 MHz, CD₃OD) δ: 8.46-8.41 (m, 1H), 7.99-7.82 (m, 1H), 7.59-7.47 (m, 1H), 7.17-7.06 (m, 3H), 3.92-3.84 (m, 1H), 3.57-3.46 (m, 2H), 3.33-3.11 (m, 2H), 2.81 (s, 3H), 2.38 (s, 3H). Chiral analysis column: Column Name: AD-3 4.6*100 mm 3 μm, Acq. Method Set: AD 15% B1, Co-Solvent: MeOH[0.2% NH3(7M in MeOH)], Flow_rate: 3.0 mL/min, Back_Pressure: 2000 psi, Column_Temperature: 40° C., retention time: 1.617 min.

Compound 143. MS (ESI): m/z 255 [M+H]⁺. ¹H NMR (500 MHz, CD₃OD) δ: 8.47-8.42 (m, 1H), 8.00-7.83 (m, 1H), 7.61-7.48 (m, 1H), 7.16-7.06 (m, 3H), 3.94-3.84 (m, 1H), 3.55-3.46 (m, 2H), 3.31-3.12 (m, 2H), 2.81 (s, 3H), 2.38 (s, 3H). Chiral analysis column: Column Name: AD-3 4.6*100 mm 3 μm; Processing Method: AS1; Acq. Method Set: AD 15% B1; Vial: 2:B,2; Co-Solvent: methanol[0.2% NH3(7M in methanol)]; Injection Volume: 5.00 μl; Channel Name: PDA Ch2 214 nm@4.8 nm; Run Time: 6.0 Minutes; Proc. Chnl. Descr.: PDA Ch2 214 nm@4.8 nm; Flow_rate: 3.0 mL/min; Temperature: 40° C.; retention time=2.172 min.

### Example 96. Synthesis of (S*)-(9-chloro-10,11-dihydrobenzo[6,7]oxepino[3,2-b]pyridin-11-yl)methanamine (Compound 144) and (R*)-(9-chloro-10,11-dihydrobenzo[6,7]oxepino[3,2-b]pyridin-11-yl)methanamine (Compound 145)

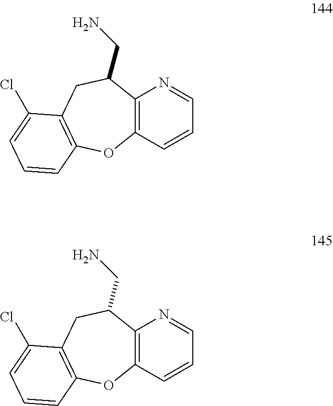 [SMILES: NC[C@@H]1Cc2c(Cl)cccc2Oc2cccnc21 ; NC[C@H]1Cc2c(Cl)cccc2Oc2cccnc21]

Compound 144 and Compound 145 were prepared using a similar procedure in Example 80.

Compound 144. (ESI): m/z=261[M+H]⁺. ¹H NMR (400 MHz, CD₃OD) δ 8.52 (dd, J=4.8, 1.2 Hz, 1H), 8.01 (dd, J=8.4, 1.6 Hz, 1H), 7.63 (dd, J=8.4, 5.2 Hz, 1H), 7.37-7.30 (m, 3H), 3.98-3.97 (m, 1H), 3.58-3.35 (m, 4H). Chiral analysis column: OZ (250*4.6 mm 5 μm); Method Set: OZ 30%1 B2; Co-Solvent: ethanol[1% NH3(7M in methanol)] Temperature: 40° C.; Flow: 3.0 mL/min. Retention time: 2.016 min.

Compound 145. MS (ESI): m/z=261[M+H]⁺. ¹H NMR (400 MHz, CD₃OD) δ 8.53 (dd, J=4.8, 1.2 Hz, 1H), 8.04 (dd, J=8.0, 1.2 Hz, 1H), 7.65 (dd, J=8.4, 4.8 Hz, 1H), 7.37-7.31 (m, 3H), 4.00-3.98 (m, 1H), 3.58-3.34 (m, 4H). Chiral analysis column: OZ (250*4.6 mm 5 μm); Method Set: OZ 30% B2; Co-Solvent: ethanol[1% NH₃ (7M in methanol)] Temperature: 40° C.; Flow: 3.0 mL/min. Retention time: 2.333 min.

### Example 97. Synthesis of (S*)-(9-chloro-10,11-dihydrobenzo[6,7]oxepino[3,2-b]pyridin-11-yl)-N-methylmethanamine (Compound 146) and (R*)-(9-chloro-10,11-dihydrobenzo[6,7]oxepino[3,2-b]pyridin-11-yl)-N-methylmethanamine (Compound 147)

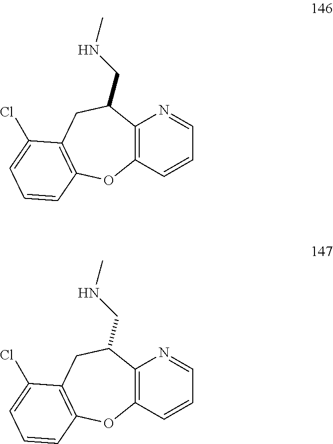 [SMILES: CNC[C@@H]1Cc2c(Cl)cccc2Oc2cccnc21 ; CNC[C@H]1Cc2c(Cl)cccc2Oc2cccnc21]

Compound 146 and Compound 147 were prepared using a similar methylation procedure in Example 63.

Compound 146. (ESI): m/z=275[M+H]⁺. ¹H NMR (400 MHz, CD₃OD) δ 8.52 (dd, J=4.8, 1.2 Hz, 1H), 8.01 (dd, J=8.4, 0.8 Hz, 1H), 7.63 (dd, J=8.4, 4.8 Hz, 1H), 7.37-7.30 (m, 3H), 4.03-4.01 (m, 1H), 3.61-3.46 (m, 3H), 3.39-3.37 (m, 1H), 2.83 (s, 3H). Chiral analysis column: IG (250*4.6 mm 5 μm); Method Set: 35% B1; Co-Solvent: methanol[0.2% NH3(7M in methanol)], Temperature: 40° C.; Flow: 3.0 mL/min. Retention time: 1.118 min.

Compound 147. (ESI): m/z=275[M+H]⁺. ¹H NMR (400 MHz, CD₃OD) δ 8.53 (dd, J=4.8, 1.2 Hz, 1H), 8.03 (dd, J=8.4, 0.8 Hz, 1H), 7.64 (dd, J=8.4, 4.8 Hz, 1H), 7.37-7.30 (m, 3H), 4.05-4.01 (m, 1H), 3.61-3.46 (m, 3H), 3.41-3.37 (m, 1H), 2.83 (s, 3H). Chiral analysis column: IG (250*4.6 mm 5 μm); Method Set: 35% B1; Co-Solvent: methanol[0.2% NH3(7M in methanol)], Temperature: 40° C.; Flow: 3.0 mL/min. Retention time: 1.496 min.

### Example 98. Synthesis of (R*)-(8-bromo-10,11-dihydrobenzo[6,7]oxepino[3,2-b]pyridin-11-yl)methanamine (Compound 148)

### Preparation of tert-butyl (R*)-((10,11-dihydrobenzo[6,7]oxepino[3,2-b]pyridin-11-yl)methyl)carbamate

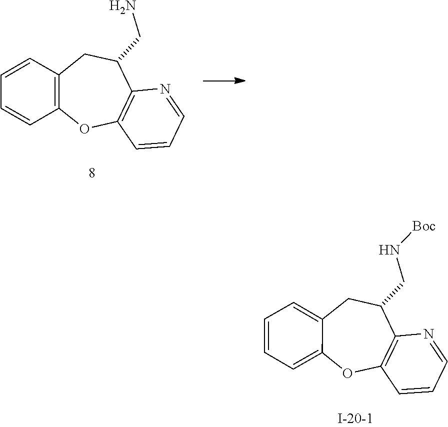 [SMILES: NC[C@H]1Cc2ccccc2Oc2cccnc21 ; CC(C)(C)OC(=O)NC[C@H]1Cc2ccccc2Oc2cccnc21]

To a solution of Compound 8 (2.1 g, 7.07 mmol) in dichloromethane (20 mL) was added di-tert-butyl dicarbonate (2.31 g, 10.6 mmol) and triethylamine (1.42 g, 14.1 mmol) at ambient temperature. Then the mixture was stirred at ambient temperature for 2 h. Water (20 mL) was added to the reaction vessel and the resulting biphasic mixture was transferred to a separatory funnel. The layers were separated and the aqueous phase was washed with dichloromethane (2×20 mL). The combined organics were dried over anhydrous sodium sulphate, filtered and concentrated in vacuo. The resulting mixture was purified by flash column chromatography with a gradient elution of petroleum ether (100%) and ethyl acetate (0%) to petroleum ether (80%) and ethyl acetate (20%) to provide I-20-1 as a colorless oil. 1.8 g. Yield: 67%. MS (ESI): m/z 327 [M+H]⁺.

### Preparation of tert-butyl (R*)-((8-bromo-10,11-dihydrobenzo[6,7]oxepino[3,2-b]pyridin-11-yl)methyl)carbamate

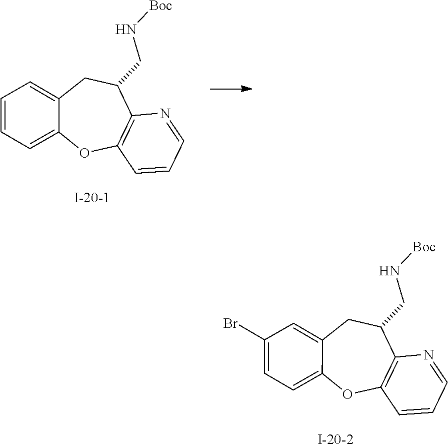 [SMILES: CC(C)(C)OC(=O)NC[C@H]1Cc2ccccc2Oc2cccnc21 ; CC(C)(C)OC(=O)NC[C@H]1Cc2cc(Br)ccc2Oc2cccnc21]

To a solution of I-20-1 (400 mg, 1.23 mmol) in acetic acid (3 mL) was added Br₂ (224 mg, 1.4 mmol). The reaction mixture was stirred at ambient temperature for 3 h. Upon the completion, ethyl acetate (20 mL) was added to the reaction vessel and the resulting mixture was transferred to a separatory funnel. The organic phase was washed with saturated sodium bicarbonate (2×10 mL) and saturated aqueous brine (2×5 mL). The combined organics were dried over anhydrous sodium sulphate, filtered and concentrated in vacuo. The resulting oil was purified by flash column chromatography with a gradient elution of petroleum ether (100%) to petroleum ether (80%) and ethyl acetate (20%) to get I-20-2 (200 mg, yield: 40%) as a white solid. (ESI) m/z: 405[M+H]⁺.

### Synthesis of (R*)-(8-bromo-10,11-dihydrobenzo[6,7]oxepino[3,2-b]pyridin-11-yl)methanamine (Compound 148)

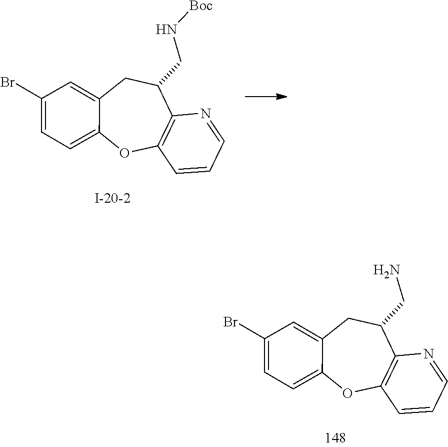 [SMILES: CC(C)(C)OC(=O)NC[C@H]1Cc2cc(Br)ccc2Oc2cccnc21 ; NC[C@H]1Cc2cc(Br)ccc2Oc2cccnc21]

A solution of compound I-20-2 (200 mg, 0.5 mmol) in 3M hydrogen chloride in methanol (5 mL) was stirred at ambient temperature for 2 h. Upon the completion, the mixture was evaporated in vacuo to dryness and then freeze-dried to yield Compound 148. MS (ESI) m/z: 305 [M+H]⁺. ¹H NMR (400 MHz, CD₃OD) δ 8.50-8.43 (m, 1H), 7.90-7.89 (m, 1H), 7.55-7.40 (m, 3H), 7.23-7.20 (m, 1H), 3.43-3.20 (m, 5H).

### Example 99. Synthesis of (R*)-(2-methyl-10,11-dihydrobenzo[6,7]oxepino[3,2-b]pyridin-11-yl)methanamine (Compound 149)

### Preparation of (R*)-11-(((tert-butoxycarbonyl)amino)methyl)-10,11-dihydrobenzo-[6,7]oxepino[3,2-b]pyridine 1-oxide

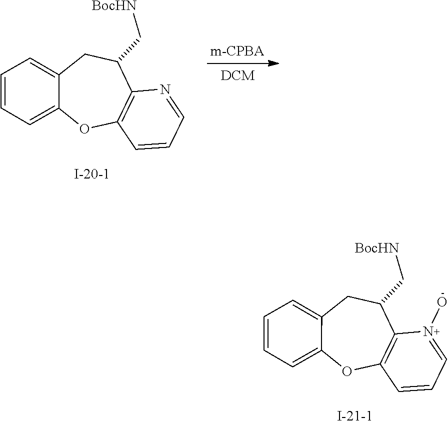

To a solution of I-20-1 (1.5 g, 4.59 mmol) in dichloromethane (20 mL) was added 3-Chloroperoxybenzoic acid (1.58 g, 9.18 mmol) at ambient temperature. Then the mixture was stirred at ambient temperature for 16 h. Upon the completion, saturated aqueous sodium bicarbonate (50 mL) was added to the reaction vessel and the resulting biphasic mixture was stirred at ambient temperature for 30 min. The layers were separated and the aqueous phase was extracted with dichloromethane (2×30 mL). The combined organics were dried over anhydrous sodium sulphate, filtered and concentrated in vacuo to give I-21-1 (1.2 g, yield: 59%) as a yellow oil. MS (ESI): m/z 343 [M+H]⁺.

### Preparation of tert-butyl (R*)-((2-chloro-10,11-dihydrobenzo[6,7]oxepino[3,2-b]pyridin-11-yl)methyl)carbamate

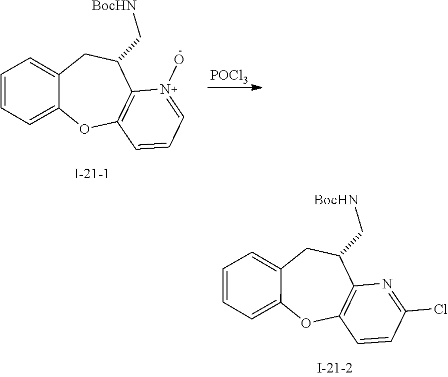

To a reaction vessel of I-21-1 (1.1 g, 3.21 mmol) was slowly added phosphoroyl trichloride (20 mL) at 0° C. Then the mixture was stirred at 100° C. for 2 h. After concentration, 1M aqueous sodium hydroxide (10 mL), di-tert-butyl dicarbonate (1.05 g, 4.8 mmol) was added to the reaction vessel and the mixture was stirred at ambient temperature for 2 h. The layers were separated and the aqueous phase was washed with dichloromethane (3×30 mL). The combined organics were dried over anhydrous sodium sulphate, filtered and concentrated in vacuo. The resulting mixture was purified by flash column chromatography with a gradient elution of petroleum ether (100%) and ethyl acetate (0%) to petroleum ether (85%) and ethyl acetate (15%) to provide I-21-2 (170 mg, yield: 12%) as a yellow oil. MS (ESI): m/z 361 [M+H]⁺.

### Preparation of tert-butyl (R*)-((2-methyl-10,11-dihydrobenzo[6,7]oxepino[3,2-b]pyridin-11-yl)methyl)carbamate

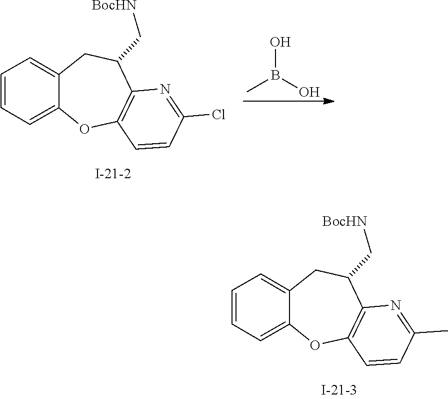

To a solution of I-21-2 (170 mg, 471 μmol) in 1,4-dioxane/water (1:1, 5 mL) was added methylboronic acid (56.3 mg, 942 μmol), 1,1′-bis(diphenylphosphino)-ferrocenepalladium(II)dichloride dichloromethane complex (76.3 mg, 94.2 μmol) and potassium carbonate (130 mg, 942 μmol) at ambient temperature under nitrogen. Then the mixture was stirred at 100° C. under microwave for 2 h. After cooling down to ambient temperature, water (10 mL) was added to the reaction vessel and the resulting biphasic mixture was transferred to a separatory funnel. The layers were separated and the aqueous phase was extracted with ethyl acetate (3×20 mL). The combined organics were dried over anhydrous sodium sulphate, filtered and concentrated in vacuo. The resulting mixture was purified by flash column chromatography with a gradient elution of petroleum ether (100%) and ethyl acetate (0%) to petroleum ether (80%) and ethyl acetate (20%) to provide I-21-3 (120 mg, yield: 67%) as a yellow solid. MS (ESI): m/z 341 [M+H]⁺.

### Synthesis of (R*)-(2-methyl-10,11-dihydrobenzo[6,7]oxepino[3,2-b]pyridin-11-yl)methanamine (Compound 149)

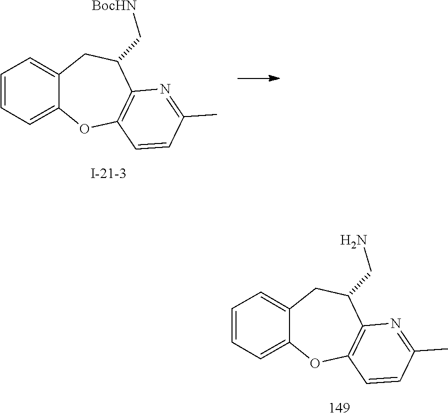 [SMILES: Cc1ccc2c(n1)[C@@H](CNC(=O)OC(C)(C)C)Cc1ccccc1O2 ; Cc1ccc2c(n1)[C@@H](CN)Cc1ccccc1O2]

A solution of I-21-3 (120 mg, 352 μmol) in 4M hydrogen chloride/ethyl acetate (10 mL) was stirred at ambient temperature for 16 h. After concentration, the residue was washed with ethyl acetate (3×2 mL). The solid was dried on freeze dryer. Compound 149 was obtained. MS (ESI): m/z 241 [M+H]⁺. ¹H NMR (500 MHz, CD₃OD) δ: 8.12 (d, J=6.4 Hz, 1H), 7.64 (d, J=6.4 Hz, 1H), 7.41 (d, J=5.6 Hz, 1H), 7.35-7.29 (m, 2H), 7.23 (t, J=6.0 Hz, 1H), 4.03-4.01 (m, 1H), 3.58-3.54 (m, 1H), 3.34-3.15 (m, 3H), 2.74 (s, 3H).

### Example 100. Synthesis of (R*)-(2-methyl-10,11-dihydrobenzo[6,7]oxepino[3,2-b]pyridin-11-yl)-N-methylmethanamine (Compound 150)

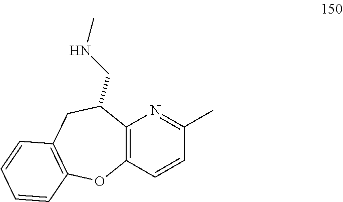 [SMILES: CNC[C@H]1Cc2ccccc2Oc2ccc(C)nc21]

Compound 150 was prepared using a similar methylation procedure described in Example 63. (ESI) m/z=255[M+H]⁺. ¹H NMR (400 MHz, CD₃OD) δ 8.23 (d, J=8.6 Hz, 1H), 7.74 (d, J=8.0 Hz, 1H), 7.46 (d, J=6.5 Hz, 1H), 7.40-7.14 (m, 3H), 4.19 (s, 1H), 3.61 (d, J=15.1 Hz, 1H), 3.40 (dd, J=12.7, 5.0 Hz, 1H), 3.30-3.23 (m, 2H), 2.79 (s, 6H).

### Example 101. Synthesis of (R*)-(2-ethyl-10,11-dihydrobenzo[6,7]oxepino[3,2-b]pyridin-11-yl)methanamine (Compound 151)

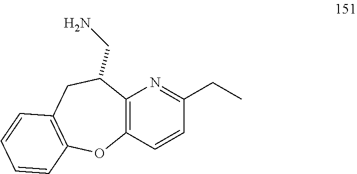 [SMILES: CCc1ccc2c(n1)[C@@H](CN)Cc1ccccc1O2]

Compound 151 was prepared using a similar procedure described in Example 99. MS (ESI) m/z: 255.2 [M+H]⁺. ¹H NMR (400 MHz, CD₃OD) δ 8.08 (d, J=8.6 Hz, 1H), 7.64 (d, J=8.7 Hz, 1H), 7.39 (d, J=7.1 Hz, 1H), 7.36-7.27 (m, 2H), 7.22 (t, J=7.5 Hz, 1H), 3.99 (s, 1H), 3.54 (dd, J=15.1, 3.3 Hz, 1H), 3.39-3.33 (m, 1H), 3.25-3.16 (m, 2H), 3.03 (q, J=7.6 Hz, 2H), 1.37 (t, J=7.6 Hz, 3H).

### Example 102. Synthesis of (R*)-(2-ethyl-10,11-dihydrobenzo[6,7]oxepino[3,2-b]pyridin-11-yl)-N-methylmethanamine (Compound 152)

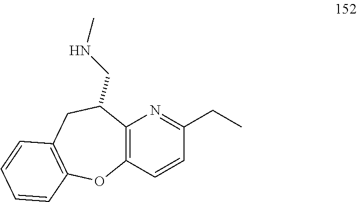 [SMILES: CCc1ccc2c(n1)[C@@H](CNC)Cc1ccccc1O2]

Compound 152 was prepared using a similar methylation procedure in Example 63. MS (ESI) m/z: 269.3 [M+H]⁺. ¹H NMR (400 MHz, CD₃OD) δ 8.13 (d, J=8.6 Hz, 1H), 7.68 (d, J=8.7 Hz, 1H), 7.41 (d, J=7.4 Hz, 1H), 7.32 (ddd, J=13.7, 8.1, 4.1 Hz, 2H), 7.23 (td, J=7.3, 1.6 Hz, 1H), 4.09 (dd, J=6.5, 3.3 Hz, 1H), 3.55 (dd, J=15.2, 3.3 Hz, 1H), 3.40 (dd, J=13.0, 6.6 Hz, 1H), 3.29-3.19 (m, 2H), 3.06 (q, J=7.6 Hz, 2H), 2.79 (s, 3H), 1.39 (t, J=7.6 Hz, 3H).

### Example 103. Synthesis of ((10S*,11S*)-10-methyl-10,11-dihydrobenzo[6,7]oxepino[3,2-b]pyridin-11-yl)methanamine (Compound 112), ((10R*,11R*)-10-methyl-10,11-dihydrobenzo[6,7]oxepino[3,2-b]pyridin-11-yl)methanamine (113), ((10S*,11R*)-10-methyl-10,11-dihydrobenzo[6,7]oxepino[3,2-b]pyridin-11-yl)methanamine (114) and ((10R*,11S*)-10-methyl-10,11-dihydrobenzo[6,7]oxepino[3,2-b]pyridin-11-yl)methanamine (115)

### Synthesis of methyl 10-methyl-10,11-dihydrobenzo[6,7]oxepino[3,2-b]pyridine-11-carboxylate

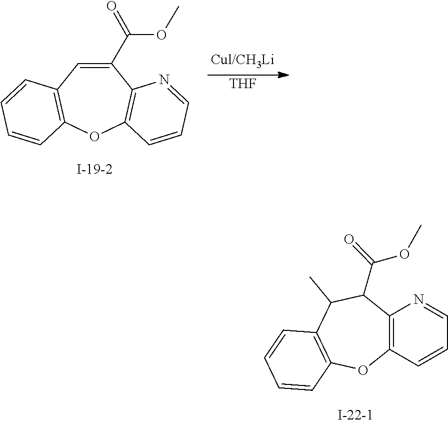 [SMILES: COC(=O)C1=Cc2ccccc2Oc2cccnc21 ; COC(=O)C1c2ncccc2Oc2ccccc2C1C ; "THF"]

To a solution of cuprous iodide (7.86 g, 41.3 mmol) in tetrahydrofuran (50 mL) was slowly added methyllithium (1.8 g, 82.7 mmol) at 0° C. Then the mixture was stirred at 0° C. for 30 min. methyl benzo [6, 7] oxepino [3,2-b]pyridine-11-carboxylate (3.5 g, 13.8 mmol) in tetrahydrofuran (50 mL) was added to the mixture and the mixture was stirred at ambient temperature for 16 h. saturated aqueous ammonium chloride (50 mL) was added to the reaction vessel and the resulting biphasic mixture was transferred to a separatory funnel. The layers were separated and the aqueous phase was extracted with ethyl acetate (3×50 mL). The combined organics were dried over anhydrous sodium sulphate, filtered and concentrated in vacuo. The resulting mixture was purified by flash column chromatography with a gradient elution of petroleum ether (100%) and ethyl acetate (0%) to petroleum ether (80%) and ethyl acetate (20%) to provide methyl 10-methyl-10,11-dihydrobenzo[6,7]oxepino[3,2-b]pyridine-11-carboxylate I-22-1. MS (ESI): m/z 270 [M+H]⁺.

### Synthesis of ((10S*,11S*)-10-methyl-10,11-dihydrobenzo[6,7]oxepino[3,2-b]pyridin-11-yl)methanamine (Compound 112), ((10R*,11R*)-10-methyl-10,11-dihydrobenzo[6,7]oxepino-[3,2-b]-pyridin-11-yl)methanamine (Compound 113), ((10S*,11R*)-10-methyl-10,11-dihydrobenzo[6,7]oxepino[3,2-b]pyridin-11-yl)methanamine (Compound 114) and ((10R*,11S*)-10-methyl-10,11-dihydrobenzo[6,7]oxepino[3,2-b]pyridin-11-yl)methanamine (Compound 115)

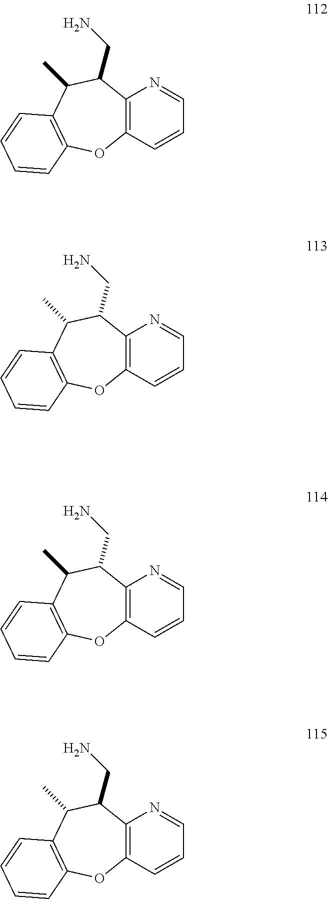 [SMILES: C[C@@H]1c2ccccc2Oc2cccnc2[C@@H]1CN ; C[C@H]1c2ccccc2Oc2cccnc2[C@H]1CN ; C[C@@H]1c2ccccc2Oc2cccnc2[C@H]1CN ; C[C@H]1c2ccccc2Oc2cccnc2[C@@H]1CN]

Compound 112, compound 113, compound 114 and compound 115 were prepared using a similar procedure described in Example 80 from 1-22-1.

Compound 112. MS (ESI): m/z 241 [M+H]⁺. ¹H NMR (400 MHz, CD₃OD) δ: 8.48 (d, J=5.2 Hz, 1H), 7.95 (d, J=8.0 Hz, 1H), 7.63-7.60 (m, 1H), 7.35-7.19 (m, 4H), 4.05-4.02 (m, 1H), 3.67-3.63 (m, 1H), 3.34-3.32 (m, 2H), 1.26 (d, J=6.8 Hz, 3H). Chiral analysis column: AD-H (250*4.6 mm 5 μm); Mobile Phase: n-Hexane (0.1% DEA):ethanol (0.1% DEA)=90:10; Temperature: 40° C.; Flow: 1.0 mL/min; Wavelength: 214 nm & 254 nm; Instrument: SHIMADZU; retention time=9.064 min.

Compound 113. MS (ESI): m/z 241 [M+H]⁺. ¹H NMR (400 MHz, CD₃OD) δ: 8.48 (d, J=5.2 Hz, 1H), 7.95 (d, J=8.0 Hz, 1H), 7.63-7.60 (m, 1H), 7.35-7.19 (m, 4H), 4.05-4.02 (m, 1H), 3.67-3.63 (m, 1H), 3.34-3.32 (m, 2H), 1.24 (d, J=6.8 Hz, 3H). Chiral analysis column: AD-H (250*4.6 mm 5 μm); Mobile Phase: n-Hexane (0.1% DEA):ethanol (0.1% DEA)=90: 10; Temperature: 40° C.; Flow: 1.0 mL/min; Wavelength: 214 nm & 254 nm; Instrument: SHIMADZU; retention time=11.478 min.

Compound 114. MS (ESI): m/z 241 [M+H]⁺. ¹HNMR (400 MHz, CD₃OD): 8.64-8.62 (m, 1H), 8.31-8.27 (m, 1H), 7.92-7.90 (m, 1H), 7.40-7.33 (m, 3H), 7.25 (t, J=5.6 Hz, 1H), 3.92-3.90 (m, 1H), 3.54-3.53 (m, 1H), 3.28-3.24 (m, 2H), 1.35 (d, J=5.6 Hz, 3H). Chiral analysis column: OJ-H (250*4.6 mm 5 μm); Mobile Phase: n-Hexane (0.11% DEA):ethanol (0.1% DEA)=90:10; Temperature: 40° C.; Flow: 1.0 mL/min; Wavelength: 214 nm & 254 nm; Instrument: SHIMADZU; Inject Volume: 5 μl; retention time=5.864 min.

Compound 115. MS (ESI): m/z 241 [M+H]⁺. ¹HNMR (400 MHz, CD₃OD): 8.64-8.62 (m, 1H), 8.31-8.27 (m, 1H), 7.92-7.90 (m, 1H), 7.40-7.33 (m, 3H), 7.25 (t, J=5.6 Hz, 1H), 3.92-3.90 (m, 1H), 3.54-3.53 (m, 1H), 3.28-3.24 (m, 2H), 1.35 (d, J=5.6 Hz, 3H). Chiral analysis column: OJ-H (250*4.6 mm 5 μm); Mobile Phase: n-Hexane (0.11% DEA):ethanol (0.1% DEA)=90:10; Temperature: 40° C.; Flow: 1.0 mL/min; Wavelength: 214 nm & 254 nm; Instrument: SHIMADZU; Inject Volume: 5 μl; retention time=10.596 min.

### Example 104. Synthesis of ((10S*,11S*)-10-methyl-10,11-dihydrobenzo[6,7]oxepino[3,2-b]pyridin-11-yl)-N-methylmethanamine (Compound 116), ((10R*,11R*)-10-methyl-10,11-dihydrobenzo[6,7]oxepino[3,2-b]pyridin-11-yl)-N-methylmethanamine (117), ((10S*,11R*)-10-methyl-10,11-dihydrobenzo[6,7]oxepino[3,2-b]pyridin-11-yl)-N-methylmethanamine (118) and ((10R*,11S*)-10-methyl-10,11-dihydrobenzo[6,7]oxepino[3,2-b]pyridin-11-yl)-N-methylmethanamine (119)

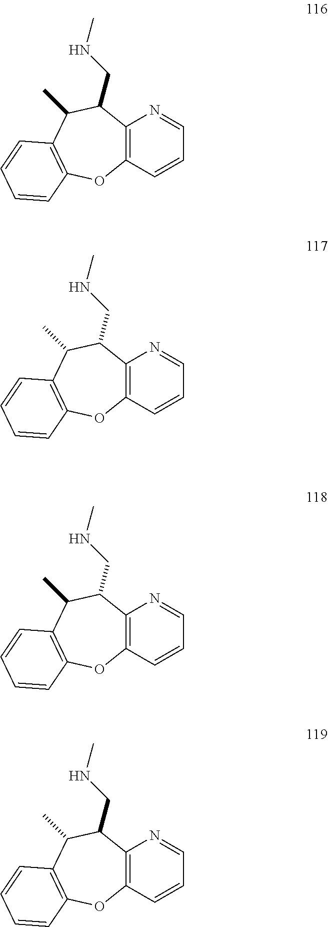 [SMILES: CNC[C@H]1c2ncccc2Oc2ccccc2[C@H]1C ; CNC[C@@H]1c2ncccc2Oc2ccccc2[C@@H]1C ; CNC[C@@H]1c2ncccc2Oc2ccccc2[C@H]1C ; CNC[C@H]1c2ncccc2Oc2ccccc2[C@@H]1C]

Compound 116, compound 117, compound 118, compound 119 were prepared using a similar methylation procedure described in Example 63.

Compound 116. MS (ESI): m/z 255 [M+H]⁺. ¹H NMR (400 MHz, CD₃OD) δ: 8.46 (d, J=4.4 Hz, 1H), 7.86 (d, J=8.4 Hz, 1H), 7.57-7.53 (m, 1H), 7.34-7.18 (m, 4H), 4.04-4.01 (m, 1H), 3.63-3.60 (m, 1H), 3.53-3.47 (m, 1H), 3.38-3.34 (m, 1H), 2.79 (s, 3H), 1.24 (d, J=7.2 Hz, 3H). Chiral analysis column: AD-H 15% methanol [0.2% NH₃ (7M in methanol)]; Temperature: 40° C.; Flow: 4.0 mL/min; retention time=1.59 min.

Compound 117. MS (ESI): m/z 255 [M+H]⁺. ¹H NMR (400 MHz, CD₃OD) δ: 8.46 (d, J=4.4 Hz, 1H), 7.86 (d, J=8.4 Hz, 1H), 7.57-7.53 (m, 1H), 7.34-7.18 (m, 4H), 4.04-4.01 (m, 1H), 3.63-3.60 (m, 1H), 3.53-3.47 (m, 1H), 3.38-3.34 (m, 1H), 2.79 (s, 3H), 1.24 (d, J=7.2 Hz, 3H). Chiral analysis column: AD-H 15% methanol [0.2% NH₃ (7M in methanol)]; Temperature: 40° C.; Flow: 4.0 mL/min; retention time=2.15 min.

Compound 118. MS (ESI): m/z 255 [M+H]⁺. ¹H NMR (400 MHz, CD₃OD) δ: 8.67 (dd, J=5.2, 1.2 Hz, 1H), 8.34 (dd, J=8.8, 1.2 Hz, 1H), 7.96 (dd, J=8.4, 1.6 Hz, 1H), 7.41-7.33 (m, 3H), 7.27-7.23 (m, 1H), 4.05-4.00 (m, 1H), 3.60-3.57 (m, 1H), 3.37-3.32 (m, 2H), 2.74 (s, 3H), 1.33 (d, J=7.6 Hz, 3H). Chiral analysis Column: AY-H (250*4.6 mm 5 μm); Mobile Phase: n-Hexane (0.1% DEA):ethanol (0.1% DEA)=70:30; Temperature: 40° C.; Flow: 1.0 mL/min; Wavelength: 214 nm & 254 nm; Instrument: SHIMADZU; retention time=5.540 min.

Compound 119. MS (ESI): m/z 255 [M+H]⁺. ¹H NMR (400 MHz, CD₃OD) δ: 8.67 (dd, J=5.2, 1.2 Hz, 1H), 8.34 (dd, J=8.8, 1.2 Hz, 1H), 7.96 (dd, J=8.4, 1.6 Hz, 1H), 7.41-7.33 (m, 3H), 7.27-7.23 (m, 1H), 4.05-4.00 (m, 1H), 3.60-3.57 (m, 1H), 3.37-3.32 (m, 2H), 2.74 (s, 3H), 1.33 (d, J=7.6 Hz, 3H). Chiral analysis Column: AY-H (250*4.6 mm 5 μm); Mobile Phase: n-Hexane (0.1% DEA):ethanol (0.1% DEA)=70:30; Temperature: 40° C.; Flow: 1.0 mL/min; Wavelength: 214 nm & 254 nm; Instrument: SHIMADZU; retention time=10.596 min. Chiral analysis column: IA (250*4.6 mm 5 μm); Mobile Phase: n-Hexane (0.1% DEA): ethanol (0.1% DEA)=90:10; Temperature: 40° C.; Flow: 1.0 mL/min. Retention time: 6.255 min.

### Cellular Assays

Exemplary compounds disclosed herein were tested in functional cell assays for TAAR1 agonism, 5-HT_{2A} antagonism, and/or 5-HT₇ antagonism.

TAAR1 Agonism Assay Protocol: cAMP HTRF Assay for Gs Coupled Receptor TAAR1 (Euroscreen FAST-0987C)

CHO-K1 cells expressing human TAAR1 receptors grown prior to the test in media without antibiotic were detached by gentle flushing with PBS-EDTA (5 mM EDTA), recovered by centrifugation, and resuspended in assay buffer (Krebs-Ringer HEPES buffer: 5 mM KCl, 1.25 mM MgSO4, 124 mM NaCl, 25 mM HEPES, 13.3 mM Glucose, 1.25 mM KH2PO4, 1.45 mM CaCl₂), 0.5 g/l BSA, supplemented with 1 mM isobutylmethyl xanthine).

Dose response curves were performed in parallel with the reference compound, tyramine.

For the TAAR1 agonist test (performed in a 384 well plate): 5 μl of cells (about 3,000 cells) were mixed with 5 μl of the test compound diluted in assay buffer and then incubated for about 30 minutes at about room temperature. After addition of the lysis buffer containing cAMP-d2 and anti-cAMP cryptate detection reagents, plates were incubated for about a 1-hour incubation at about room temperature, and fluorescence ratios were measured according to the manufacturer specifications, with the HTRF (Homogeneous Time Resolved Fluorescence) kit (cAMP Gs dynamic kit, Cisbio Bioassays, 62AM4PEJ).

Compounds were tested at the following nanomolar concentrations, in duplicate: 0.3 nM, 1 nM, 3 nM, 10 nM, 30 nM, 100 nM, 300 nM, 1,000 nM, 3,000 nM, and 10,000 nM.

The results of the TAAR1 Agonism Assay are reported in Table 1. “A” compounds had an EC₅₀ of <1 μM in the TAAR1 Agonism Assay; “B” compounds had an EC₅₀ of from 1 μM to less than 10 μM in the TAAR1 Agonism Assay; and “C” compounds had an EC₅₀ of ≥10 μM in the TAAR1 Agonism Assay.

### 5HT_{2A} Antagonism Assay Protocol (Euroscreen FAST-05051)

CHO-K1 cells expressing the human 5-HT_{2A} receptor grown to mid-log phase in culture media without antibiotics, were detached with PBS-EDTA, centrifuged, and resuspended in the IP-One Gq kit (Cisbio Bioassays, 62IPAPEC) stimulation buffer.

For antagonist testing in a 384 well plate: 5 μl of cells (20,000 cells) were mixed with 5 μl of a mix of test compound and reference agonist (for a final assay concentration corresponding to its EC₈₀) diluted in stimulation buffer. The plate was incubated for 60 minutes at 37° C. in a humidified atmosphere of 95% air with 5% CO2, then 5 μL of IP1-d2 and anti-IP1 cryptate detection reagents were added to each well, the plates were incubated for about 1 hour at about room temperature. Fluorescence ratios are measured according to the manufacturer's specification (IP-One Gq kit (Cisbio Bioassays, 62IPAPEC).

Dose response curves were performed in parallel with reference compounds (e.g., α-methyl-5-HT. Compounds were tested at the following nanomolar concentrations, in duplicate: 0.3 nM, 1 nM, 3 nM, 10 nM, 30 nM, 100 nM, 300 nM, 1,000 nM, 3,000 nM, and 10,000 nM

The results of the 5HT_{2A} Antagonism Assay are reported in Table 1. “A” compounds had an IC₅₀ of <1 μM in the 5HT_{2A} Antagonism Assay; “B” compounds had an IC₅₀ of from 1 μM to less than 10 μM in the 5HT_{2A} Antagonism Assay; and “C” compounds had an IC₅₀ of ≥10 μM in the 5HT_{2A} Antagonism Assay.

### 5HT₇ Antagonism Assay Protocol (Euroscreen FAST-0499C)

CHO-K1 cells expressing the human 5-HT₇ receptor, grown prior to the test in media without antibiotic, were detached by gentle flushing with PBS-EDTA (5 mM EDTA), recovered by centrifugation and resuspended in assay buffer (Krebs-Ringer HEPES buffer: 5 mM KCl, 1.25 mM MgSO4, 124 mM NaCl, 25 mM HEPES, 13.3 mM Glucose, 1.25 mM KH2PO4, 1.45 mM CaCl₂), 0.5 g/l BSA, supplemented with 1 mM IBMX).

Dose response curves were performed in parallel with reference compounds (e.g., 5-carboxamidotryptamine (5-CT)).

For antagonist testing in a 384 well plate: 5 μl of cells (3000 cells) were mixed with 5 μl of a mix of test compound and reference agonist (for a final assay concentration corresponding to its EC₈₀) diluted in assay buffer. The plates were then incubated for about 30 minutes at about room temperature. After addition of the lysis buffer containing cAMP-d2 and anti-cAMP cryptate detection reagents, plates were incubated for about 1 hour at about room temperature, and fluorescence ratios were measured according to the manufacturer's specifications (HTRF kit: cAMP Gs dynamic kit, Cisbio Bioassays, 62AM4PEJ).

Compounds were tested at the following nanomolar concentrations, in duplicate: 0.3 nM, 1 nM, 3 nM, 10 nM, 30 nM, 100 nM, 300 nM, 1,000 nM, 3,000 nM, and 10,000 nM.

The results of the 5HT₇ Antagonism Assay are reported in Table 1. “A” compounds had an IC₅₀ of <1 μM in the 5HT₇ Antagonism Assay; “B” compounds had an IC₅₀ of from 1 μM to less than 10 μM in the 5HT₇ Antagonism Assay; and “C” compounds had an IC₅₀ of ≥10 μM in the 5HT₇ Antagonism Assay.

TABLE 1
Functional Cell Assays for TAARI Agonism, 5-HT_{2A} Antagonism, and/or
5-HT₇ Antagonism.

| | | TAAR1 | 5-HT_{2A} | 5-HT₇ |
|---|---|---|---|---|
| Compound No. | Compound Structure | (EC₅₀) | (IC₅₀) | (IC₅₀) |
| 1 | 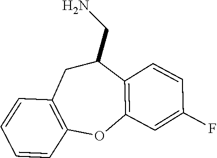 [SMILES: NC[C@@H]1Cc2ccccc2Oc2cc(F)ccc21] | B | A | C |
| 2 | 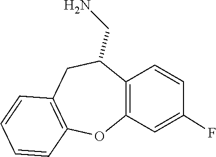 [SMILES: NC[C@H]1Cc2ccccc2Oc2cc(F)ccc21] | A | A | C |
| 3 | 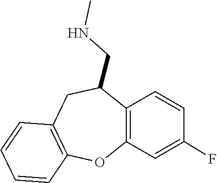 [SMILES: CNC[C@@H]1Cc2ccccc2Oc2cc(F)ccc21] | C | A | B |
| 4 | 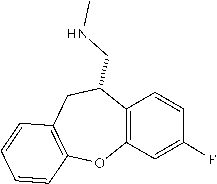 [SMILES: CNC[C@H]1Cc2ccccc2Oc2cc(F)ccc21] | A | A | B |
| 92 | 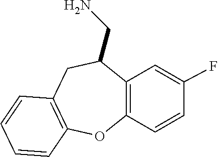 [SMILES: NC[C@@H]1Cc2ccccc2Oc2ccc(F)cc21] | A | A | C |
| 93 | 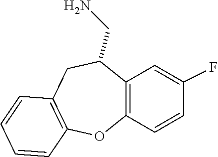 [SMILES: NC[C@H]1Cc2ccccc2Oc2ccc(F)cc21] | B | | |
| 94 | 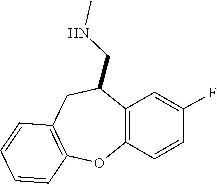 [SMILES: CNC[C@@H]1Cc2ccccc2Oc2ccc(F)cc21] | A | A | B |
| 95 | 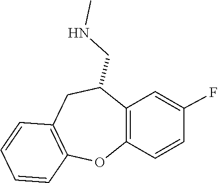 [SMILES: CNC[C@H]1Cc2ccccc2Oc2ccc(F)cc21] | B | A | B |
| 80 | 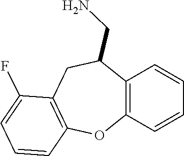 [SMILES: NC[C@@H]1Cc2c(F)cccc2Oc2ccccc21] | A | A | C |
| 81 | 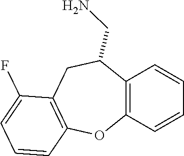 [SMILES: NC[C@H]1Cc2c(F)cccc2Oc2ccccc21] | A | A | C |
| 82 | 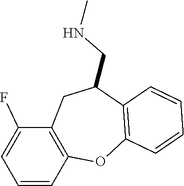 [SMILES: CNC[C@@H]1Cc2c(F)cccc2Oc2ccccc21] | A | A | B |
| 83 | 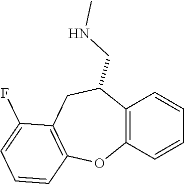 [SMILES: CNC[C@H]1Cc2c(F)cccc2Oc2ccccc21] | C | A | B |
| 84 | 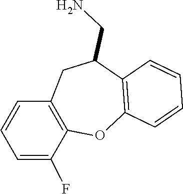 [SMILES: NC[C@@H]1Cc2cccc(F)c2Oc2ccccc21] | A | C | C |
| 85 | 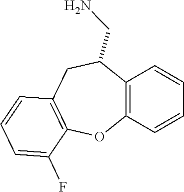 [SMILES: NC[C@H]1Cc2cccc(F)c2Oc2ccccc21] | A | B | C |
| 86 | 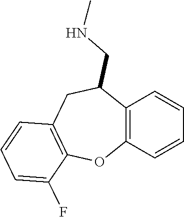 [SMILES: CNC[C@@H]1Cc2cccc(F)c2Oc2ccccc21] | A | A | B |
| 87 | 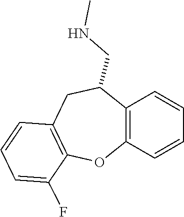 [SMILES: CNC[C@H]1Cc2cccc(F)c2Oc2ccccc21] | A | A | B |
| 88 | 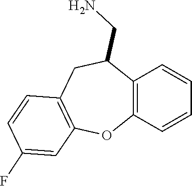 [SMILES: NC[C@@H]1Cc2ccc(F)cc2Oc2ccccc21] | B | | |
| 89 | 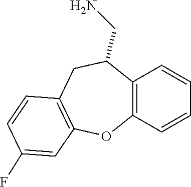 [SMILES: NC[C@H]1Cc2ccc(F)cc2Oc2ccccc21] | A | A | C |
| 90 | 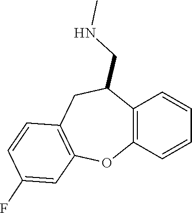 [SMILES: CNC[C@@H]1Cc2ccc(F)cc2Oc2ccccc21] | C | A | B |
| 91 | 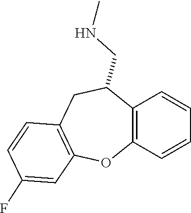 [SMILES: CNC[C@H]1Cc2ccc(F)cc2Oc2ccccc21] | A | A | A |
| 96 | 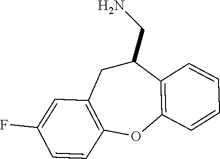 [SMILES: NC[C@@H]1Cc2cc(F)ccc2Oc2ccccc21] | A | A | C |
| 97 | 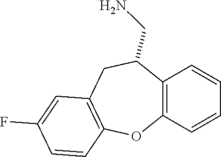 [SMILES: NC[C@H]1Cc2cc(F)ccc2Oc2ccccc21] | C | | |
| 98 | 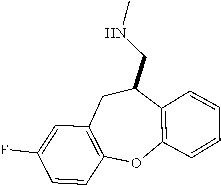 [SMILES: CNC[C@@H]1Cc2cc(F)ccc2Oc2ccccc21] | A | A | B |
| 99 | 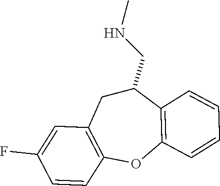 [SMILES: CNC[C@H]1Cc2cc(F)ccc2Oc2ccccc21] | C | | |
| 100 | 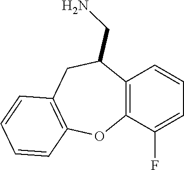 [SMILES: NC[C@@H]1Cc2ccccc2Oc2c(F)cccc21] | A | A | C |
| 101 | 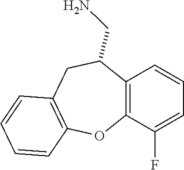 [SMILES: NC[C@H]1Cc2ccccc2Oc2c(F)cccc21] | A | C | C |
| 102 | 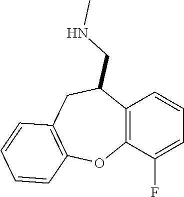 [SMILES: CNC[C@@H]1Cc2ccccc2Oc2c(F)cccc21] | A | A | C |
| 103 | 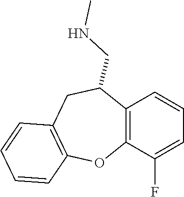 [SMILES: CNC[C@H]1Cc2ccccc2Oc2c(F)cccc21] | B | | |
| 104 | 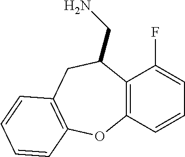 [SMILES: NC[C@@H]1Cc2ccccc2Oc2cccc(F)c21] | A | B | C |
| 105 | 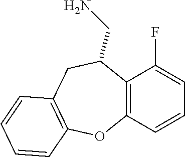 [SMILES: NC[C@H]1Cc2ccccc2Oc2cccc(F)c21] | B | C | C |
| 106 | 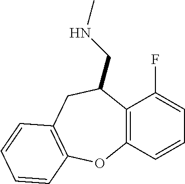 [SMILES: CNC[C@@H]1Cc2ccccc2Oc2cccc(F)c21] | A | A | C |
| 107 | 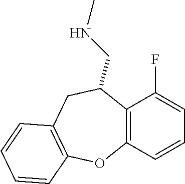 [SMILES: CNC[C@H]1Cc2ccccc2Oc2cccc(F)c21] | C | B | C |
| 5 | 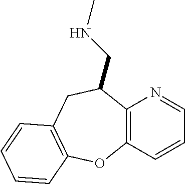 [SMILES: CNC[C@@H]1Cc2ccccc2Oc2cccnc21] | A | A | A |
| 6 | 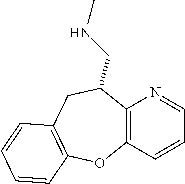 [SMILES: CNC[C@H]1Cc2ccccc2Oc2cccnc21] | A | B | C |
| 7 | 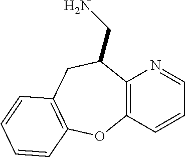 [SMILES: NC[C@@H]1Cc2ccccc2Oc2cccnc21] | A | C | B |
| 8 | 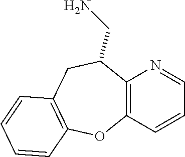 [SMILES: NC[C@H]1Cc2ccccc2Oc2cccnc21] | A | C | C |
| 108 | 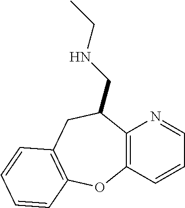 [SMILES: CCNC[C@@H]1Cc2ccccc2Oc2cccnc21] | A | | |
| 109 | 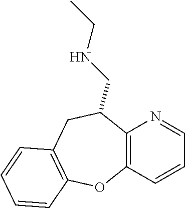 [SMILES: CCNC[C@H]1Cc2ccccc2Oc2cccnc21] | A | B | C |
| 110 | 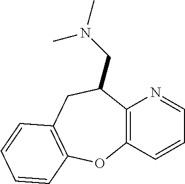 [SMILES: CN(C)C[C@@H]1Cc2ccccc2Oc2cccnc21] | C | | |
| 111 | 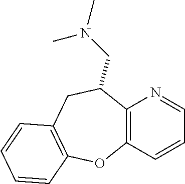 [SMILES: CN(C)C[C@H]1Cc2ccccc2Oc2cccnc21] | A | B | C |
| 112 | 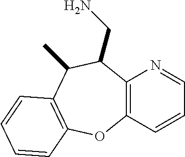 [SMILES: C[C@@H]1c2ccccc2Oc2cccnc2[C@@H]1CN] | A | C | C |
| 113 | 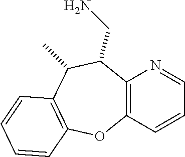 [SMILES: C[C@H]1c2ccccc2Oc2cccnc2[C@H]1CN] | C | | |
| 114 | 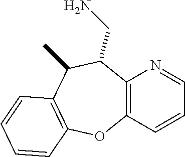 [SMILES: C[C@@H]1c2ccccc2Oc2cccnc2[C@H]1CN] | A | B | C |
| 115 | 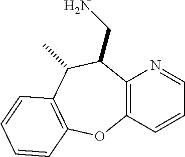 [SMILES: C[C@H]1c2ccccc2Oc2cccnc2[C@@H]1CN] | B | C | C |
| 116 | 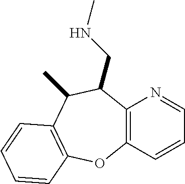 [SMILES: CNC[C@H]1c2ncccc2Oc2ccccc2[C@H]1C] | A | B | C |
| 117 | 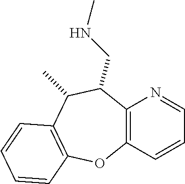 [SMILES: CNC[C@@H]1c2ncccc2Oc2ccccc2[C@@H]1C] | C | | |
| 118 | 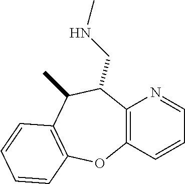 [SMILES: CNC[C@@H]1c2ncccc2Oc2ccccc2[C@H]1C] | A | B | C |
| 119 | 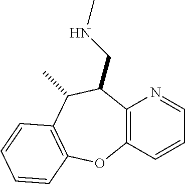 [SMILES: CNC[C@H]1c2ncccc2Oc2ccccc2[C@@H]1C] | B | B | C |
| 120 | 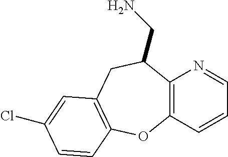 [SMILES: NC[C@@H]1Cc2cc(Cl)ccc2Oc2cccnc21] | A | B | C |
| 121 | 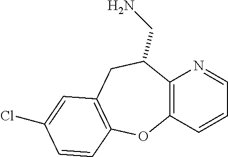 [SMILES: NC[C@H]1Cc2cc(Cl)ccc2Oc2cccnc21] | A | A | A |
| 122 | 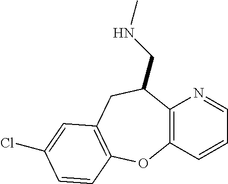 [SMILES: CNC[C@@H]1Cc2cc(Cl)ccc2Oc2cccnc21] | A | A | B |
| 123 | 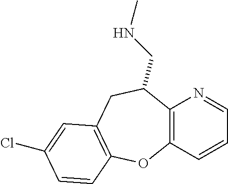 [SMILES: CNC[C@H]1Cc2cc(Cl)ccc2Oc2cccnc21] | A | A | A |
| 124 | 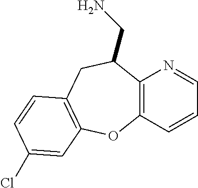 [SMILES: NC[C@@H]1Cc2ccc(Cl)cc2Oc2cccnc21] | A | B | C |
| 125 | 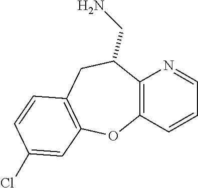 [SMILES: NC[C@H]1Cc2ccc(Cl)cc2Oc2cccnc21] | A | C | C |
| 126 | 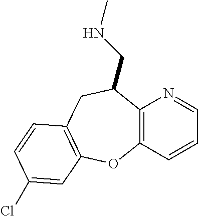 [SMILES: CNC[C@@H]1Cc2ccc(Cl)cc2Oc2cccnc21] | A | A | C |
| 127 | 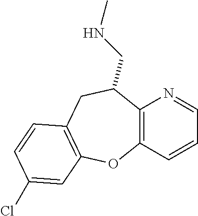 [SMILES: CNC[C@H]1Cc2ccc(Cl)cc2Oc2cccnc21] | A | A | B |
| 128 | 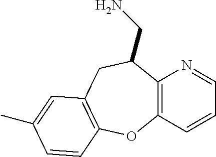 [SMILES: Cc1ccc2c(c1)C[C@@H](CN)c1ncccc1O2] | A | B | B |
| 129 | 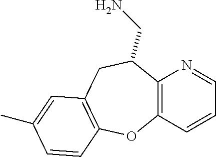 [SMILES: Cc1ccc2c(c1)C[C@H](CN)c1ncccc1O2] | A | A | A |
| 130 | 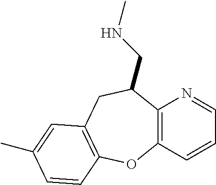 [SMILES: CNC[C@@H]1Cc2cc(C)ccc2Oc2cccnc21] | A | A | B |
| 131 | 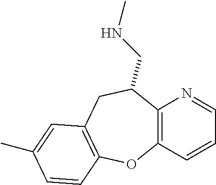 [SMILES: CNC[C@H]1Cc2cc(C)ccc2Oc2cccnc21] | A | A | A |
| 132 | 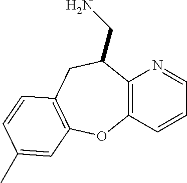 [SMILES: Cc1ccc2c(c1)Oc1cccnc1[C@H](CN)C2] | A | C | C |
| 133 | 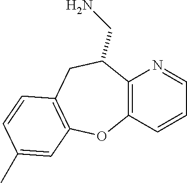 [SMILES: Cc1ccc2c(c1)Oc1cccnc1[C@@H](CN)C2] | A | B | C |
| 134 | 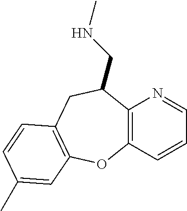 [SMILES: CNC[C@@H]1Cc2ccc(C)cc2Oc2cccnc21] | A | A | B |
| 135 | 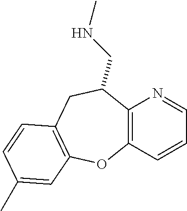 [SMILES: CNC[C@H]1Cc2ccc(C)cc2Oc2cccnc21] | A | A | B |
| 136 | 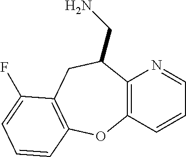 [SMILES: NC[C@@H]1Cc2c(F)cccc2Oc2cccnc21] | A | B | C |
| 137 | 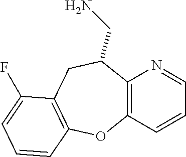 [SMILES: NC[C@H]1Cc2c(F)cccc2Oc2cccnc21] | A | C | C |
| 138 | 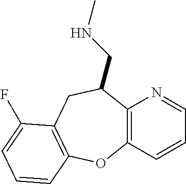 [SMILES: CNC[C@@H]1Cc2c(F)cccc2Oc2cccnc21] | A | A | C |
| 139 | 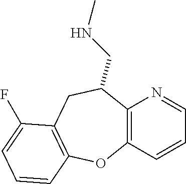 [SMILES: CNC[C@H]1Cc2c(F)cccc2Oc2cccnc21] | B | | |
| 140 | 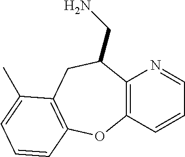 [SMILES: Cc1cccc2c1C[C@@H](CN)c1ncccc1O2] | A | C | C |
| 141 | 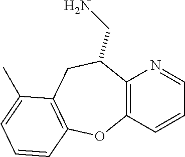 [SMILES: Cc1cccc2c1C[C@H](CN)c1ncccc1O2] | A | A | C |
| 142 | 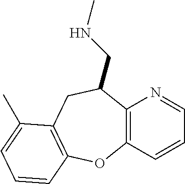 [SMILES: CNC[C@@H]1Cc2c(C)cccc2Oc2cccnc21] | B | | |
| 143 | 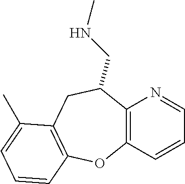 [SMILES: CNC[C@H]1Cc2c(C)cccc2Oc2cccnc21] | A | A | C |
| 144 | 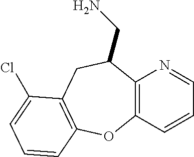 [SMILES: NC[C@@H]1Cc2c(Cl)cccc2Oc2cccnc21] | A | A | C |
| 145 | 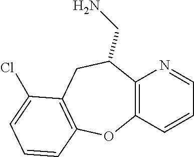 [SMILES: NC[C@H]1Cc2c(Cl)cccc2Oc2cccnc21] | A | A | C |
| 146 | 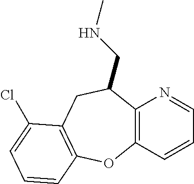 [SMILES: CNC[C@@H]1Cc2c(Cl)cccc2Oc2cccnc21] | C | | |
| 147 | 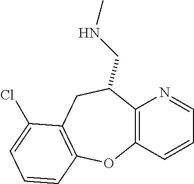 [SMILES: CNC[C@H]1Cc2c(Cl)cccc2Oc2cccnc21] | A | A | C |
| 148 | 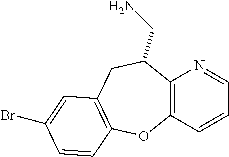 [SMILES: NC[C@H]1Cc2cc(Br)ccc2Oc2cccnc21] | A | | |
| 149 | 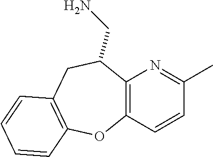 [SMILES: Cc1ccc2c(n1)[C@@H](CN)Cc1ccccc1O2] | A | A | C |
| 150 | 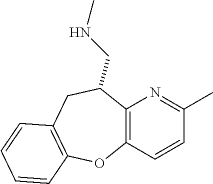 [SMILES: CNC[C@H]1Cc2ccccc2Oc2ccc(C)nc21] | A | A | B |
| 151 | 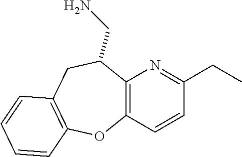 [SMILES: CCc1ccc2c(n1)[C@@H](CN)Cc1ccccc1O2] | A | A | C |
| 152 | 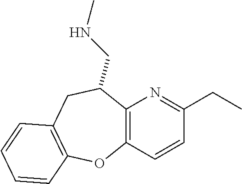 [SMILES: CCc1ccc2c(n1)[C@@H](CNC)Cc1ccccc1O2] | A | A | B |
| 9 | 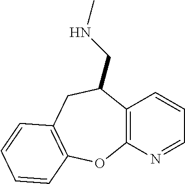 [SMILES: CNC[C@@H]1Cc2ccccc2Oc2ncccc21] | | | |
| 10 | 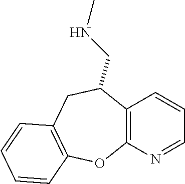 [SMILES: CNC[C@H]1Cc2ccccc2Oc2ncccc21] | | | |
| 11 | 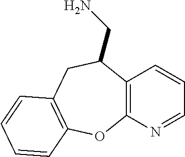 [SMILES: NC[C@@H]1Cc2ccccc2Oc2ncccc21] | | | |
| 12 | 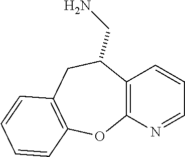 [SMILES: NC[C@H]1Cc2ccccc2Oc2ncccc21] | | | |
| 13 | 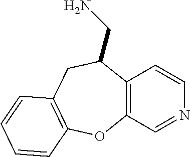 [SMILES: NC[C@@H]1Cc2ccccc2Oc2cnccc21] | | | |
| 14 | 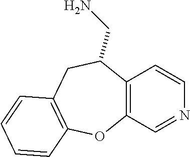 [SMILES: NC[C@H]1Cc2ccccc2Oc2cnccc21] | | | |
| 15 | 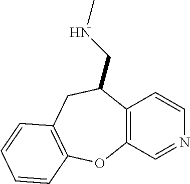 [SMILES: CNC[C@@H]1Cc2ccccc2Oc2cnccc21] | | | |
| 16 | 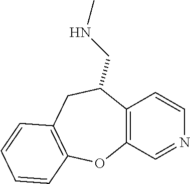 [SMILES: CNC[C@H]1Cc2ccccc2Oc2cnccc21] | | | |
| 17 | 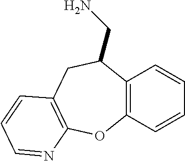 [SMILES: NC[C@@H]1Cc2cccnc2Oc2ccccc21] | A | A | C |
| 18 | 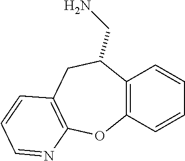 [SMILES: NC[C@H]1Cc2cccnc2Oc2ccccc21] | C | C | C |
| 19 | 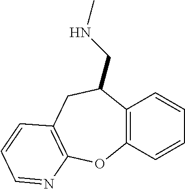 [SMILES: CNC[C@@H]1Cc2cccnc2Oc2ccccc21] | B | B | C |
| 20 | 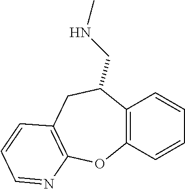 [SMILES: CNC[C@H]1Cc2cccnc2Oc2ccccc21] | C | C | C |
| 21 | 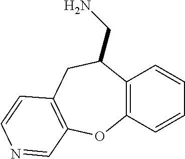 [SMILES: NC[C@@H]1Cc2ccncc2Oc2ccccc21] | A | B | C |
| 22 | 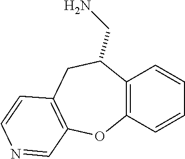 [SMILES: NC[C@H]1Cc2ccncc2Oc2ccccc21] | B | B | C |
| 23 | 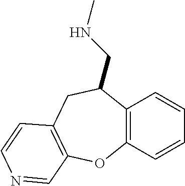 [SMILES: CNC[C@@H]1Cc2ccncc2Oc2ccccc21] | A | A | A |
| 24 | 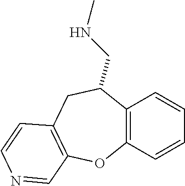 [SMILES: CNC[C@H]1Cc2ccncc2Oc2ccccc21] | C | A | B |
| 153 | 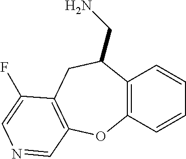 [SMILES: NC[C@@H]1Cc2c(F)cncc2Oc2ccccc21] | A | A | C |
| 154 | 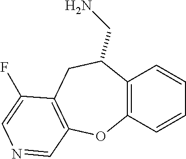 [SMILES: NC[C@H]1Cc2c(F)cncc2Oc2ccccc21] | C | B | C |
| 27 | 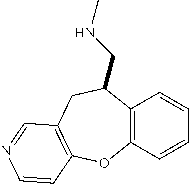 [SMILES: CNC[C@@H]1Cc2cnccc2Oc2ccccc21] | | | |
| 28 | 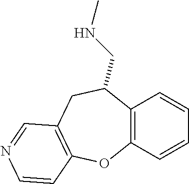 [SMILES: CNC[C@H]1Cc2cnccc2Oc2ccccc21] | | | |
| 29 | 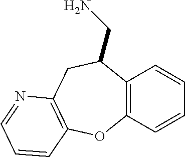 [SMILES: NC[C@@H]1Cc2ncccc2Oc2ccccc21] | B | B | C |
| 30 | 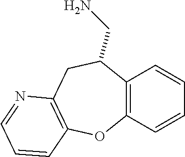 [SMILES: NC[C@H]1Cc2ncccc2Oc2ccccc21] | A | B | C |
| 31 | 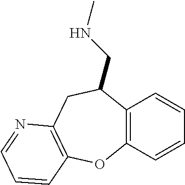 [SMILES: CNC[C@@H]1Cc2ncccc2Oc2ccccc21] | C | A | C |
| 32 | 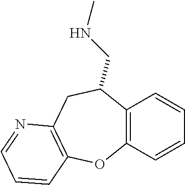 [SMILES: CNC[C@H]1Cc2ncccc2Oc2ccccc21] | A | A | C |
| 155 | 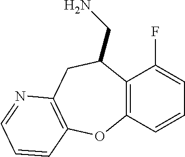 [SMILES: NC[C@@H]1Cc2ncccc2Oc2cccc(F)c21] | B | | |
| 156 | 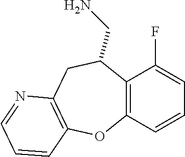 [SMILES: NC[C@H]1Cc2ncccc2Oc2cccc(F)c21] | A | C | C |
| 157 | 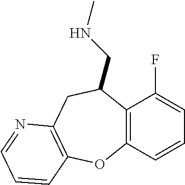 [SMILES: CNC[C@@H]1Cc2ncccc2Oc2cccc(F)c21] | C | | |
| 158 | 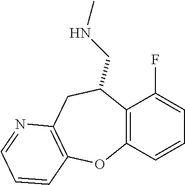 [SMILES: CNC[C@H]1Cc2ncccc2Oc2cccc(F)c21] | B | C | C |
| 159 | 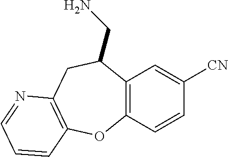 [SMILES: N#Cc1ccc2c(c1)[C@H](CN)Cc1ncccc1O2] | B | C | C |
| 160 | 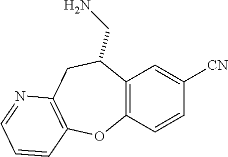 [SMILES: N#Cc1ccc2c(c1)[C@@H](CN)Cc1ncccc1O2] | A | A | B |
| 161 | 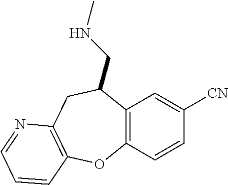 [SMILES: CNC[C@@H]1Cc2ncccc2Oc2ccc(C#N)cc21] | C | B | C |
| 162 | 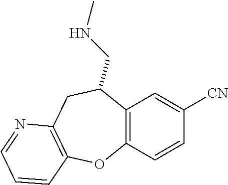 [SMILES: CNC[C@H]1Cc2ncccc2Oc2ccc(C#N)cc21] | C | B | C |
| 163 | 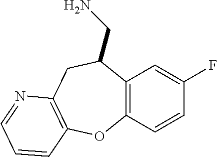 [SMILES: NC[C@@H]1Cc2ncccc2Oc2ccc(F)cc21] | B | B | C |
| 164 | 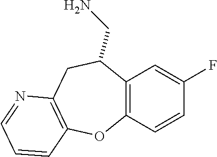 [SMILES: NC[C@H]1Cc2ncccc2Oc2ccc(F)cc21] | A | B | C |
| 165 | 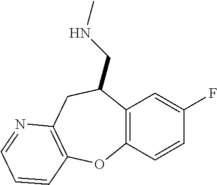 [SMILES: CNC[C@@H]1Cc2ncccc2Oc2ccc(F)cc21] | C | A | C |
| 166 | 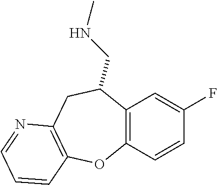 [SMILES: CNC[C@H]1Cc2ncccc2Oc2ccc(F)cc21] | A | A | B |
| 167 | 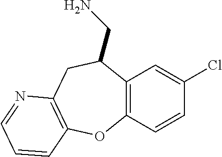 [SMILES: NC[C@@H]1Cc2ncccc2Oc2ccc(Cl)cc21] | A | B | C |
| 168 | 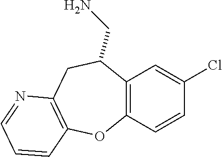 [SMILES: NC[C@H]1Cc2ncccc2Oc2ccc(Cl)cc21] | B | | |
| 169 | 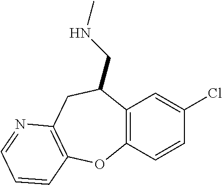 [SMILES: CNC[C@@H]1Cc2ncccc2Oc2ccc(Cl)cc21] | C | | |
| 170 | 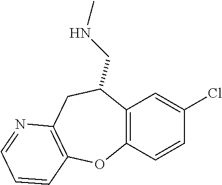 [SMILES: CNC[C@H]1Cc2ncccc2Oc2ccc(Cl)cc21] | A | A | A |
| 171 | 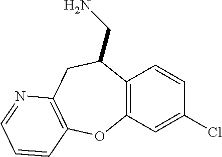 [SMILES: NC[C@@H]1Cc2ncccc2Oc2cc(Cl)ccc21] | B | A | C |
| 172 | 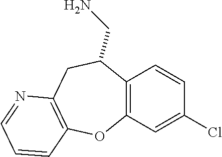 [SMILES: NC[C@H]1Cc2ncccc2Oc2cc(Cl)ccc21] | A | A | C |
| 173 | 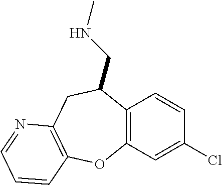 [SMILES: CNC[C@@H]1Cc2ncccc2Oc2cc(Cl)ccc21] | C | A | C |
| 174 | 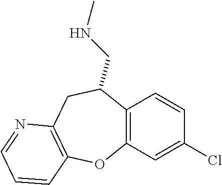 [SMILES: CNC[C@H]1Cc2ncccc2Oc2cc(Cl)ccc21] | C | A | B |
| 175 | 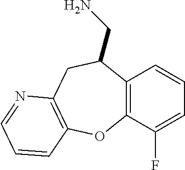 [SMILES: NC[C@@H]1Cc2ncccc2Oc2c(F)cccc21] | B | | |
| 176 | 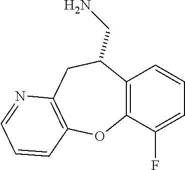 [SMILES: NC[C@H]1Cc2ncccc2Oc2c(F)cccc21] | A | C | C |
| 177 | 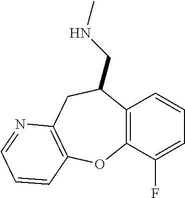 [SMILES: CNC[C@@H]1Cc2ncccc2Oc2c(F)cccc21] | C | | |
| 178 | 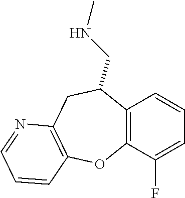 [SMILES: CNC[C@H]1Cc2ncccc2Oc2c(F)cccc21] | A | | |
| 33 | 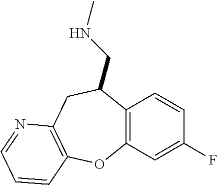 [SMILES: CNC[C@@H]1Cc2ncccc2Oc2cc(F)ccc21] | C | | |
| 34 | 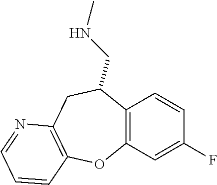 [SMILES: CNC[C@H]1Cc2ncccc2Oc2cc(F)ccc21] | A | | |
| 35 | 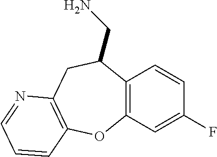 [SMILES: NC[C@@H]1Cc2ncccc2Oc2cc(F)ccc21] | B | | |
| 36 | 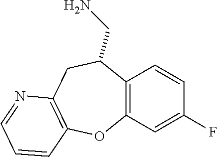 [SMILES: NC[C@H]1Cc2ncccc2Oc2cc(F)ccc21] | A | A | C |
| 37 | 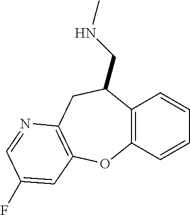 [SMILES: CNC[C@@H]1Cc2ncc(F)cc2Oc2ccccc21] | C | | |
| 38 | 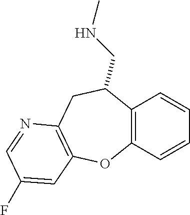 [SMILES: CNC[C@H]1Cc2ncc(F)cc2Oc2ccccc21] | B | A | B |
| 39 | 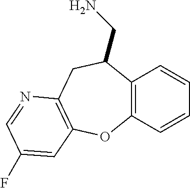 [SMILES: NC[C@@H]1Cc2ncc(F)cc2Oc2ccccc21] | B | A | C |
| 40 | 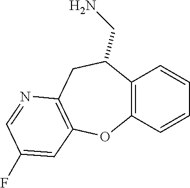 [SMILES: NC[C@H]1Cc2ncc(F)cc2Oc2ccccc21] | A | A | C |
| 179 | 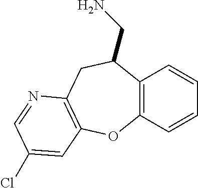 [SMILES: NC[C@@H]1Cc2ncc(Cl)cc2Oc2ccccc21] | B | B | C |
| 180 | 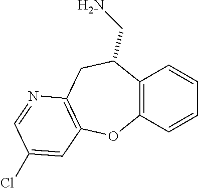 [SMILES: NC[C@H]1Cc2ncc(Cl)cc2Oc2ccccc21] | A | B | C |
| 41 | 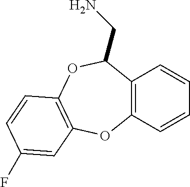 [SMILES: NC[C@@H]1Oc2ccc(F)cc2Oc2ccccc21] | A | A | B |
| 42 | 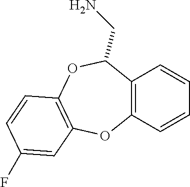 [SMILES: NC[C@H]1Oc2ccc(F)cc2Oc2ccccc21] | A | A | C |
| 43 | 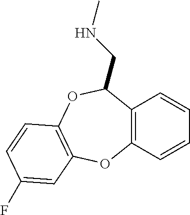 [SMILES: CNC[C@@H]1Oc2ccc(F)cc2Oc2ccccc21] | A | A | A |
| 44 | 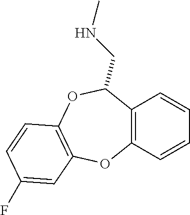 [SMILES: CNC[C@H]1Oc2ccc(F)cc2Oc2ccccc21] | A | A | A |
| 45 | 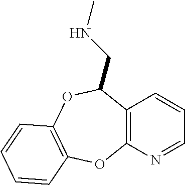 [SMILES: CNC[C@@H]1Oc2ccccc2Oc2ncccc21] | C | B | C |
| 46 | 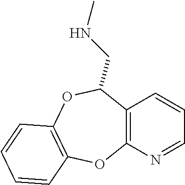 [SMILES: CNC[C@H]1Oc2ccccc2Oc2ncccc21] | B | B | C |
| 47 | 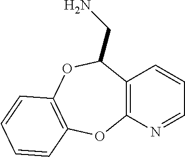 [SMILES: NC[C@@H]1Oc2ccccc2Oc2ncccc21] | B | C | C |
| 48 | 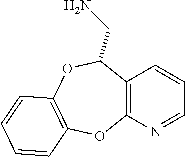 [SMILES: NC[C@H]1Oc2ccccc2Oc2ncccc21] | B | C | C |
| 49 | 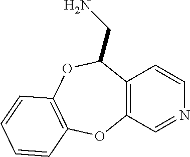 [SMILES: NC[C@@H]1Oc2ccccc2Oc2cnccc21] | B | C | C |
| 50 | 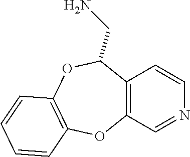 [SMILES: NC[C@H]1Oc2ccccc2Oc2cnccc21] | A | C | C |
| 51 | 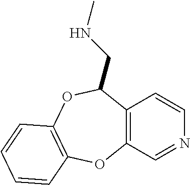 [SMILES: CNC[C@@H]1Oc2ccccc2Oc2cnccc21] | B | B | C |
| 52 | 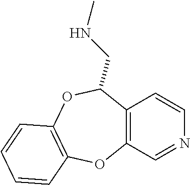 [SMILES: CNC[C@H]1Oc2ccccc2Oc2cnccc21] | C | B | C |
| 53 | 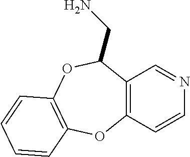 [SMILES: NC[C@@H]1Oc2ccccc2Oc2ccncc21] | B | C | C |
| 54 | 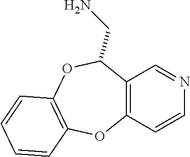 [SMILES: NC[C@H]1Oc2ccccc2Oc2ccncc21] | B | C | C |
| 55 | 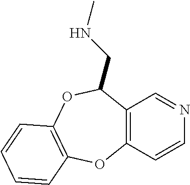 [SMILES: CNC[C@@H]1Oc2ccccc2Oc2ccncc21] | C | C | C |
| 56 | 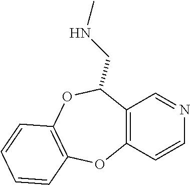 [SMILES: CNC[C@H]1Oc2ccccc2Oc2ccncc21] | C | C | C |
| 57 | 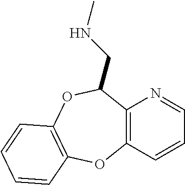 [SMILES: CNC[C@@H]1Oc2ccccc2Oc2cccnc21] | A | B | C |
| 58 | 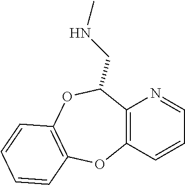 [SMILES: CNC[C@H]1Oc2ccccc2Oc2cccnc21] | A | B | B |
| 59 | 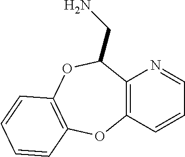 [SMILES: NC[C@@H]1Oc2ccccc2Oc2cccnc21] | A | C | C |
| 60 | 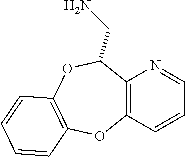 [SMILES: NC[C@H]1Oc2ccccc2Oc2cccnc21] | A | C | C |
| 61 | 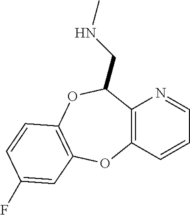 [SMILES: CNC[C@@H]1Oc2ccc(F)cc2Oc2cccnc21] | | | |
| 62 | 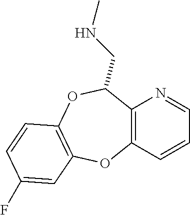 [SMILES: CNC[C@H]1Oc2ccc(F)cc2Oc2cccnc21] | | | |
| 63 | 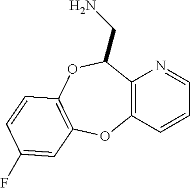 [SMILES: NC[C@@H]1Oc2ccc(F)cc2Oc2cccnc21] | | | |
| 64 | 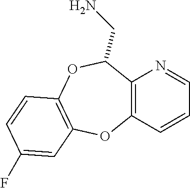 [SMILES: NC[C@H]1Oc2ccc(F)cc2Oc2cccnc21] | | | |
| 65 | 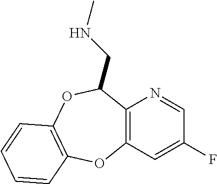 [SMILES: CNC[C@@H]1Oc2ccccc2Oc2cc(F)cnc21] | | | |
| 66 | 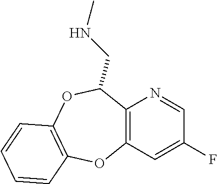 [SMILES: CNC[C@H]1Oc2ccccc2Oc2cc(F)cnc21] | | | |
| 67 | 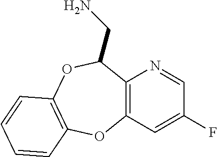 [SMILES: NC[C@@H]1Oc2ccccc2Oc2cc(F)cnc21] | | | |
| 68 | 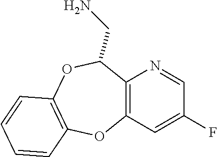 [SMILES: NC[C@H]1Oc2ccccc2Oc2cc(F)cnc21] | | | |
| 69 | 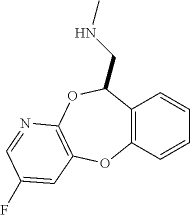 [SMILES: CNC[C@@H]1Oc2ncc(F)cc2Oc2ccccc21] | | | |
| 70 | 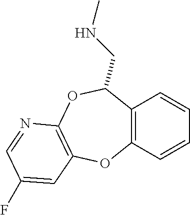 [SMILES: CNC[C@H]1Oc2ncc(F)cc2Oc2ccccc21] | | | |
| 71 | 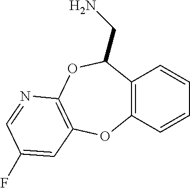 [SMILES: NC[C@@H]1Oc2ncc(F)cc2Oc2ccccc21] | | | |
| 72 | 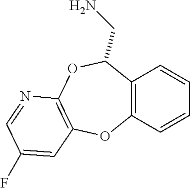 [SMILES: NC[C@H]1Oc2ncc(F)cc2Oc2ccccc21] | | | |
| 73 | 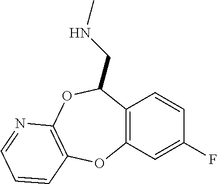 [SMILES: CNC[C@@H]1Oc2ncccc2Oc2cc(F)ccc21] | | | |
| 74 | 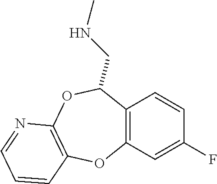 [SMILES: CNC[C@H]1Oc2ncccc2Oc2cc(F)ccc21] | | | |
| 75 | 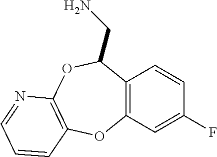 [SMILES: NC[C@@H]1Oc2ncccc2Oc2cc(F)ccc21] | | | |
| 76 | 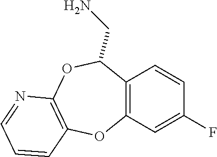 [SMILES: NC[C@H]1Oc2ncccc2Oc2cc(F)ccc21] | | | |
| 77 | 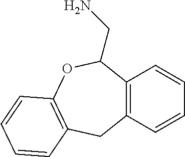 [SMILES: NCC1Oc2ccccc2Cc2ccccc21] | A | B | C |
| 78 | 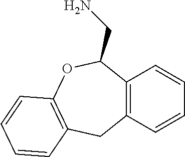 [SMILES: NC[C@@H]1Oc2ccccc2Cc2ccccc21] | | | |
| 79 | 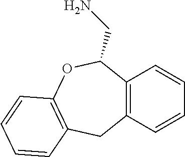 [SMILES: NC[C@H]1Oc2ccccc2Cc2ccccc21] | | | |

“A” = EC₅₀ (for TAAR1) or IC₅₀ (for 5-HT_{2A} or 5-HT₇) less than 1 μM;
“B” = EC₅₀ or IC₅₀ greater than or equal to 1 μM and less than 10 μM;
“C” = EC₅₀ or IC₅₀ greater than or equal to 10 μM.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A compound of Formula I 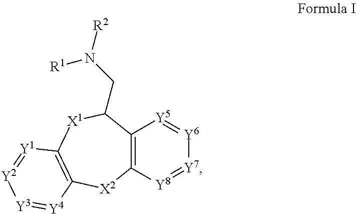
or a pharmaceutically acceptable salt thereof, wherein:
one of X¹ and X² is O, and the other is independently C(R³)₂ or O;
each R³ is independently H, (C₁-C₄)alkyl, (C₂-C₄)alkenyl, or (C₂-C₄)alkynyl;
Y¹, Y², Y³, Y⁴, Y⁵, Y⁶, Y⁷, and Y⁸ are each independently C(R⁴) or N, and no more than one of Y¹, Y², Y³, Y⁴, Y⁵, Y⁶, Y⁷, and Y⁸ is N;
each R⁴ is independently H, halogen, —CN, (C₁-C₄)alkyl, (C₂-C₄)alkenyl, (C₂-C₄)alkynyl, (C₁-C₄)haloalkyl, (C₁-C₄)alkoxy, or (C₁-C₄)haloalkoxy; and
R¹ and R² are each independently H, (C₁-C₄)alkyl, or (C₂-C₄)alkenyl.

2. The compound of claim 1, wherein X¹ is C(R³)₂ and X² is O.

3. The compound of claim 1, wherein X¹ and X² are each O.

4. The compound of claim 1, wherein X¹ is O and X² is C(R³)₂.

5. The compound of any one of claims 1-4, wherein each R³ is independently H or (C₁-C₄)alkyl.

6. The compound of claim 5, wherein each R³ is independently H or methyl.

7. The compound of claim 6, wherein each R³ is H.

8. The compound of any one of claims 1-7, wherein Y¹, Y², Y³, Y⁴, Y⁵, Y⁶, Y⁷, and Y⁸ are each C(R⁴).

9. The compound of any one of claims 1-7, wherein Y¹ is N.

10. The compound of any one of claims 1-7, wherein Y² is N.

11. The compound of any one of claims 1-7, wherein Y³ is N.

12. The compound of any one of claims 1-7, wherein Y⁴ is N.

13. The compound of any one of claims 1-7, wherein Y⁵ is N.

14. The compound of any one of claims 1-7, wherein Y⁶ is N.

15. The compound of any one of claims 1-7, wherein Y⁷ is N.

16. The compound of any one of claims 1-7, wherein Y⁸ is N.

17. The compound of any one of claims 1-16, wherein each R⁴ is independently H, halogen, —CN, (C₁-C₄)alkyl or (C₁-C₄)alkoxy.

18. The compound of claim 17, wherein each R⁴ is independently H or halogen.

19. The compound of claim 18, wherein each R⁴ is independently H or F.

20. The compound of claim 19, wherein each R⁴ is H.

21. The compound of any one of claims 1-16, wherein one R⁴ is halogen and the rest are H.

22. The compound of claim 21, wherein one R⁴ is F and the rest are H.

23. The compound of any one of claims 1-22, wherein R¹ and R² are each independently H or (C₁-C₄)alkyl.

24. The compound of claim 23, wherein R¹ and R² are each independently H or methyl.

25. The compound of any one of claims 1-23, wherein R¹ is H and R² is (C₁-C₄)alkyl.

26. The compound of claim 25, wherein R¹ is H and R² is methyl.

27. The compound of any one of claims 1-26, having Formula I(A) 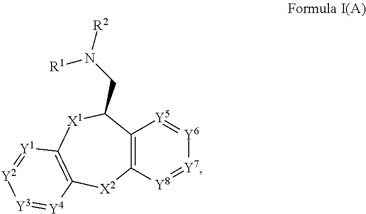
or a pharmaceutically acceptable salt thereof.

28. The compound of any one of claims 1-26, having Formula I(B) 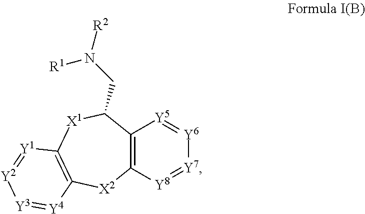
or a pharmaceutically acceptable salt thereof.

29. The compound of claim 1, 27, or 28, wherein:
X¹ is C(R³)₂ or O, and X² is O;
each R³ is independently H or methyl;
Y¹, Y², Y³, Y⁴, Y⁵, Y⁶, Y⁷, and Y⁸ are each independently C(R⁴) or N, and no more than one of Y¹, Y², Y³, Y⁴, Y⁵, Y⁶, Y⁷, and Y⁸ are N;
one R⁴ is H, halogen, —CN, (C₁-C₄)alkyl or (C₁-C₄)alkoxy, and the rest are H;
R¹ is H; and
R² is (C₁-C₄)alkyl.

30. The compound of claim 29, wherein X¹ is C(R³)₂ and X² is O.

31. The compound of claim 29 or 30, wherein each R³ is H.

32. The compound of claim 29, wherein X¹ and X² are each O.

33. The compound of any one of claims 29-32, wherein Y¹, Y², Y³, Y⁴, Y⁵, Y⁶, Y⁷, and Y⁸ are each C(R⁴).

34. The compound of any one of claims 29-32, wherein one of Y¹, Y², Y³, Y⁴, Y⁵, Y⁶, Y⁷, and Y⁸ is N, and the rest are each C(R⁴).

35. The compound of claim 34, wherein one of Y¹, Y³ and Y⁵ is N.

36. The compound of claim 35, wherein Y¹ is N.

37. The compound of claim 34, wherein one of Y⁵, Y⁶, Y⁷ and Y⁸ is N.

38. The compound of claim 35 or 37, wherein Y⁵ is N.

39. The compound of any one of claims 29-38, wherein each R⁴ is H.

40. The compound of any one of claims 29-38, wherein one R⁴ is halogen, —CN or (C₁-C₄)alkyl, and the rest are H.

41. The compound of claim 40, wherein one R⁴ is fluoro, chloro, bromo, methyl, ethyl or cyano, and the rest are H.

42. The compound of any one of claims 29-38, wherein one R⁴ is halogen, and the rest are H.

43. The compound of claim 41 or 42, wherein one R⁴ is fluoro, and the rest are H.

44. The compound of any one of claims 29-43, wherein R² is methyl.

45. The compound of claim 1, having the following formula: 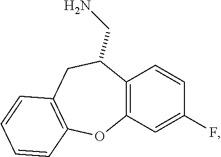 [SMILES: NC[C@H]1Cc2ccccc2Oc2cc(F)ccc21]
or a pharmaceutically acceptable salt thereof.

46. The compound of claim 1, having the following formula: 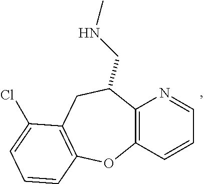 [SMILES: CNC[C@H]1Cc2c(Cl)cccc2Oc2cccnc21]
or a pharmaceutically acceptable salt thereof.

47. The compound of claim 1, having the following formula: 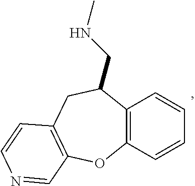 [SMILES: CNC[C@@H]1Cc2ccncc2Oc2ccccc21]
or a pharmaceutically acceptable salt thereof.

48. The compound of claim 1, having the following formula: 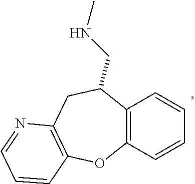 [SMILES: CNC[C@H]1Cc2ncccc2Oc2ccccc21]
or a pharmaceutically acceptable salt thereof.

49. The compound of claim 1, having the following formula: 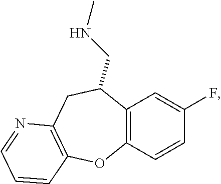 [SMILES: CNC[C@H]1Cc2ncccc2Oc2ccc(F)cc21]
or a pharmaceutically acceptable salt thereof.

50. A pharmaceutical composition comprising a compound of any one of claims 1-49, or a pharmaceutically acceptable salt thereof, and one or more pharmaceutically acceptable excipients.

51. A pharmaceutical combination, comprising a compound of any one of claims 1-49, or a pharmaceutically acceptable salt thereof, and one or more additional therapeutic agents.

52. A method of treating a neurological or psychiatric disease or disorder in a subject in need thereof, comprising administering to the subject a therapeutically effective amount of a compound according to any one of claims 1-49, or a pharmaceutically acceptable salt thereof, or pharmaceutical composition of claim 50.

53. The method of claim 52, wherein the neurological or psychiatric disease or disorder is schizophrenia.

54. The method of claim 52, wherein the neurological or psychiatric disease or disorder is a bipolar disorder.

55. The method of claim 52, wherein the neurological or psychiatric disease or disorder is Parkinson's disease.

56. The method of claim 55, wherein the neurological or psychiatric disease or disorder is Alzheimer's disease.

57. The method of claim 55, wherein the neurological or psychiatric disease or disorder is autism spectrum disorder.

58. The method of claim 55, wherein the neurological or psychiatric disease or disorder is a substance-related or addictive disorder.

59. The method of claim 55, wherein the neurological or psychiatric disease or disorder is a metabolic disease.

60. A method of agonizing TAAR1 in a subject in need thereof, comprising administering to the subject a compound according to any one of claims 1-49, or a pharmaceutically acceptable salt thereof, a pharmaceutical composition of claim 50 or a pharmaceutical combination of claim 51 in an amount sufficient to agonize TAAR1 in the subject.

61. The method of claim 60, wherein the compound, or a pharmaceutically acceptable salt thereof, pharmaceutical composition or pharmaceutical combination selectively agonizes TAAR1 in the subject.

62. The method of claim 60, wherein the compound, or a pharmaceutically acceptable salt thereof, pharmaceutical composition or pharmaceutical combination antagonizes 5-HT2A.

63. The method of claim 60 or 62, wherein the compound, or a pharmaceutically acceptable salt thereof, pharmaceutical composition or pharmaceutical combination antagonizes 5-HT7.

64. A method of antagonizing 5-HT2A, 5-HT7, or 5-HT2A and 5-HT7 in a subject in need thereof, comprising administering to the subject a compound according to any one of claims 1-49, or a pharmaceutically acceptable salt thereof, a pharmaceutical composition of claim 50 or a pharmaceutical combination of claim 51 in an amount sufficient to antagonize 5-HT2A, 5-HT7, or 5-HT2A and 5-HT7, respectively, in the subject.

65. The method of any one of claims 52-64, further comprising administering to the subject one or more additional therapeutic agents.
